(19) United States
(12) Patent Application Publication
(10) Pub. No.: US 2024/0245634 A1
(43) Pub. Date: Jul. 25, 2024

(54) PCNA INHIBITORS AND EGFR INHIBITORS FOR CANCER TREATMENT

(71) Applicant: City of Hope, Duarte, CA (US)

(72) Inventors: Robert Lingeman, Duarte, CA (US); Linda H. Malkas, Duarte, CA (US); Robert J. Hickey, Duarte, CA (US)

(21) Appl. No.: 18/288,692

(22) PCT Filed: Apr. 29, 2022
(86) PCT No.: PCT/US2022/026928

§ 371 Date: Oct. 27, 2023

Related U.S. Application Data
(60) Provisional application No. 63/182,408, filed on Apr. 30, 2021.

(51) Int. Cl. A61K 31/167; A61K 31/4725; A61K 31/506; A61K 31/517; A61K 31/5377; A61P 35/00

(52) U.S. Cl. CPC A61K 31/167; A61K 31/4725; A61K 31/506; A61K 31/517; A61K 31/5377; A61P 35/00

(57) ABSTRACT

Described herein are, inter alia, methods of treating cancer using an EGFR-TK inhibitor and a PCNA inhibitor, and pharmaceutical compositions comprising an EGFR-TK inhibitor and a PCNA inhibitor.

### CROSS-REFERENCE TO RELATED APPLICATIONS

This application claims the benefit of priority to U.S. Application No. 63/182,408 filed Apr. 30, 2021, the disclosure of which is incorporated by reference herein in its entirety.

### BACKGROUND

Lung cancer is the most common cancer worldwide, with non-small cell lung cancer (NSCLC) accounting for about 85% of lung cancer cases. In Western countries, 10-15% non-small cell lung cancer (NSCLC) patients express epidermal growth factor receptor (EGFR) mutations in their tumors and Asian countries have reported rates as high as 30-40%. The predominant oncogenic EGFR mutations (L858R and ex19del) account for about 90% of EGFR NSCLC. EGFR Exon 20 insertion mutations (Ex20ins) were described to account for 4-10% of all EGFR mutations in patients. EGFR Exon 20 insertion mutations include EGFR 20 duplication mutations.

EGFR-mutant patients are given an EGFR inhibitor as first line therapy. However, most patients develop acquired resistance. In up to 50% of NSCLC patients harboring a primary EGFR mutation treated with first generation reversible EGFR tyrosine kinase inhibitors (EGFR-TK inhibitors), also referred to as first-generation EGFR-TK inhibitors, such as erlotinib, gefitinib and icotinib, a secondary “gatekeeper” T790M mutation develops. Second-generation EGFR-TK inhibitors (such as afatinib and dacomitinib) have been developed to overcome this mechanism of resistance. These are irreversible agents that covalently bind to cysteine 797 at the EGFR ATP site. Second generation EGFR-TK inhibitors are potent on both activating (L858R, ex19del) and acquired T790M mutations in pre-clinical models. Their clinical efficacy has however proven to be limited.

This has led to the development of third-generation EGFR TKIs which are WT EGFR sparing, and also have relative equal potency for activating EGFR mutations (L858R, ex19del) and acquired T790M. Third generation EGFR TKIs such as osimertinib and rociletinib have been developed. Osimertinib (TAGRISSO®, AstraZeneca) has been approved for the treatment of patients with metastatic epidermal growth factor receptor (EGFR) T790M mutation-positive non-small cell lung cancer (NSCLC), who have progressed on or after EGFR tyrosine kinase inhibitor (TKI) therapy.

Treatment with EGFR-TK inhibitors has, however, not been shown to definitively translate into prolonged overall survival. Hence, there is a need for additional treatment options for patients with EGFR cancers. The disclosure is directed to these, as well as other, important ends.

### BRIEF SUMMARY

Provided herein are methods of treating cancer by administering to a subject an effective amount of an EGFR-TK inhibitor and an effective amount of a proliferating cell nuclear antigen (PCNA) inhibitor. In embodiments, the PCNA inhibitor is compound of Formula (I) or a pharmaceutically acceptable salt thereof:

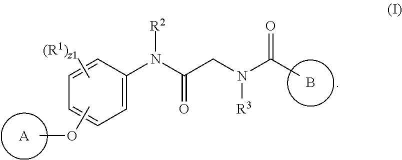

Provided herein are methods of treating cancer by administering to a subject an effective amount of an EGFR-TK inhibitor and an effective amount of a compound of Formula (A) or a pharmaceutically acceptable salt thereof:

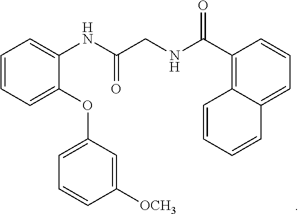 [SMILES: COc1cccc(Oc2ccccc2NC(=O)CNC(=O)c2cccc3ccccc23)c1]

Provided herein are pharmaceutical compositions comprising an EGFR-TK inhibitor, a PCNA inhibitor, and a pharmaceutically acceptable excipient. In embodiments, the PCNA inhibitor is a compound of Formula (I) or a pharmaceutically acceptable salt thereof. In embodiments, the PCNA inhibitor is a compound of Formula (A) or a pharmaceutically acceptable salt thereof.

These and other embodiments of the disclosure are provided in detail herein.

### BRIEF DESCRIPTION OF THE DRAWINGS

FIGS. 1A-1D show the results of AOH1996 tested in combination with the EGFR tyrosine kinase inhibitors (TKI) gefitinib, afatanib, neratinib, and erlotinib. The combination was most effective to kill the MCF7 cell line, a breast cancer cell line model. In FIGS. 1B-1C at dose 5/3.7, the top line is AOH1996 and the middle line is afatinib.

FIGS. 2A-2F show the results of two dose ranges of gefitinib combined with AOH1996 to test for increases in efficacy in H358, H3122, and H2228 non-small cell lung cancer (NSCLC) cell lines that express wild type EGFR. In FIGS. 2A-2F, the square represents getifinib, the circle represents AOH1996, and the triangle represents the combination of getifinib and AOH1996. A line containing both circles and squares represents AOH1996, as the square is a circle with the standard deviation lines above and below.

FIG. 3 is a survey of AOH1996 and gefitinib GI50 doses on a panel of NSCLC cell lines that express wild type EGFR. GI50 values for the H358, H3122 and H2228 cell lines were derived from dose response curves performed in our lab. GI50 values for the rest of the cell lines were obtained through the National Cancer Institute's Developmental Therapeutics Program.

FIGS. 4A-4C are dose response assays comparing AOH1996 alone, osimertinib alone, and AOH1996 and osimertinib in combination on NSCLC cell lines with wild type EGFR.

FIGS. 5A-5F show AOH1996/osimertinib dose response assays on NSCLC cell lines with mutated EGFR. The HCC827 and H1975 cell lines have an EGFR L858R mutation which activates EGFR and sensitizes the cell lines to EGFR TKIs, The H1975 cell line has an additional T790M mutation which confers resistance to first and second generation EGFR TKIs but not third generation TKIs such as osimertinib. The HCC827-R and H1975-R cell lines have acquired resistance to osimertinib, and these were more sensitive to osimertinib combined with AOH1996 than to either drug alone (FIGS. 5B, 5D). FIG. 5E: characterizations of NSCLC that has acquired resistance to osimertinib have found many genomic alterations that contribute to resistance. Some of those alterations are present in the NCI60 cell line and represented in the chart. Each circle represent cell lines with the resistance conferring genomic alteration listed on the x-axis. The BRAF and KRAS mutant cell lines had a noticeable skew in distribution towards AOH1996 sensitivity. FIG. 5F: two cell lines (14837, 14838) engineered to express oncogenic KRAS in the presence of doxycycline (Dox) were treated with escalating doses of AOH1996 in a dose response assay. The two cell lines were sensitive to AOH1996 when mutant KRAS is expressed but not when expression was suppressed.

FIGS. 6A-6C show isolation of chromatin fractions from HCC827 cells treated with AOH1996 or osimertinib alone, or in combination. FIG. 6A: Growth curve of HCC827 cells treated with 500 nM of AOH1996 or 4 nM of osimertinib alone or in combination. Chromatin fractionation was performed on a parallel set of treated cells at the 24 h time point. FIGS. 6B-6C: Following fractionation the samples were separated by polyacrylamide gel electrophoresis and immunoblotted to detect PCNA. Ponceau S was used to stain the blot for total protein to evaluate loading and transfer consistency between samples. The data shows the combination of AOH1996 with osimertinib to treat HCC827 cells resulted in accelerated loss of PCNA from chromatin.

FIGS. 7A-7B show EGFR targeted antibodies used in the treatment of colorectal cancer (CRC). CRC cell lines are particularly sensitive to AOH1996. CRC with KRAS and BRAF activating mutations are often difficult to treat but cell lines with mutant KRAS and BRAF are responsive to AOH1996. FIG. 7A: IC50 of CRC cell lines relative to the rest of the NCI60 cell lines. FIG. 7B: AOH1996 IC50 on CRC cell lines classified by presence and type of Ras-Raf-Mek-Erk pathway mutation. IC50s for the NCI60 cell lines was determined by the NCI Developmental Therapeutics Program. Mutation status of CRC cell lines was found in the Cellosaurus database.

FIGS. 8A-80 show that the combination of AOH1996 and osimertinib is more effective at killing NSCLC cell lines with wild type EGFR (H3122, H358) and mutated EGFR (HCC827, H1975) than monotherapy with either drug alone.

FIG. 9: MDA-MB-468 cells were serum starved for 24 hours and then treated with AOH1996 and osimertinib for 30 minutes before stimulating the cells by adding EGF for 15 minutes. The DMSO control cells show a typical staining pattern for cells in early/mid and late S phase. AOH1996 treated cells accumulated EGFR at the cell membrane, lost punctate staining of PCNA in the nucleus, and had increased PCNA localization to the cytoplasm. Cells treated with both drugs had apparently disorganized localization and diminished EGFR fluorescence and PCNA nuclear staining that was often segmented.

### DETAILED DESCRIPTION

Unless defined otherwise, technical and scientific terms used herein have the same meaning as commonly understood by a person of ordinary skill in the art. See, e.g., Singleton et al., Dictionary of Microbiology and Molecular Biology, 2nd ed., J. Wiley & Sons (New York, NY 1994); Sambrook et al.; Molecular Cloning, A Laboratory Manual, Cold Springs Harbor Press (Cold Springs Harbor, NY 1989), Any methods, devices and materials similar or equivalent to those described herein can be used in the practice of this disclosure. The chemical structures and formulae set forth herein are constructed according to the standard rules of chemical valency known in the chemical arts. The following definitions are provided to facilitate understanding of certain terms used frequently herein and are not meant to limit the scope of the present disclosure.

The term “proliferating cell nuclear antigen” or “PCNA” refers to about a 29 kDa protein that self assembles into a protein complex consisting of 3 subunits of individual PCNA proteins. Together these joined PCNA molecules form a DNA clamp that acts as a processivity factor for DNA polymerase b in eukaryotic cells. The term “PCNA” may refer to the nucleotide sequence or protein sequence of human PCNA (e.g., Entrez 5111, Uniprot P12004, RefSeq NM 002592, or RefSeq NP 002583). The term “PCNA” includes both the wild-type form of the nucleotide sequences or proteins as well as any mutants thereof. In embodiments, the PCNA has the nucleotide sequence corresponding to reference number GI:33239449, corresponding to RefSeq NM_002592.2, corresponding to reference number GI:4505641, or corresponding to RefSeq NP_002583.1.

The term “AOH1996” refers to the compound of Formula (A) having the structure:

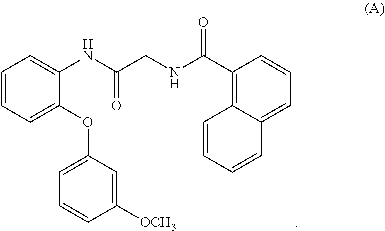 [SMILES: COc1cccc(Oc2ccccc2NC(=O)CNC(=O)c2cccc3ccccc23)c1]

In embodiments, the compound of Formula (A) is in the form of a pharmaceutically acceptable salt.

The term “EGFR protein” or “EGFR” as used herein includes any of the recombinant or naturally-occurring forms of epidermal growth factor receptor (EGFR) also known as ErbB-1 or HER1 in humans, or variants or homologs thereof that maintain EGFR activity (e.g. within at least 50%, 80%, 90%, 95%, 96%, 97%, 98%, 99% or 100% activity compared to EGFR). In embodiments, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring EGFR protein. In embodiments, the EGFR protein is substantially identical to the protein identified by the UniProt reference number P00533 or a variant or homolog having substantial identity thereto. The epidermal growth factor receptor (EGFR) is a receptor tyrosine kinase that binds EGF family ligands and activates several major pathways including the RAS-RAF-MEK-ERK pathway, the PI3K-AKT pathway, the PLCgamma-PKC pathway, and STAT pathway. In addition to its role on the cell surface, EGFR is also active in the nucleus where it plays a role in cell proliferation, DNA repair, and chemo-resistance. EGFR signaling is often upregulated in cancers.

The term “EGFR mutations” refer to mutations in the EGFR protein. Exemplary mutations in the EGFR protein include L858R, ex19del, T790M, and Ex20ins. Ex20ins (or Ex 20 insertion mutations) include single insertion mutations and duplication mutations.

The term “tyrosine kinase” refers to enzymes that activate proteins by signal transduction cascades. The proteins are activated by adding a phosphate group from ATP to the tyrosine residues of the proteins, referred to as phosphorylation.

The term “inhibition,” “inhibit,” “inhibiting” and the like in reference to a protein-inhibitor interaction means negatively affecting (e.g. decreasing) the activity or function of the protein relative to the activity or function of the protein in the absence of the inhibitor. In embodiments inhibition means negatively affecting (e.g. decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

The terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In embodiments, expression or activity is 1.5-fold, 2-fold, 3-fold, 4-fold, 5-fold, 10-fold or lower than the expression or activity in the absence of the antagonist.

The term “EGFR-TK inhibitor” or “epidermal growth factor receptor-tyrosine kinase inhibitor” or “EGFR TKI” refers to tyrosine kinase inhibitors that inhibit or block the activation of downstream signaling induced by EGFR through binding to the ATP-binding sites (e.g., tyrosine kinase inhibitors bind to EGFR and inhibit the binding of ATP to the tyrosine kinase domain of EGFR). EGFR-TK inhibitors can be used to treat cancers having EGFR mutations and/or aberrant activation of EGFR. Exemplary EGFR-TK inhibitors include osimertinib, gefitinib, afatinib, neratinib, erlotinib, rociletinib, olmutinib, lazertinib, nazartinib, naquotinib, mavelertinib, abivertinib, olafertinib, alflutinib, amivantamb, tarloxitinib, mobocertinib, savolitinib, capmatinib, cetuximab, panitumumab, lapatinib, dacomitinib, necitumumab, vandetanib, icotininib, canertinib, allitinib, varlitinib, tesevatinib, pelitinib, sapitinib, EAI045 (CAS Number 1942114-09-1 or 2-(5-fluoro-2-hydroxyphenyl)-2-(3-oxo-1H-isoindol-2-yl)-N-(1,3-thiazol-2-yl)acetamide)), TAK-285 (N-[2-[4-[3-chloro-4-[3-(trifluoromethyl)phenoxy]-anilino]pyrrolo[3,2-d]pyrimidin-5-yl]ethyl]-3-hydroxy-3-methylbutanamide), AG-1478 (tyrphostin AG 1478 or N-(3-chlorophenyl)-6,7-dimethoxy-4-quinazolinanine), AEE788 (6-{4-[(4-ethyl-1-piperazinyl)methyl]phenyl}-N-[(1R)-1-phenylethyl]-1H-pyrrolo[2,3-d]pyrimidin-4-amine), CUDC-101 (7-[[4-(3-ethynylphenylamino)-7-methoxyquinazolin-6-yl]oxy]-N-hydroxyheptanamide), WZ8040 (N-[3-[[5-chloro-2-[[4-(4-methyl-1-piperazinyl)phenyl]amino]-4-pyrimidinyl]thio]phenyl]-2-propenamide), WZ4002 (N-[3-[[5-chloro-2-[[2-methoxy-4-(4-methyl-1-piperazinyl)phenyl]amino]-4-pyrimidinyl]oxy]phenyl]-2-propenamide), WZ3146 (N-[3-[[5-chloro-2-[[4-(4-methyl-1-piperazinyl)phenyl]amino]-4-pyrimidinyl]oxy]phenyl]-2-propenamide), AG-490 ((2E)-2-cyano-3-(3,4-dihydroxyphenyl)-N-(phenylmethyl)-2-propenamide), and PD153035 (N-(3-bromophenyl)-6,7-dimethoxy-4-quinazolinamine). The EGFR-TK inhibitors described herein can be in the form of a pharmaceutically acceptable salt.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g. by administering a compound or using a method as described herein), results in reduction of the disease or one or more disease symptoms.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components.

The term “alkyl,” by itself or as part of another substituent, means, unless otherwise stated, a straight (i.e., unbranched) or branched non-cyclic carbon chain (or carbon), or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched non-cyclic chain, or combinations thereof, including at least one carbon atom and at least one heteroatom (e.g., O, N, P, Si, or S), and wherein the nitrogen and sulfur atoms may optionally be oxidized, and the nitrogen heteroatom may optionally be quaternized. The heteroatom(s) (e.g., O, N, P, S, or Si) may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom. A heteroalkyl moiety may include two optionally different heteroatoms. A heteroalkyl moiety may include three optionally different heteroatoms. A heteroalkyl moiety may include four optionally different heteroatoms.

The term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, non-aromatic cyclic versions of “alkyl” and “heteroalkyl,” respectively, wherein the carbons making up the ring or rings do not necessarily need to be bonded to a hydrogen due to all carbon valencies participating in bonds with non-hydrogen atoms. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, 3-hydroxy-cyclobut-3-enyl-1,2, dione, 1H-1,2,4-triazolyl-5(4H)-one, 4H-1,2,4-triazolyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. A heterocycloalkyl moiety may include one ring heteroatom (e.g., O, N, S, Si, or P). A heterocycloalkyl moiety may include two optionally different ring heteroatoms. A heterocycloalkyl moiety may include three optionally different ring heteroatoms.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together; wherein one ring has 5 members and the other ring has 6 members, and wherein at least one ring is a heteroaryl ring. Likewise, a 6,6-fused ring heteroarylene refers to two rings fused together; wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. Non-limiting examples of aryl and heteroaryl groups include pyridinyl, pyrimidinyl, thiophenyl, thienyl, furanyl, indolyl, benzoxadiazolyl, benzodioxolyl, benzodioxanyl, thianaphthanyl, pyrrolopyridinyl, indazolyl, quinolinyl, quinoxalinyl, pyridopyrazinyl, quinazolinonyl, benzoisoxazolyl, imidazopyridinyl, benzofuranyl, benzothienyl, benzothiophenyl, phenyl, naphthyl, biphenyl, pyrrolyl, pyrazolyl, imidazolyl, pyrazinyl, oxazolyl, isoxazolyl, thiazolyl, furylthienyl, pyridyl, pyrimidyl, benzothiazolyl, purinyl, benzimidazolyl, isoquinolyl, thiadiazolyl, oxadiazolyl, pyrrolyl, diazolyl, triazolyl, tetrazolyl, benzothiadiazolyl, isothiazolyl, pyrazolopyrimidinyl, pyrrolopyrimidinyl, benzotriazolyl, benzoxazolyl, or quinolyl. The examples above may be substituted or unsubstituted and divalent radicals of each heteroaryl example above are non-limiting examples of heteroarylene. A heteroaryl moiety may include one ring heteroatom. A heteroaryl moiety may include two optionally different ring heteroatoms. A heteroaryl moiety may include three optionally different ring heteroatoms. A heteroaryl moiety may include four optionally different ring heteroatoms. An aryl moiety may have a single ring. An aryl moiety may have two optionally different rings. An aryl moiety may have three optionally different rings. An aryl moiety may have four optionally different rings. A heteroaryl moiety may have one ring. A heteroaryl moiety may have two optionally different rings. A heteroaryl moiety may have three optionally different rings.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substitutents described herein.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

Each of the above terms (e.g., “alkyl,” “heteroalkyl,”, “cycloalkyl”, “heterocycloalkyl”, “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R′, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR′″, —NR—C(NR′R″)═NR″, —S(O)R′, —S(O)₂R′, —S(O)₂NR′R″, —NRSO₂R′, —NR′NR″R′″, —ONR′R″, —NR′C═(O)NR″NR′″R″″, —CN, —NO₂, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound of the invention includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered it ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C═(O)NR″NR′″R″″, —CN, —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R′″, and R″″ groups when more than one of these groups is present.

Two or more substituents may optionally be joined to form aryl, heteroaryl, cycloalkyl, or heterocycloalkyl groups. Such so-called ring-forming substituents are typically, though not necessarily, found attached to a cyclic base structure. In embodiments, the ring-forming substituents are attached to adjacent members of the base structure. For example, two ring-forming substituents attached to adjacent members of a cyclic base structure create a fused ring structure. In embodiments, the ring-forming substituents are attached to a single member of the base structure. For example, two ring-forming substituents attached to a single member of a cyclic base structure create a spirocyclic structure. In embodiments, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond; and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′— (C″R″R′″)_{d}—, where s and d are independently integers of from 0 to 3, and X is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

Where substituent groups are specified by their conventional chemical formulae, written from left to right, they equally encompass the chemically identical substituents that would result from writing the structure from right to left, e.g., —CH₂O— is equivalent to —OCH₂—.

As used herein, the terms “heteroatom” or “ring heteroatom” are meant to include, oxygen (O), nitrogen (N), sulfur (S), phosphorus (P), and silicon (Si).

A “substituent group,” as used herein, means a group selected from the following moieties: (A) oxo, halogen, —CF₃, —CN, —OH, —NH₂, —COOH, —CONH₂; —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and (B) alkyl, heteroalkyl, cycloalkyl, heterocycloalkyl, aryl, heteroaryl, substituted with at least one substituent selected from: (i) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and (ii) alkyl, heteroalkyl, cycloalkyl, heterocycloalkyl, aryl, heteroaryl, substituted with at least one substituent selected from: (a) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and (b) alkyl, heteroalkyl, cycloalkyl, heterocycloalkyl, aryl, heteroaryl, substituted with at least one substituent selected from: oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In embodiments, each substituted group described in the compounds herein is substituted with at least one substituent group. In embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In embodiments, at least one or all of these groups are substituted with at least one size-limited substituent group. In other embodiments, at least one or all of these groups are substituted with at least one lower substituent group.

In embodiments of the compounds herein, each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene.

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present invention contain both basic and acidic functionalities that allow the compounds to be converted into either base or acid addition salts. Other pharmaceutically acceptable carriers known to those of skill in the art are suitable for the present invention. Salts tend to be more soluble in aqueous or other protonic solvents than are the corresponding free base forms.

The compounds described herein may exist as salts, such as with pharmaceutically acceptable acids. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known in the art.

The neutral forms of the compounds are regenerated by contacting the salt with a base or acid and isolating the parent compound in the conventional manner. The parent form of the compound differs from the various salt forms in certain physical properties, such as solubility in polar solvents.

In embodiments, compounds described herein can exist in unsolvated forms and solvated forms, including hydrated forms. In general, the solvated forms are equivalent to unsolvated forms and are encompassed within the scope of the present invention. Certain compounds may exist in multiple crystalline or amorphous forms. In general, all physical forms are equivalent for the uses contemplated by the present invention and are intended to be within the scope of the present invention.

Certain compounds possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present invention. The compounds do not include those which are known in the art to be too unstable to synthesize and/or isolate. The disclosure includes compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers. Unless otherwise stated, structures depicted herein are meant to include all stereochemical forms of the structure, i.e., the R and S configurations for each asymmetric center.

The term “isomers” refers to compounds having the same number and kind of atoms, and hence the same molecular weight, but differing in respect to the structural arrangement or configuration of the atoms.

The term “tautomer” refers to one of two or more structural isomers which exist in equilibrium and which are readily converted from one isomeric form to another. It will be apparent to one skilled in the art that certain compounds may exist in tautomeric forms, all such tautomeric forms of the compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

The symbol “ [symbol] ” and “-” denotes the point of attachment of a chemical moiety to the remainder of a molecule or chemical formula.

The terms “a” or “an,” as used herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls. Moreover, where a moiety is substituted with an R substituent, the group may be referred to as “R-substituted.” Where a moiety is R-substituted, the moiety is substituted with at least one R substituent and each R substituent is optionally different.

“Control” or “control experiment” or “standard control” is used in accordance with its plain ordinary meaning and refers to an experiment in which the subjects or reagents of the experiment are treated as in a parallel experiment except for omission of a procedure, reagent, or variable of the experiment. In instances, the control is used as a standard of comparison in evaluating experimental effects. In embodiments, a control is the same experiment or treatment method in the absence of a compound (e.g., as described herein) used in the non-control experiment or treatment method being compared to the control.

The term “activation”, “activate”, “activating” and the like in reference to a protein-activator (e.g. agonist) interaction means positively affecting (e.g. increasing) the activity or function of the protein relative to the activity or function of the protein in the absence of the activator (e.g. compound described herein). Thus, activation may include, at least in part, partially or totally increasing stimulation, increasing or enabling activation, or activating, sensitizing, or up-regulating signal transduction or enzymatic activity or the amount of a protein decreased in a disease. Activation may include, at least in part, partially or totally increasing stimulation, increasing or enabling activation, or activating, sensitizing, or up-regulating signal transduction or enzymatic activity or the amount of a protein.

The term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value.

### Compounds

Provided herein are PCNA inhibitors and pharmaceutically acceptable salts thereof. Provided herein are PCNS inhibitors or pharmaceutically acceptable salts thereof of Formula (I):

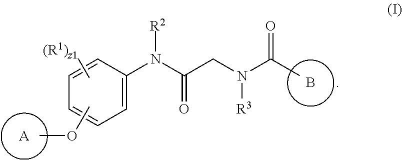

Ring A is a substituted or unsubstituted phenyl or a substituted or unsubstituted 5 to 6 membered heteroaryl. Ring B is a substituted or unsubstituted naphthyl, a substituted or unsubstituted quinolinyl, or a substituted or unsubstituted isoquinolinyl.

R¹ is independently hydrogen, halogen, —CX¹₃, —CHX¹₂, —CH₂X¹, —CN, —SO₂Cl, —SOₙ₁R¹⁰, —SOᵥ₁NR⁷R⁸, —NHNR⁷R⁸, —ONR⁷R⁸, —NHC═(O)NHNR⁷R⁸, —NHC═(O)NR⁷R⁸, —N(O)ₘ₁, —NR⁷R⁸, —C(O)R⁹, —C(O)—OR⁹, —C(O)NR⁷R⁸, —OR¹⁰, —NR⁷SO₂R¹⁰, —NR⁷C═(O)R⁹, —NR⁷C(O)—OR⁹, —NR⁷OR⁹, —OCX¹₃, —OCHX¹₂, —OCH₂X¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl. In embodiments, R¹ is independently a halogen, —CX¹₃, —CHX¹₂, —CH₂X¹, —CN, —SOₙ₁R¹⁰, —SOᵥ₁NR⁷R⁸, —NHNH₂, —ONR⁷R⁸, —NHC═(O)NHNH₂, —NHC═(O)NR⁷R⁸, —N(O)ₘ₁, —NR⁷R⁸, —C(O)R⁹, —C(O)—OR⁹, —C(O)NR⁷R⁸, —OR¹⁰, —NR⁷SO₂R¹⁰, —NR⁷C═(O)R⁹, —NR⁷C(O)—OR⁹, —NR⁷OR⁹, —OCX¹₃, —OCHX¹₂, —OCH₂X¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl. It is understood that when z1 is 0, then R¹ is hydrogen.

R² is hydrogen, halogen, —CX²₃. —CHX²₂, —CH₂X², —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCX²₃, —OCHX²₂, —OCH₂X², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R³ is hydrogen, halogen, —CX³₃, —CHX³₂, —CH₂X³, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCX³₃, —OCHX³₂, —OCH₂X³, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁷, R⁸, R⁹, and R¹⁰ are independently hydrogen, halogen, —CX^{A}₃, —CHX^{A}₂, —CH₂X^{A}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCX^{A}₃, —OCHX^{A}₂, —OCH₂X^{A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁷ and R⁸ substituents bonded to the same nitrogen atom may optionally be joined to form a substituted or unsubstituted heterocycloalkyl or substituted or unsubstituted heteroaryl.

The symbol z1 is an integer from 0 to 4. The symbols m1 and v1 are independently an integer 1 or 2. The symbol n1 is an integer from 0 to 4. The symbols X¹, X², X³, and X^{A} are independently —Cl, —Br, —I, or —F.

In embodiments, the PCNS inhibitor is a compound having the formula:

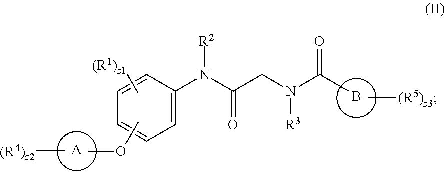

wherein R¹, R², R³, Ring A, Ring B, and z1 are as described herein, including in compounds of formula (I) and including in embodiments. In embodiments, Ring A is phenyl (substituted or unsubstituted with R⁴) or 5 to 6 membered heteroaryl (substituted or unsubstituted with R⁴) and Ring B is naphthyl (substituted or unsubstituted with R⁵), quinolinyl (substituted or unsubstituted with R⁵), or isoquinolinyl (substituted or unsubstituted with R⁵).

R⁴ is independently halogen, —CX⁴₃, —CHX⁴₂, —CH₂X⁴, —CN, —SO₂Cl, —SOₙ₄R¹⁴, —SOᵥ₄NR¹¹R¹², —NHNR¹¹R¹², —ONR¹¹R¹², —NHC═(O)NHNR¹¹R¹², —NHC═(O)NR¹¹R¹², —N(O)ₘ₄, —NR¹¹R¹², —C(O)R¹³, —C(O)—OR¹³, —C(O)NR¹¹R¹², —OR¹⁴, —NR¹¹SO₂R¹⁴, —NR¹¹C═(O)R¹³, —NR¹¹C(O)—OR¹³, —NR¹¹OR¹³, —OCX⁴₃, —OCHX⁴₂, —OCH₂X⁴, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R⁴ substituents may optionally be joined to form a substituted or unsubstituted cycloalkyl, substituted or unsubstituted heterocycloalkyl, substituted or unsubstituted aryl, or substituted or unsubstituted heteroaryl. In embodiments, R⁴ is independently a halogen, —CX⁴₃, —CHX⁴₂, —CH₂X⁴, —CN, —SOₙ₄R¹⁴, —SOᵥ₄NR¹¹R¹², —NHNR¹¹R¹², —ONR¹¹R¹², —NHC═(O)NHNR¹¹R¹², —NHC═(O)NR¹¹R¹², —N(O)ₘ₄, —NR¹¹R¹², —C(O)R¹³, —C(O)—OR¹³, —C(O)NR¹¹R¹², —OR¹⁴, —NR¹¹SO₂R¹⁴, —NR¹¹C═(O)R¹³, —NR¹¹C(O)—OR¹³, —NR¹¹OR¹³, —OCX⁴₃, —OCHX⁴₂, —OCH₂X⁴ substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R⁴ substituents may optionally be joined to form a substituted or unsubstituted cycloalkyl, substituted or unsubstituted heterocycloalkyl, substituted or unsubstituted aryl, or substituted or unsubstituted heteroaryl. It is understood that when z2 is 0, then R⁴ is hydrogen.

R⁵ is independently halogen, —CX⁵₃, —CHX⁵₂, —CH₂X⁵, —CN, —SO₂Cl, —SOₙ₅R¹⁸, —SOᵥ₅NR¹⁵R¹⁶, —NHNR¹⁵R¹⁶, —ONR¹⁵R¹⁶, —NHC═(O)NHNR¹⁵R¹⁶, —NHC═(O)NR¹⁵R¹⁶, —N(O)ₘ₅, —NR¹⁵R¹⁶, —C(O)R¹⁷, —C(O)—OR¹⁷, —C(O)NR¹⁵R¹⁶, —OR¹⁸, —NR¹¹SO₂R¹⁸, —NR¹⁵C═(O)R¹⁷, —NR¹⁵C(O)—OR¹⁷, —NR¹⁵OR¹¹, —OCX⁵₃, —OCHX⁵₂, —OCH₂X⁵, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R⁵ substituents may optionally be joined to form a substituted or unsubstituted cycloalkyl, substituted or unsubstituted heterocycloalkyl, substituted or unsubstituted aryl, or substituted or unsubstituted heteroaryl. In embodiments, R⁵ is independently a halogen, —CX⁵₃, —CHX⁵₂, —CH₂X⁵, —CN, —SOₙ₅R¹⁸, —SOᵥ₅NR¹⁵R¹⁶, —NHNR¹⁵R¹⁶, —ONR¹⁵R¹⁶, —NHC═(O)NHNR¹⁵R¹⁶, —NHC═(O)NR¹⁵R¹⁶, —N(O)ₘ₅, —NR¹⁵R¹⁶, —C(O)R¹⁷, —C(O)—OR¹⁷, —C(O)NR¹⁵R¹⁶, —OR¹⁸, —NR¹⁵SO₂R¹⁸, —NR¹⁵C═(O)R¹⁷, —NR¹⁵C(O)—OR¹⁷, —NR¹⁵OR¹⁷, —OCX⁵₃—OCHX⁵₃, —OCH₂X⁵ substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R⁵ substituents may optionally be joined to form a substituted or unsubstituted cycloalkyl, substituted or unsubstituted heterocycloalkyl, substituted or unsubstituted aryl, or substituted or unsubstituted heteroaryl. It is understood that when z3 is 0, then R⁵ is hydrogen.

R¹¹, R¹², R¹³, and R¹⁴ are independently hydrogen, halogen, —CX^{B}₃, —CHX^{B}₂, —CH₂X^{B}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{B}₃, —OCHX^{B}₂, —OCH₂X^{B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹¹ and R¹² substituents bonded to the same nitrogen atom may optionally be joined to form a substituted or unsubstituted heterocycloalkyl or substituted or unsubstituted heteroaryl.

R¹⁵, R¹⁶, R¹⁷, and R¹⁸ are independently hydrogen, halogen, —CX^{C}₃, —CHX^{C}₂, —CH₂X^{C}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{C}₃, —OCHX^{C}₂, —OCH₂X^{C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹⁵ and R¹⁶ substituents bonded to the same nitrogen atom may optionally be joined to form a substituted or unsubstituted heterocycloalkyl or substituted or unsubstituted heteroaryl.

The symbol z2 is an integer from 0 to 5. The symbol z3 is an integer from 0 to 7. The symbols m4, m5, v4 and v5 are independently an integer 1 or 2. The symbols n4 and n5 are independently an integer from 0 to 4. The symbols X⁴, X⁵, X^{B}, and X^{C} are independently —Cl, —Br, —I, or —F.

In embodiments, Ring A is substituted phenyl. In embodiments, Ring A is unsubstituted phenyl. In embodiments, Ring A is phenyl. In embodiments, Ring A is a substituted 5 to 6 membered heteroaryl. In embodiments, Ring A is an unsubstituted 5 to 6 membered heteroaryl. In embodiments, Ring A is a 5 to 6 membered heteroaryl. In embodiments, Ring A is a substituted thienyl. In embodiments, Ring A is an unsubstituted thienyl. In embodiments, Ring A is a thienyl. In embodiments, Ring A is a 2-thienyl. In embodiments, Ring A is a 3-thienyl. In embodiments, Ring A is a substituted pyridyl. In embodiments, Ring A is an unsubstituted pyridyl. In embodiments, Ring A is a pyridyl. In embodiments, Ring A is a 2-pyridyl. In embodiments, Ring A is a 3-pyridyl. In embodiments, Ring A is a 4-pyridyl. In embodiments, Ring A is unsubstituted pyrrolyl. In embodiments, Ring A is substituted pyrrolyl. In embodiments, Ring A is pyrrolyl. In embodiments, Ring A is unsubstituted furanyl. In embodiments, Ring A is substituted furanyl. In embodiments, Ring A is furanyl. In embodiments, Ring A is unsubstituted pyrazolyl. In embodiments, Ring A is substituted pyrazolyl. In embodiments, Ring A is pyrazolyl. In embodiments, Ring A is unsubstituted imidazolyl. In embodiments, Ring A is substituted imidazolyl. In embodiments, Ring A is imidazolyl. In embodiments, Ring A is unsubstituted oxazolyl. In embodiments, Ring A is substituted oxazolyl. In embodiments, Ring A is oxazolyl. In embodiments, Ring A is unsubstituted isoxazolyl. In embodiments, Ring A is substituted isoxazolyl. In embodiments, Ring A is isoxazolyl. In embodiments, Ring A is unsubstituted thiazolyl. In embodiments, Ring A is substituted thiazolyl. In embodiments, Ring A is thiazolyl. In embodiments, Ring A is unsubstituted triazolyl. In embodiments, Ring A is substituted triazolyl. In embodiments, Ring A is triazolyl. In embodiments, Ring B is a substituted naphthyl. In embodiments, Ring B is unsubstituted naphthyl. In embodiments, Ring B is a naphthyl. In embodiments, Ring B is a 1-naphthyl. In embodiments, Ring B is a 2-naphthyl. In embodiments, Ring B is a quinolinyl. In embodiments, Ring B is a substituted quinolinyl. In embodiments, Ring B is unsubstituted quinolinyl. In embodiments, Ring B is an isoquinolinyl. In embodiments, Ring B is a substituted isoquinolinyl. In embodiments, Ring B is unsubstituted isoquinolinyl. In embodiments, Ring B is a 1-isoquinolinyl. In embodiments, Ring B is a 3-isoquinolinyl. In embodiments, Ring B is a 4-isoquinolinyl.

In embodiments, R¹ is independently halogen, —CF₃, —CHF₂, —OCF₃, —OCHF₂, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl. In embodiments, R¹ is independently halogen, —CF₃, —OH, —NH₂, —SH, substituted or unsubstituted C₁-C₄ alkyl, substituted or unsubstituted 2 to 4 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R′ is independently halogen, —OH, —NH₂; —SH, unsubstituted C₁-C₄ alkyl, or unsubstituted 2 to 4 membered heteroalkyl. In embodiments, R¹ is independently halogen, —OH, unsubstituted methyl, or unsubstituted methoxy. In embodiments, R¹ is independently halogen. In embodiments, R¹ is independently —CF₃. In embodiments, R¹ is independently —CHF₂, In embodiments, R¹ is independently —CH₂F. In embodiments, R¹ is independently —OCF₃. In embodiments, R¹ is independently —OCHF₂. In embodiments, R¹ is independently —OCH₂F. In embodiments, R¹ is independently substituted or unsubstituted C₁-C₈ alkyl. In embodiments, R¹ is independently substituted or unsubstituted 2 to 8 membered heteroalkyl. In embodiments, R¹ is independently substituted or unsubstituted C₃-C₈ cycloalkyl. In embodiments, R¹ is independently substituted or unsubstituted 3 to 8 membered heterocycloalkyl. In embodiments, R¹ is independently substituted or unsubstituted C₆-C₁₀ aryl. In embodiments, R¹ is independently substituted or unsubstituted 5 to 10 membered heteroaryl. In embodiments, R¹ is independently —OH. In embodiments, R¹ is independently —NH₂. In embodiments, R¹ is independently —SH. In embodiments, R¹ is independently substituted or unsubstituted C₁-C₄ alkyl. In embodiments, R¹ is independently substituted or unsubstituted 2 to 4 membered heteroalkyl. In embodiments, R¹ is independently substituted or unsubstituted C₃-C₆ cycloalkyl. In embodiments, R¹ is independently substituted or unsubstituted 3 to 6 membered heterocycloalkyl. In embodiments, R¹ is independently substituted or unsubstituted phenyl. In embodiments, R¹ is independently substituted or unsubstituted 5 to 6 membered heteroaryl.

In embodiments, R¹ is independently substituted C₁-C₈ alkyl. In embodiments, R¹ is independently substituted 2 to 8 membered heteroalkyl. In embodiments, R′ is independently substituted C₃-C₈ cycloalkyl. In embodiments, R¹ is independently substituted 3 to 8 membered heterocycloalkyl. In embodiments, R¹ is independently substituted C₆-C₁₀ aryl. In embodiments, R¹ is independently substituted 5 to 10 membered heteroaryl. In embodiments, R¹ is independently substituted C₁-C₄ alkyl. In embodiments, R¹ is independently substituted to 4 membered heteroalkyl. In embodiments, R¹ is independently substituted C₃-C₆ cycloalkyl. In embodiments, R¹ is independently substituted 3 to 6 membered heterocycloalkyl. In embodiments, R¹ is independently substituted phenyl. In embodiments, R¹ is independently substituted 5 to 6 membered heteroaryl. In embodiments, R¹ is independently unsubstituted C₁-C₈ alkyl. In embodiments, R¹ is independently unsubstituted 2 to 8 membered heteroalkyl. In embodiments, R¹ is independently unsubstituted C₃-C₈ cycloalkyl. In embodiments, R¹ is independently unsubstituted 3 to 8 membered heterocycloalkyl. In embodiments, R¹ is independently unsubstituted C₆-C₁₀ aryl. In embodiments, R¹ is independently unsubstituted 5 to 10 membered heteroaryl. In embodiments, R¹ is independently unsubstituted C₁-C₄ alkyl. In embodiments, R¹ is independently unsubstituted 2 to 4 membered heteroalkyl. In embodiments, R¹ is independently unsubstituted C₃-C₆ cycloalkyl. In embodiments, R′ is independently unsubstituted 3 to 6 membered heterocycloalkyl. In embodiments, R¹ is independently unsubstituted phenyl. In embodiments, R¹ is independently unsubstituted 5 to 6 membered heteroaryl. In embodiments, R¹ is independently unsubstituted methyl. In embodiments, R¹ is independently unsubstituted ethyl. In embodiments, R′ is independently unsubstituted isopropyl. In embodiments, R¹ is independently unsubstituted tert-butyl. In embodiments, R′ is independently unsubstituted methoxy. In embodiments, R¹ is independently unsubstituted ethoxy. In embodiments, R¹ is independently —F. In embodiments, R¹ is independently —Cl. In embodiments, R¹ is independently —Br. In embodiments, R¹ is independently —I. In embodiments, R¹ is independently hydrogen. In embodiments, R¹ is independently halogen, —CF₃, —CHF₂, —CH₂F, —OCF₃, —OCHF₂, —OCH₂F, —OH, —NH₂, —SH, unsubstituted C₁-C₄ alkyl, or unsubstituted 2 to 4 membered heteroalkyl.

In embodiments, z1 is 1. In embodiments, z1 is 0. In embodiments, z1 is 2. In embodiments, z1 is 3. In embodiments, z1 is 4.

In embodiments, R² is hydrogen, —CX²₃, —CHX²₂, —CH₂X², —CN, —C(O)H, —C(O)OH, —C(O)NH₂, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments R² is hydrogen, unsubstituted methyl, unsubstituted ethyl, or unsubstituted isopropyl. In embodiments, R² is hydrogen. In embodiments, R² is unsubstituted methyl. In embodiments, R² is unsubstituted ethyl. In embodiments, R² is unsubstituted isopropyl. In embodiments, R² is unsubstituted tert-butyl.

In embodiments, R^{Z} is hydrogen, halogen, —CX²₃, —CHX²₂, —CH₂X², —CN, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R³ is hydrogen, —CX²₃, —CHX²₂, —CH₂X², —CN, —C(O)H, —C(O)OH, —C(O)NH₂, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R³ is hydrogen, unsubstituted methyl, unsubstituted ethyl, or unsubstituted isopropyl. In embodiments, R³ is hydrogen. In embodiments, R³ is unsubstituted methyl. In embodiments, R³ is unsubstituted ethyl. In embodiments, R³ is unsubstituted isopropyl. In embodiments, R³ is unsubstituted tert-butyl. In embodiments, R³ is hydrogen, halogen, —CX³₃, —CHX³₂, —CH₂X³, —CN, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁴ is independently halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl. In embodiments, R⁴ is independently halogen, —CF₃, —OH, —NH₂, —SH, substituted or unsubstituted C₁-C₄ alkyl, substituted or unsubstituted 2 to 4 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R⁴ is independently halogen, —OH, —NH₂, —SH, unsubstituted C₁-C₄ alkyl, or unsubstituted 2 to 4 membered heteroalkyl. In embodiments, R⁴ is independently halogen, —OH, unsubstituted methyl, or unsubstituted methoxy. In embodiments, R⁴ is independently halogen. In embodiments, R⁴ is independently —OH. In embodiments, R⁴ is independently unsubstituted methyl. In embodiments, R⁴ is independently unsubstituted methoxy. In embodiments, R⁴ is independently unsubstituted ethyl. In embodiments, R⁴ is independently —F. In embodiments, R⁴ is independently —Cl. In embodiments, R⁴ is independently —Br. In embodiments, R⁴ is independently —I. In embodiments, R⁴ is independently —CF₃. In embodiments, R⁴ is independently —NH₂. In embodiments, R⁴ is independently —SH. In embodiments, R⁴ is independently unsubstituted isopropyl. In embodiments, R⁴ is independently unsubstituted tert-butyl. In embodiments, R⁴ is independently unsubstituted ethoxy. In embodiments, R⁴ is independently unsubstituted propoxy.

In embodiments, R⁴ is independently a halogen. In embodiments, R⁴ is independently —CX⁴₃. In embodiments, R⁴ is independently —CHX⁴₂. In embodiments, R⁴ is independently —CH₂X⁴. In embodiments, R⁴ is independently —CN. In embodiments, R⁴ is independently —SOₙ₄R¹⁴. In embodiments, R⁴ is independently —SR¹⁴. In embodiments, R⁴ is independently —SOᵥ₄NR¹¹R¹². In embodiments, R⁴ is independently —NHNR¹¹R¹². In embodiments, R⁴ is independently —ONR¹¹R¹². In embodiments, R⁴ is independently —NHC═(O)NHNR¹¹R¹². In embodiments, R⁴ is independently —NHC═(O)NR¹¹R¹². In embodiments, R⁴ is independently —N(O)ₘ₄. In embodiments, R⁴ is independently —NR¹¹R¹². In embodiments, R⁴ is independently —C(O)R¹³. In embodiments, R⁴ is independently —C(O)—OR¹³. In embodiments, R⁴ is independently —C(O)NR¹¹R¹². In embodiments, R⁴ is independently —OR¹⁴. In embodiments, R⁴ is independently —NR¹¹SO₂R¹⁴. In embodiments, R⁴ is independently —NR¹¹C═(O)R¹³. In embodiments, R⁴ is independently —NR¹¹C(O)—OR¹³. In embodiments, R⁴ is independently —NR¹¹OR¹³. In embodiments, R⁴ is independently —OCX⁴₃. In embodiments, R⁴ is independently —OCHX⁴₂. In embodiments, R⁴ is independently —OCH₂X⁴. In embodiments, R⁴ is independently —CF₃. In embodiments, R⁴ is independently —CHF₂. In embodiments, R⁴ is independently —CH₂F. In embodiments, R⁴ is independently —SO₂CH₃. In embodiments, R⁴ is independently —SO₂NH₂. In embodiments, R⁴ is independently —SH. In embodiments, R⁴ is independently —N(O)₂. In embodiments, R⁴ is independently —NH₂. In embodiments, R⁴ is independently —C(O)CH₃. In embodiments, R⁴ is independently —C(O)OH. In embodiments, R⁴ is independently —C(O)NH₂. In embodiments, R⁴ is independently —OH. In embodiments, R⁴ is independently —OCF₃, In embodiments, R⁴ is independently —OCHF₂. In embodiments, R⁴ is independently —OCH₂F.

In embodiments, R⁴ is independently halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl. In embodiments, R⁴ is independently halogen, —CF₃, —OH, —NH₂, —SH, substituted or unsubstituted C₁-C₄ alkyl, substituted or unsubstituted 2 to 4 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R⁴ is independently halogen, —CF₃, —CHF₂, —CH₂F, —OCF₃, —OCHF₂, —OCH₂F, —OH, —NH₂, —SH, unsubstituted C₁-C₄ alkyl, or unsubstituted 2 to 4 membered heteroalkyl.

In embodiments, R⁴ is independently substituted or unsubstituted alkyl. In embodiments, R⁴ is independently substituted or unsubstituted heteroalkyl. In embodiments, R⁴ is independently substituted or unsubstituted cycloalkyl. In embodiments, R⁴ is independently substituted or unsubstituted heterocycloalkyl. In embodiments, R⁴ is independently substituted or unsubstituted aryl. In embodiments, Ra is independently substituted or unsubstituted heteroaryl. In embodiments, two adjacent R⁴ substituents may optionally be joined to form a substituted or unsubstituted cycloalkyl. In embodiments, two adjacent R⁴ substituents may optionally be joined to form a substituted or unsubstituted heterocycloalkyl. In embodiments, two adjacent R⁴ substituents may optionally be joined to form a substituted or unsubstituted aryl. In embodiments, two adjacent R⁴ substituents may optionally be joined to form a substituted or unsubstituted heteroaryl.

In embodiments, R⁴ is independently substituted or unsubstituted alkyl (e.g. C₁-C₈ alkyl, C₁-C₆ alkyl, or C₁-C₄ alkyl), substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), substituted or unsubstituted cycloalkyl (e.g. C₃-C₈ cycloalkyl, C₄-C₈ cycloalkyl, or C₅-C₆ cycloalkyl), substituted or unsubstituted heterocycloalkyl (e.g. 3 to 8 membered heterocycloalkyl, 4 to 8 membered heterocycloalkyl, or 5 to 6 membered heterocycloalkyl), substituted or unsubstituted aryl (e.g. C₆-C₁₀ aryl or C₆ aryl), or substituted or unsubstituted heteroaryl (e.g. 5 to 10 membered heteroaryl, 5 to 9 membered heteroaryl, or 5 to 6 membered heteroaryl). In embodiments, R⁴ is independently substituted alkyl (e.g. C₁-C₈ alkyl, C₁-C₆ alkyl, or C₁-C₄ alkyl), substituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), substituted cycloalkyl (e.g. C₃-C₈ cycloalkyl, C₄-C₈ cycloalkyl, or C₅-C₆ cycloalkyl), substituted heterocycloalkyl (e.g. 3 to 8 membered heterocycloalkyl, 4 to 8 membered heterocycloalkyl, or 5 to 6 membered heterocycloalkyl), substituted aryl (e.g. C₆-C₁₀ aryl or C₆ aryl), or substituted heteroaryl (e.g. 5 to 10 membered heteroaryl, 5 to 9 membered heteroaryl, or 5 to 6 membered heteroaryl). In embodiments, R⁴ is independently unsubstituted alkyl (e.g. C₁-C₈ alkyl, C₁-C₆ alkyl, or C₁-C₄ alkyl), unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), unsubstituted cycloalkyl (e.g. C₃-C₈ cycloalkyl, C₄-C₈ cycloalkyl, or C₅-C₆ cycloalkyl), unsubstituted heterocycloalkyl (e.g. 3 to 8 membered heterocycloalkyl, 4 to 8 membered heterocycloalkyl, or 5 to 6 membered heterocycloalkyl), unsubstituted aryl (e.g. C₆-C₁₀ aryl or C₆ aryl), or unsubstituted heteroaryl (e.g. 5 to 10 membered heteroaryl, 5 to 9 membered heteroaryl, or 5 to 6 membered heteroaryl).

In embodiments, R¹⁴ is independently hydrogen, —CX^{B}₃, —CHX^{B}₂, —CH₂X^{B}, —CN, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R¹⁴ is independently hydrogen. In embodiments, R¹⁴ is independently —CX^{B}3. In embodiments, R¹⁴ is independently —CHX^{B}₂. In embodiments, R¹⁴ is independently —CH₂X^{B}. In embodiments, R¹⁴ is independently —CN. In embodiments, R¹⁴ is independently —COOH. In embodiments, R¹⁴ is independently —CONH₂. In embodiments, R¹⁴ is independently substituted or unsubstituted alkyl. In embodiments, R¹⁴ is independently substituted or unsubstituted heteroalkyl. In embodiments, R¹⁴ is independently substituted or unsubstituted cycloalkyl In embodiments, R¹⁴ is independently substituted or unsubstituted heterocycloalkyl. In embodiments, R¹⁴ is independently substituted or unsubstituted aryl. In embodiments, R¹⁴ is independently substituted or unsubstituted heteroaryl. In embodiments, R¹⁴ is independently substituted alkyl. In embodiments, R¹⁴ is independently substituted heteroalkyl. In embodiments, R¹⁴ is independently substituted cycloalkyl. In embodiments, R¹⁴ is independently substituted heterocycloalkyl. In embodiments, R¹⁴ is independently substituted aryl. In embodiments, R¹⁴ is independently substituted heteroaryl. In embodiments, R¹⁴ is independently unsubstituted alkyl. In embodiments, R¹⁴ is independently unsubstituted heteroalkyl. In embodiments, R¹⁴ is independently unsubstituted cycloalkyl. In embodiments, R¹⁴ is independently unsubstituted heterocycloalkyl. In embodiments, R¹⁴ is independently unsubstituted aryl. In embodiments, R¹⁴ is independently unsubstituted heteroaryl. In embodiments, R¹⁴ is independently substituted or unsubstituted C₁-C₄ alkyl. In embodiments, R¹⁴ is independently substituted or unsubstituted 2 to 4 membered heteroalkyl. In embodiments, R¹⁴ is independently substituted or unsubstituted C₃-C₆ cycloalkyl. In embodiments, R¹⁴ is independently substituted or unsubstituted 3 to 6 membered heterocycloalkyl. In embodiments, R¹⁴ is independently substituted or unsubstituted phenyl. In embodiments, R¹⁴ is independently substituted or unsubstituted 5 to 6 membered heteroaryl. In embodiments, R¹⁴ is independently substituted C₁-C₄ alkyl. In embodiments, R¹⁴ is independently substituted 2 to 4 membered heteroalkyl. In embodiments, R¹⁴ is independently substituted C₃-C₆ cycloalkyl. In embodiments, R¹⁴ is independently substituted 3 to 6 membered heterocycloalkyl. In embodiments, R¹⁴ is independently substituted phenyl. In embodiments, R¹⁴ is independently substituted 5 to 6 membered heteroaryl. In embodiments, R¹⁴ is independently unsubstituted C₁-C₄ alkyl. In embodiments, R¹⁴ is independently unsubstituted 2 to 4 membered heteroalkyl. In embodiments, R¹⁴ is independently unsubstituted C₃-C₆ cycloalkyl. In embodiments, R¹⁴ is independently unsubstituted 3 to 6 membered heterocycloalkyl. In embodiments, R¹⁴ is independently unsubstituted phenyl. In embodiments, R¹⁴ is independently unsubstituted 5 to 6 membered heteroaryl. In embodiments, R¹⁴ is hydrogen or unsubstituted methyl.

In embodiments, R¹⁴ is substituted or unsubstituted pyrazolyl. In embodiments, R¹⁴ is substituted or unsubstituted pyridyl. In embodiments, R¹⁴ is substituted or unsubstituted imidazolyl. In embodiments, R¹⁴ is substituted or unsubstituted oxazolyl. In embodiments, R¹⁴ is substituted or unsubstituted isoxazolyl. In embodiments, R¹⁴ is substituted or unsubstituted thiazolyl. In embodiments, R¹⁴ is substituted or unsubstituted furanyl. In embodiments, R¹⁴ is substituted or unsubstituted pyrrolyl. In embodiments, R¹⁴ is substituted or unsubstituted thienyl. In embodiments, R¹⁴ is substituted pyrazolyl. In embodiments, R¹⁴ is substituted pyridyl. In embodiments, R¹⁴ is substituted imidazolyl. In embodiments, R¹⁴ is substituted oxazolyl. In embodiments, R¹⁴ is substituted isoxazolyl. In embodiments, R¹⁴ is substituted thiazolyl. In embodiments, R¹⁴ is substituted furanyl. In embodiments, R¹⁴ is substituted pyrrolyl. In embodiments, R¹⁴ is substituted thienyl. In embodiments, R¹⁴ is unsubstituted pyrazolyl. In embodiments, R¹⁴ is unsubstituted pyridyl. In embodiments, R¹⁴ is unsubstituted imidazolyl. In embodiments, R¹⁴ is unsubstituted oxazolyl. In embodiments, R¹⁴ is unsubstituted isoxazolyl. In embodiments, R¹⁴ is unsubstituted thiazolyl. In embodiments, R¹⁴ is unsubstituted furanyl. In embodiments, R¹⁴ is unsubstituted pyrrolyl. In embodiments, R¹⁴ is unsubstituted thienyl.

In embodiments, R¹⁴ is independently hydrogen or unsubstituted alkyl. In embodiments, R¹⁴ is independently hydrogen or unsubstituted C₁-C₆ alkyl. In embodiments, R¹⁴ is independently hydrogen or unsubstituted C₁-C₅ alkyl. In embodiments, R¹⁴ is independently hydrogen or unsubstituted C₁-C₄ alkyl. In embodiments, R¹⁴ is independently hydrogen or unsubstituted C₁-C₃ alkyl. In embodiments, R¹⁴ is independently hydrogen or unsubstituted C₁-C₂ alkyl. In embodiments, R¹⁴ is independently hydrogen or unsubstituted C₂-C₆ alkyl. In embodiments, R¹⁴ is independently hydrogen or unsubstituted C₂-C₅ alkyl. In embodiments, R¹⁴ is independently hydrogen or unsubstituted C₂-C₄ alkyl. In embodiments, R¹⁴ is independently hydrogen or unsubstituted C₂-C₃ alkyl. In embodiments, R¹⁴ is independently hydrogen or unsubstituted C₃-C₆ alkyl. In embodiments, R¹⁴ is independently hydrogen or unsubstituted C₄-C₆ alkyl. In embodiments, R¹⁴ is independently hydrogen or unsubstituted C₅-C₆ alkyl. In embodiments, R¹⁴ is independently hydrogen. In embodiments, R¹⁴ is independently unsubstituted alkyl. In embodiments, R¹⁴ is independently unsubstituted C₁-C₆ alkyl. In embodiments, R¹⁴ is independently unsubstituted C₁-C₅ alkyl. In embodiments, R¹⁴ is independently unsubstituted C₁-C₄ alkyl. In embodiments, R¹⁴ is independently unsubstituted C₁-C₃ alkyl. In embodiments, R¹⁴ is independently unsubstituted C₁-C₂ alkyl. In embodiments, R¹⁴ is independently unsubstituted C₂-C₆ alkyl. In embodiments, R¹⁴ is independently unsubstituted C₂-C₅ alkyl. In embodiments, R¹⁴ is independently unsubstituted C₂-C₄ alkyl. In embodiments, R¹⁴ is independently unsubstituted C₂-C₃ alkyl. In embodiments, R¹⁴ is independently unsubstituted C₃-C₆ alkyl. In embodiments, R¹⁴ is independently unsubstituted C₄-C₆ alkyl. In embodiments, R¹⁴ is independently unsubstituted C₅-C₆ alkyl. In embodiments, R¹⁴ is independently —CF₃. In embodiments, R¹⁴ is independently —CHF₂. In embodiments, R¹⁴ is independently —CH₂F. In embodiments, R¹⁴ is independently —CCl₃. In embodiments, R¹⁴ is independently —CHCl₂. In embodiments, R¹⁴ is independently —CH₂Cl. In embodiments, R¹⁴ is independently —CBr₃. In embodiments, R¹⁴ is independently —CHBr₂. In embodiments, R¹⁴ is independently —CH₂Br. In embodiments, R¹⁴ is independently —CI₃. In embodiments, R¹⁴ is independently —CHI₂. In embodiments, R¹⁴ is independently —CH₂I. In embodiments, R¹⁴ is independently unsubstituted C₁-C₄ haloalkyl. In embodiments, R¹⁴ is independently unsubstituted C₁-C₃ haloalkyl. In embodiments, R¹⁴ is independently unsubstituted C₁-C₂ haloalkyl. In embodiments, R¹⁴ is independently unsubstituted C₂-C₆ haloalkyl. In embodiments, R¹⁴ is independently unsubstituted C₂-C₅ haloalkyl. In embodiments, R¹⁴ is independently unsubstituted C₂-C₄ haloalkyl. In embodiments, R¹⁴ is independently unsubstituted C₂-C₃ haloalkyl. In embodiments, R¹⁴ is independently unsubstituted methyl. In embodiments, R¹⁴ is independently unsubstituted ethyl. In embodiments, R¹⁴ is independently unsubstituted propyl. In embodiments, R¹⁴ is independently unsubstituted isopropyl. In embodiments, R¹⁴ is independently unsubstituted butyl. In embodiments, R¹⁴ is independently unsubstituted isobutyl. In embodiments, R¹⁴ is independently unsubstituted tert-butyl.

In embodiments, z2 is 1. In embodiments, z2 is 0. In embodiments, z2 is 2. In embodiments, z2 is 3. In embodiments, z2 is 4. In embodiments, z2 is 5.

In embodiments, R⁵ is independently halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈cycloalkyl, substituted or unsubstituted 3 to 8 membered heterocycloalkyl, substituted or unsubstituted C₆-C₁₀ aryl, or substituted or unsubstituted 5 to 10 membered heteroaryl. In embodiments, R⁵ is independently halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl or substituted or unsubstituted 2 to 8 membered heteroalkyl.

In embodiments, R⁵ is independently halogen, —CF₃, —OH, —NH₂, —SH, substituted or unsubstituted C₁-C₄ alkyl, substituted or unsubstituted 2 to 4 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R⁵ is independently halogen, —OH, —NH₂, —SH, unsubstituted C₁-C₄ alkyl, or unsubstituted 2 to 4 membered heteroalkyl. In embodiments, R⁵ is independently halogen, —OH, unsubstituted methyl, or unsubstituted methoxy. In embodiments, R⁵ is independently halogen. In embodiments, R⁵ is independently —OH. In embodiments, R⁵ is independently unsubstituted methyl. In embodiments, R⁵ is independently unsubstituted methoxy. In embodiments, R⁵ is independently unsubstituted ethyl. In embodiments, R⁵ is independently —F. In embodiments, R⁵ is independently —Cl. In embodiments, R⁵ is independently —Br. In embodiments, R⁵ is independently —I. In embodiments, R⁵ is independently —CF₃. In embodiments, R⁵ is independently —NH₂. In embodiments, R⁵ is independently —SH. In embodiments, R⁵ is independently unsubstituted isopropyl. In embodiments, R⁵ is independently unsubstituted tert-butyl. In embodiments, R⁵ is independently unsubstituted ethoxy. In embodiments, R⁵ is independently unsubstituted propoxy.

In embodiments, R⁵ is independently substituted or unsubstituted alkyl (e.g. C₁-C₈ alkyl, C₁-C₆ alkyl, or C₁-C₄ alkyl), substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), substituted or unsubstituted cycloalkyl (e.g. C₃-C₈ cycloalkyl, C₄-C₈ cycloalkyl, or C₅-C₆ cycloalkyl), substituted or unsubstituted heterocycloalkyl (e.g. 3 to 8 membered heterocycloalkyl, 4 to 8 membered heterocycloalkyl, or 5 to 6 membered heterocycloalkyl), substituted or unsubstituted aryl (e.g. C₆-C₁₀ aryl or C₆ aryl), or substituted or unsubstituted heteroaryl (e.g. 5 to 10 membered heteroaryl, 5 to 9 membered heteroaryl, or 5 to 6 membered heteroaryl). In embodiments, R⁵ is independently substituted alkyl (e.g. C₁-C₈ alkyl, C₁-C₆ alkyl, or C₁-C₄ alkyl), substituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), substituted cycloalkyl (e.g. C₃-C₈ cycloalkyl, C₄-C₈ cycloalkyl, or C₅-C₆ cycloalkyl), substituted heterocycloalkyl (e.g. 3 to 8 membered heterocycloalkyl, 4 to 8 membered heterocycloalkyl, or 5 to 6 membered heterocycloalkyl), substituted aryl (e.g. C₆-C₁₀ aryl or C₆ aryl), or substituted heteroaryl (e.g. 5 to 10 membered heteroaryl, 5 to 9 membered heteroaryl, or 5 to 6 membered heteroaryl). In embodiments, R⁵ is independently unsubstituted alkyl (e.g. C₁-C₈ alkyl, C₁-C₆ alkyl, or C₁-C₄ alkyl), unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), unsubstituted cycloalkyl (e.g. C₃-C₈ cycloalkyl, C₄-C₈ cycloalkyl, or C₅-C₆ cycloalkyl), unsubstituted heterocycloalkyl (e.g. 3 to 8 membered heterocycloalkyl, 4 to 8 membered heterocycloalkyl, or 5 to 6 membered heterocycloalkyl), unsubstituted aryl (e.g. C₆-C₁₀ aryl or C₆ aryl), or unsubstituted heteroaryl (e.g. 5 to 10 membered heteroaryl, 5 to 9 membered heteroaryl, or 5 to 6 membered heteroaryl). In embodiments, R⁵ is independently halogen, —CF₃, —CHF₂, —CH₂F, —OCF₃, —OCHF₂, —OCH₂F, —OH, —NH₂, —SH, unsubstituted C₁-C₄ alkyl, or unsubstituted 2 to 4 membered heteroalkyl. In embodiments, R⁵ is unsubstituted C₁-C₄ alkyl, or unsubstituted 2 to 4 membered heteroalkyl. In embodiments, R⁵ is unsubstituted C₁-C₄ alkyl. In embodiments, R⁵ is unsubstituted 2 to 4 membered heteroalkyl.

In embodiments, R⁵ is independently unsubstituted alkyl. In embodiments, R⁵ is independently unsubstituted C₁-C₆ alkyl. In embodiments, R⁵ is independently unsubstituted C₁-C₅ alkyl. In embodiments, R⁵ is independently unsubstituted C₁-C₄ alkyl. In embodiments, R⁵ is independently unsubstituted C₁-C₃ alkyl. In embodiments, R⁵ is independently unsubstituted C₁-C₂ alkyl. In embodiments, R⁵ is independently unsubstituted C₂-C₆ alkyl. In embodiments, R⁵ is independently unsubstituted C₂-C₅ alkyl. In embodiments, R⁵ is independently unsubstituted C₂-C₄ alkyl. In embodiments, R⁵ is independently unsubstituted C₂-C₃ alkyl. In embodiments, R⁵ is independently unsubstituted C₃-C₆ alkyl. In embodiments, R⁵ is independently unsubstituted C₄-C₆ alkyl. In embodiments, R⁵ is independently unsubstituted C₅-C₆ alkyl.

In embodiments, z3 is 1. In embodiments, z3 is 0. In embodiments, z3 is 2. In embodiments, z3 is 3. In embodiments, z3 is 4. In embodiments, z3 is 5. In embodiments, z3 is 6. In embodiments, z3 is 7.

In embodiments, R¹¹, R¹², R¹³ or R¹⁴ is independently hydrogen, —CX^{B}₃, —CHX^{B}₂, —CH₂X^{B}, —CN, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R¹¹, R¹², R¹³, or R¹⁴ is independently hydrogen. In embodiments, R¹¹, R¹², R¹³ or R¹⁴ is independently —CX^{B}3. In embodiments, R¹¹, R¹², R¹³ or R¹⁴ is independently —CHX^{B}2. In embodiments, R¹¹, R¹², R¹³, or R¹⁴ is independently —CH₂X^{B}. In embodiments, R¹¹, R¹², R¹³ or R¹⁴ is independently —CN. In embodiments, R¹¹, R¹², R¹³ or R¹⁴ is independently —COOH. In embodiments, R¹¹, R¹², R¹³, or R¹⁴ is independently —CONH₂. In embodiments, R¹¹, R¹², R¹³ or R¹⁴ is independently substituted or unsubstituted alkyl. In embodiments, R¹¹, R¹², R¹³ or R¹⁴ is independently substituted or unsubstituted heteroalkyl. In embodiments, R¹¹, R¹², R¹³ or R¹⁴ is independently substituted or unsubstituted cycloalkyl. In embodiments, R¹¹, R¹², R¹³ or R¹⁴ is independently substituted or unsubstituted heterocycloalkyl. In embodiments, R¹¹, R¹², R¹³ or R¹⁴ is independently substituted or unsubstituted aryl. In embodiments, R¹¹, Rlz R¹³ or R¹⁴ is independently substituted or unsubstituted heteroaryl. In embodiments, R¹¹, Rlz R¹³ or R¹⁴ is independently substituted alkyl. In embodiments, R¹¹, R¹², R¹³ or R¹⁴ is independently substituted heteroalkyl. In embodiments, R¹¹, R¹², R¹³ or R¹⁴ is independently substituted cycloalkyl. In embodiments, R¹¹, R¹², R¹³ or R¹⁴ is independently substituted heterocycloalkyl. In embodiments, R¹¹, R¹², R¹³, or R¹⁴ is independently substituted aryl. In embodiments, R¹¹, R¹², R¹³, or R¹⁴ is independently substituted heteroaryl. In embodiments, R¹¹, R¹², R¹³ or R¹⁴ is independently unsubstituted alkyl. In embodiments, R¹¹, R¹², R¹³ or R¹⁴ is independently unsubstituted heteroalkyl. In embodiments, R¹¹, R¹², R¹³ or R¹⁴ is independently unsubstituted cycloalkyl. In embodiments, R¹¹, R¹², R¹³, or R¹⁴ is independently unsubstituted heterocycloalkyl. In embodiments, R¹¹, R¹², R¹³ or R¹⁴ is independently unsubstituted aryl. In embodiments, R¹¹, R¹², R¹³, or R¹⁴ is independently unsubstituted heteroaryl. In embodiments, R¹¹, R¹², R¹³, or R¹⁴ is independently substituted or unsubstituted C₁-C₄ alkyl. In embodiments, R¹¹, R¹², R¹³, or R¹⁴ is independently substituted or unsubstituted 2 to 4 membered heteroalkyl. In embodiments, R¹¹, R¹², R¹³, or R¹⁴ is independently substituted or unsubstituted C₃-C₆ cycloalkyl. In embodiments, R¹¹, R¹², R¹³ or R¹⁴ is independently substituted or unsubstituted 3 to 6 membered heterocycloalkyl. In embodiments, R¹¹, R¹², R¹³ or R¹⁴ is independently substituted or unsubstituted phenyl. In embodiments, R¹¹, R¹², R¹³ or R¹⁴ is independently substituted or unsubstituted 5 to 6 membered heteroaryl. In embodiments, R¹¹, R¹², R¹³ or R¹⁴ is independently substituted C₁-C₄ alkyl. In embodiments, R¹¹, R¹², R¹³ or R¹⁴ is independently substituted 2 to 4 membered heteroalkyl. In embodiments, R¹¹, R¹², R¹³ or R¹⁴ is independently substituted C₃-C₆ cycloalkyl. In embodiments, R¹¹, R¹², R¹³ or R¹⁴ is independently substituted 3 to 6 membered heterocycloalkyl. In embodiments, R¹¹, R¹², R¹³ or R¹⁴ is independently substituted phenyl. In embodiments, R¹¹, R¹², R¹³ or R¹⁴ is independently substituted 5 to 6 membered heteroaryl. In embodiments; R¹¹, R¹², R¹³ or R¹⁴ is independently unsubstituted C₁-C₄ alkyl. In embodiments, R¹¹, R¹², R¹³ or R¹⁴ is independently unsubstituted 2 to 4 membered heteroalkyl. In embodiments, R¹¹, R¹², R¹³ or R¹⁴ is independently unsubstituted C₃-C₆ cycloalkyl. In embodiments, R¹¹, R¹², R¹³ or R¹⁴ is independently unsubstituted 3 to 6 membered heterocycloalkyl. In embodiments, R¹¹, R¹², R¹³ or R¹⁴ is independently unsubstituted phenyl. In embodiments, R¹¹, R¹², R¹³ or R¹⁴ is independently unsubstituted 5 to 6 membered heteroaryl.

In embodiments, R¹¹ and R¹² substituents bonded to the same nitrogen atom may optionally be joined to form a substituted or unsubstituted heterocycloalkyl. In embodiments, R¹¹ and R¹² substituents bonded to the same nitrogen atom may optionally be joined to form a substituted or unsubstituted heteroaryl. In embodiments, R¹¹ and R¹² substituents bonded to the same nitrogen atom may optionally be joined to form a substituted heterocycloalkyl. In embodiments, R¹¹ and R¹² substituents bonded to the same nitrogen atom may optionally be joined to form a substituted heteroaryl. In embodiments, R¹¹ and R¹² substituents bonded to the same nitrogen atom may optionally be joined to form an unsubstituted heterocycloalkyl. In embodiments, R¹¹ and R¹² substituents bonded to the same nitrogen atom may optionally be joined to form an unsubstituted heteroaryl. In embodiments, R¹¹ and R¹² substituents bonded to the same nitrogen atom may optionally be joined to form a substituted or unsubstituted 3 to 6 membered heterocycloalkyl. In embodiments, R¹¹ and R¹² substituents bonded to the same nitrogen atom may optionally be joined to form a substituted or unsubstituted 5 to 6 membered heteroaryl. In embodiments, R¹¹ and R¹² substituents bonded to the same nitrogen atom may optionally be joined to form a substituted 3 to 6 membered heterocycloalkyl. In embodiments, R¹¹ and R¹² substituents bonded to the same nitrogen atom may optionally be joined to form a substituted 5 to 6 membered heteroaryl. In embodiments, R¹¹ and R¹² substituents bonded to the same nitrogen atom may optionally be joined to form an unsubstituted 3 to 6 membered heterocycloalkyl. In embodiments, R¹¹ and R¹² substituents bonded to the same nitrogen atom may optionally be joined to form an unsubstituted 5 to 6 membered heteroaryl.

In embodiments, R¹⁵, R¹⁶, R¹⁷, or R¹⁸ is independently hydrogen, —CX^{C}₃, —CHX^{C}₂, —CH₂X^{C}, —CN, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R¹⁵, R¹⁶, R¹⁷, or R¹⁸ is independently hydrogen. In embodiments, R¹⁵, R¹⁶, R¹⁷, or R¹⁸ is independently —CX^{C}₃. In embodiments, R¹⁵, R¹⁶, R¹⁷, or R¹⁸ is independently —CHX^{C}₂. In embodiments, R¹⁵, R¹⁶, R¹⁷, or R¹⁸ is independently —CH₂X^{C}. In embodiments, R¹⁵, R¹⁶, R¹⁷, or R¹⁸ is independently —CN. In embodiments, R¹⁵, R¹⁶, R¹⁷, or R¹⁸ is independently —COOH. In embodiments, R¹⁵, R¹⁶, R¹⁷, or R¹⁸ is independently —CONH₂. In embodiments, R¹⁵, R¹⁶, R¹⁷, or R¹⁸ is independently substituted or unsubstituted alkyl. In embodiments, R¹⁵, R¹⁶, R¹⁷, or R¹⁸ is independently substituted or unsubstituted heteroalkyl. In embodiments, R¹⁵, R¹⁶, R¹⁷, or R¹⁸ is independently substituted or unsubstituted cycloalkyl. In embodiments, R¹⁵, R¹⁶, R¹⁷, or R¹⁸ is independently substituted or unsubstituted heterocycloalkyl. In embodiments, R¹⁵, R¹⁶, R¹⁷, or Rig is independently substituted or unsubstituted aryl. In embodiments, R's R¹⁶, R¹⁷, or Rig is independently substituted or unsubstituted heteroaryl. In embodiments, R's R¹⁶, R¹⁷, or R¹⁸ is independently substituted alkyl. In embodiments, R¹⁵, R¹⁶, R¹⁷, or R¹⁸ is independently substituted heteroalkyl. In embodiments, R¹⁵, R¹⁶, R¹⁷, or R¹⁸ is independently substituted cycloalkyl. In embodiments, R¹⁵,R¹⁶ R¹⁷, or R¹⁸ is independently substituted heterocycloalkyl. In embodiments, R¹⁵, R¹⁶, R¹⁷, or R¹⁸ is independently substituted aryl. In embodiments, R¹⁵, R¹⁶, R¹⁷, or R¹⁸ is independently substituted heteroaryl. In embodiments, R¹⁵, R¹⁶, R¹⁷, or R¹⁸ is independently unsubstituted alkyl. In embodiments, R¹⁵, R¹⁶, R¹⁷, or R¹⁸ is independently unsubstituted heteroalkyl. In embodiments, R¹⁵, R¹⁶, R¹⁷, or R¹⁸ is independently unsubstituted cycloalkyl. In embodiments, R¹⁵, R¹⁶, R¹⁷, or R¹⁸ is independently unsubstituted heterocycloalkyl. In embodiments, R¹⁵, R¹⁶, R¹⁷, or Rig is independently unsubstituted aryl. In embodiments, R¹⁵, R¹⁶, R¹⁷, or R¹⁸ is independently unsubstituted heteroaryl. In embodiments, R¹⁵, R¹⁶, R¹⁷, or R¹⁸ is independently substituted or unsubstituted C₁-C₄ alkyl. In embodiments, R¹⁵, R¹⁶, R¹⁷, or R¹⁸ is independently substituted or unsubstituted 2 to 4 membered heteroalkyl. In embodiments, R¹⁵, R¹⁶, R¹⁷, or R¹⁸ is independently substituted or unsubstituted C₃-C₆ cycloalkyl. In embodiments, R¹⁵, R¹⁶, R¹⁷, or R¹⁸ is independently substituted or unsubstituted 3 to 6 membered heterocycloalkyl. In embodiments, R¹⁵, R¹⁶, R¹⁷, or R¹⁸ is independently substituted or unsubstituted phenyl. In embodiments, R¹⁵, R¹⁶, R¹⁷, or R¹⁸ is independently substituted or unsubstituted 5 to 6 membered heteroaryl. In embodiments, R¹⁵, R¹⁶, R¹⁷, or R¹⁸ is independently substituted C₁-C₄ alkyl. In embodiments, R¹⁵, R¹⁶, R¹⁷, or R¹⁸ is independently substituted 2 to 4 membered heteroalkyl. In embodiments, R¹⁵, R¹⁶, R¹⁷, or R¹⁸ is independently substituted C₃-C₆ cycloalkyl. In embodiments, R¹⁵, R¹⁶, R¹⁷, or R¹⁸ is independently substituted 3 to 6 membered heterocycloalkyl. In embodiments, R¹⁵, R¹⁶, R¹⁷, or R¹⁸ is independently substituted phenyl. In embodiments, R¹⁵, R¹⁶, R¹⁷, or R¹⁸ is independently substituted 5 to 6 membered heteroaryl. In embodiments, R¹⁵, R¹⁶, R¹⁷, or R¹⁸ is independently unsubstituted C₁-C₄ alkyl. In embodiments, R¹⁵, R¹⁶, R¹⁷, or R¹⁸ is independently unsubstituted 2 to 4 membered heteroalkyl. In embodiments, R¹⁵, R¹⁶, R¹⁷, or R¹⁸ is independently unsubstituted C₃-C₆ cycloalkyl. In embodiments, R¹⁵, R¹⁶, R¹⁷, or R¹⁸ is independently unsubstituted 3 to 6 membered heterocycloalkyl. In embodiments, R¹⁵, R¹⁶, R¹⁷, or R¹⁸ is independently unsubstituted phenyl. In embodiments, R¹⁵, R¹⁶, R¹⁷, or R¹⁸ is independently unsubstituted 5 to 6 membered heteroaryl.

In embodiments, R¹⁵ and R¹⁶ substituents bonded to the same nitrogen atom may optionally be joined to form a substituted or unsubstituted heterocycloalkyl. In embodiments, R¹⁵ and R¹⁶ substituents bonded to the same nitrogen atom may optionally be joined to form a substituted or unsubstituted heteroaryl. In embodiments, R¹⁵ and R¹⁶ substituents bonded to the same nitrogen atom may optionally be joined to form a substituted heterocycloalkyl. In embodiments, R¹⁵ and R¹⁶ substituents bonded to the same nitrogen atom may optionally be joined to form a substituted heteroaryl. In embodiments, R¹⁵ and R¹⁶ substituents bonded to the same nitrogen atom may optionally be joined to form an unsubstituted heterocycloalkyl. In embodiments, R¹⁵ and R¹⁶ substituents bonded to the same nitrogen atom may optionally be joined to form an unsubstituted heteroaryl. In embodiments, R¹⁵ and R¹⁶ substituents bonded to the same nitrogen atom may optionally be joined to form a substituted or unsubstituted 3 to 6 membered heterocycloalkyl. In embodiments, R¹⁵ and R¹⁶ substituents bonded to the same nitrogen atom may optionally be joined to form a substituted or unsubstituted 5 to 6 membered heteroaryl. In embodiments, R¹⁵ and R¹⁶ substituents bonded to the same nitrogen atom may optionally be joined to form a substituted 3 to 6 membered heterocycloalkyl. In embodiments, R¹⁵ and R¹⁶ substituents bonded to the same nitrogen atom may optionally be joined to form a substituted 5 to 6 membered heteroaryl. In embodiments, R¹⁵ and R¹⁶ substituents bonded to the same nitrogen atom may optionally be joined to form an unsubstituted 3 to 6 membered heterocycloalkyl. In embodiments, R¹⁵ and R¹⁶ substituents bonded to the same nitrogen atom may optionally be joined to form an unsubstituted 5 to 6 membered heteroaryl.

In embodiments, m1 is 1. In embodiments, m1 is 2. In embodiments, v1 is 1. In embodiments, v1 is 2. In embodiments, m4 is 1. In embodiments, m4 is 2. In embodiments; m5 is 1. In embodiments, m5 is 2. In embodiments, v4 is 1. In embodiments, v4 is 2. In embodiments, v5 is 1. In embodiments, v5 is 2. In embodiments, n1 is 0. In embodiments, n1 is 1. In embodiments, n1 is 2. In embodiments, n1 is 3. In embodiments, n1 is 4. In embodiments, n4 is 0. In embodiments, n4 is 1. In embodiments, n4 is 2. In embodiments, n4 is 3. In embodiments, n4 is 4. In embodiments, n5 is 0. In embodiments, n5 is 1. In embodiments, n5 is 2. In embodiments, n5 is 3. In embodiments, n5 is 4.

In embodiments, X¹ is independently —Cl. In embodiments, X¹ is independently —Br. In embodiments, X¹ is independently —I. In embodiments, X¹ is independently —F. In embodiments, X² is independently —Cl. In embodiments, X² is independently —Br. In embodiments, X² is independently —I. In embodiments, X² is independently —F. In embodiments, X³ is independently —Cl. In embodiments, X³ is independently —Br. In embodiments, X³ is independently —I. In embodiments, X³ is independently —F. In embodiments, X⁴ is independently —Cl. In embodiments, X⁴ is independently —Br. In embodiments, X⁴ is independently —I. In embodiments, X⁴ is independently —F. In embodiments, X⁵ is independently —Cl. In embodiments, X⁵ is independently —Br. In embodiments, X⁵ is independently —I. In embodiments, X⁵ is independently —F. In embodiments, X^{A} is independently —Cl. In embodiments, X^{A} is independently —Br. In embodiments, X^{A} is independently —I. In embodiments, X^{A} is independently —F. In embodiments, X^{B} is independently —Cl. In embodiments, X^{B} is independently —Br. In embodiments, X^{B} is independently —I. In embodiments, X^{B} is independently —F. In embodiments, X^{C} is independently —Cl. In embodiments, X^{C} is independently —Br. In embodiments, X^{C} is independently —I. In embodiments, X^{C} is independently —F.

In embodiments, the PCNS inhibitor is a compound having the formula:

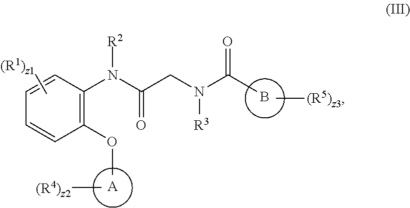

wherein R¹, R², R³, R⁴, R⁵, Ring A, Ring B, z1, z2, and z3 are as described herein, including in compounds of formula (I) and (II). In embodiments, z1 is 0. In embodiments, z2 is 0. In embodiments, z3 is 0. In embodiments, R² is hydrogen. In embodiments, R³ is hydrogen.

In embodiments, the PCNS inhibitor is a compound having the formula:

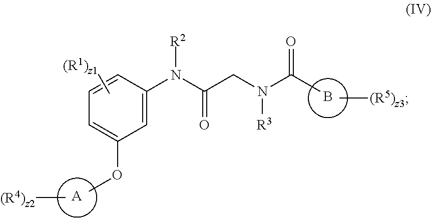

wherein R¹, R², R¹, R⁴, R⁵, Ring A, Ring B, z1, z2, and z3 are as described herein, including in compounds of formula (I) and (II).

In embodiments, the PCNS inhibitor is a compound having the formula:

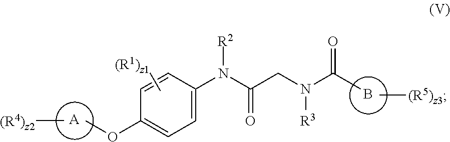

wherein R¹, R², R³, R⁴, R⁵, Ring A, Ring B, z1, z2, and z3 are as described herein, including in compounds of formula (I) and (II).

In embodiments, the PCNS inhibitor is a compound having the formula:

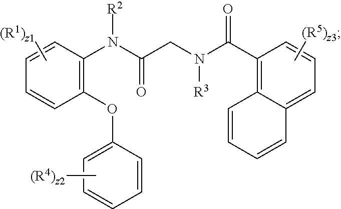

wherein R¹, R², R³, R⁴, R⁵, z1, z2, and z3 are as described herein, including in compounds of formula (I) to (V).

In embodiments, the PCNS inhibitor is a compound having the formula:

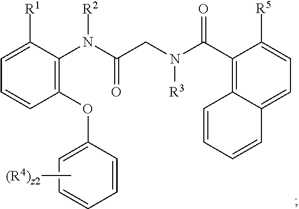

wherein R¹, R², R³, R⁴, R⁵, and z2 are as described herein, including in compounds of formula (I) to (V).

In embodiments, the PCNS inhibitor is a compound having the formula:

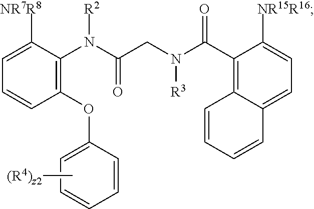

wherein R², R³, R⁴, R⁷, R⁸, R¹⁵, R¹⁶, and z2 are as described herein, including in compounds of formula (I) to (V).

In embodiments, the PCNS inhibitor is a compound having the formula:

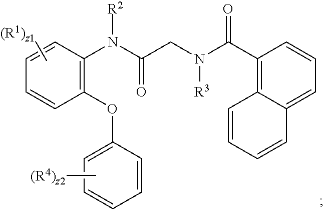

wherein R¹, R², R³, R⁴, z1, and z2 are as described herein, including in compounds of formula (I) to (V).

In embodiments, the PCNS inhibitor is a compound having the formula:

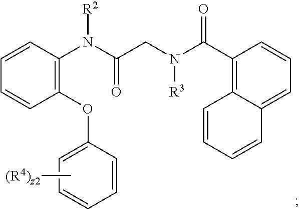

wherein R², R³, R⁴, and z2 are as described herein, including in compounds of formula (I) to (V).

In embodiments, the PCNS inhibitor is a compound having the formula:

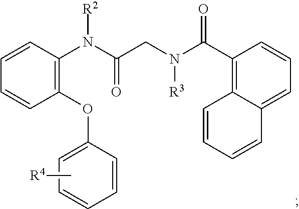

wherein R², R³, and R⁴ are as described herein, including in compounds of formula (I) to (V).

In embodiments, the PCNS inhibitor is a compound having the formula:

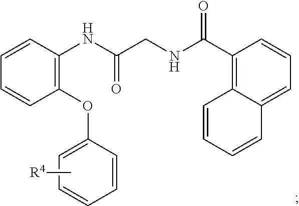

R⁴ is as described herein, including in compounds of formula (I) to (V). In embodiments, R⁴ is independently —OR¹⁴. In embodiments, R⁴ is independently —SR¹⁴. In embodiments, R¹⁴ is independently hydrogen or unsubstituted alkyl. In embodiments, R¹⁴ is independently hydrogen or unsubstituted C₁-C₆ alkyl. In embodiments, R¹⁴ is independently hydrogen or unsubstituted C₁-C₅ alkyl. In embodiments, R¹⁴ is independently hydrogen or unsubstituted C₁-C₄ alkyl. In embodiments, R¹⁴ is independently hydrogen or unsubstituted C₁-C₃ alkyl. In embodiments, R¹⁴ is independently hydrogen or unsubstituted C₁-C₂ alkyl. In embodiments, R¹⁴ is independently hydrogen or unsubstituted C₂-C₆ alkyl. In embodiments, R¹⁴ is independently hydrogen or unsubstituted C₂-C₅ alkyl. In embodiments, R¹⁴ is independently hydrogen or unsubstituted C₂-C₄ alkyl. In embodiments, R¹⁴ is independently hydrogen or unsubstituted C₂-C₃ alkyl. In embodiments, R¹⁴ is independently hydrogen or unsubstituted C₃-C₆ alkyl. In embodiments, R¹⁴ is independently hydrogen or unsubstituted C₄-C₆ alkyl. In embodiments, R¹⁴ is independently hydrogen or unsubstituted C₅-C₆ alkyl. In embodiments, R¹⁴ is independently hydrogen. In embodiments, R¹⁴ is independently unsubstituted alkyl. In embodiments, R¹⁴ is independently unsubstituted C₁-C₆ alkyl. In embodiments, R¹⁴ is independently unsubstituted C₁-C₅ alkyl. In embodiments, R¹⁴ is independently unsubstituted C₁-C₄ alkyl. In embodiments, R¹⁴ is independently unsubstituted C₁-C₃ alkyl. In embodiments, R¹⁴ is independently unsubstituted C₁-C₂ alkyl. In embodiments, R¹⁴ is independently unsubstituted C₂-C₆ alkyl. In embodiments, R¹⁴ is independently unsubstituted C₂-C₅ alkyl. In embodiments, R¹⁴ is independently unsubstituted C₂-C₄ alkyl. In embodiments, R¹⁴ is independently unsubstituted C₂-C₃ alkyl. In embodiments, R¹⁴ is independently unsubstituted C₃-C₆ alkyl. In embodiments, R¹⁴ is independently unsubstituted C₄-C₆ alkyl. In embodiments, R¹⁴ is independently unsubstituted C₅-C₆ alkyl. In embodiments, R¹⁴ is independently unsubstituted methyl. In embodiments, R¹⁴ is independently unsubstituted ethyl. In embodiments, R¹⁴ is independently unsubstituted propyl. In embodiments, R¹⁴ is independently unsubstituted isopropyl. In embodiments, R¹⁴ is independently unsubstituted tert-butyl.

In embodiments, the PCNS inhibitor is a compound having the formula:

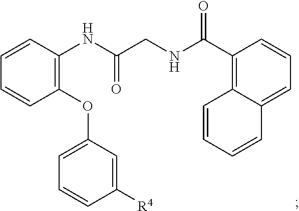

wherein R⁴ is as described herein, including in compounds of formula (I) to (V). In embodiments, R⁴ is independently —OR¹⁴. In embodiments, R⁴ is independently —SR¹⁴. In embodiments, R¹⁴ is independently hydrogen or unsubstituted alkyl. In embodiments, R¹⁴ is independently hydrogen or unsubstituted C₁-C₆ alkyl. In embodiments, R¹⁴ is independently hydrogen or unsubstituted C₁-C₅ alkyl. In embodiments, R¹⁴ is independently hydrogen or unsubstituted C₁-C₄ alkyl. In embodiments, R¹⁴ is independently hydrogen or unsubstituted C₁-C₃ alkyl. In embodiments, R¹⁴ is independently hydrogen or unsubstituted C₁-C₂ alkyl. In embodiments, R¹⁴ is independently hydrogen or unsubstituted C₂-C₆ alkyl. In embodiments, R¹⁴ is independently hydrogen or unsubstituted C₂-C₅ alkyl. In embodiments, R¹⁴ is independently hydrogen or unsubstituted C₂-C₄ alkyl. In embodiments, R¹⁴ is independently hydrogen or unsubstituted C₂-C₃ alkyl. In embodiments, R¹⁴ is independently hydrogen or unsubstituted C₃-C₆ alkyl. In embodiments, R¹⁴ is independently hydrogen or unsubstituted C₄-C₆ alkyl. In embodiments, R¹⁴ is independently hydrogen or unsubstituted C₅-C₆ alkyl. In embodiments, R¹⁴ is independently hydrogen. In embodiments, R¹⁴ is independently unsubstituted alkyl. In embodiments, R¹⁴ is independently unsubstituted C₁-C₆ alkyl. In embodiments, R¹⁴ is independently unsubstituted C₁-C₅ alkyl. In embodiments, R¹⁴ is independently unsubstituted C₁-C₄ alkyl. In embodiments, R¹⁴ is independently unsubstituted C₁-C₃ alkyl. In embodiments, R¹⁴ is independently unsubstituted C₁-C₂ alkyl. In embodiments, R¹⁴ is independently unsubstituted C₂-C₆ alkyl. In embodiments, R¹⁴ is independently unsubstituted C₂-C₅ alkyl. In embodiments, R¹⁴ is independently unsubstituted C₂-C₄ alkyl. In embodiments, R¹⁴ is independently unsubstituted C₂-C₃ alkyl. In embodiments, R¹⁴ is independently unsubstituted C₃-C₆ alkyl. In embodiments, R¹⁴ is independently unsubstituted C₄-C₆ alkyl. In embodiments, R¹⁴ is independently unsubstituted C₅-C₆ alkyl. In embodiments, R¹⁴ is independently unsubstituted methyl. In embodiments, R¹⁴ is independently unsubstituted ethyl. In embodiments, R¹⁴ is independently unsubstituted propyl. In embodiments, R¹⁴ is independently unsubstituted isopropyl. In embodiments, R¹⁴ is independently unsubstituted tert-butyl.

In embodiments, the PCNS inhibitor is a compound having the formula:

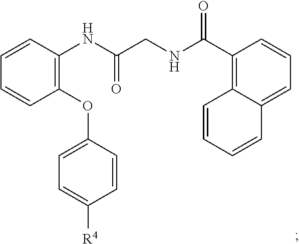

wherein R⁴ is as described herein, including in compounds of formula (I) to (V). In embodiments, R⁴ is independently —OR¹⁴. In embodiments, R⁴ is independently —SR¹⁴. In embodiments, R¹⁴ is independently hydrogen or unsubstituted alkyl. In embodiments, R¹⁴ is independently hydrogen or unsubstituted C₁-C₆ alkyl, In embodiments, R¹⁴ is independently hydrogen or unsubstituted C₁-C₅ alkyl. In embodiments, R¹⁴ is independently hydrogen or unsubstituted C₁-C₄ alkyl. In embodiments, R¹⁴ is independently hydrogen or unsubstituted C₁-C₃ alkyl. In embodiments, R¹⁴ is independently hydrogen or unsubstituted C₁-C₂ alkyl. In embodiments, R¹⁴ is independently hydrogen or unsubstituted C₂-C₆ alkyl. In embodiments, R¹⁴ is independently hydrogen or unsubstituted C₂-C₅ alkyl. In embodiments, R¹⁴ is independently hydrogen or unsubstituted C₂-C₄ alkyl. In embodiments, R¹⁴ is independently hydrogen or unsubstituted C₂-C₃ alkyl. In embodiments, R¹⁴ is independently hydrogen or unsubstituted C₃-C₆ alkyl. In embodiments, R¹⁴ is independently hydrogen or unsubstituted C₄-C₆ alkyl. In embodiments, R¹⁴ is independently hydrogen or unsubstituted C₅-C₆ alkyl. In embodiments, R¹⁴ is independently hydrogen. In embodiments, R¹⁴ is independently unsubstituted alkyl. In embodiments, R¹⁴ is independently unsubstituted C₁-C₆ alkyl. In embodiments, R¹⁴ is independently unsubstituted C₁-C₅ alkyl. In embodiments, R¹⁴ is independently unsubstituted C₁-C₄ alkyl. In embodiments, R¹⁴ is independently unsubstituted C₁-C₃ alkyl. In embodiments, R¹⁴ is independently unsubstituted C₁-C₂ alkyl. In embodiments, R¹⁴ is independently unsubstituted C₂-C₆ alkyl. In embodiments, R¹⁴ is independently unsubstituted C₂-C₅ alkyl. In embodiments, R¹⁴ is independently unsubstituted C₂-C₄ alkyl. In embodiments, R¹⁴ is independently unsubstituted C₂-C₃ alkyl. In embodiments, R¹⁴ is independently unsubstituted C₃-C₆ alkyl. In embodiments, R¹⁴ is independently unsubstituted C₄-C₆ alkyl. In embodiments, R¹⁴ is independently unsubstituted C₅-C₆ alkyl. In embodiments, R¹⁴ is independently unsubstituted methyl. In embodiments, R¹⁴ is independently unsubstituted ethyl. In embodiments, R¹⁴ is independently unsubstituted propyl. In embodiments, R¹⁴ is independently unsubstituted isopropyl. In embodiments, R¹⁴ is independently unsubstituted tert-butyl.

In embodiments, the PCNS inhibitor is a compound having the formula:

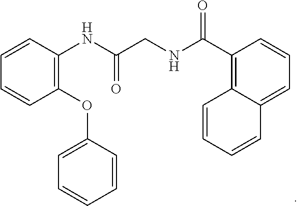 [SMILES: O=C(CNC(=O)c1cccc2ccccc12)Nc1ccccc1Oc1ccccc1]

In embodiments, the PCNS inhibitor is a compound having the formula:

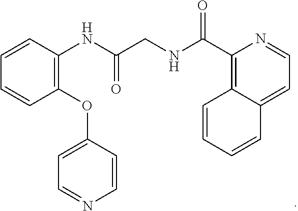 [SMILES: O=C(CNC(=O)c1nccc2ccccc12)Nc1ccccc1Oc1ccncc1]

In embodiments, the PCNS inhibitor is a compound having the formula:

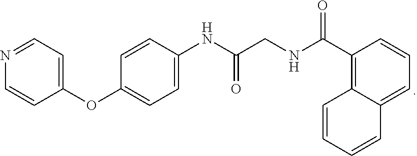 [SMILES: O=C(CNC(=O)c1cccc2ccccc12)Nc1ccc(Oc2ccncc2)cc1]

In embodiments, the PCNS inhibitor is a compound having the formula:

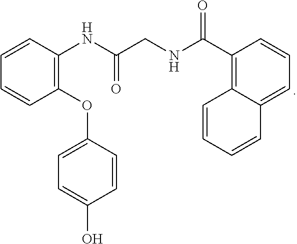 [SMILES: O=C(CNC(=O)c1cccc2ccccc12)Nc1ccccc1Oc1ccc(O)cc1]

In embodiments, the PCNS inhibitor is a compound having the formula:

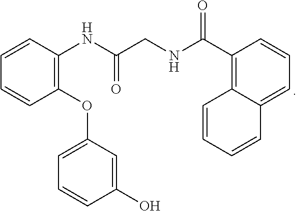 [SMILES: O=C(CNC(=O)c1cccc2ccccc12)Nc1ccccc1Oc1cccc(O)c1]

In embodiments, the PCNS inhibitor is a compound having the formula:

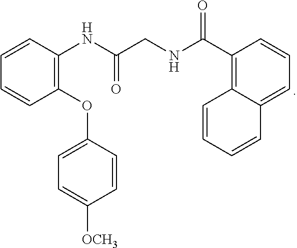 [SMILES: COc1ccc(Oc2ccccc2NC(=O)CNC(=O)c2cccc3ccccc23)cc1]

In embodiments, the PCNS inhibitor is a compound having the formula:

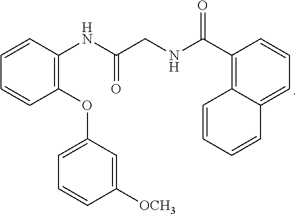 [SMILES: COc1cccc(Oc2ccccc2NC(=O)CNC(=O)c2cccc3ccccc23)c1]

In embodiments, R¹ is independently hydrogen, oxo, halogen, —CX¹₃, —CHX¹₂, —OCH₂X¹, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX¹₃, —OCHX¹₂, —OCH₂X¹, R³⁰-substituted or unsubstituted alkyl (e.g. C₁-C₈ alkyl, C₁-C₆ alkyl, or C₁-C₄ alkyl), R³⁰-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R³⁰-substituted or unsubstituted cycloalkyl (e.g. C₃-C₈ cycloalkyl, C₄-C₈ cycloalkyl, or C₅-C₆ cycloalkyl), R³⁰-substituted or unsubstituted heterocycloalkyl (e.g. 3 to 8 membered heterocycloalkyl, 4 to 8 membered heterocycloalkyl, or 5 to 6 membered heterocycloalkyl). R³⁰-substituted or unsubstituted aryl (e.g. C₆-C₁₀ aryl or C₆ aryl), or R³⁰-substituted or unsubstituted heteroaryl (e.g. 5 to 10 membered heteroaryl, 5 to 9 membered heteroaryl, or 5 to 6 membered heteroaryl). X¹ is halogen. In embodiments, X¹ is F. In embodiments, R¹ is independently halogen, —CX¹₃, —CHX¹₂, —OCH₂X¹, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX¹₃, —OCHX¹₂, —OCH₂X¹, R³⁰-substituted or unsubstituted alkyl (e.g. C₁-C₈ alkyl, C₁-C₆ alkyl, or C₁-C₄ alkyl), R³⁰-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R³⁰-substituted or unsubstituted cycloalkyl (e.g. C₃-C₈ cycloalkyl, C₄-C₈ cycloalkyl, or C₅-C₆ cycloalkyl), R³⁰-substituted or unsubstituted heterocycloalkyl (e.g. 3 to 8 membered heterocycloalkyl, 4 to 8 membered heterocycloalkyl, or 5 to 6 membered heterocycloalkyl). R³⁰-substituted or unsubstituted aryl (e.g. C₆-C₁₀ aryl or C₆ aryl), or R³⁰-substituted or unsubstituted heteroaryl (e.g. 5 to 10 membered heteroaryl, 5 to 9 membered heteroaryl, or 5 to 6 membered heteroaryl).

R³⁰ is independently oxo, halogen, —CX³⁰₃, —CHX³⁰₂, —CH₂X³⁰, —OCH₂X³⁰, —OCHX³⁰₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX³⁰₃, R³¹-substituted or unsubstituted alkyl (e.g. C₁-C₈ alkyl, C₁-C₆ alkyl, or C₁-C₄ alkyl), R³¹-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R³¹-substituted or unsubstituted cycloalkyl (e.g. C₃-C₈ cycloalkyl, C₄-C₈ cycloalkyl, or C₅-C₆ cycloalkyl), R³¹-substituted or unsubstituted heterocycloalkyl (e.g. 3 to 8 membered heterocycloalkyl, 4 to 8 membered heterocycloalkyl, or 5 to 6 membered heterocycloalkyl), R³¹-substituted or unsubstituted aryl (e.g. C₆-C₁₀ aryl or C₆ aryl), or R³¹-substituted or unsubstituted heteroaryl (e.g. 5 to 10 membered heteroaryl, 5 to 9 membered heteroaryl, or 5 to 6 membered heteroaryl). X³⁰ is halogen. In embodiments, X³⁰ is F.

R³¹ is independently oxo, halogen, —CX³¹₃, —CHX³¹₂, —CH₂X³¹, —OCH₂X³¹, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX³¹₃, —OCHX³¹₂, R³²-substituted or unsubstituted alkyl (e.g. C₁-C₈ alkyl, C₁-C₆ alkyl, or C₁-C₄ alkyl), R³²-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R³²-substituted or unsubstituted cycloalkyl (e.g. C₃-C₈ cycloalkyl, C₄-C₈ cycloalkyl, or C₅-C₆ cycloalkyl), R³²-substituted or unsubstituted heterocycloalkyl (e.g. 3 to 8 membered heterocycloalkyl, 4 to 8 membered heterocycloalkyl, or 5 to 6 membered heterocycloalkyl), R³²-substituted or unsubstituted aryl (e.g. C₆-C₁₀ aryl or C₆ aryl), or R³²-substituted or unsubstituted heteroaryl (e.g. 5 to 10 membered heteroaryl, 5 to 9 membered heteroaryl, or 5 to 6 membered heteroaryl). X³¹ is halogen. In embodiments, X³¹ is F.

In embodiments, R² is hydrogen, oxo, halogen, —CX²₃, —CHX²₂, —CH₂X², —OCH₂X², —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX²₃, —OCHX²₂, R³³-substituted or unsubstituted alkyl (e.g. C₁-C₈ alkyl, C₁-C₆ alkyl, or C₁-C₄ alkyl), R³³-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R³³-substituted or unsubstituted cycloalkyl (e.g. C₃-C₈ cycloalkyl, C₄-C₈ cycloalkyl, or C₅-C₆ cycloalkyl), R³³-substituted or unsubstituted heterocycloalkyl (e.g. 3 to 8 membered heterocycloalkyl, 4 to 8 membered heterocycloalkyl, or 5 to 6 membered heterocycloalkyl), R³³-substituted or unsubstituted aryl (e.g. C₆-C₁₀ aryl or C₆ aryl), or R³³-substituted or unsubstituted heteroaryl (e.g. 5 to 10 membered heteroaryl, 5 to 9 membered heteroaryl, or 5 to 6 membered heteroaryl). X² is halogen. In embodiments, X² is F.

In embodiments, R² is halogen, —CX²₃, —CHX²₂, —OCH₂X², —CH₂X², —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX²₃, —OCHX²₂, R³³-substituted or unsubstituted alkyl (e.g. C₁-C₈ alkyl, C₁-C₆ alkyl, or C₁-C₄ alkyl), R³³-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R³³-substituted or unsubstituted cycloalkyl (e.g. C₃-C₈ cycloalkyl, C₄-C₈ cycloalkyl, or C₅-C₆ cycloalkyl), R³³-substituted or unsubstituted heterocycloalkyl (e.g. 3 to 8 membered heterocycloalkyl, 4 to 8 membered heterocycloalkyl, or 5 to 6 membered heterocycloalkyl), R³³-substituted or unsubstituted aryl (e.g. C₆-C₁₀ aryl or C₆ aryl), or R³³-substituted or unsubstituted heteroaryl (e.g. 5 to 10 membered heteroaryl, 5 to 9 membered heteroaryl, or 5 to 6 membered heteroaryl). In embodiments, R² is hydrogen.

R³³ is independently oxo, halogen, —CX³³₃, —CHX³³₂, —CHX³³₂, —OCH₂X³³, —OCHX³³₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX³³₃, —OCHX³³₂, R³⁴-substituted or unsubstituted alkyl (e.g. C₁-C₈ alkyl, C₁-C₆ alkyl, or C₁-C₄ alkyl), R³⁴-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R³⁴-substituted or unsubstituted cycloalkyl (e.g. C₃-C₈ cycloalkyl, C₄-C₈ cycloalkyl, or C₅-C₆ cycloalkyl), R³⁴-substituted or unsubstituted heterocycloalkyl (e.g. 3 to 8 membered heterocycloalkyl, 4 to 8 membered heterocycloalkyl, or 5 to 6 membered heterocycloalkyl), R³⁴-substituted or unsubstituted aryl (e.g. C₆-C₁₀ aryl or C₆ aryl), or R³⁴-substituted or unsubstituted heteroaryl (e.g. 5 to 10 membered heteroaryl, 5 to 9 membered heteroaryl, or 5 to 6 membered heteroaryl). X³³ is halogen. In embodiments, X³³ is F.

R³⁴ is independently oxo, halogen, —CX³⁴₃, —CHX³⁴₂, —CH₂X³⁴₂, —OCH₂X³⁴, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX³⁴₃, —OCHX³⁴₂, R³⁵-substituted or unsubstituted alkyl (e.g. C₁-C₈ alkyl, C₁-C₆ alkyl, or C₁-C₄ alkyl), R³⁵-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R³⁵-substituted or unsubstituted cycloalkyl (e.g. C₃-C₈ cycloalkyl, C₄-C₈ cycloalkyl, or C₅-C₆ cycloalkyl), R³⁵-substituted or unsubstituted heterocycloalkyl (e.g. 3 to 8 membered heterocycloalkyl, 4 to 8 membered heterocycloalkyl, or 5 to 6 membered heterocycloalkyl), R³⁵-substituted or unsubstituted aryl (e.g. C₆-C₁₀ aryl or C₆ aryl), or R³⁵-substituted or unsubstituted heteroaryl (e.g. 5 to 10 membered heteroaryl, 5 to 9 membered heteroaryl, or 5 to 6 membered heteroaryl). X³⁴ is halogen. In embodiments, X³⁴ is F.

In embodiments, R³ is hydrogen, oxo, halogen, —CX³₃, —CHX³₂, —CH₂X³, —OCH₂X³, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX³₃, —OCHX³₂, R³⁶-substituted or unsubstituted alkyl (e.g. C₁-C₈ alkyl, C₁-C₆ alkyl, or C₁-C₄ alkyl), R³⁶-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R³⁶-substituted or unsubstituted cycloalkyl (e.g. C₃-C₈ cycloalkyl, C₄-C₈ cycloalkyl, or C₅-C₆ cycloalkyl), R³⁶-substituted or unsubstituted heterocycloalkyl (e.g. 3 to 8 membered heterocycloalkyl, 4 to 8 membered heterocycloalkyl, or 5 to 6 membered heterocycloalkyl), R³⁶-substituted or unsubstituted aryl (e.g. C₆-C₁₀ aryl or C₆ aryl), or R³⁶-substituted or unsubstituted heteroaryl (e.g. 5 to 10 membered heteroaryl, 5 to 9 membered heteroaryl, or 5 to 6 membered heteroaryl). X³ is halogen. In embodiments, X³ is F. In embodiments, R³ is halogen, —CX³₃, —CHX³₂, —CH₂X³, —OCH₂X³, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX³₃, —OCHX³₂, R³⁶-substituted or unsubstituted alkyl (e.g. C₁-C₈ alkyl, C₁-C₆ alkyl, or C₁-C₄ alkyl), R³⁶-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R³⁶-substituted or unsubstituted cycloalkyl (e.g. C₃-C₈ cycloalkyl, C₄-C₈ cycloalkyl, or C₅-C₆ cycloalkyl), R³⁶-substituted or unsubstituted heterocycloalkyl (e.g. 3 to 8 membered heterocycloalkyl, 4 to 8 membered heterocycloalkyl, or 5 to 6 membered heterocycloalkyl), R³⁶-substituted or unsubstituted aryl (e.g. C₆-C₁₀ aryl or C₆ aryl), or R³⁶-substituted or unsubstituted heteroaryl (e.g. 5 to 10 membered heteroaryl, 5 to 9 membered heteroaryl, or 5 to 6 membered heteroaryl). In embodiments, R³ is hydrogen.

R³⁶ is independently oxo, halogen, —CX³⁶₃, —CHX³⁶₂, —CH₂X³⁶, —OCH₂X³⁶, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX³⁶₃, —OCHX³⁶₂, R³⁷-substituted or unsubstituted alkyl (e.g. C₁-C₈ alkyl, C₁-C₆ alkyl, or C₁-C₄ alkyl), R³⁷-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R³⁷-substituted or unsubstituted cycloalkyl (e.g. C₃-C₈ cycloalkyl, C₄-C₈ cycloalkyl, or C₅-C₆ cycloalkyl), R³⁷-substituted or unsubstituted heterocycloalkyl (e.g. 3 to 8 membered heterocycloalkyl, 4 to 8 membered heterocycloalkyl, or 5 to 6 membered heterocycloalkyl), R³⁷-substituted or unsubstituted aryl (e.g. C₆-C₁₀ aryl or C₆ aryl), or R³⁷-substituted or unsubstituted heteroaryl (e.g. 5 to 10 membered heteroaryl, 5 to 9 membered heteroaryl, or 5 to 6 membered heteroaryl). X³⁶ is halogen. In embodiments, X³⁶ is F.

R³⁷ is independently oxo, halogen, —CX³⁷₃, —CHX³⁷₂, —CH₂X³⁷, —OCH₂X³⁷, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX³⁷3, —OCHX³⁷₂, R³⁸-substituted or unsubstituted alkyl (e.g. C₁-C₈ alkyl, C₁-C₆ alkyl, or C₁-C₄ alkyl), R³⁸-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R³⁸-substituted or unsubstituted cycloalkyl (e.g. C₃-C₈ cycloalkyl, C₄-C₈ cycloalkyl, or C₅-C₆ cycloalkyl), R³⁸-substituted or unsubstituted heterocycloalkyl (e.g. 3 to 8 membered heterocycloalkyl, 4 to 8 membered heterocycloalkyl, or 5 to 6 membered heterocycloalkyl), R³⁸-substituted or unsubstituted aryl (e.g. C₆-C₁₀ aryl or C₆ aryl), or R³⁸-substituted or unsubstituted heteroaryl (e.g. 5 to 10 membered heteroaryl, 5 to 9 membered heteroaryl, or 5 to 6 membered heteroaryl). X³⁷ is halogen. In embodiments, X³⁷ is F.

In embodiments, R⁴ is independently hydrogen, oxo, halogen, —CX⁴₃, —CHX⁴₂, —CH₂X⁴, —OCH₂X⁴, —CN, —OH, —NH₂, —COOH, —CONH₂; —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁴₃, —OCHX⁴₂, R³⁹-substituted or unsubstituted alkyl (e.g. C₁-C₈ alkyl, C₁-C₆ alkyl, or C₁-C₄ alkyl), R³⁹-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R³⁹-substituted or unsubstituted cycloalkyl (e.g. C₃-C₈ cycloalkyl, C₄-C₈ cycloalkyl, or C₅-C₆ cycloalkyl), R³⁹-substituted or unsubstituted heterocycloalkyl (e.g. 3 to 8 membered heterocycloalkyl, 4 to 8 membered heterocycloalkyl, or 5 to 6 membered heterocycloalkyl), R³⁹-substituted or unsubstituted aryl (e.g. C₆-C₁₀ aryl or C₆ aryl), or R³⁹-substituted or unsubstituted heteroaryl (e.g. 5 to 10 membered heteroaryl, 5 to 9 membered heteroaryl, or 5 to 6 membered heteroaryl). X⁴ is halogen. In embodiments, X⁴ is F. In embodiments, R⁴ is independently halogen, —CX⁴₃, —CHX⁴₂, —CH₂X⁴, —OCH₂X⁴, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁴₃, —OCHX⁴₂, R³⁹-substituted or unsubstituted alkyl (e.g. C₁-C₈ alkyl, C₁-C₆ alkyl, or C₁-C₄ alkyl), R³⁹-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R³⁹-substituted or unsubstituted cycloalkyl (e.g. C₃-C₈ cycloalkyl, C₄-C₈ cycloalkyl, or C₅-C₆ cycloalkyl), R³⁹-substituted or unsubstituted heterocycloalkyl (e.g. 3 to 8 membered heterocycloalkyl, 4 to 8 membered heterocycloalkyl, or 5 to 6 membered heterocycloalkyl), R³⁹-substituted or unsubstituted aryl (e.g. C₆-C₁₀ aryl or C₆ aryl), or R³⁹-substituted or unsubstituted heteroaryl (e.g. 5 to 10 membered heteroaryl, 5 to 9 membered heteroaryl, or 5 to 6 membered heteroaryl).

R³⁹ is independently oxo, halogen, —CX³⁹₃, —CHX³⁹₂, —CH₂X³⁹, —OCH₂X³⁹, —OCHX³⁹₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX³⁹₃, —OCHX³⁹₂, R⁴⁰-substituted or unsubstituted alkyl (e.g. C₁-C₈ alkyl, C₁-C₆ alkyl, or C₁-C₄ alkyl), R⁴⁰-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R⁴⁰-substituted or unsubstituted cycloalkyl (e.g. C₃-C₈ cycloalkyl, C₄-C₈ cycloalkyl, or C₅-C₆ cycloalkyl), R⁴⁰-substituted or unsubstituted heterocycloalkyl (e.g. 3 to 8 membered heterocycloalkyl, 4 to 8 membered heterocycloalkyl, or 5 to 6 membered heterocycloalkyl), R⁴⁰-substituted or unsubstituted aryl (e.g. C₆-C₁₀ aryl or C₆ aryl), or R⁴⁰-substituted or unsubstituted heteroaryl (e.g. 5 to 10 membered heteroaryl, 5 to 9 membered heteroaryl, or 5 to 6 membered heteroaryl). X³⁹ is halogen. In embodiments, X³⁹ is F.

R⁴⁰ is independently oxo, halogen, —CX⁴⁰₃, —CHX⁴⁰₂, —CH₂X⁴⁰, —OCH₂X⁴⁰, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁴⁰₃, —OCHX⁴⁰₂, R^{4.1}-substituted or unsubstituted alkyl (e.g. C₁-C₈ alkyl, C₁-C₆ alkyl, or C₁-C₄ alkyl), R^{4.1}-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R^{4.1}-substituted or unsubstituted cycloalkyl (e.g. C₃-C₈ cycloalkyl, C₄-C₈ cycloalkyl, or C₅-C₆ cycloalkyl), R⁴¹-substituted or unsubstituted heterocycloalkyl (e.g. 3 to 8 membered heterocycloalkyl, 4 to 8 membered heterocycloalkyl, or 5 to 6 membered heterocycloalkyl), R^{4.1}-substituted or unsubstituted aryl (e.g. C₆-C₁₀ aryl or C₆ aryl), or R^{4.1}-substituted or unsubstituted heteroaryl (e.g. 5 to 10 membered heteroaryl, 5 to 9 membered heteroaryl, or 5 to 6 membered heteroaryl). X⁴⁰ is halogen. In embodiments, X⁴⁰ is F.

In embodiments, R⁵ is independently hydrogen, oxo, halogen, —CX⁵₃, —CHX⁵₂, —CH₂X⁵, —OCH₂X⁵, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁵₃, —OCHX⁵₂, R^{4.2}-substituted or unsubstituted alkyl (e.g. C₈-C₈ alkyl, C₈-C₆ alkyl, or C₈-C₄ alkyl), R^{4.2}-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R^{4.2}-substituted or unsubstituted cycloalkyl (e.g. C₃-C₈ cycloalkyl, C₄-C₈ cycloalkyl, or C₅-C₆ cycloalkyl), R^{4.2}-substituted or unsubstituted heterocycloalkyl (e.g. 3 to 8 membered heterocycloalkyl, 4 to 8 membered heterocycloalkyl, or to 6 membered heterocycloalkyl). R^{4.2}-substituted or unsubstituted aryl (e.g. C₆-C₁₀ aryl or C₆ aryl), or R^{4.2}-substituted or unsubstituted heteroaryl (e.g. 5 to 10 membered heteroaryl, 5 to 9 membered heteroaryl, or 5 to 6 membered heteroaryl). X⁵ is halogen. In embodiments, X⁵ is F. In embodiments, R⁵ is independently halogen, —CX⁵₃, —CHX⁵₂, —CH₂X⁵, —OCH₂X⁵, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁵₃, —OCHX⁵₂, R^{4.2}-substituted or unsubstituted alkyl (e.g. C₁-C₈ alkyl, C₁-C₆ alkyl, or C₁-C₄ alkyl), R^{4.2}-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R^{4.2}-substituted or unsubstituted cycloalkyl (e.g. C₃-C₈ cycloalkyl, C₄-C₈ cycloalkyl, or C₅-C₆ cycloalkyl), R^{4.2}-substituted or unsubstituted heterocycloalkyl (e.g. 3 to 8 membered heterocycloalkyl, 4 to 8 membered heterocycloalkyl, or 5 to 6 membered heterocycloalkyl), R^{4.2}-substituted or unsubstituted aryl (e.g. C₆-C₁₀ aryl or C₆ aryl), or R^{4.2}-substituted or unsubstituted heteroaryl (e.g. 5 to 10 membered heteroaryl, 5 to 9 membered heteroaryl, or 5 to 6 membered heteroaryl).

R^{4.2} is independently oxo, halogen, —CX⁴²₃, —CHX⁴²₂, —CH₂X⁴², —OCH₂X⁴², —OCHX⁴²₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁴²₃, —OCHX⁴²₂, R^{4.3}-substituted or unsubstituted alkyl (e.g. C₁-C₈ alkyl, C₁-C₆ alkyl, or C₁-C₄ alkyl), R^{4.3}-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R^{4.3}-substituted or unsubstituted cycloalkyl (e.g. C₃-C₈ cycloalkyl, C₄-C₈ cycloalkyl, or C₅-C₆ cycloalkyl), R^{4.3}-substituted or unsubstituted heterocycloalkyl (e.g. 3 to 8 membered heterocycloalkyl, 4 to 8 membered heterocycloalkyl, or 5 to 6 membered heterocycloalkyl), R^{4.3}-substituted or unsubstituted aryl (e.g. C₆-C₁₀ aryl or C₆ aryl), or R^{4.3}-substituted or unsubstituted heteroaryl (e.g. 5 to 10 membered heteroaryl, 5 to 9 membered heteroaryl, or 5 to 6 membered heteroaryl). X⁴² is halogen. In embodiments, X⁴² is F.

R^{4.3} is independently oxo, halogen, —CX⁴³3, —CHX⁴³2, —CH₂X⁴³, —OCH₂X⁴³, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁴³₃, —OCHX⁴³₂, R^{4.4}-substituted or unsubstituted alkyl (e.g. C₁-C₈ alkyl, C₁-C₆ alkyl, or C₁-C₄ alkyl), R^{4.4}-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R^{4.4}-substituted or unsubstituted cycloalkyl (e.g. C₃-C₈ cycloalkyl, C₄-C₈ cycloalkyl, or C₅-C₆ cycloalkyl), R^{4.4}-substituted or unsubstituted heterocycloalkyl (e.g. 3 to 8 membered heterocycloalkyl, 4 to 8 membered heterocycloalkyl, or 5 to 6 membered heterocycloalkyl), R^{4.4}-substituted or unsubstituted aryl (e.g. C₆-C₁₀ aryl or C₆ aryl), or R^{4.4}-substituted or unsubstituted heteroaryl (e.g. 5 to 10 membered heteroaryl, 5 to 9 membered heteroaryl, or 5 to 6 membered heteroaryl). X⁴³ is halogen. In embodiments, X⁴³ is F.

In embodiments, R⁷ is independently hydrogen, oxo, halogen, —CX⁷₃, —CHX⁷₂, —CH₂X⁷, —OCH₂X⁷, —CN, —OH, —NH₂, —COOH, —CONH₂; —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁷₃, —OCHX⁷₂, R⁴⁸-substituted or unsubstituted alkyl (e.g. C₁-C₈ alkyl, C₁-C₆ alkyl, or C₁-C₄ alkyl), R⁴⁸-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R⁴⁸-substituted or unsubstituted cycloalkyl (e.g. C₃-C₈ cycloalkyl, C₄-C₈ cycloalkyl, or C₅-C₆ cycloalkyl), R⁴⁸-substituted or unsubstituted heterocycloalkyl (e.g. 3 to 8 membered heterocycloalkyl, 4 to 8 membered heterocycloalkyl, or 5 to 6 membered heterocycloalkyl), R⁴⁸-substituted or unsubstituted aryl (e.g. C₆-C₁₀ aryl or C₆ aryl), or R⁴⁸-substituted or unsubstituted heteroaryl (e.g. 5 to 10 membered heteroaryl, 5 to 9 membered heteroaryl, or 5 to 6 membered heteroaryl). X⁷ is halogen. In embodiments, X⁷ is F. In embodiments, R⁷ and R⁸ substituents bonded to the same nitrogen atom may optionally be joined to form a R⁴⁸-substituted or unsubstituted heterocycloalkyl (e.g. 3 to 8 membered heterocycloalkyl, 4 to 8 membered heterocycloalkyl, or 5 to 6 membered heterocycloalkyl), or R⁴⁸-substituted or unsubstituted heteroaryl (e.g. 5 to 10 membered heteroaryl, 5 to 9 membered heteroaryl, or 5 to 6 membered heteroaryl).

In embodiments, R⁷ is independently hydrogen or unsubstituted alkyl. In embodiments, R⁷ is independently hydrogen or unsubstituted C₁-C₆ alkyl. In embodiments, R⁷ is independently hydrogen or unsubstituted C₁-C₅ alkyl. In embodiments, R⁷ is independently hydrogen or unsubstituted C₁-C₄ alkyl. In embodiments, R⁷ is independently hydrogen or unsubstituted C₁-C₃ alkyl. In embodiments, R⁷ is independently hydrogen or unsubstituted C₁-C₂ alkyl. In embodiments, R⁷ is independently hydrogen or unsubstituted C₂-C₆ alkyl. In embodiments, R⁷ is independently hydrogen or unsubstituted C₂-C₅ alkyl. In embodiments, R⁷ is independently hydrogen or unsubstituted C₂-C₄ alkyl. In embodiments, R⁷ is independently hydrogen or unsubstituted C₂-C₃ alkyl. In embodiments, R⁷ is independently hydrogen or unsubstituted C₃-C₆ alkyl. In embodiments, R′ is independently hydrogen or unsubstituted C₄-C₆ alkyl. In embodiments, R⁷ is independently hydrogen or unsubstituted C₅-C₆ alkyl. In embodiments, R⁷ is independently hydrogen. In embodiments, R⁷ is independently unsubstituted alkyl. In embodiments, R⁷ is independently unsubstituted C₁-C₆ alkyl. In embodiments, R⁷ is independently unsubstituted C₁-C₅ alkyl. In embodiments, R⁷ is independently unsubstituted C₁-C₄ alkyl. In embodiments, R⁷ is independently unsubstituted C₁-C₃ alkyl. In embodiments, R⁷ is independently unsubstituted C₁-C₂ alkyl. In embodiments, R⁷ is independently unsubstituted C₂-C₆ alkyl. In embodiments, R⁷ is independently unsubstituted C₂-C₅ alkyl. In embodiments, R⁷ is independently unsubstituted C₂-C₄ alkyl. In embodiments, R⁷ is independently unsubstituted C₂-C₃ alkyl. In embodiments, R⁷ is independently unsubstituted C₃-C₆ alkyl. In embodiments, R⁷ is independently unsubstituted C₄-C₆ alkyl. In embodiments, R⁷ is independently unsubstituted C₅-C₆ alkyl. In embodiments, R⁷ is hydrogen. In embodiments, R⁷ is independently hydrogen, halogen, —CX⁷₃, —CHX⁷₂, —CH₂X⁷, —CN, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁷ and R⁸ substituents bonded to the same nitrogen atom may optionally be joined to form a substituted or unsubstituted heterocycloalkyl or substituted or unsubstituted heteroaryl.

R⁴⁸ is independently oxo, halogen, —CX⁴⁸3, —CHX⁴⁸₂, —CH₂X⁴⁸, —OCH₂X⁴⁸, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁴⁸₃, —OCHX⁴⁸₂, R⁴⁹-substituted or unsubstituted alkyl (e.g. C₁-C₈ alkyl, C₁-C₆ alkyl, or C₁-C₄ alkyl), R⁴⁹-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R⁴⁹-substituted or unsubstituted cycloalkyl (e.g. C₃-C₈ cycloalkyl, C₄-C₈ cycloalkyl, or C₅-C₆ cycloalkyl), R⁴⁹-substituted or unsubstituted heterocycloalkyl (e.g. 3 to 8 membered heterocycloalkyl, 4 to 8 membered heterocycloalkyl, or 5 to 6 membered heterocycloalkyl), R⁴⁹-substituted or unsubstituted aryl (e.g. C₆-C₁₀ aryl or C₆ aryl), or R⁴⁹-substituted or unsubstituted heteroaryl (e.g. 5 to 10 membered heteroaryl, 5 to 9 membered heteroaryl, or 5 to 6 membered heteroaryl). X⁴⁸ is halogen. In embodiments, X⁴⁸ is F.

R⁴⁹ is independently oxo, halogen, —CX⁴⁹₃, —CHX⁴⁹₂, —CH₂X⁴⁹, —OCH₂X⁴⁹, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁴⁹₃, —OCHX⁴⁹₂, R⁵⁰-substituted or unsubstituted alkyl (e.g. C₁-C₈ alkyl, C₁-C₆ alkyl, or C₁-C₄ alkyl), R⁵⁰-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R⁵⁰-substituted or unsubstituted cycloalkyl (e.g. C₃-C₈ cycloalkyl, C₄-C₈ cycloalkyl, or C₅-C₆ cycloalkyl), R⁵⁰-substituted or unsubstituted heterocycloalkyl (e.g. 3 to 8 membered heterocycloalkyl, 4 to 8 membered heterocycloalkyl, or 5 to 6 membered heterocycloalkyl), R⁵⁰-substituted or unsubstituted aryl (e.g. C₆-C₁₀ aryl or C₆ aryl), or R⁵⁰-substituted or unsubstituted heteroaryl (e.g. 5 to 10 membered heteroaryl, 5 to 9 membered heteroaryl, or 5 to 6 membered heteroaryl). X⁴⁹ is halogen. In embodiments, X⁴⁹ is F.

In embodiments, R⁸ is independently hydrogen, oxo, halogen, —CX⁸₃, —CHX⁸₂, —CH₂X⁸, —OCH₂X⁸, —CN, —OH, —NH₂, —COOH, —CONH₂; —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁸₃, —OCHX⁸₂, R^{5.1}-substituted or unsubstituted alkyl (e.g. C₁-C₈ alkyl, C₁-C₆ alkyl, or C₁-C₄ alkyl), R^{5.1}-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R^{5.1}-substituted or unsubstituted cycloalkyl (e.g. C₃-C₈ cycloalkyl, C₄-C₈ cycloalkyl, or C₅-C₆ cycloalkyl), R^{5.1}-substituted or unsubstituted heterocycloalkyl (e.g. 3 to 8 membered heterocycloalkyl, 4 to 8 membered heterocycloalkyl, or 5 to 6 membered heterocycloalkyl), R^{5.1}-substituted or unsubstituted aryl (e.g. C₆-C₁₀ aryl or C₆ aryl), or R^{5.1}-substituted or unsubstituted heteroaryl (e.g. 5 to 10 membered heteroaryl, 5 to 9 membered heteroaryl, or 5 to 6 membered heteroaryl). X⁸ is halogen. In embodiments, X⁸ is F. In embodiments, X⁷ is F. In embodiments, R⁷ and R⁸ substituents bonded to the same nitrogen atom may optionally be joined to form a R^{5.1}-substituted or unsubstituted heterocycloalkyl (e.g. 3 to 8 membered heterocycloalkyl, 4 to 8 membered heterocycloalkyl, or 5 to 6 membered heterocycloalkyl) or R^{5.1}-substituted or unsubstituted heteroaryl (e.g. 5 to 10 membered heteroaryl, 5 to 9 membered heteroaryl, or 5 to 6 membered heteroaryl).

In embodiments, R⁸ is independently hydrogen or unsubstituted alkyl. In embodiments, R⁸ is independently hydrogen or unsubstituted C₁-C₆ alkyl. In embodiments, R⁸ is independently hydrogen or unsubstituted C₁-C₅ alkyl. In embodiments, R⁸ is independently hydrogen or unsubstituted C₁-C₄ alkyl. In embodiments, R⁸ is independently hydrogen or unsubstituted C₁-C₃ alkyl. In embodiments, R⁸ is independently hydrogen or unsubstituted C₁-C₂ alkyl. In embodiments, R⁸ is independently hydrogen or unsubstituted C₂-C₆ alkyl. In embodiments, R⁸ is independently hydrogen or unsubstituted C₂-C₅ alkyl. In embodiments, R⁸ is independently hydrogen or unsubstituted C₂-C₄ alkyl. In embodiments, R⁸ is independently hydrogen or unsubstituted C₂-C₃ alkyl. In embodiments, R⁸ is independently hydrogen or unsubstituted C₃-C₆ alkyl. In embodiments, R⁸ is independently hydrogen or unsubstituted C₄-C₆ alkyl. In embodiments, R⁸ is independently hydrogen or unsubstituted C₅-C₆ alkyl. In embodiments, R⁸ is independently hydrogen. In embodiments, R⁸ is independently unsubstituted alkyl. In embodiments, R⁸ is independently unsubstituted C₁-C₆ alkyl. In embodiments, R⁸ is independently unsubstituted C₁-C₅ alkyl. In embodiments, R⁸ is independently unsubstituted C₁-C₄ alkyl. In embodiments, R⁸ is independently unsubstituted C₁-C₃ alkyl. In embodiments, R⁸ is independently unsubstituted C₁-C₂ alkyl. In embodiments, R⁸ is independently unsubstituted C₂-C₆ alkyl. In embodiments, R⁸ is independently unsubstituted C₂-C₅ alkyl. In embodiments, R⁸ is independently unsubstituted C₂-C₄ alkyl. In embodiments, R⁸ is independently unsubstituted C₂-C₃ alkyl. In embodiments, R⁸ is independently unsubstituted C₃-C₆ alkyl. In embodiments, R⁸ is independently unsubstituted C₄-C₆ alkyl. In embodiments, R⁸ is independently unsubstituted C₅-C₆ alkyl. In embodiments, R⁸ is hydrogen. In embodiments, R⁸ is independently hydrogen, halogen, —CX⁸₃, —CHX⁸₂, —CH₂X⁸, —CN, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{5.1} is independently oxo, halogen, —CX⁵¹₃, —CHX⁵¹₂, —CH₂X⁵¹, —OCH₂X⁵¹, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁵¹₃, —OCHX⁵¹₂, R^{5.2}-substituted or unsubstituted alkyl (e.g. C₁-C₈ alkyl, C₁-C₆ alkyl, or C₁-C₄ alkyl), R^{5.2}-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R^{5.2}-substituted or unsubstituted cycloalkyl (e.g. C₃-C₈ cycloalkyl, C₄-C₈ cycloalkyl, or C₅-C₆ cycloalkyl), R^{5.2}-substituted or unsubstituted heterocycloalkyl (e.g. 3 to 8 membered heterocycloalkyl, 4 to 8 membered heterocycloalkyl, or 5 to 6 membered heterocycloalkyl), R^{5.2}-substituted or unsubstituted aryl (e.g. C₆-C₁₀ aryl or C₆ aryl), or R^{5.2}-substituted or unsubstituted heteroaryl (e.g. 5 to 10 membered heteroaryl, 5 to 9 membered heteroaryl, or 5 to 6 membered heteroaryl). X⁵¹ is halogen. In embodiments, X⁵¹ is F.

R^{5.2} is independently oxo, halogen, —CX⁵²₃, —CHX⁵²₂, —CH₂X⁵², —OCH₂X⁵², —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁵²₃, —OCHX⁵²₂, R⁵³-substituted or unsubstituted alkyl (e.g. C₁-C₈ alkyl, C₁-C₆ alkyl, or C₁-C₄ alkyl), R⁵³-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R⁵³-substituted or unsubstituted cycloalkyl (e.g. C₃-C₈ cycloalkyl, C₄-C₈ cycloalkyl, or C₅-C₆ cycloalkyl), R⁵³-substituted or unsubstituted heterocycloalkyl (e.g. 3 to 8 membered heterocycloalkyl, 4 to 8 membered heterocycloalkyl, or 5 to 6 membered heterocycloalkyl), R⁵³-substituted or unsubstituted aryl (e.g. C₆-C₁₀ aryl or C₆ aryl), or R⁵³-substituted or unsubstituted heteroaryl (e.g. 5 to 10 membered heteroaryl, 5 to 9 membered heteroaryl, or 5 to 6 membered heteroaryl). X⁵² is halogen. In embodiments, X⁵² is F.

In embodiments, R⁹ is independently hydrogen, oxo, halogen, —CX⁹₃, —CHX⁹₂, —CH₂X⁹, —OCH₂X⁹, —CN, —OH, —NH₂, —COOH, —CONH₂; —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁹₃, —OCHX⁹₂, R⁵⁴-substituted or unsubstituted alkyl (e.g. C₁-C₈ alkyl, C₁-C₆ alkyl, or C₁-C₄ alkyl), R⁵⁴-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R⁵⁴-substituted or unsubstituted cycloalkyl (e.g. C₃-C₈ cycloalkyl, C₄-C₈ cycloalkyl, or C₅-C₆ cycloalkyl), R⁵⁴-substituted or unsubstituted heterocycloalkyl (e.g. 3 to 8 membered heterocycloalkyl, 4 to 8 membered heterocycloalkyl, or 5 to 6 membered heterocycloalkyl), R⁵⁴-substituted or unsubstituted aryl (e.g. C₆-C₁₀ aryl or C₆ aryl), or R⁵⁴-substituted or unsubstituted heteroaryl (e.g. 5 to 10 membered heteroaryl, 5 to 9 membered heteroaryl, or 5 to 6 membered heteroaryl). X⁹ is halogen. In embodiments, X⁹ is F. In embodiments, R⁹ is hydrogen. In embodiments, R⁹ is independently hydrogen, halogen, —CX⁹₃, —CHX⁹₂, —CH₂X⁹, —CN, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁵⁴ is independently oxo, halogen, —CX⁵⁴₃, —CHX⁵⁴₂, —CH₂X⁵⁴, —OCH₂X⁵⁴, —OCHX⁵⁴₂—CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁵⁴₃, —OCHX⁵⁴₂, R⁵⁵-substituted or unsubstituted alkyl (e.g. C₁-C₈ alkyl, C₁-C₆ alkyl, or C₈-C₄ alkyl), R⁵⁵-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R⁵⁵-substituted or unsubstituted cycloalkyl (e.g. C₃-C₈ cycloalkyl, C₄-C₈ cycloalkyl, or C₅-C₆ cycloalkyl), R⁵⁵-substituted or unsubstituted heterocycloalkyl (e.g. 3 to 8 membered heterocycloalkyl, 4 to 8 membered heterocycloalkyl, or 5 to 6 membered heterocycloalkyl), R⁵⁵-substituted or unsubstituted aryl (e.g. C₆-C₁₀ aryl or C₆ aryl), or R⁵⁵-substituted or unsubstituted heteroaryl (e.g. 5 to 10 membered heteroaryl, 5 to 9 membered heteroaryl, or 5 to 6 membered heteroaryl). X⁵⁴ is halogen. In embodiments, X⁵⁴ is F.

R⁵⁵ is independently oxo, halogen, —CX⁵⁵₃, —CHX⁵⁵₂, —CH₂X⁵⁵, —OCH₂X⁵⁵, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁵⁵₃, —OCHX⁵⁵₂, R⁵⁶-substituted or unsubstituted alkyl (e.g. C₁-C₈ alkyl, C₁-C₆ alkyl, or C₁-C₄ alkyl), R⁵⁶-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R⁵⁶-substituted or unsubstituted cycloalkyl (e.g. C₃-C₈ cycloalkyl, C₄-C₈ cycloalkyl, or C₅-C₆ cycloalkyl), R⁵⁶-substituted or unsubstituted heterocycloalkyl (e.g. 3 to 8 membered heterocycloalkyl, 4 to 8 membered heterocycloalkyl, or 5 to 6 membered heterocycloalkyl), R⁵⁶-substituted or unsubstituted aryl (e.g. C₆-C₁₀ aryl or C₆ aryl), or R⁵⁶-substituted or unsubstituted heteroaryl (e.g. 5 to 10 membered heteroaryl, 5 to 9 membered heteroaryl, or 5 to 6 membered heteroaryl). X^{ss} is halogen. In embodiments; X⁵⁵ is F.

In embodiments, R¹⁰ is independently hydrogen, oxo, halogen, —CX¹⁰₃, —CHX¹⁰₂—CH₂X¹⁰, —OCH₂X¹⁰, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX¹⁰₃, —OCHX¹⁰₂, R⁵⁷-substituted or unsubstituted alkyl (e.g. C₁-C₈ alkyl, C₁-C₆ alkyl, or C₁-C₄ alkyl), R⁵⁷-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R⁵⁷-substituted or unsubstituted cycloalkyl (e.g. C₃-C₈ cycloalkyl, C₄-C₈ cycloalkyl, or C₅-C₆ cycloalkyl), R⁵⁷-substituted or unsubstituted heterocycloalkyl (e.g. 3 to 8 membered heterocycloalkyl, 4 to 8 membered heterocycloalkyl, or 5 to 6 membered heterocycloalkyl), R⁵⁷-substituted or unsubstituted aryl (e.g. C₆-C₁₀ aryl or C₆ aryl), or R⁵⁷-substituted or unsubstituted heteroaryl (e.g. 5 to 10 membered heteroaryl, 5 to 9 membered heteroaryl, or 5 to 6 membered heteroaryl). X¹⁰ is halogen. In embodiments, X¹⁰ is F. In embodiments, R¹⁰ is hydrogen. In embodiments, R¹⁰, is independently hydrogen, halogen, —CX¹⁰₃, —CHX¹⁰₂, —CH₂X¹⁰, —CN, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁵⁷ is independently oxo, halogen, —CX⁵⁷₃, —CHX⁵⁷₂, —CH₂X⁵⁷, —OCH₂X⁵⁷, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁵⁷₃, —OCHX¹⁷₂, R⁵⁸-substituted or unsubstituted alkyl (e.g. C₁-C₈ alkyl, C₁-C₆ alkyl, or C₁-C₄ alkyl), R⁵⁸-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R⁵⁸-substituted or unsubstituted cycloalkyl (e.g. C₃-C₈ cycloalkyl, C₄-C₈ cycloalkyl, or C₅-C₆ cycloalkyl), R⁵⁸-substituted or unsubstituted heterocycloalkyl (e.g. 3 to 8 membered heterocycloalkyl, 4 to 8 membered heterocycloalkyl, or 5 to 6 membered heterocycloalkyl), R⁵⁸-substituted or unsubstituted aryl (e.g. C₆-C₁₀ aryl or C₆ aryl), or R⁵⁸-substituted or unsubstituted heteroaryl (e.g. 5 to 10 membered heteroaryl, 5 to 9 membered heteroaryl, or 5 to 6 membered heteroaryl). X⁵⁷ is halogen. In embodiments, X⁵⁷ is F.

R⁵⁸ is independently oxo, halogen, —CX⁵⁸₃, —CHX⁵⁸₂, —CH₂X⁵⁸, —OCH₂X⁵⁸, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁵⁸₃, —OCHX⁵⁸₂, R⁵⁹-substituted or unsubstituted alkyl (e.g. C₁-C₈ alkyl, C₁-C₆ alkyl, or C₁-C₄ alkyl), R⁵⁹-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R⁵⁹-substituted or unsubstituted cycloalkyl (e.g. C₃-C₈ cycloalkyl, C₄-C₈ cycloalkyl, or C₅-C₆ cycloalkyl), R⁵⁹-substituted or unsubstituted heterocycloalkyl (e.g. 3 to 8 membered heterocycloalkyl, 4 to 8 membered heterocycloalkyl, or 5 to 6 membered heterocycloalkyl), R⁵⁹-substituted or unsubstituted aryl (e.g. C₆-C₁₀ aryl or C₆ aryl), or R⁵⁹-substituted or unsubstituted heteroaryl (e.g. 5 to 10 membered heteroaryl, 5 to 9 membered heteroaryl, or 5 to 6 membered heteroaryl). X⁵⁸ is halogen. In embodiments, X⁵⁸ is F.

In embodiments, R¹¹ is independently hydrogen, oxo, halogen, —CX¹¹3, —CHX¹¹₂, —CH₂X¹¹, —OCH₂X¹¹, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX¹¹₃, —OCHX¹¹₂, R⁶⁰-substituted or unsubstituted alkyl (e.g. C₁-C₈ alkyl, C₁-C₆ alkyl, or C₁-C₄ alkyl), R⁶⁰-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R⁶⁰-substituted or unsubstituted cycloalkyl (e.g. C₃-C₈ cycloalkyl, C₄-C₈ cycloalkyl, or C₅-C₆ cycloalkyl), R⁶⁰-substituted or unsubstituted heterocycloalkyl (e.g. 3 to 8 membered heterocycloalkyl, 4 to 8 membered heterocycloalkyl, or 5 to 6 membered heterocycloalkyl), R⁶⁰-substituted or unsubstituted aryl (e.g. C₆-C₁₀ aryl or C₆ aryl), or R⁶⁰-substituted or unsubstituted heteroaryl (e.g. 5 to 10 membered heteroaryl, 5 to 9 membered heteroaryl, or 5 to 6 membered heteroaryl). X¹¹ is halogen. In embodiments, X¹¹ is F. In embodiments, R¹¹ and R¹² substituents bonded to the same nitrogen atom may optionally be joined to form a R⁶⁰-substituted or unsubstituted heterocycloalkyl (e.g. 3 to 8 membered heterocycloalkyl, 4 to 8 membered heterocycloalkyl, or 5 to 6 membered heterocycloalkyl) or Rho-substituted or unsubstituted heteroaryl (e.g. 5 to 10 membered heteroaryl, 5 to 9 membered heteroaryl, or 5 to 6 membered heteroaryl). In embodiments, R¹¹ is hydrogen. In embodiments, R¹¹ is independently hydrogen, halogen, —CX¹¹₃, —CHX¹¹₂, —CH₂X¹¹, —CN, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl; or substituted or unsubstituted heteroaryl.

R⁶⁰ is independently oxo, halogen, —CX⁶⁰₃, —CHX⁶⁰₂, —CH₂X⁶⁰, —OCH₂X⁶⁰, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁶⁰3, —OCHX⁶⁰₂, R⁶¹-substituted or unsubstituted alkyl (e.g. C₁-C₈ alkyl, C₁-C₆ alkyl, or C₁-C₄ alkyl), R⁶¹-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R⁶¹-substituted or unsubstituted cycloalkyl (e.g. C₃-C₈ cycloalkyl, C₄-C₈ cycloalkyl, or C₅-C₆ cycloalkyl), R⁶¹-substituted or unsubstituted heterocycloalkyl (e.g. 3 to 8 membered heterocycloalkyl, 4 to 8 membered heterocycloalkyl, or 5 to 6 membered heterocycloalkyl), R⁶¹-substituted or unsubstituted aryl (e.g. C₆-C₁₀ aryl or C₆ aryl), or R⁶¹-substituted or unsubstituted heteroaryl (e.g. 5 to 10 membered heteroaryl, 5 to 9 membered heteroaryl, or 5 to 6 membered heteroaryl). X⁶⁰ is halogen. In embodiments, X⁶⁰ is F.

R⁶¹ is independently oxo, halogen, —CX⁶¹₃, —CHX⁶¹₂, —CH₂X⁶¹, —OCH₂X⁶¹, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁶¹3, —OCHX⁶¹₂, R⁶²-substituted or unsubstituted alkyl (e.g. C₁-C₈ alkyl, C₁-C₆ alkyl, or C₁-C₄ alkyl), R⁶²-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R⁶²-substituted or unsubstituted cycloalkyl (e.g. C₃-C₈ cycloalkyl, C₄-C₈ cycloalkyl, or C₅-C₆ cycloalkyl), R⁶²-substituted or unsubstituted heterocycloalkyl (e.g. 3 to 8 membered heterocycloalkyl, 4 to 8 membered heterocycloalkyl, or 5 to 6 membered heterocycloalkyl), R⁶²-substituted or unsubstituted aryl (e.g. C₆-C₁₀ aryl or C₆ aryl), or R⁶²-substituted or unsubstituted heteroaryl (e.g. 5 to 10 membered heteroaryl, 5 to 9 membered heteroaryl, or 5 to 6 membered heteroaryl). X⁶¹ is halogen. In embodiments, X⁶¹ is F.

In embodiments, R¹² is independently hydrogen, oxo, halogen, —CX¹²₃, —CHX¹²₂, —CH₂X¹², —OCH₂X¹², —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX¹²₃, —OCHX¹²₂, R⁶³-substituted or unsubstituted alkyl (e.g. C₁-C₈ alkyl, C₁-C₆ alkyl, or C₁-C₄ alkyl), R⁶³-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R⁶³-substituted or unsubstituted cycloalkyl (e.g. C₃-C₈ cycloalkyl, C₄-C₈ cycloalkyl, or C₅-C₆ cycloalkyl), R⁶³-substituted or unsubstituted heterocycloalkyl (e.g. 3 to 8 membered heterocycloalkyl, 4 to 8 membered heterocycloalkyl, or 5 to 6 membered heterocycloalkyl), R⁶³-substituted or unsubstituted aryl (e.g. C₆-C₁₀ aryl or C₆ aryl), or R⁶³-substituted or unsubstituted heteroaryl (e.g. 5 to 10 membered heteroaryl, 5 to 9 membered heteroaryl, or 5 to 6 membered heteroaryl). X¹² is halogen. In embodiments, X¹² is F. In embodiments, R¹¹ and R¹² substituents bonded to the same nitrogen atom may optionally be joined to form a R⁶³-substituted or unsubstituted heterocycloalkyl (e.g. 3 to 8 membered heterocycloalkyl, 4 to 8 membered heterocycloalkyl, or 5 to 6 membered heterocycloalkyl) or R⁶³-substituted or unsubstituted heteroaryl (e.g. 5 to 10 membered heteroaryl, 5 to 9 membered heteroaryl, or 5 to 6 membered heteroaryl). In embodiments, R¹² is hydrogen. In embodiments, R¹² is independently hydrogen, halogen, —CX¹²3, —CHX¹²₂, —CH₂X¹², —CN, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁶³ is independently oxo, halogen, —CX⁶³₃, —CHX⁶³₂, —CH₂X⁶³, —OCH₂X⁶³, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁶³₃, —OCHX⁶³2, R⁶⁴-substituted or unsubstituted alkyl (e.g. C₁-C₈ alkyl, C₁-C₆ alkyl, or C₁-C₄ alkyl), R⁶⁴-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R⁶⁴-substituted or unsubstituted cycloalkyl (e.g. C₃-C₈ cycloalkyl, C₄-C₈ cycloalkyl, or C₅-C₆ cycloalkyl), R⁶⁴-substituted or unsubstituted heterocycloalkyl (e.g. 3 to 8 membered heterocycloalkyl, 4 to 8 membered heterocycloalkyl, or 5 to 6 membered heterocycloalkyl), R⁶⁴-substituted or unsubstituted aryl (e.g. C₆-C₁₀ aryl or C₆ aryl), or R⁶⁴-substituted or unsubstituted heteroaryl (e.g. 5 to 10 membered heteroaryl, 5 to 9 membered heteroaryl, or 5 to 6 membered heteroaryl). X⁶³ is halogen. In embodiments, X⁶³ is F.

R⁶⁴ is independently oxo, halogen, —CX⁶⁴₃, —CHX⁶⁴₂, —CH₂X⁶⁴, —OCH₂X⁶⁴, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁶⁴₃, —OCHX⁶⁴₂, R⁶⁵-substituted or unsubstituted alkyl (e.g. C₁-C₈ alkyl, C₁-C₆ alkyl, or C₁-C₄ alkyl), R⁶⁵-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R⁶⁵-substituted or unsubstituted cycloalkyl (e.g. C₃-C₈ cycloalkyl, C₄-C₈ cycloalkyl, or C₅-C₆ cycloalkyl), R⁶⁵-substituted or unsubstituted heterocycloalkyl (e.g. 3 to 8 membered heterocycloalkyl, 4 to 8 membered heterocycloalkyl, or 5 to 6 membered heterocycloalkyl), R⁶⁵-substituted or unsubstituted aryl (e.g. C₆-C₁₀ aryl or C₆ aryl), or R⁶⁵-substituted or unsubstituted heteroaryl (e.g. 5 to 10 membered heteroaryl, 5 to 9 membered heteroaryl, or 5 to 6 membered heteroaryl). X⁶⁴ is halogen. In embodiments, X⁶⁴ is F.

In embodiments, R¹³ is independently hydrogen, oxo, halogen, —CX¹³3, —CHX¹³₂, —CH₂X¹³, —OCH₂X¹³, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX¹³3, —OCHX¹³2, R⁶⁶-substituted or unsubstituted alkyl (e.g. C₁-C₈ alkyl, C₁-C₆ alkyl, or C₁-C₄ alkyl), R⁶⁶-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R⁶⁶-substituted or unsubstituted cycloalkyl (e.g. C₃-C₈ cycloalkyl, C₄-C₈ cycloalkyl, or C₅-C₆ cycloalkyl), R⁶⁶-substituted or unsubstituted heterocycloalkyl (e.g. 3 to 8 membered heterocycloalkyl, 4 to 8 membered heterocycloalkyl, or 5 to 6 membered heterocycloalkyl), R⁶⁶-substituted or unsubstituted aryl (e.g. C₆-C₁₀ aryl or C₆ aryl), or R⁶⁶-substituted or unsubstituted heteroaryl (e.g. 5 to 10 membered heteroaryl, 5 to 9 membered heteroaryl, or 5 to 6 membered heteroaryl). X¹³ is halogen. In embodiments, X¹³ is F. In embodiments, R¹³ is hydrogen. In embodiments, R¹³ is independently hydrogen, halogen, —CX¹³3, —CHX¹³2, —CH₂X¹³, —CN, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁶⁶ is independently oxo, halogen, —CX⁶⁶₃, —CHX⁶⁶₂, —CH₂X⁶⁶, —OCH₂X⁶⁶, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁶⁶₃, —OCHX⁶⁶₂, R⁶⁷-substituted or unsubstituted alkyl (e.g. C₁-C₈ alkyl, C₁-C₆ alkyl, or C₁-C₄ alkyl), R⁶⁷-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R⁶⁷-substituted or unsubstituted cycloalkyl (e.g. C₃-C₈ cycloalkyl, C₄-C₈ cycloalkyl, or C₅-C₆ cycloalkyl), R⁶¹-substituted or unsubstituted heterocycloalkyl (e.g. 3 to 8 membered heterocycloalkyl, 4 to 8 membered heterocycloalkyl, or 5 to 6 membered heterocycloalkyl), R⁶⁷-substituted or unsubstituted aryl (e.g. C₆-C₁₀ aryl or C₆ aryl), or R⁶⁷-substituted or unsubstituted heteroaryl (e.g. 5 to 10 membered heteroaryl, 5 to 9 membered heteroaryl, or 5 to 6 membered heteroaryl). X⁶⁶ is halogen. In embodiments, X⁶⁶ is F.

R⁶⁷ is independently oxo, halogen, —CX⁶⁷₃, —CHX⁶⁷₂, —CH₂X⁶⁷, —OCH₂X⁶⁷, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁶⁷3, —OCHX⁶⁷2, R⁶⁸-substituted or unsubstituted alkyl (e.g. C₁-C₈ alkyl, C₁-C₆ alkyl, or C₁-C₄ alkyl), R^{6$}-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R⁶⁸-substituted or unsubstituted cycloalkyl (e.g. C₃-C₈ cycloalkyl, C₄-C₈ cycloalkyl, or C₅-C₆ cycloalkyl), R⁶⁸-substituted or unsubstituted heterocycloalkyl (e.g. 3 to 8 membered heterocycloalkyl, 4 to 8 membered heterocycloalkyl, or 5 to 6 membered heterocycloalkyl), R⁶⁸-substituted or unsubstituted aryl (e.g. C₆-C₁₀ aryl or C₆ aryl), or R⁶⁸-substituted or unsubstituted heteroaryl (e.g. 5 to 10 membered heteroaryl, 5 to 9 membered heteroaryl, or 5 to 6 membered heteroaryl). X⁶⁷ is halogen. In embodiments, X⁶⁷ is F.

In embodiments, R¹⁴ is independently hydrogen, oxo, halogen, —CX¹⁴₃, —CHX¹⁴₂—CH₂X¹⁴, —OCH₂X¹⁴, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX¹⁴₃, —OCHX¹⁴₂, R⁶⁹-substituted or unsubstituted alkyl (e.g. C₁-C₈ alkyl, C₁-C₆ alkyl, or C₁-C₄ alkyl), R⁶⁹-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R⁶⁹-substituted or unsubstituted cycloalkyl (e.g. C₃-C₈ cycloalkyl, C₄-C₈ cycloalkyl, or C₅-C₆ cycloalkyl), R⁶⁹-substituted or unsubstituted heterocycloalkyl (e.g. 3 to 8 membered heterocycloalkyl, 4 to 8 membered heterocycloalkyl, or 5 to 6 membered heterocycloalkyl), R⁶⁹-substituted or unsubstituted aryl (e.g. C₆-C₁₀ aryl or C₆ aryl), or R⁶⁹-substituted or unsubstituted heteroaryl (e.g. 5 to 10 membered heteroaryl, 5 to 9 membered heteroaryl, or 5 to 6 membered heteroaryl). X¹⁴ is halogen. In embodiments, X¹⁴ is F. In embodiments, R¹⁴ is hydrogen. In embodiments, R¹⁴ is independently hydrogen, halogen, —CX¹⁴₃—CHX¹⁴₂—CH₂X¹⁴, —CN, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁶⁹ is independently oxo, halogen, —CX⁶⁹₃, —CHX⁶⁹₂, —CH₂X⁶⁹, —OCH₂X⁶⁹, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁶⁹3, —OCHX⁶⁹₂, R⁷⁰-substituted or unsubstituted alkyl (e.g. C₁-C₈ alkyl, C₁-C₆ alkyl, or C₁-C₄ alkyl), R⁷⁰-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R⁷⁰-substituted or unsubstituted cycloalkyl (e.g. C₃-C₈ cycloalkyl, C₄-C₈ cycloalkyl, or C₅-C₆ cycloalkyl), R⁷⁰-substituted or unsubstituted heterocycloalkyl (e.g. 3 to 8 membered heterocycloalkyl, 4 to 8 membered heterocycloalkyl, or 5 to 6 membered heterocycloalkyl), R⁷⁰-substituted or unsubstituted aryl (e.g. C₆-C₁₀ aryl or C₆ aryl), or R⁷⁰-substituted or unsubstituted heteroaryl (e.g. 5 to 10 membered heteroaryl, 5 to 9 membered heteroaryl, or 5 to 6 membered heteroaryl). X⁶⁹ is halogen. In embodiments, X⁶⁹ is F.

R⁷⁰ is independently oxo, halogen, —CX⁷⁰₃, —CHX⁷⁰₂, —CH₂X⁷⁰, —OCH₂X⁷⁰, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁷⁰₃, —OCHX⁷⁰₂, R⁷¹-substituted or unsubstituted alkyl (e.g. C₁-C₈ alkyl, C₁-C₆ alkyl, or C₁-C₄ alkyl), R⁷¹-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R⁷¹-substituted or unsubstituted cycloalkyl (e.g. C₃-C₈ cycloalkyl, C₄-C₈ cycloalkyl, or C₅-C₆ cycloalkyl), R⁷¹-substituted or unsubstituted heterocycloalkyl (e.g. 3 to 8 membered heterocycloalkyl, 4 to 8 membered heterocycloalkyl, or 5 to 6 membered heterocycloalkyl), R⁷¹-substituted or unsubstituted aryl (e.g. C₆-C₁₀ aryl or C₆ aryl), or R⁷¹-substituted or unsubstituted heteroaryl (e.g. 5 to 10 membered heteroaryl, 5 to 9 membered heteroaryl, or 5 to 6 membered heteroaryl). X⁷⁰ is halogen. In embodiments, X⁷⁰ is F.

In embodiments, R¹⁵ is independently hydrogen, oxo, halogen, —CX¹⁵₃, —CHX¹⁵₂, —CH₂X¹⁵, —OCH₂X¹⁵, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX¹⁵₃, —OCHX¹⁵₂, R⁷²-substituted or unsubstituted alkyl (e.g. C₁-C₈ alkyl, C₁-C₆ alkyl, or C₁-C₄ alkyl), R⁷²-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R⁷²-substituted or unsubstituted cycloalkyl (e.g. C₃-C₈ cycloalkyl, C₄-C₈ cycloalkyl, or C₅-C₆ cycloalkyl), R⁷²-substituted or unsubstituted heterocycloalkyl (e.g. 3 to 8 membered heterocycloalkyl, 4 to 8 membered heterocycloalkyl, or 5 to 6 membered heterocycloalkyl), R⁷²-substituted or unsubstituted aryl (e.g. C₆-C₁₀ aryl or C₆ aryl), or R⁷²-substituted or unsubstituted heteroaryl (e.g. 5 to 10 membered heteroaryl, 5 to 9 membered heteroaryl, or 5 to 6 membered heteroaryl). X¹⁵ is halogen. In embodiments, X¹⁵ is F. In embodiments, R¹⁵ and R¹⁶ substituents bonded to the same nitrogen atom may optionally be joined to form a R⁷²-substituted or unsubstituted heterocycloalkyl (e.g. 3 to 8 membered heterocycloalkyl, 4 to 8 membered heterocycloalkyl, or 5 to 6 membered heterocycloalkyl) or R⁷²-substituted or unsubstituted heteroaryl (e.g. 5 to 10 membered heteroaryl, 5 to 9 membered heteroaryl, or 5 to 6 membered heteroaryl). In embodiments, R¹⁵ is hydrogen. In embodiments, R¹⁵ is independently hydrogen, halogen, —CX¹⁵₃, —CHX¹⁵₂, —CH₂X¹⁵, —CN, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹⁵ is independently hydrogen or unsubstituted alkyl. In embodiments, R¹⁵ is independently hydrogen or unsubstituted C₁-C₆ alkyl. In embodiments, R¹⁵ is independently hydrogen or unsubstituted C₁-C₅ alkyl. In embodiments, R¹⁵ is independently hydrogen or unsubstituted C₁-C₄ alkyl. In embodiments, R¹⁵ is independently hydrogen or unsubstituted C₁-C₃ alkyl. In embodiments, R¹⁵ is independently hydrogen or unsubstituted C₁-C₂ alkyl. In embodiments, R¹⁵ is independently hydrogen or unsubstituted C₂-C₆ alkyl. In embodiments, R¹⁵ is independently hydrogen or unsubstituted C₂-C₅ alkyl. In embodiments, R¹⁵ is independently hydrogen or unsubstituted C₂-C₄ alkyl. In embodiments, R¹⁵ is independently hydrogen or unsubstituted C₂-C₃ alkyl. In embodiments, R¹⁵ is independently hydrogen or unsubstituted C₃-C₆ alkyl. In embodiments, R¹⁵ is independently hydrogen or unsubstituted C₄-C₆ alkyl. In embodiments, R¹⁵ is independently hydrogen or unsubstituted C₅-C₆ alkyl. In embodiments, R¹⁵ is independently hydrogen. In embodiments, R¹⁵ is independently unsubstituted alkyl. In embodiments, R¹⁵ is independently unsubstituted C₁-C₆ alkyl. In embodiments, R¹⁵ is independently unsubstituted C₁-C₅ alkyl. In embodiments, R¹⁵ is independently unsubstituted C₁-C₄ alkyl. In embodiments, R¹⁵ is independently unsubstituted C₁-C₃ alkyl. In embodiments, R¹⁵ is independently unsubstituted C₁-C₂ alkyl. In embodiments, R¹⁵ is independently unsubstituted C₂-C₆ alkyl. In embodiments, R¹⁵ is independently unsubstituted C₂-C₅ alkyl. In embodiments, R¹⁵ is independently unsubstituted C₂-C₄ alkyl. In embodiments, R¹⁵ is independently unsubstituted C₂-C₃ alkyl. In embodiments, R¹⁵ is independently unsubstituted C₃-C₆ alkyl. In embodiments, R¹⁵ is independently unsubstituted C₄-C₆ alkyl. In embodiments, R¹⁵ is independently unsubstituted C₅-C₆ alkyl.

R⁷² is independently oxo, halogen, —CX⁷²3, —CHX⁷²2, —CH₂X⁷², —OCH₂X⁷², —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁷²₃, —OCHX⁷²₂, R⁷³-substituted or unsubstituted alkyl (e.g. C₁-C₈ alkyl, C₁-C₆ alkyl, or C₁-C₄ alkyl), R⁷³-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R⁷³-substituted or unsubstituted cycloalkyl (e.g. C₃-C₈ cycloalkyl, C₄-C₈ cycloalkyl, or C₅-C₆ cycloalkyl), R⁷³-substituted or unsubstituted heterocycloalkyl (e.g. 3 to 8 membered heterocycloalkyl, 4 to 8 membered heterocycloalkyl, or 5 to 6 membered heterocycloalkyl), R⁷³-substituted or unsubstituted aryl (e.g. C₆-C₁₀ aryl or C₆ aryl), or R⁷³-substituted or unsubstituted heteroaryl (e.g. 5 to 10 membered heteroaryl, 5 to 9 membered heteroaryl, or 5 to 6 membered heteroaryl). X⁷² is halogen. In embodiments, X⁷² is F.

R⁷³ is independently oxo, halogen, —CX⁷³₃, —CHX⁷³₂, —CH₂X¹³, —OCH₂X⁷³, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁷³3, —OCHX⁷³₂, R⁷⁴-substituted or unsubstituted alkyl (e.g. C₁-C₈ alkyl, C₁-C₆ alkyl, or C₁-C₄ alkyl), R⁷⁴-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R⁷⁴-substituted or unsubstituted cycloalkyl (e.g. C₃-C₈ cycloalkyl, C₄-C₈ cycloalkyl, or C₅-C₆ cycloalkyl), R⁷⁴-substituted or unsubstituted heterocycloalkyl (e.g. 3 to 8 membered heterocycloalkyl, 4 to 8 membered heterocycloalkyl, or 5 to 6 membered heterocycloalkyl), R⁷⁴-substituted or unsubstituted aryl (e.g. C₆-C₁₀ aryl or C₆ aryl), or R⁷⁴-substituted or unsubstituted heteroaryl (e.g. 5 to 10 membered heteroaryl, 5 to 9 membered heteroaryl, or 5 to 6 membered heteroaryl). X⁷³ is halogen. In embodiments, X⁷³ is F.

In embodiments, R¹⁶ is independently hydrogen, oxo, halogen, —CX¹⁶₃, —CHX¹⁶₂—CH₂X¹⁶, —OCH₂X¹⁶, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX¹⁶₃, —OCHX¹⁶₂, R⁷⁵-substituted or unsubstituted alkyl (e.g. C₁-C₈ alkyl, C₁-C₆ alkyl, or C₁-C₄ alkyl), R⁷⁵-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R⁷⁵-substituted or unsubstituted cycloalkyl (e.g. C₃-C₈ cycloalkyl, C₄-C₈ cycloalkyl, or C₅-C₆ cycloalkyl), R⁷⁵-substituted or unsubstituted heterocycloalkyl (e.g. 3 to 8 membered heterocycloalkyl, 4 to 8 membered heterocycloalkyl, or 5 to 6 membered heterocycloalkyl), R⁷⁵-substituted or unsubstituted aryl (e.g. C₆-C₁₀ aryl or C₆ aryl), or R⁷⁵-substituted or unsubstituted heteroaryl (e.g. 5 to 10 membered heteroaryl, 5 to 9 membered heteroaryl, or 5 to 6 membered heteroaryl). X¹⁶ is halogen. In embodiments, X¹⁶ is F. In embodiments, R¹⁵ and R¹⁶ substituents bonded to the same nitrogen atom may optionally be joined to form a R⁷⁵-substituted or unsubstituted heterocycloalkyl (e.g. 3 to 8 membered heterocycloalkyl, 4 to 8 membered heterocycloalkyl, or to 6 membered heterocycloalkyl) or R⁷⁵-substituted or unsubstituted heteroaryl (e.g. 5 to 10 membered heteroaryl, 5 to 9 membered heteroaryl, or 5 to 6 membered heteroaryl). In embodiments, R¹⁶ is hydrogen. In embodiments, R¹⁶ is independently hydrogen, halogen, —CX¹⁶₃, —CHX¹⁶₂, —CH₂X¹⁶, —CN, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹⁶ is independently hydrogen or unsubstituted alkyl. In embodiments, R¹⁶ is independently hydrogen or unsubstituted C₁-C₆ alkyl. In embodiments, R¹⁶ is independently hydrogen or unsubstituted C₁-C₅ alkyl. In embodiments, R¹⁶ is independently hydrogen or unsubstituted C₁-C₄ alkyl. In embodiments, R¹⁶ is independently hydrogen or unsubstituted C₁-C₃ alkyl. In embodiments, R¹⁶ is independently hydrogen or unsubstituted C₁-C₂ alkyl. In embodiments, R¹⁶ is independently hydrogen or unsubstituted C₂-C₆ alkyl. In embodiments, R¹⁶ is independently hydrogen or unsubstituted C₂-C₅ alkyl. In embodiments, R¹⁶ is independently hydrogen or unsubstituted C₂-C₄ alkyl. In embodiments, R¹⁶ is independently hydrogen or unsubstituted C₂-C₃ alkyl. In embodiments, R¹⁶ is independently hydrogen or unsubstituted C₃-C₆ alkyl. In embodiments, R¹⁶ is independently hydrogen or unsubstituted C₄-C₆ alkyl. In embodiments, R¹⁶ is independently hydrogen or unsubstituted C₅-C₆ alkyl. In embodiments, R¹⁶ is independently hydrogen. In embodiments, R¹⁶ is independently unsubstituted alkyl. In embodiments, R¹⁶ is independently unsubstituted C₁-C₆ alkyl. In embodiments, R¹⁶ is independently unsubstituted C₁-C₅ alkyl. In embodiments, R¹⁶ is independently unsubstituted C₁-C₄ alkyl. In embodiments, R¹⁶ is independently unsubstituted C₁-C₃ alkyl. In embodiments, R¹⁶ is independently unsubstituted C₁-C₂ alkyl. In embodiments, R¹⁶ is independently unsubstituted C₂-C₆ alkyl. In embodiments, R¹⁶ is independently unsubstituted C₂-C₅ alkyl. In embodiments, R¹⁶ is independently unsubstituted C₂-C₄ alkyl. In embodiments, R¹⁶ is independently unsubstituted C₂-C₃ alkyl. In embodiments, R¹⁶ is independently unsubstituted C₃-C₆ alkyl. In embodiments, R¹⁶ is independently unsubstituted C₄-C₆ alkyl. In embodiments, R¹⁶ is independently unsubstituted C₅-C₆ alkyl.

R's is independently oxo, halogen, —CX⁷⁵₃, —CHX⁷⁵₂, —CH₂X⁷⁵, —OCH₂X⁷⁵, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁷⁵3, —OCHX⁷⁵₂, R⁷⁶-substituted or unsubstituted alkyl (e.g. C₁-C₈ alkyl, C₁-C₆ alkyl, or C₁-C₄ alkyl), R⁷⁶-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R⁷⁶-substituted or unsubstituted cycloalkyl (e.g. C₃-C₈ cycloalkyl, C₄-C₈ cycloalkyl, or C₅-C₆ cycloalkyl), R⁷⁶-substituted or unsubstituted heterocycloalkyl (e.g. 3 to 8 membered heterocycloalkyl, 4 to 8 membered heterocycloalkyl, or 5 to 6 membered heterocycloalkyl), R⁷⁶-substituted or unsubstituted aryl (e.g. C₆-C₁₀ aryl or C₆ aryl), or R⁷⁶-substituted or unsubstituted heteroaryl (e.g. 5 to 10 membered heteroaryl, 5 to 9 membered heteroaryl, or 5 to 6 membered heteroaryl). X⁷⁵ is halogen. In embodiments, X⁷⁵ is F.

R⁷⁶ is independently oxo, halogen, —CX⁷⁶₃, —CHX⁷⁶₂, —CH₂X⁷⁶, —OCH₂X⁷⁶, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁷⁶3, —OCHX⁷⁶₂, R⁷⁷-substituted or unsubstituted alkyl (e.g. C₁-C₈ alkyl, C₁-C₆ alkyl, or C₁-C₄ alkyl), R⁷⁷-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R⁷¹-substituted or unsubstituted cycloalkyl (e.g. C₃-C₈ cycloalkyl, C₄-C₈ cycloalkyl, or C₅-C₆ cycloalkyl), R⁷⁷-substituted or unsubstituted heterocycloalkyl (e.g. 3 to 8 membered heterocycloalkyl, 4 to 8 membered heterocycloalkyl, or 5 to 6 membered heterocycloalkyl), R⁷⁷-substituted or unsubstituted aryl (e.g. C₆-C₁₀ aryl or C₆ aryl), or R⁷⁷-substituted or unsubstituted heteroaryl (e.g. 5 to 10 membered heteroaryl, 5 to 9 membered heteroaryl, or 5 to 6 membered heteroaryl). X⁷⁶ is halogen. In embodiments, X⁷⁶ is F.

In embodiments, R¹⁷ is independently hydrogen, oxo, halogen, —CX¹⁷₃, —CHX¹⁷₂, —CH₂X¹⁷, —OCH₂X¹⁷, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX¹⁷3, —OCHX¹⁷2, R⁷⁸-substituted or unsubstituted alkyl (e.g. C₁-C₈ alkyl, C₁-C₆ alkyl, or C₁-C₄ alkyl), R⁷⁸-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R⁷⁸-substituted or unsubstituted cycloalkyl (e.g. C₃-C₈ cycloalkyl, C₄-C₈ cycloalkyl, or C₅-C₆ cycloalkyl), R⁷⁸-substituted or unsubstituted heterocycloalkyl (e.g. 3 to 8 membered heterocycloalkyl, 4 to 8 membered heterocycloalkyl, or 5 to 6 membered heterocycloalkyl), R⁷⁸-substituted or unsubstituted aryl (e.g. C₆-C₁₀ aryl or C₆ aryl), or R⁷⁸-substituted or unsubstituted heteroaryl (e.g. 5 to 10 membered heteroaryl, 5 to 9 membered heteroaryl, or 5 to 6 membered heteroaryl). X¹⁷ is halogen. In embodiments, X¹⁷ is F. In embodiments, R¹⁷ is hydrogen. In embodiments, R¹⁷ is independently hydrogen, halogen, —CX¹⁷₃—CHX¹⁷₂—CH₂X¹⁷, —CN, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁷⁸ is independently oxo, halogen, —CX⁷⁸₃, —CHX⁷⁸₂, —CH₂X¹⁸, —OCH₂X⁸, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁷⁸3, —OCHX⁷⁸₂, R⁷⁹-substituted or unsubstituted alkyl (e.g. C₁-C₈ alkyl, C₁-C₆ alkyl, or C₁-C₄ alkyl), R⁷⁹-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R⁷⁹-substituted or unsubstituted cycloalkyl (e.g. C₃-C₈ cycloalkyl, C₄-C₈ cycloalkyl, or C₅-C₆ cycloalkyl), R⁷⁹-substituted or unsubstituted heterocycloalkyl (e.g. 3 to 8 membered heterocycloalkyl, 4 to 8 membered heterocycloalkyl, or 5 to 6 membered heterocycloalkyl), R⁷⁹-substituted or unsubstituted aryl (e.g. C₆-C₁₀ aryl or C₆ aryl), or R⁷⁹-substituted or unsubstituted heteroaryl (e.g. 5 to 10 membered heteroaryl, 5 to 9 membered heteroaryl, or 5 to 6 membered heteroaryl). X⁷⁸ is halogen. In embodiments, X⁷⁸ is F.

R⁷⁹ is independently oxo, halogen, —CX⁷⁹₃, —CHX⁷⁹₂, —CH₂X¹⁹, —OCH₂X⁷⁹, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁷⁹₃, —OCHX⁷⁹₂, R⁸⁰-substituted or unsubstituted alkyl (e.g. C₁-C₈ alkyl, C₁-C₆ alkyl, or C₁-C₄ alkyl), R⁸⁰-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R⁸⁰-substituted or unsubstituted cycloalkyl (e.g. C₃-C₈ cycloalkyl, C₄-C₈ cycloalkyl, or C₅-C₆ cycloalkyl), R⁸⁰-substituted or unsubstituted heterocycloalkyl (e.g. 3 to 8 membered heterocycloalkyl, 4 to 8 membered heterocycloalkyl, or 5 to 6 membered heterocycloalkyl), R⁸⁰-substituted or unsubstituted aryl (e.g. C₆-C₁₀ aryl or C₆ aryl), or R⁸⁰-substituted or unsubstituted heteroaryl (e.g. 5 to 10 membered heteroaryl, 5 to 9 membered heteroaryl, or 5 to 6 membered heteroaryl). X⁷⁹ is halogen. In embodiments; X⁷⁹ is F.

In embodiments, R¹⁸ is independently hydrogen, oxo, halogen, —CX¹⁸3, —CHX¹⁸₂, —CH₂X¹⁸, —OCH₂X¹⁸, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX¹⁸₃, —OCHX¹⁸₂, R⁸¹-substituted or unsubstituted alkyl (e.g. C₁-C₈ alkyl, C₁-C₆ alkyl, or C₁-C₄ alkyl), R⁸¹-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R⁸¹-substituted or unsubstituted cycloalkyl (e.g. C₃-C₈ cycloalkyl, C₄-C₈ cycloalkyl, or C₅-C₆ cycloalkyl), R⁸¹-substituted or unsubstituted heterocycloalkyl (e.g. 3 to 8 membered heterocycloalkyl, 4 to 8 membered heterocycloalkyl, or 5 to 6 membered heterocycloalkyl), R⁸¹-substituted or unsubstituted aryl (e.g. C₆-C₁₀ aryl or C₆ aryl), or R^{8′}-substituted or unsubstituted heteroaryl (e.g. 5 to 10 membered heteroaryl, 5 to 9 membered heteroaryl, or 5 to 6 membered heteroaryl). X¹⁸ is halogen. In embodiments, X¹⁸ is F. In embodiments, R¹⁸ is hydrogen. In embodiments, R¹⁸ is independently hydrogen, halogen, —CX¹⁸₃, —CHX¹⁸₂, —CH₂X¹⁸, —CN, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁸¹ is independently oxo, halogen, —CX⁸¹₃, —CHX⁸¹₂, —CH₂X⁸¹, —OCH₂X⁸¹, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁸¹₃, —OCHX⁸¹₂, R⁸²-substituted or unsubstituted alkyl (e.g. C₁-C₈ alkyl, C₁-C₆ alkyl, or C₁-C₄ alkyl), R⁸²-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R⁸²-substituted or unsubstituted cycloalkyl (e.g. C₃-C₈ cycloalkyl, C₄-C₈ cycloalkyl, or C₅-C₆ cycloalkyl), R⁸²-substituted or unsubstituted heterocycloalkyl (e.g. 3 to 8 membered heterocycloalkyl, 4 to 8 membered heterocycloalkyl, or 5 to 6 membered heterocycloalkyl), R⁸²-substituted or unsubstituted aryl (e.g. C₆-C₁₀ aryl or C₆ aryl), or R⁸²-substituted or unsubstituted heteroaryl (e.g. 5 to 10 membered heteroaryl, 5 to 9 membered heteroaryl, or 5 to 6 membered heteroaryl). X⁸¹ is halogen. In embodiments, X⁸¹ is F.

R⁸² is independently oxo, halogen, —CX⁸²₃, —CHX⁸²₂, —CH₂X⁸², —OCH₂X⁸², —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁸²₃, —OCHX¹²₂, R⁸³-substituted or unsubstituted alkyl (e.g. C₁-C₈ alkyl, C₁-C₆ alkyl, or C₁-C₄ alkyl), R⁸³-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R⁸³-substituted or unsubstituted cycloalkyl (e.g. C₃-C₈ cycloalkyl, C₄-C₈ cycloalkyl, or C₅-C₆ cycloalkyl), R⁸³-substituted or unsubstituted heterocycloalkyl (e.g. 3 to 8 membered heterocycloalkyl, 4 to 8 membered heterocycloalkyl, or 5 to 6 membered heterocycloalkyl), R⁸³-substituted or unsubstituted aryl (e.g. C₆-C₁₀ aryl or C₆ aryl), or R⁸³-substituted or unsubstituted heteroaryl (e.g. 5 to 10 membered heteroaryl, 5 to 9 membered heteroaryl, or 5 to 6 membered heteroaryl). X⁸² is halogen. In embodiments, X⁸² is F.

R³², R³⁵, R³⁸, R^{4.1}, R^{4.4}, R⁵⁰, R⁵³, R⁵⁶, R⁵⁹, R⁶², R⁶⁵, R⁶⁸, R⁷¹, R⁷⁴, R⁷⁷, R⁸⁰ and R⁸³ are independently hydrogen, oxo, halogen, —CF₃, —CHF₂, —CH₂F, —OCH₂F, —OCF₃, —OCHF₂, —CCl₃, —CHCl₂, —CH₂Cl, —OCH₂Cl, —OCCl₃, —OCHCl₂, —CBr₃, —CHBr₂, —CH₂Br. —OCH₂Br, —OCBr₃, —OCHBr₂, —CI₃, —CHI₂, —CH₂I, —OCH₂I, —OCI₃, —OCHI₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g. C₁-C₈ alkyl, C₁-C₆ alkyl, or C₁-C₄ alkyl), unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), unsubstituted cycloalkyl (e.g. C₃-C₈ cycloalkyl, C₄-C₈ cycloalkyl, or C₅-C₆ cycloalkyl), unsubstituted heterocycloalkyl (e.g. 3 to 8 membered heterocycloalkyl, 4 to 8 membered heterocycloalkyl, or 5 to 6 membered heterocycloalkyl), unsubstituted aryl (e.g. C₆-C₁₀ aryl or C₆ aryl), or unsubstituted heteroaryl (e.g. 5 to 10 membered heteroaryl, 5 to 9 membered heteroaryl, or 5 to 6 membered heteroaryl). In embodiments, R³², R³⁵, R³⁸, R^{4.1}, R^{4.4}, R⁵⁰, R⁵³, R⁵⁶, R⁵⁹, R⁶², R⁶⁵, R⁶⁸, R⁷¹, R⁷⁴, R⁷⁷, R⁸⁰, and R⁸³ are independently oxo, halogen, —CF₃, —CHF₂, —CH₂F, —OCH₂F, —OCF₃, —OCHF₂, —CCl₃, —CHCl₂, —CH₂Cl, —OCH₂Cl, —OCCl₃, —OCHCl₂, —CBr₃, —CHBr₂, —CH₂Br, —OCH₂Br, —OCBr₃, —OCHBr₂, —CI₃, —CHI₂, —CH₂I, —OCH₂I, —OCI₃, —OCHI₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g. C₁-C₈ alkyl, C₁-C₆ alkyl, or C₁-C₄ alkyl), unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), unsubstituted cycloalkyl (e.g. C₃-C₈ cycloalkyl, C₄-C₈ cycloalkyl, or C₅-C₆ cycloalkyl), unsubstituted heterocycloalkyl (e.g. 3 to 8 membered heterocycloalkyl, 4 to 8 membered heterocycloalkyl, or 5 to 6 membered heterocycloalkyl), unsubstituted aryl (e.g. C₆-C₁₀ aryl or C₆ aryl), or unsubstituted heteroaryl (e.g. 5 to 10 membered heteroaryl, 5 to 9 membered heteroaryl, or 5 to 6 membered heteroaryl).

In embodiments, a compound as described herein may include multiple instances of R¹, R⁴, R⁵, R⁷, R⁸, R⁹, R¹⁰, R¹¹, R¹², R¹³, R¹⁴, R¹⁵, R¹⁶, R¹⁷, R¹⁸, R¹⁹, and/or other variables. In such embodiments, each variable may optional be different and be appropriately labeled to distinguish each group for greater clarity. For example, where each R¹, R⁴, R⁵, R¹, R⁸, R⁹, R¹⁰, R¹¹, R¹², R¹³, R¹⁴, R¹⁵, R¹⁶, R¹⁷, R¹⁸, and/or R¹⁹ is different, they may be referred to, for example, as R^{1.1}, R^{1.2}, R^{1.3}, R^{1.4}, R^{1.5}, R^{4.1}, R^{4.2}, R^{4.3}, R^{4.4}, R^{4.5}, R^{5.1}, R^{5.2}, R^{5.3}, R^{5.4}, R^{5.5}, R^{5.6}, R^{5.7}, R^{7.1}, R^{7.2}, R^{7.3}, R^{7.4}, R^{7.5}, R^{7.6}, R^{7.7}, R^{7.8}, R^{7.9}, R^{7.10}, R^{7.11}, R^{7.12}, R^{7.13}, R^{7.14}, R^{7.15}, R^{7.16}, R^{7.17}, R^{7.18}, R^{7.19}, R^{7.20}, R^{7.21}, R^{7.22}, R^{7.23}, R^{7.24}, R^{7.25}, R^{7.26}, R^{7.27}, R^{7.28}, R^{7.29}, R^{7.30}, R^{7.31}, R^{7.32}, R^{7.33}, R^{7.34}, R^{7.35}, R^{7.36}, R^{7.37}, R^{7.38}, R^{7.39}, R^{7.40}, R^{7.41}, R^{7.42}, R^{8.1}, R^{8.2}, R^{8.3}, R^{8.4}, R^{8.5}, R^{8.6}, R^{8.7}, R^{8.8}, R^{8.9}, R^{8.10}, R^{8.11}, R^{8.12}, R^{8.13}, R^{8.14}, R^{8.15}, R^{8.16}, R^{8.17}, R^{8.18}, R^{8.19}, R^{8.20}, R^{8.21}, R^{8.22}, R^{8.23}, R^{8.24}, R^{8.25}, R^{8.26}, R^{8.27}, R^{8.28}, R^{8.29}, R^{8.30}, R^{8.31}, R^{8.32}, R^{8.33}, R^{8.34}, R^{8.35}, R^{8.36}, R^{8.37}, R^{8.38}, R^{8.39}, R^{8.40}, R^{8.41}, R^{8.42}, R^{9.1}, R^{9.2}, R^{9.3}, R^{9.4}, R^{9.5}, R^{9.6}, R^{9.7}, R^{9.8}, R^{9.9}, R^{9.10}, R^{9.11}, R^{9.12}, R^{9.13}, R^{9.14}, R^{9.15}, R^{9.16}, R^{9.17}, R^{9.18}, R^{9.19}, R^{9.20}, R^{9.21}, R^{9.22}, R^{9.23}, R^{9.24}, R^{9.25}, R^{9.26}, R^{9.27}, R^{9.28}, R^{9.29}, R^{9.30}, R^{9.31}, R^{9.32}, R^{9.33}, R^{9.34}, R^{9.35}, R^{9.36}, R^{9.37}, R^{9.38}, R^{9.39}, R^{9.40}, R^{9.41}, R^{9.42}, R^{10.1}, R^{10.2}, R^{10.3}, R^{10.4}, R^{10.5}, R^{10.6}, R^{10.7}, R^{10.8}, R^{10.9}, R^{10.10}, R^{10.11}, R^{10.12}, R^{10.13}, R^{10.14}, R^{10.15}, R^{10.16}, R^{10.17}, R^{10.18}, R^{10.19}, R^{10.20}, R^{10.21}, R^{10.22}, R^{10.23}, R^{10.24}, R^{10.25}, R^{10.26}, R^{10.27}, R^{10.28}, R^{10.29}, R^{10.30}, R^{10.31}, R^{10.32}, R^{10.33}, R^{10.34}, R^{10.35}, R^{10.36}, R^{10.37}, R^{10.38}, R^{10.39}, R^{10.40}, R^{10.41}, R^{10.42}, R^{11.1}, R^{11.2}, R^{11.3}, R^{11.4}, R^{11.5}, R^{11.6}, R^{11.7}, R^{11.8}, R^{11.9}, R^{11.10}, R^{11.11}, R^{11.12}, R^{11.13}, R^{11.14}, R^{11.15}, R^{11.16}, R^{11.17}, R^{11.18}, R^{11.19}, R^{11.20}, R^{11.21}, R^{11.22}, R^{11.23}, R^{11.24}, R^{11.25}, R^{11.26}, R^{11.27}, R^{11.28}, R^{11.29}, R^{11.30}, R^{11.31}, R^{11.32}, R^{11.33}, R^{11.34}, R^{11.35}, R^{11.36}, R^{11.37}, R^{11.38}, R^{11.39}, R^{11.40}, R^{11.41}, R^{11.42}, R^{12.1}, R^{12.2}, R^{12.3}, R^{12.4}, R^{12.5}, R^{12.6}, R^{12.7}, R^{12.8}, R^{12.9}, R^{12.10}, R^{12.11}, R^{12.12}, R^{12.13}, R^{12.14}, R^{12.15}, R^{12.16}, R^{12.17}, R^{12.18}, R^{12.19}, R^{12.20}, R^{12.21}, R^{12.22}, R^{12.23}, R^{12.24}, R^{12.25}, R^{12.26}, R^{12.27}, R^{12.28}, R^{12.29}, R^{12.30}, R^{12.31}, R^{12.32}, R^{12.33}, R^{12.34}, R^{12.35}, R^{12.36}, R^{12.37}, R^{12.39}, R^{12.39}, R^{12.40}, R^{12.41}, R^{12.42}, R^{13.1}, R^{13.2}, R^{13.3}, R^{13.4}, R^{13.5}, R^{13.6}, R^{13.7}, R^{13.8}, R^{13.9}, R^{13.10}, R^{13.11}, R^{13.12}, R^{13.13}, R^{13.14}, R^{13.15}, R^{13.16}, R^{13.17}, R^{13.18}, R^{13.19}, R^{13.20}, R^{13.21}, R^{13.22}, R^{13.23}, R^{13.24}, R^{13.25}, R^{13.26}, R^{13.27}, R^{13.28}, R^{13.29}, R^{13.30}, R^{13.31}, R^{13.32}, R^{13.33}, R^{13.34}, R^{13.35}, R^{13.36}, R^{13.37}, R^{13.38}, R^{13.39}, R^{13.40}, R^{13.41}, R^{13.42}, R^{14.1}, R^{14.2}, R^{14.3}, R^{14.4}, R^{14.5}, R^{14.6}, R^{14.7}, R^{14.8}, R^{14.9}, R^{14.10}, R^{14.11}, R^{14.12}, R^{14.13}, R^{14.14}, R^{14.15}, R^{14.16}, R^{14.17}, R^{14.18}, R^{14.19}, R^{14.20}, R^{14.21}, R^{14.22}, R^{14.23}, R^{14.24}, R^{14.25}, R^{14.26}, R^{14.27}, R^{14.28}, R^{14.29}, R^{14.30}, R^{14.31}, R^{14.32}, R^{14.33}, R^{14.34}, R^{14.35}, R^{14.36}, R^{14.37}, R^{14.38}, R^{14.39}, R^{14.40}, R^{14.41}, R^{14.42}, R^{15.1}, R^{15.2}, R^{15.3}, R^{15.4}, R^{15.5}, R^{15.6}, R^{15.7}, R^{15.8}, R^{15.9}, R^{15.10}, R^{15.11}, R^{15.12}, R^{15.13}, R^{15.14}, R^{15.15}, R^{15.16}, R^{15.17}, R^{15.18}, R^{15.19}, R^{15.20}, R^{15.21}, R^{15.22}, R^{15.23}, R^{15.24}, R^{15.25}, R^{15.26}, R^{15.27}, R^{15.28}, R^{15.29}, R^{15.30}, R^{15.31}, R^{15.32}, R^{15.33}, R^{15.34}, R^{15.35}, R^{15.36}, R^{15.37}, R^{15.38}, R^{15.39}, R^{15.40}, R^{15.41}, R^{15.42}, R^{16.1}, R^{16.2}, R^{16.3}, R^{16.4}, R^{16.5}, R^{16.6}, R^{16.7}, R^{16.8}, R^{16.9}, R^{16.10}, R^{16.11}, R^{16.12}, R^{16.13}, R^{16.14}, R^{16.15}, R^{16.16}, R^{16.17}, R^{16.18}, R^{16.19}, R^{16.20}, R^{16.21}, R^{16.22}, R^{16.23}, R^{16.24}, R^{16.25}, R^{16.26}, R^{16.27}, R^{16.29}, R^{16.29}, R^{16.30}, R^{16.31}, R^{16.32}, R^{16.33}, R^{16.34}, R^{16.35}, R^{16.36}, R^{16.37}, R^{16.38}, R^{16.39}, R^{16.40}, R^{16.41}, R^{16.42}, R^{17.1}, R^{17.2}, R^{17.3}, R^{17.4}, R^{17.5}, R^{17.6}, R^{17.7}, R^{17.8}, R^{17.9}, R^{17.10}, R^{17.11}, R^{17.12}, R^{17.13}, R^{17.14}, R^{17.15}, R^{17.16}, R^{17.17}, R^{17.18}, R^{17.19}, R^{17.20}, R^{17.21}, R^{17.22}, R^{17.23}, R^{17.24}, R^{17.25}, R^{17.26}, R^{17.27}, R^{17.29}, R^{17.29}, R^{17.30}, R^{17.31}, R^{17.32}, R^{17.33}, R^{17′34}, R^{17.35}, R^{17.36}, R^{17.37}, R^{17.38}, R^{17.39}, R¹⁷⁴⁰, R^{17.41}, R^{17.42}, R^{18.1}, R^{18.2}, R^{18.3}, R^{18.4}, R^{18.5}, R^{18.6}, R^{18.7}, R^{18.8}, R^{18.9}, R^{18.10}, R^{18.11}, R^{18.12}, R^{18.13}, R^{18.14}, R^{18.15}, R^{18.16}, R^{18.17}, R^{18.18}, R^{18.19}, R^{18.20}, R^{18.21}, R^{18.22}, R^{18.23}, R^{18.24}, R^{18.25}, R^{18.26}, R^{18.27}, R^{18.28}, R^{18.29}, R^{18.30}, R^{18.31}, R^{18.32}, R^{18.33}, R^{18.34}, R^{18.35}, R^{18.36}, R^{18.37}, R^{18.38}, R^{18.39}, R^{18.40}, R^{18.41}, R^{18.42} respectively, wherein the definition of R¹ is assumed by R^{1.1}, R^{1.2}, R^{1.3}, R^{1.4}, R^{1.5}; R⁴ is assumed by R^{4.1}, R^{4.2}, R^{4.3}, R^{4.4}, R^{4.5}; R⁵ is assumed by R^{5.1}, R^{5.2}, R^{5.3}, R^{5.4}, R^{5.5}, R^{5.6}, R^{5.7}; R⁷ is assumed by R^{7.1}, R^{7.2}, R^{7.3}, R^{7.4}, R^{7.5}, R^{7.6}, R^{7.7}, R⁷⁸, R^{7.9}, R^{7.10}, R^{7.11}, R^{7.12}, R^{7.13}, R^{7.14}, R^{7.15}, R^{7.16}, R^{7.17}, R^{7.18}, R^{7.19}, R^{7.20}, R^{7.21}, R^{7.22}, R^{7.23}. R^{7.24}, R^{7.25}, R^{7.26}, R^{7.27}, R^{7.28}, R^{7.29}, R^{7.30}, R^{7.31}, R^{7.32}, R^{7.33}, R^{7.34}, R^{7.35}, R^{7.36}, R^{7.37}, R^{7.38}, R^{7.39}, R^{7.40}, R^{7.41}, R^{7.42}; R⁸ is assumed by R^{8.1}, R^{8.2}, R^{8.3}, R^{8.4}, R^{8.5}, R^{8.6}, R^{8.7}, R^{8.8}, R^{8.9}, R^{8.10}, R^{8.11}, R^{8.12}, R^{8.13}, R^{8.14}, R^{8.15}, R^{8.16}, R^{8.17}, R^{8.18}, R^{8.19}, R^{8.20}, R^{8.21}, R^{8.22}, R^{8.23}, R^{8.24}, R^{8.25}, R^{8.26}, R^{8.27}, R^{8.28}, R^{8.29}, R^{8.30}, R^{8.31}, R^{8.32}, R^{8.33}, R^{8.34}, R^{8.35}, R^{8.36}, R^{8.37}, R^{8.38}, R^{8.39}, R^{8.40}, R^{8.41}, R^{8.42}; R⁹ is assumed by R^{9.1}, R^{9.2}, R^{9.3}, R^{9.4}, R^{9.5}, R^{9.6}, R^{9.7}, R^{9.8}, R^{9.9}, R^{9.10}, R^{9.11}, R^{9.12}, R^{9.13}, R^{9.14}, R^{9.15}, R^{9.16}, R^{9.17}, R^{9.18}, R^{9.19}, R^{9.20}, R^{9.21}, R^{9.22}, R^{9.23}, R^{9.24}, R^{9.25}, R^{9.26}, R^{9.27}, R^{9.28}, R^{9.29}, R^{9.30}, R^{9.31}, R^{9.32}, R^{9.33}, R^{9.34}, R^{9.35}, R^{9.36}, R^{9.37}, R^{9.38}, R^{9.39}, R^{9.40}, R^{9.41}, R^{9.42}, R¹⁰ is assumed by R^{10.1}, R^{10.2}, R^{10.3}, R^{10.4}, R^{10.5}. R^{10.6}. R^{10.7}, R^{10.8}, R^{10.9}, R^{10.10}, R^{10.11}, R^{10.12}, R^{10.13}, R^{10.14}, R^{10.15}, R^{10.16}, R^{10.17}, R^{10.18}, R^{10.19}, R^{10.20}, R^{10.21}, R^{10.22}, R^{10.23}, R^{10.24}, R^{10.25}, R^{10.26}, R^{10.27}, R^{10.28}, R^{10.29}, R^{10.30}, R^{10.31}, R^{10.32}, R^{10.33}, R^{10.34}, R^{10.35}, R^{10.36}, R^{10.37}, R^{10.38}, R^{10.39}, R^{10.40}, R^{10.41}, R^{10.42}; R¹¹ is assumed by R^{11.1}, R^{11.2}, R^{11.3}, R^{11.4}, R^{11.5}, R^{11.6}, R^{11.7}, R^{11.8}, R^{11.9}, R^{11.10}, R^{11.11}, R^{11.12}, R^{11.13}, R^{11.14}, R^{11.15}, R^{11.16}, R^{11.17}, R^{11.18}, R^{11.19}, R^{11.20}, R^{11.21}, R^{11.22}, R^{11.23}, R^{11.24}, R^{11.25}, R^{11.26}, R^{11.27}, R^{11.28}, R^{11.29}, R^{11.30}, R^{11.31}, R^{11.32}, R^{11.33}, R^{11.34}, R^{11.35}, R^{11.36}, R^{11.37}, R^{11.38}, R^{11.39}, R^{11.40}, R^{11.41}, R^{11.42}; R¹² is assumed by R^{12.1}, R^{12.2}, R^{12.3}, R^{12.4}, R^{12.5}, R^{12.6}, R^{12.7}, R^{12.8}, R^{12.9}, R^{12.10}, R^{12.11}, R^{12.12}, R^{12.13}, R^{12.14}, R^{12.15}, R^{12.16}, R^{12.17}, R^{12.18}, R^{12.19}, R^{12.20}, R^{12.21}, R^{12.22}, R^{12.23}, R^{12.24}, R^{12.25}, R^{12.26}, R^{12.27}, R^{12.28}, R^{12.29}, R^{12.30}, R^{12.31}, R^{12.32}, R^{12.33}, R^{12.34}, R^{12.35}, R^{12.36}, R^{12.37}, R^{12.38}, R^{12.39}, R^{12.40}, R^{12.41}, R^{12.42}; R¹³ is assumed by R^{13.1}, R^{13.2}, R^{13.3}, R^{13.4}, R^{13.5}, R^{13.6}, R^{13.7}, R^{13.8}, R^{13.9}, R^{13.10}, R^{13.11}, R^{13.12}, R^{13.13}, R^{13.14}, R^{13.15}, R^{13.16}, R^{13.17}, R^{13.18}, R^{13.19}, R^{13.20}, R^{13.21}, R^{13.22}, R^{13.23}, R^{13.24}, R^{13.25}, R^{13.26}, R^{13.27}, R^{13.28}, R^{13.29}, R^{13.30}, R^{13.31}, R^{13.32}, R^{13.33}, R^{13.34}, R^{13.35}, R^{13.36}, R^{13.37}, R^{13.38}, R^{13.39}, R^{13.40}, R^{13.41}, R^{13.42}; R¹⁴ is assumed by R^{14.1}, R^{14.2}, R^{14.3}, R^{14.4}, R^{14.5}, R^{14.6}, R^{14.7}, R^{14.8}, R^{14.9}, R^{14.10}, R^{14.11}, R^{14.12}, R^{14.13}, R^{14.14}, R^{14.15}, R^{14.16}, R^{14.17}, R^{14.18}, R^{14.19}, R^{14.20}, R^{14.21}, R^{14.22}, R^{14.23}, R^{14.24}, R^{14.25}, R^{14.26}, R^{14.27}, R^{14.28}, R^{14.29}, R^{14.30}, R^{14.31}, R^{14.32}, R^{14.33}, R^{14.34}, R^{14.35}, R^{14.36}, R^{14.37}, R^{14.38}, R^{14.39}, R^{14.40}, R^{14.41}, R^{14.42}; R¹⁵ is assumed by R^{15.1}, R^{15.2}, R^{15.3}, R^{15.4}, R^{15.5}, R^{15.6}, R^{15.7}, R^{15.8}, R^{15.9}, R^{15.10}, R^{15.11}, R^{15.12}, R^{15.13}, R^{15.14}, R^{15.15}, R^{15.16}, R^{15.17}, R^{15.18}, R^{15.19}, R^{15.20}, R^{15.21}, R^{15.22}, R^{15.23}, R^{15.24}, R^{15.25}, R^{15.26}, R^{15.27}, R^{15.28}, R^{15.29}, R^{15.30}, R^{15.31}, R^{15.32}, R^{15.33}, R^{15.34}, R^{15.35}, R^{15.36}, R^{15.37}, R^{15.38}, R^{15.39}, R^{15.40}. R^{15.41}, R^{15.42}; R¹⁶ is assumed by R^{16.1}, R^{16.2}, R¹⁶³, R^{16.4}, R^{16.5}, R^{16.6}, R^{16.7}, R^{16.8}, R^{16.9}, R^{16.10}, R^{16.11}, R^{16.12}, R^{16.13}, R^{16.14}, R^{16.15}, R^{16.16}, R^{16.17}, R^{16.18}, R^{16.19}, R^{16.20}, R^{16.21}, R^{16.22}, R^{16.23}, R^{16.24}, R^{16.25}, R^{16.26}, R^{16.27}, R^{16.28}, R^{16.29}, R^{16.30}, R^{16.31}, R^{16.32}, R^{16.33}, R^{16.34}, R^{16.35}, R^{16.36}, R^{16.37}, R^{16.38}, R^{16.39}. R^{16.40}, R^{16.41}, R^{16.42}; R¹⁷ is assumed by R^{17.1}. R^{17.2}, R^{17.3}, R^{17.4}, R^{17.5}, R^{17.6}, R^{17.7}, R^{17.8}, R^{17.9}, R¹⁷¹⁰, R^{17.11}, R^{17.12}, R^{17.13}, R^{17.14}, R^{17.15}, R^{17.16}, R^{17.17}, R^{17.18}, R^{17.19}, R^{17.20}, R^{17.21}, R^{17.22}, R^{17.23}, R^{17.24}, R^{17.25}, R^{17.26}, R^{17.27}, R^{17.28}, R^{17.29}, R^{17.30}, R^{17.31}, R^{17.32}, R^{17.33}, R^{17.34}, R^{17.35}, R^{17.36}, R^{17.37}, R^{17.38}. R^{17.39}, R^{17.40}, R^{17.41}, R^{17.42}; and/or R¹⁸ is assumed by R^{18.1}, R^{18.2}, R^{18.3}, R^{18.4}, R^{18.5}, R^{18.6}, R^{18.7}, R^{18.8}, R^{18.9}, R^{18.10}, R^{18.11}, R^{18.12}, R^{18.13}, R^{18.14}, R^{18.15}, R^{18.16}, R^{18.17}, R^{18.18}, R^{18.19}, R^{18.20}, R^{18.21}, R^{18.22}, R^{18.23}, R^{18.24}, R^{18.25}, R^{18.26}, R^{18.27}, R^{18.28}, R^{18.29}, R^{18.30}, R^{18.31}, R^{18.32}, R^{18.33}, R^{18.34}, R^{18.35}, R^{18.36}, R^{18.37}, R^{18.38}, R^{18.39}, R^{18.40}, R^{18.41}, R^{18.42}. The variables used within a definition of R¹, R⁴, R⁵, R⁷, R⁸, R⁹, R¹⁰, R¹¹, R¹¹, R¹², R¹³, R¹⁴, R¹⁵, R¹⁶, R¹⁷, R¹⁸, R¹⁹, and/or other variables that appear at multiple instances and are different may similarly be appropriately labeled to distinguish each group for greater clarity.

In embodiments, the PCNS inhibitor is a compound having the formula:

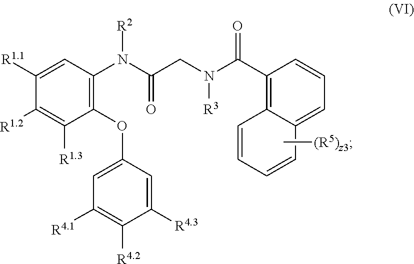

wherein R², R³, R⁵, and z3 are as described herein, including in compounds of formula (I) to (V). R^{1.1}, R^{1.2}, and R^{1.3} are each independently a moiety of R¹ as described herein, including in embodiments. R^{4.1}, R^{4.2}, and R^{4.3} are each independently a moiety of R⁴ as described herein, including in embodiments. In embodiment; z3 is 0. In embodiments, one or more of R^{1.1}, R^{1.2}, R^{1.3}, R^{4.1}, R^{4.2}, R^{4.3}, R² and/or R³ are hydrogen. In embodiments, R^{1.1}, R^{1.2} and/or R^{1.3} are hydrogen. In embodiments, R^{4.1}, R^{4.2} and/or R^{4.3} are hydrogen. In embodiments, R² is hydrogen. In embodiments, R³ is hydrogen. In embodiments, R^{4.1} is hydrogen, R^{4.2} is —OH, and R^{4.3} is hydrogen. In embodiments, R^{4.1} is hydrogen. R^{4.2} is hydrogen, and R^{4.3} is —OH. In embodiments, R^{4.1} is hydrogen, R^{4.2} is unsubstituted methoxy, and R^{4.3} is hydrogen. In embodiments, R^{4.1} is hydrogen, R^{4.2} is hydrogen, and R^{4.3} is unsubstituted methoxy. It will be understood that R⁵ is/are a floating substituent and may be positioned on either or both rings.

In embodiments, the PCNS inhibitor is a compound having the formula:

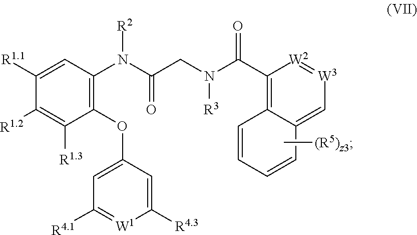

wherein R², R³, R⁵, and z3 are as described herein, including in compounds of formula (I) to (V). It will be understood that R⁵ is/are a floating substituent and may be positioned on either or both rings. R^{1.1}, R^{1.2}, and R^{1.3} are each independently a moiety of R¹ as described herein, including in embodiments. R^{4.1} and R^{4.3} are each independently a moiety of R⁴ as described herein, including in embodiments.

W¹ is N or C(R^{4.2}). W² is N or C(R^{5.1}). W³ is N or C(R^{5.2}). R^{5.1} and R^{5.2} are each independently a moiety of R⁵ as described herein, including in embodiments. R^{4.2} is independently a moiety of R⁴ as described herein, including in embodiments. In embodiments, W¹ is N. In embodiments, W² is N. In embodiments, W³ is N. In embodiments, W¹ is C(R^{4.2}). In embodiments, W² is C(R^{5.1}). In embodiments, W³ is C(R^{5.2}). In embodiments, W¹ is CH. In embodiments, W² is CH. In embodiments, W³ is CH.

In embodiments, the PCNS inhibitor is a compound having the formula:

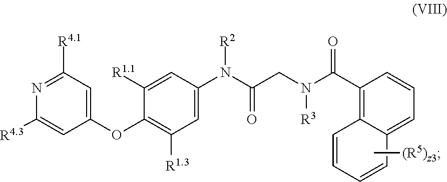

wherein R², R³, R⁵, and z3 are as described herein, including in compounds of formula (I) to (V). It will be understood that R⁵ is/are a floating substituent and may be positioned on either or both rings. R^{1.1} and R¹³ are each independently a moiety of R¹ as described herein, including in embodiments. R^{4.1} and R^{4.3} are each independently a moiety of R⁴ as described herein, including in embodiments.

In embodiments, the PCNS inhibitor is a compound having the formula:

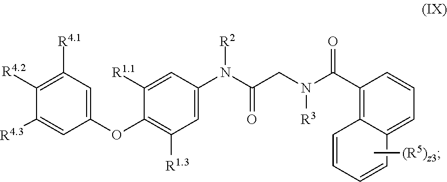

wherein R², R³, R⁵, and z3 are as described herein, including in compounds of formula (I) to (V). It will be understood that R⁵ is/are a floating substituent and may be positioned on either or both rings. R^{1.1} and R¹³ are each independently a moiety of R¹ as described herein, including in embodiments. R^{4.1}, R^{4.2}, and R^{4.3} are each independently a moiety of R⁴ as described herein, including in embodiments.

In embodiments, the PCNS inhibitor is a compound having the formula:

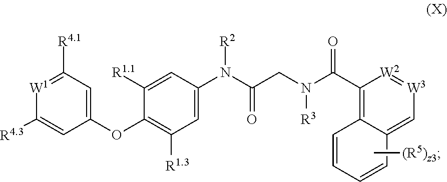

wherein R², R³, R⁵, and z3 are as described herein, including in compounds of formula (I) to (V). It will be understood that R⁵ is/are a floating substituent and may be positioned on either or both rings. R^{1.1} and R¹³ are each independently a moiety of R¹ as described herein, including in embodiments. R^{4.1} and R^{4.3} are each independently a moiety of R⁴ as described herein, including in embodiments.

W¹ is N or C(R^{4.2}). W² is N or C(R^{5.1}) W³ is N or C(R^{5.2}). R^{5.1} and R^{5.2} are each independently a moiety of R⁵ as described herein, including in embodiments. R^{4.2} is independently a moiety of R⁴ as described herein, including in embodiments. In embodiments, W¹ is N. In embodiments, W² is N. In embodiments, W³ is N. In embodiments, W¹ is C(R^{4.2}). In embodiments, W² is C(R^{5.1}). In embodiments, W³ is C(R^{1.2}). In embodiments, W¹ is CH. In embodiments, W² is CH. In embodiments, W³ is CH.

In embodiments of the compounds of formula (VI) to (X), R² is hydrogen. n embodiments of the compounds of formula (VI) to (X), R³ is hydrogen. In embodiments of the compounds of formula (VI) to (X), R² and R³ are hydrogen.

In embodiments, R¹¹ is independently halogen. In embodiments, R^{1.1} is independently —CF₃. In embodiments, R^{1.1} is independently —CHF₂. In embodiments, R^{1.1} is independently —CH₂F. In embodiments, R^{1.1} is independently —OCF₃. In embodiments, R^{1.1} is independently —OCHF₂. In embodiments, R^{1.1} is independently —OCH₂F. In embodiments, R^{1.1} is independently —OH. In embodiments, R^{1.1} is independently —NH₂. In embodiments, R^{1.1} is independently —SH. In embodiments, R^{1.1} is independently substituted or unsubstituted C₁-C₄ alkyl. In embodiments, R^{1.1} is independently substituted or unsubstituted 2 to 4 membered heteroalkyl. In embodiments, R^{1.1} is independently substituted or unsubstituted C₃-C₆ cycloalkyl. In embodiments, R^{1.1} is independently substituted or unsubstituted 3 to 6 membered heterocycloalkyl. In embodiments, R^{1.1} is independently substituted or unsubstituted phenyl. In embodiments, R^{1.1} is independently substituted or unsubstituted 5 to 6 membered heteroaryl. In embodiments, R^{1.1} is independently substituted C₁-C₄ alkyl. In embodiments, R^{1.1} is independently substituted to 4 membered heteroalkyl. In embodiments, R^{1.1} is independently substituted C₃-C₆ cycloalkyl. In embodiments, R^{1.1} is independently substituted 3 to 6 membered heterocycloalkyl. In embodiments, R^{1.1} is independently substituted phenyl. In embodiments, R^{1.1} is independently substituted 5 to 6 membered heteroaryl. In embodiments, R^{1.1} is independently unsubstituted C₁-C₄ alkyl. In embodiments, R^{1.1} is independently unsubstituted 2 to 4 membered heteroalkyl. In embodiments, R^{1.1} is independently unsubstituted C₃-C₆ cycloalkyl. In embodiments, R^{1.1} is independently unsubstituted 3 to 6 membered heterocycloalkyl. In embodiments, R^{1.1} is independently unsubstituted phenyl. In embodiments, R^{1.1} is independently unsubstituted 5 to 6 membered heteroaryl. In embodiments, R^{1.1} is independently unsubstituted methyl. In embodiments, R^{1.1} is independently unsubstituted ethyl. In embodiments, R^{1.1} is independently unsubstituted isopropyl. In embodiments, R^{1.1} is independently unsubstituted tert-butyl. In embodiments, R^{1.1} is independently unsubstituted methoxy. In embodiments, R^{1.1} is independently unsubstituted ethoxy. In embodiments, R¹¹ is independently —F. In embodiments; R^{1.1} is independently —Cl. In embodiments, R^{1.1} is independently —Br. In embodiments, R^{1.1} is independently —I. In embodiments, R^{1.1} is independently hydrogen.

In embodiments, R^{1.2} is independently halogen. In embodiments, R^{1.2} is independently —CF₃. In embodiments, R^{1.2} is independently —CHF₂. In embodiments, R^{1.2} is independently —CH₂F. In embodiments, R¹² is independently —OCF₃. In embodiments, R^{1.2} is independently —OCHF₂. In embodiments, R^{1.2} is independently —OCH₂F. In embodiments, R^{1.2} is independently —OH. In embodiments, R^{1.2} is independently —NH₂. In embodiments, R^{1.2} is independently —SH. In embodiments, R^{1.2} is independently substituted or unsubstituted C₁-C₄ alkyl. In embodiments, R^{1.2} is independently substituted or unsubstituted 2 to 4 membered heteroalkyl. In embodiments, R^{1.2} is independently substituted or unsubstituted C₃-C₆ cycloalkyl. In embodiments, R^{1.2} is independently substituted or unsubstituted 3 to 6 membered heterocycloalkyl. In embodiments, R^{1.2} is independently substituted or unsubstituted phenyl. In embodiments, R^{1.2} is independently substituted or unsubstituted 5 to 6 membered heteroaryl. In embodiments, R^{1.2} is independently substituted C₁-C₄ alkyl. In embodiments, R¹² is independently substituted to 4 membered heteroalkyl. In embodiments, R¹² is independently substituted C₃-C₆ cycloalkyl. In embodiments, R¹² is independently substituted 3 to 6 membered heterocycloalkyl. In embodiments, R^{1.2} is independently substituted phenyl. In embodiments, R¹² is independently substituted 5 to 6 membered heteroaryl. In embodiments, R¹² is independently unsubstituted C₁-C₄ alkyl. In embodiments, R¹² is independently unsubstituted 2 to 4 membered heteroalkyl. In embodiments, R¹² is independently unsubstituted C₃-C₆ cycloalkyl. In embodiments, R^{1.2} is independently unsubstituted 3 to 6 membered heterocycloalkyl. In embodiments, R^{1.2} is independently unsubstituted phenyl. In embodiments, R^{1.2} is independently unsubstituted 5 to 6 membered heteroaryl. In embodiments, R^{1.2} is independently unsubstituted methyl. In embodiments, R^{1.2} is independently unsubstituted ethyl. In embodiments, R^{1.2} is independently unsubstituted isopropyl. In embodiments, R^{1.2} is independently unsubstituted tert-butyl. In embodiments, R^{1.2} is independently unsubstituted methoxy. In embodiments, R^{1.2} is independently unsubstituted ethoxy. In embodiments, R^{1.2} is independently —F. In embodiments; R^{1.2} is independently —Cl. In embodiments, R^{1.2} is independently —Br. In embodiments, R^{1.2} is independently —I. In embodiments, R^{1.2} is independently hydrogen.

In embodiments, R^{1.3} is independently halogen. In embodiments, R^{1.3} is independently —CF₃. In embodiments, R^{1.3} is independently —CHF₂. In embodiments, R^{1.3} is independently —CH₂F. In embodiments, R^{1.3} is independently —OCF₃. In embodiments, R^{1.3} is independently —OCHF₂. In embodiments, R^{1.3} is independently —OCH₂F. In embodiments, R^{1.3} is independently —OH. In embodiments, R^{1.3} is independently —NH₂. In embodiments, R^{1.3} is independently —SH. In embodiments, R^{1.3} is independently substituted or unsubstituted C₁-C₄ alkyl. In embodiments, R^{1.3} is independently substituted or unsubstituted 2 to 4 membered heteroalkyl. In embodiments, R^{1.3} is independently substituted or unsubstituted C₃-C₆ cycloalkyl. In embodiments, R^{1.3} is independently substituted or unsubstituted 3 to 6 membered heterocycloalkyl. In embodiments, R^{1.3} is independently substituted or unsubstituted phenyl. In embodiments, R^{1.3} is independently substituted or unsubstituted 5 to 6 membered heteroaryl. In embodiments, R^{1.3} is independently substituted C₁-C₄ alkyl. In embodiments, R^{1.3} is independently substituted to 4 membered heteroalkyl. In embodiments, R¹³ is independently substituted C₃-C₆ cycloalkyl. In embodiments, R¹³ is independently substituted 3 to 6 membered heterocycloalkyl. In embodiments, R^{1.3} is independently substituted phenyl. In embodiments, R^{1.3} is independently substituted 5 to 6 membered heteroaryl. In embodiments, R^{1.3} is independently unsubstituted C₁-C₄ alkyl. In embodiments, R^{1.3} is independently unsubstituted 2 to 4 membered heteroalkyl. In embodiments, R^{1.3} is independently unsubstituted C₃-C₆ cycloalkyl. In embodiments, R^{1.3} is independently unsubstituted 3 to 6 membered heterocycloalkyl. In embodiments, R^{1.3} is independently unsubstituted phenyl. In embodiments, R^{1.3} is independently unsubstituted 5 to 6 membered heteroaryl. In embodiments, R^{1.3} is independently unsubstituted methyl. In embodiments, R^{1.3} is independently unsubstituted ethyl. In embodiments, R^{1.3} is independently unsubstituted isopropyl. In embodiments, R^{1.3} is independently unsubstituted tert-butyl. In embodiments, R^{1.3} is independently unsubstituted methoxy. In embodiments, R^{1.3} is independently unsubstituted ethoxy. In embodiments, R^{1.3} is independently —F. In embodiments, R^{1.3} is independently —Cl. In embodiments, R^{1.3} is independently —Br. In embodiments, R^{1.3} is independently —I. In embodiments, R^{1.3} is independently hydrogen.

In embodiments, R^{1.4} is independently halogen. In embodiments, R^{1.4} is independently —CF₃. In embodiments, R^{1.4} is independently —CHF₂. In embodiments, R^{1.4} is independently —CH₂F. In embodiments, R^{1.4} is independently —OCF₃. In embodiments, R^{1.4} is independently —OCHF₂. In embodiments, R^{1.4} is independently —OCH₂F. In embodiments, R^{1.4} is independently —OH. In embodiments, R^{1.4} is independently —NH₂. In embodiments, R¹⁴ is independently —SH. In embodiments, R^{1.4} is independently substituted or unsubstituted C₁-C₄ alkyl. In embodiments, R^{1.4} is independently substituted or unsubstituted 2 to 4 membered heteroalkyl. In embodiments, R^{1.4} is independently substituted or unsubstituted C₃-C₆ cycloalkyl. In embodiments, R^{1.4} is independently substituted or unsubstituted 3 to 6 membered heterocycloalkyl. In embodiments, R^{1.4} is independently substituted or unsubstituted phenyl. In embodiments, R^{1.4} is independently substituted or unsubstituted 5 to 6 membered heteroaryl. In embodiments, R^{1.4} is independently substituted C₁-C₄ alkyl. In embodiments, R^{1.4} is independently substituted to 4 membered heteroalkyl. In embodiments, R^{1.4} is independently substituted C₃-C₆ cycloalkyl. In embodiments, R^{1.4} is independently substituted 3 to 6 membered heterocycloalkyl. In embodiments, R^{1.4} is independently substituted phenyl. In embodiments, R^{1.4} is independently substituted 5 to 6 membered heteroaryl. In embodiments, R^{1.4} is independently unsubstituted C₁-C₄ alkyl. In embodiments, R^{1.4} is independently unsubstituted 2 to 4 membered heteroalkyl. In embodiments, R¹⁴ is independently unsubstituted C₃-C₆ cycloalkyl. In embodiments, R^{1.4} is independently unsubstituted 3 to 6 membered heterocycloalkyl. In embodiments, R^{1.4} is independently unsubstituted phenyl. In embodiments, R^{1.4} is independently unsubstituted 5 to 6 membered heteroaryl. In embodiments, R^{1.4} is independently unsubstituted methyl. In embodiments, R^{1.4} is independently unsubstituted ethyl. In embodiments, R^{1.4} is independently unsubstituted isopropyl. In embodiments, R^{1.4} is independently unsubstituted tert-butyl. In embodiments, R^{1.4} is independently unsubstituted methoxy. In embodiments, R^{1.4} is independently unsubstituted ethoxy. In embodiments, R^{1.4} is independently —F. In embodiments; R^{1.4} is independently —Cl. In embodiments, R^{1.4} is independently —Br. In embodiments, R^{1.4} is independently —I. In embodiments, R^{1.4} is independently hydrogen.

In embodiments, R^{4.1} is independently halogen, —CF₃, —OH, —NH₂, —SH, substituted or unsubstituted C₁-C₄ alkyl, substituted or unsubstituted 2 to 4 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R^{4.1} is independently halogen, —OH, —NH₂, —SH, unsubstituted C₁-C₄ alkyl, or unsubstituted 2 to 4 membered heteroalkyl. In embodiments, R^{4.1} is independently halogen, —OH, unsubstituted methyl, or unsubstituted methoxy. In embodiments, R^{4.1} is independently halogen. In embodiments, R^{4.1} is independently —OH. In embodiments, R^{4.1} is independently unsubstituted methyl. In embodiments, R^{4.1} is independently unsubstituted methoxy. In embodiments, R^{4.1} is independently unsubstituted ethyl. In embodiments, R^{4.1} is independently —F. In embodiments; R^{4.1} is independently —Cl. In embodiments, R^{4.1} is independently —Br. In embodiments, R^{4.1} is independently —I. In embodiments, R^{4.1} is independently —CF₃. In embodiments, R^{4.1} is independently —NH₂. In embodiments, R^{4.1} is independently —SH. In embodiments, R^{4.1} is independently hydrogen. In embodiments, R^{4.1} is independently unsubstituted isopropyl. In embodiments, R^{4.1} is independently unsubstituted ethoxy. In embodiments, R^{4.1} is independently unsubstituted tert-butyl. In embodiments, R^{4.1} is independently unsubstituted propoxy.

In embodiments, R^{4.2} is independently halogen, —CF₃, —OH, —NH₂, —SH, substituted or unsubstituted C₁-C₄ alkyl, substituted or unsubstituted 2 to 4 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R^{4.2} is independently halogen, —OH, —NH₂, —SH, unsubstituted C₁-C₄ alkyl, or unsubstituted 2 to 4 membered heteroalkyl. In embodiments, R^{4.2} is independently halogen, —OH, unsubstituted methyl, or unsubstituted methoxy. In embodiments, R^{4.2} is independently halogen. In embodiments, R^{4.2} is independently —OH. In embodiments, R^{4.2} is independently unsubstituted methyl. In embodiments, R^{4.2} is independently unsubstituted methoxy. In embodiments, R^{4.2} is independently unsubstituted ethoxy. In embodiments, R^{4.2} is independently unsubstituted ethyl. In embodiments, R^{4.2} is independently —F. In embodiments, R^{4.2} is independently —Cl. In embodiments, R^{4.2} is independently —Br. In embodiments, R^{4.2} is independently —I. In embodiments, R^{4.2} is independently —CF₃. In embodiments, R^{4.2} is independently —NH₂. In embodiments, R^{4.2} is independently —SH. In embodiments, R^{4.2} is independently hydrogen. In embodiments, R^{4.2} is independently unsubstituted isopropyl. In embodiments, R^{4.2} is independently unsubstituted ethoxy. In embodiments, R^{4.2} is independently unsubstituted tert-butyl. In embodiments; R^{4.2} is independently unsubstituted propoxy.

In embodiments, R^{4.3} is independently halogen, —CF₃, —OH, —NH₂, —SH, substituted or unsubstituted C₁-C₄ alkyl, substituted or unsubstituted 2 to 4 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R^{4.3} is independently halogen, —OH, —NH₂, —SH, unsubstituted C₁-C₄ alkyl, or unsubstituted 2 to 4 membered heteroalkyl. In embodiments, R^{4.3} is independently halogen, —OH, unsubstituted methyl, or unsubstituted methoxy. In embodiments, R^{4.3} is independently halogen. In embodiments, R^{4.3} is independently —OH. In embodiments, R^{4.3} is independently unsubstituted methyl. In embodiments, R^{4.3} is independently unsubstituted methoxy. In embodiments, R^{4.3} is independently unsubstituted ethyl. In embodiments, R^{4.3} is independently —F. In embodiments. R^{4.3} is independently —Cl. In embodiments, R^{4.3} is independently —Br. In embodiments, R^{4.3} is independently —I. In embodiments, R^{4.3} is independently —CF₃. In embodiments, R^{4.3} is independently —NH₂. In embodiments, R^{4.3} is independently —SH. In embodiments, R^{4.3} is independently hydrogen. In embodiments, R^{4.3} is independently unsubstituted isopropyl. In embodiments, R^{4.3} is independently unsubstituted ethoxy. In embodiments, R^{4.3} is independently unsubstituted tert-butyl. In embodiments, R^{4.3} is independently unsubstituted propoxy.

In embodiments, R^{4.4} is independently halogen, —CF₃, —OH, —NH₂, —SH, substituted or unsubstituted C₁-C₄ alkyl, substituted or unsubstituted 2 to 4 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R^{4.4} is independently halogen, —OH, —NH₂, —SH, unsubstituted C₁-C₄ alkyl, or unsubstituted 2 to 4 membered heteroalkyl. In embodiments, R^{4.4} is independently halogen, —OH, unsubstituted methyl, or unsubstituted methoxy. In embodiments, R^{4.4} is independently halogen. In embodiments, R^{4.4} is independently —OH. In embodiments, R^{4.4} is independently unsubstituted methyl. In embodiments, R^{4.4} is independently unsubstituted methoxy. In embodiments, R^{4.4} is independently unsubstituted ethyl. In embodiments, R^{4.4} is independently —F. In embodiments; R^{4.4} is independently —Cl. In embodiments, R^{4.4} is independently —Br. In embodiments, R^{4.4} is independently —I. In embodiments, R^{4.4} is independently —CF₃. In embodiments, R^{4.4} is independently —NH₂. In embodiments, R^{4.4} is independently —SH. In embodiments, R^{4.4} is independently hydrogen. In embodiments, R^{4.4} is independently unsubstituted isopropyl. In embodiments, R^{4.4} is independently unsubstituted ethoxy. In embodiments, R^{4.4} is independently unsubstituted tert-butyl. In embodiments, R^{4.4} is independently unsubstituted propoxy.

In embodiments, R^{4.5} is independently halogen, —CF₃, —OH, —NH₂, —SH, substituted or unsubstituted C₁-C₄ alkyl, substituted or unsubstituted 2 to 4 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R^{4.5} is independently halogen, —OH, —NH₂, —SH, unsubstituted C₁-C₄ alkyl, or unsubstituted 2 to 4 membered heteroalkyl. In embodiments, R^{4.5} is independently halogen, —OH, unsubstituted methyl, or unsubstituted methoxy. In embodiments, R^{4.5} is independently halogen. In embodiments, R^{4.5} is independently —OH. In embodiments, R^{4.5} is independently unsubstituted methyl. In embodiments, R^{4.5} is independently unsubstituted methoxy. In embodiments, R^{4.5} is independently unsubstituted ethyl. In embodiments, R^{4.5} is independently —F. In embodiments. R^{4.5} is independently —Cl. In embodiments, R^{4.5} is independently —Br. In embodiments, R^{4.5} is independently —I. In embodiments, R^{4.5} is independently —CF₃. In embodiments, R^{4.5} is independently —NH₂. In embodiments, R^{4.5} is independently —SH. In embodiments, R^{4.5} is independently hydrogen. In embodiments, R^{4.5} is independently unsubstituted isopropyl. In embodiments, R^{4.5} is independently unsubstituted ethoxy. In embodiments, R^{4.5} is independently unsubstituted tert-butyl. In embodiments, R^{4.5} is independently unsubstituted propoxy.

In embodiments, R^{5.1} is independently halogen, —CF₃, —OH, —NH₂, —SH, substituted or unsubstituted C₁-C₄ alkyl, substituted or unsubstituted 2 to 4 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R^{5.1} is independently halogen, —OH, —NH₂, —SH, unsubstituted C₁-C₄ alkyl, or unsubstituted 2 to 4 membered heteroalkyl. In embodiments, R^{5.1} is independently halogen, —OH, unsubstituted methyl, or unsubstituted methoxy. In embodiments, R^{5.1} is independently halogen. In embodiments, R^{5.1} is independently —OH. In embodiments, R^{5.1} is independently unsubstituted methyl. In embodiments, R^{5.1} is independently unsubstituted methoxy. In embodiments, R^{5.1} is independently unsubstituted ethyl. In embodiments, R^{5.1} is independently —F. In embodiments; R^{5.1} is independently —Cl. In embodiments, R^{5.1} is independently —Br. In embodiments, R^{5.1} is independently —I. In embodiments, R^{5.1} is independently —CF₃. In embodiments, R^{5.1} is independently —NH₂. In embodiments, R^{5.1} is independently —SH. In embodiments, R^{5.1} is independently hydrogen. In embodiments, R^{5.2} is independently unsubstituted isopropyl. In embodiments, R^{5.1} is independently unsubstituted ethoxy. In embodiments, R^{5.1} is independently unsubstituted tert-butyl. In embodiments, R^{5.1} is independently unsubstituted propoxy.

In embodiments, R^{5.2} is independently halogen, —CF₃, —OH, —NH₂, —SH, substituted or unsubstituted C₁-C₄ alkyl, substituted or unsubstituted 2 to 4 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R^{5.2} is independently halogen, —OH, —NH₂, —SH, unsubstituted C₁-C₄ alkyl, or unsubstituted 2 to 4 membered heteroalkyl. In embodiments, R^{5.2} is independently halogen, —OH, unsubstituted methyl, or unsubstituted methoxy. In embodiments, R^{5.2} is independently halogen. In embodiments, R^{5.2} is independently —OH. In embodiments, R^{5.2} is independently unsubstituted methyl. In embodiments, R^{5.2} is independently unsubstituted methoxy. In embodiments, R^{5.2} is independently unsubstituted ethyl. In embodiments, R^{5.2} is independently —F. In embodiments. R^{5.22} is independently —Cl. In embodiments, R^{5.2} is independently —Br. In embodiments, R^{5.2} is independently —I. In embodiments, R^{5.2} is independently —CF₃. In embodiments, R^{5.2} is independently —NH₂. In embodiments, R^{5.2} is independently —SH. In embodiments, R^{5.2} is independently hydrogen. In embodiments, R^{5.2} is independently unsubstituted isopropyl. In embodiments, R^{5.2} is independently unsubstituted ethoxy. In embodiments, R^{5.2} is independently unsubstituted tert-butyl. In embodiments, R^{5.2} is independently unsubstituted propoxy.

In embodiments, W¹ is N. In embodiments, W¹ is C(R^{4.2}). In embodiments, W² is N. In embodiments, W² is C(R^{5.1}). In embodiments, W³ is N. In embodiments, W³ is C(R^{5.2}). In embodiments, W¹ is C(H). In embodiments, W² is C(H). In embodiments, W³ is C(H).

In embodiments, R^{1.1} and R^{1.3} are —I. In embodiments, R^{1.1} and R^{1.3} are —F. In embodiments, R^{1.1} and R^{1.3} are —Br. In embodiments, R^{1.1} and R^{1.3} are —Cl. In embodiments, R^{1.1} and R^{1.3} are unsubstituted methyl. In embodiments, R^{1.1} and R^{1.3} are —CF₃. In embodiments, R^{1.1} and R^{1.3} are —NH₂. In embodiments, R^{1.1} and R^{1.3} are —OH. In embodiments, R^{1.1} and R^{1.3} are unsubstituted methoxy. In embodiments, R^{1.1} and R¹³ are halogen. In embodiments, R^{1.1} and R^{1.3} are unsubstituted C₁-C₄ alkyl. In embodiments, R^{1.1} and R^{1.3} are substituted C₁-C₄ alkyl. In embodiments, R^{1.1} and R¹³ are halogen substituted C₁-C₄ alkyl. In embodiments, R¹¹ and R^{1.3} are unsubstituted C₁-C₂ alkyl. In embodiments, R^{1.1} and R¹³ are substituted C₁-C₂ alkyl. In embodiments, R^{1.1} and R^{1.3} are halogen substituted C₁-C₂ alkyl.

In embodiments, R^{4.1} and R^{4.3} are —I. In embodiments, R^{4.1} and R^{4.3} are —F. In embodiments, R^{4.1} and R^{4.3} are —Br. In embodiments, R^{4.1} and R^{4.3} are —Cl. In embodiments, R^{4.1} and R^{4.3} are unsubstituted methyl. In embodiments, R^{4.1} and R^{4.3} are —CF₃. In embodiments, R^{4.1} and R^{4.3} are —NH₂. In embodiments, R^{4.1} and R^{4.3} are —OH. In embodiments, R^{4.1} and R^{4.3} are unsubstituted methoxy. In embodiments, R^{4.1} and R^{4.3} are halogen. In embodiments, R^{4.1} and R^{4.3} are unsubstituted C₁-C₄ alkyl. In embodiments, R^{4.1} and R^{4.3} are substituted C₁-C₄ alkyl. In embodiments, R^{4.1} and R^{4.3} are halogen substituted C₁-C₄ alkyl. In embodiments, R^{4.1} and R^{4.3} are unsubstituted C₁-C₂ alkyl. In embodiments, R^{4.1} and R^{4.3} are substituted C₁-C₂ alkyl. In embodiments, R^{4.1} and R^{4.3} are halogen substituted C₁-C₂ alkyl.

In embodiments, the PCNS inhibitor is a compound having the formula:

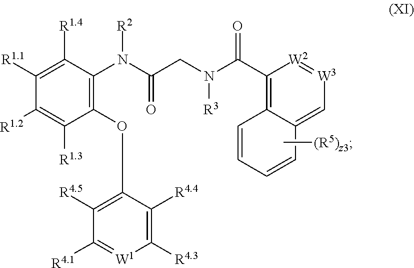

wherein R², R³, R⁵, and z3 are as described herein, including in compounds of formula (I) to (V). R^{1.1}, R^{1.2}, R^{1.3} and R^{1.4} are each independently a moiety of R¹ as described herein, including in embodiments. In embodiments, R^{1.1}, R^{1.1}, R^{1.3} and/or R^{1.4} are hydrogen. In embodiments, R^{4.1}, R^{4.2}, R^{4.3}, R^{4.4}, and/or R^{4.5} are hydrogen. In embodiments, R² is hydrogen. In embodiments, R³ is hydrogen. In embodiments R^{1.1} is halogen. In embodiments R¹² is halogen. In embodiments R¹³ is halogen. In embodiments R^{1.4} is halogen. In embodiments R^{1.1} is —Cl. In embodiments R^{1.2} is —Cl. In embodiments R^{1.3} is —Cl. In embodiments R^{1.4} is —Cl. In embodiments R^{1.1} is —F. In embodiments R^{1.2} is —F. In embodiments R^{1.3} is —F. In embodiments R^{1.4} is —F. In embodiments R^{1.2}, R^{1.3}, and R^{1.4} are hydrogen and R^{1.1} is halogen. In embodiments R^{1.1}, R^{1.3}, and R^{1.4} are hydrogen and R^{1.2} is halogen. In embodiments R^{1.2}, R^{1.1}, and R^{1.4} are hydrogen and R^{1.3} is halogen. In embodiments R^{1.2}, R^{1.3}, and R^{1.1} are hydrogen and R^{1.4} is halogen. In embodiments R^{1.2}, R^{1.3}, and R^{1.4} are hydrogen and R^{1.1} is —Cl. In embodiments R^{1.1}, R^{1.3} and R^{1.4} are hydrogen and R^{1.2} is —Cl. In embodiments R^{1.2}, R^{1.1}, and R^{1.4} are hydrogen and R^{1.3} is —Cl. In embodiments R^{1.2}, R^{1.3}, and R^{1.1} are hydrogen and R^{1.4} is —Cl. In embodiments R^{1.2}, R^{1.3}, and R^{1.4} are hydrogen and R^{1.1} is —F. In embodiments R^{1.1}, R^{1.3} and R^{1.4} are hydrogen and R^{1.2} is —F. In embodiments R^{1.2}, R^{1.1}, and R^{1.4} are hydrogen and R^{1.3} is —F. In embodiments R^{1.2}. R^{1.3}, and R^{1.1} are hydrogen and R^{1.4} is —F. W¹ is N or C(R^{4.2}). W² is N or C(R^{5.1}) W³ is N or C(R^{5.2}). In embodiments, W¹ is N. In embodiments, W² is N. In embodiments, W³ is N. In embodiments, W¹ is C(R^{4.2}). In embodiments, W² is C(R^{5.1}). In embodiments, W³ is C(R^{5.2}). In embodiments, W¹ is CH. In embodiments, W² is CH. In embodiments, W³ is CH. R^{5.1} and R^{5.2} are each independently a moiety of R⁵ as described herein, including in embodiments. In embodiment, z3 is 0. R^{4.1}, R^{4.2}, R^{4.3}, R^{4.4} and R^{4.5} are each independently a moiety of R⁴ as described herein, including in embodiments. In embodiments, R^{4.1} is unsubstituted methoxy. In embodiments, R^{4.2} is unsubstituted methoxy. In embodiments, R^{4.3} is unsubstituted methoxy. In embodiments, R^{4.4} is unsubstituted methoxy. In embodiments, R^{4.5} is unsubstituted methoxy. In embodiments, R^{4.2}, R^{4.3}, R^{4.4}, and R^{4.5} are hydrogen and R^{4.1} is unsubstituted methoxy. In embodiments, R^{4.2}, R^{4.3}, R^{4.4} and R^{4.5} are hydrogen and R^{4.2} is unsubstituted methoxy. In embodiments, R^{4.2}, R^{4.1}, R^{4.4} and R^{4.5} are hydrogen and R^{4.3} is unsubstituted methoxy. In embodiments, R^{4.2}, R^{4.3}, R^{4.1} and R^{4.5} are hydrogen and R^{4.4} is unsubstituted methoxy. In embodiments, R^{4.2}, R^{4.3}, R^{4.4} and R^{4.1} are hydrogen and R^{4.5} is unsubstituted methoxy. In embodiments, R^{4.1} is unsubstituted ethoxy. In embodiments, R^{4.2} is unsubstituted ethoxy. In embodiments, R^{4.3} is unsubstituted ethoxy. In embodiments, R^{4.4} is unsubstituted ethoxy. In embodiments, R^{4.5} is unsubstituted ethoxy. In embodiments, R^{4.2}, R^{4.3}, R^{4.4}, and R^{4.5} are hydrogen and R^{4.1} is unsubstituted ethoxy. In embodiments, R^{4.1}, R^{4.3}, R^{4.4}, and R^{4.5} are hydrogen and R^{4.2} is unsubstituted ethoxy. In embodiments, R^{4.2}, R^{4.1}, R^{4.4} and R^{4.5} are hydrogen and R^{4.3} is unsubstituted ethoxy. In embodiments, R^{4.2}, R^{4.3}, R^{4.1} and R^{4.5} are hydrogen and R^{4.4} is unsubstituted ethoxy. In embodiments, R^{4.2}, R^{4.3}, R^{4.4}, and R^{4.1} are hydrogen and R^{4.5} is unsubstituted ethoxy. In embodiments, R^{4.1} is —OH. In embodiments R^{4.2} is —OH. In embodiments, R^{4.3} is —OH. In embodiments, R^{4.4} is —OH. In embodiments, R^{4.5} is —OH. In embodiments, R^{4.2}, R^{4.3}, R^{4.4} and R^{4.5} are hydrogen and R^{4.1} is —OH. In embodiments, R^{4.1}, R^{4.3}, R^{4.4}, and R^{4.5} are hydrogen and R^{4.2} is —OH. In embodiments, R^{4.2}, R^{4.1}, R^{4.4}, and R^{4.5} are hydrogen and R^{4.3} is —OH. In embodiments, R^{4.2}, R^{4.3}, R^{4.1} and R^{4.5} are hydrogen and R^{4.4} is —OH. In embodiments, R^{4.2}, R^{4.3}, R^{4.4} and R^{4.1} are hydrogen and R^{4.5} is —OH. In embodiments, R^{4.1} is halogen. In embodiments, R^{4.2} is halogen. In embodiments, R^{4.3} is halogen. In embodiments, R^{4.4} is halogen. In embodiments, R^{4.5} is halogen. In embodiments, R^{4.2}, R^{4.3}, R^{4.4}, and R^{4.5} are hydrogen and R^{4.1} is halogen. In embodiments, R^{4.1}, R^{4.3}, R^{4.4}, and R^{4.5} are hydrogen and R^{4.2} is halogen. In embodiments, R^{4.2}, R^{4.1}, R^{4.4} and R^{4.5} are hydrogen and R^{4.3} is halogen. In embodiments, R^{4.2}, R^{4.3}, R^{4.1} and R^{4.5} are hydrogen and R^{4.4} is halogen. In embodiments, R^{4.2}, R^{4.3}, R^{4.4}, and R^{4.1} are hydrogen and R^{4.5} is halogen. In embodiments, R^{4.1} is unsubstituted methyl. In embodiments, R^{4.2} is unsubstituted methyl. In embodiments, R^{4.3} is unsubstituted methyl. In embodiments, R^{4.4} is unsubstituted methyl. In embodiments, R^{4.5} is unsubstituted methyl. In embodiments, R^{4.2}, R^{4.3}, R^{4.4}, and R^{4.5} are hydrogen and R^{4.1} is unsubstituted methyl. In embodiments, R^{4.1}, R^{4.3}, R^{4.4} and R^{4.5} are hydrogen and R^{4.2} is unsubstituted methyl. In embodiments, R^{4.2}, R^{4.1}, R^{4.4}, and R^{4.5} are hydrogen and R^{4.3} is unsubstituted methyl. In embodiments, R^{4.2}, R^{4.3}, R^{4.1} and R^{4.5} are hydrogen and R^{4.4} is unsubstituted methyl. In embodiments, R^{4.2}, R^{4.3}, R^{4.4}, and R^{4.1} are hydrogen and R^{4.5} is unsubstituted methyl. In embodiments, one or more of R^{1.1}, R^{1.2}, R^{1.3}, R^{1.4}, R^{4.1}, R^{4.2}, R^{4.3}, R^{4.4}, R^{4.5}, R² and/or R³ are hydrogen. It will be understood that R⁵ is/are a floating substituent and may be positioned on either or both rings.

In embodiments, the PCNS inhibitor is a compound having the formula:

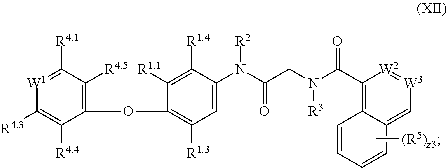

wherein R^{1.1}, R^{1.3}, R^{1.4}, R², R³, R^{4.1}, R^{4.3}, R^{4.4}, R^{4.5}, R⁵, W¹, W², W³, and z3 are as described herein, including in compounds of formula (I) to (XI). It will be understood that R⁵ is/are a floating substituent and may be positioned on either or both rings. R^{1.1}, R^{1.3}, and R^{1.4} are each independently a moiety of R¹ as described herein, including in embodiments. R^{4.1}, R^{4.3}, R^{4.4}, and R^{4.5} are each independently a moiety of R⁴ as described herein, including in embodiments. In embodiments, R^{1.1}, R^{1.3} and/or R^{1.4} are hydrogen. In embodiments, R^{4.1}, R^{4.2}, R^{4.3}, R^{4.4} and/or R^{4.5} are hydrogen. In embodiments, R² is hydrogen. In embodiments, R³ is hydrogen. In embodiments R^{1.1} is halogen. In embodiments R¹³ is halogen. In embodiments R¹⁴ is halogen. In embodiments R^{1.1} is —Cl. In embodiments R^{1.3} is —Cl. In embodiments R^{1.4} is —Cl. In embodiments R^{1.1} is —F. In embodiments R^{1.3} is —F. In embodiments R^{1.4} is —F. In embodiments R^{1.3} and R^{1.4} are hydrogen and R^{1.1} is halogen. In embodiments R^{1.1} and R^{1.4} are hydrogen and R^{1.3} is halogen. In embodiments R^{1.3} and R^{1.1} are hydrogen and R^{1.4} is halogen. In embodiments R¹³ and R¹⁴ are hydrogen and R^{1.1} is —Cl. In embodiments R^{1.1} and R^{1.4} are hydrogen and R^{1.3} is —Cl. In embodiments R^{1.3} and R^{1.1} are hydrogen and R^{1.4} is —Cl. In embodiments R^{1.3} and R^{1.4} are hydrogen and R^{1.1} is —F. In embodiments R^{1.1} and R^{1.4} are hydrogen and R¹³ is —F. In embodiments R^{1.3} and R^{1.1} are hydrogen and R¹⁴ is —F. W¹ is N or C(R^{4.2}). W² is N or C(R^{5.1}) W³ is N or C(R^{5.2}). In embodiments, W¹ is N. In embodiments, W² is N. In embodiments, W³ is N. In embodiments, W¹ is C(R^{4.2}). In embodiments, W² is C(R^{5.1}). In embodiments, W³ is C(R^{5.2}). In embodiments, W¹ is CH. In embodiments, W² is CH. In embodiments, W³ is CH. R^{5.1} and R^{5.2} are each independently a moiety of R⁵ as described herein, including in embodiments. In embodiment, z3 is 0. R^{4.1}, R^{4.2}, R^{4.3}, R^{4.4}, and R^{4.5} are each independently a moiety of R⁴ as described herein, including in embodiments. In embodiments, R^{4′} is unsubstituted methoxy. In embodiments, R^{4.2} is unsubstituted methoxy. In embodiments, R^{4.3} is unsubstituted methoxy. In embodiments, R^{4.4} is unsubstituted methoxy. In embodiments, R^{4.5} is unsubstituted methoxy. In embodiments, R^{4.2}, R^{4.3}, R^{4.4} and R^{4.5} are hydrogen and R^{4.1} is unsubstituted methoxy. In embodiments, R^{4.1}, R^{4.3}, R^{4.4} and R^{4.5} are hydrogen and R^{4.2} is unsubstituted methoxy. In embodiments, R^{4.2}, R^{4.1}, R^{4.4} and R^{4.5} are hydrogen and R^{4.3} is unsubstituted methoxy. In embodiments, R^{4.2}, R^{4.3}, R^{4.1}, and R^{4.5} are hydrogen and R^{4.4} is unsubstituted methoxy. In embodiments, R^{4.2}, R^{4.3}, R^{4.4} and R^{4.5} are hydrogen and R^{4.5} is unsubstituted methoxy. In embodiments, R^{4.1} is unsubstituted ethoxy. In embodiments, R^{4.2} is unsubstituted ethoxy. In embodiments, R^{4.3} is unsubstituted ethoxy. In embodiments, R^{4.4} is unsubstituted ethoxy. In embodiments, R^{4.5} is unsubstituted ethoxy. In embodiments, R^{4.2}, R^{4.3}, R^{4.4}, and R^{4.5} are hydrogen and R^{4.1} is unsubstituted ethoxy. In embodiments, R^{4.1}, R^{4.3}, R^{4.4} and R^{4.5} are hydrogen and R^{4.2} is unsubstituted ethoxy. In embodiments, R^{4.2}, R^{4.1}, R^{4.4} and R^{4.5} are hydrogen and R^{4.3} is unsubstituted ethoxy. In embodiments, R^{4.2}, R^{4.3}, R^{4.1} and R^{4.5} are hydrogen and R^{4.4} is unsubstituted ethoxy. In embodiments, R^{4.2}, R^{4.3}, R^{4.4} and R^{4.1} are hydrogen and R^{4.5} is unsubstituted ethoxy. In embodiments, R^{4.1} is —OH. In embodiments R^{4.2} is —OH. In embodiments, R^{4.3} is —OH. In embodiments, R^{4.4} is —OH. In embodiments, R^{4.5} is —OH. In embodiments, R^{4.2}, R^{4.3}, R^{4.4}, and R^{4.5} are hydrogen and R^{4.1} is —OH. In embodiments, R^{4.1}, R^{4.3}, R^{4.4}, and R^{4.5} are hydrogen and R^{4.2} is —OH. In embodiments, R^{4.2}, R^{4.1}, R^{4.4}, and R^{4.5} are hydrogen and R^{4.3} is —OH. In embodiments, R^{4.2}, R^{4.3}, R^{4.1}, and R^{4.5} are hydrogen and R^{4.4} is —OH. In embodiments, R^{4.2}, R^{4.3}, R^{4.4} and R^{4.1} are hydrogen and R^{4.5} is —OH. In embodiments, R^{4.1} is halogen. In embodiments, R^{4.2} is halogen. In embodiments, R^{4.3} is halogen. In embodiments, R^{4.4} is halogen. In embodiments, R^{4.5} is halogen. In embodiments, R^{4.2}, R^{4.3}, R^{4.4}, and R^{4.5} are hydrogen and R^{4.1} is halogen. In embodiments, R^{4.1}, R^{4.3}, R^{4.4} and R^{4.5} are hydrogen and R^{4.2} is halogen. In embodiments, R^{4.2}, R^{4.1}, R^{4.4}, and R^{4.5} are hydrogen and R^{4.3} is halogen. In embodiments, R^{4.2}, R^{4.3}, R^{4.1} and R^{4.5} are hydrogen and R^{4.4} is halogen. In embodiments, R^{4.2}, R^{4.3}, R^{4.4}, and R^{4.1} are hydrogen and R^{4.5} is halogen. In embodiments, R^{4.1} is unsubstituted methyl. In embodiments, R^{4.2} is unsubstituted methyl. In embodiments, R^{4.3} is unsubstituted methyl. In embodiments, R^{4.4} is unsubstituted methyl. In embodiments, R^{4.5} is unsubstituted methyl. In embodiments, R^{4.2}, R^{4.3}, R^{4.4}, and R^{4.5} are hydrogen and R^{4.1} is unsubstituted methyl. In embodiments, R^{4.1}, R^{4.3}, R^{4.4}, and R^{4.5} are hydrogen and R^{4.2} is unsubstituted methyl. In embodiments, R^{4.2}, R^{4.1}, R^{4.4}, and R^{4.5} are hydrogen and R^{4.3} is unsubstituted methyl. In embodiments, R^{4.2}, R^{4.3}, R^{4.1} and R^{4.5} are hydrogen and R^{4.4} is unsubstituted methyl. In embodiments, R^{4.2}, R^{4.3}, R^{4.4} and R^{4.1} are hydrogen and R^{4.5} is unsubstituted methyl. In embodiments, one or more of R^{1.1}, R^{1.3}, R^{1.4}, R^{4.1}, R^{4.2}, R^{4.3}, R^{4.4}, R^{4.5}, R² and/or R³ are hydrogen. It will be understood that R⁵ is/are a floating substituent and may be positioned on either or both rings.

In embodiments, the PCNS inhibitor is a compound having the formula:

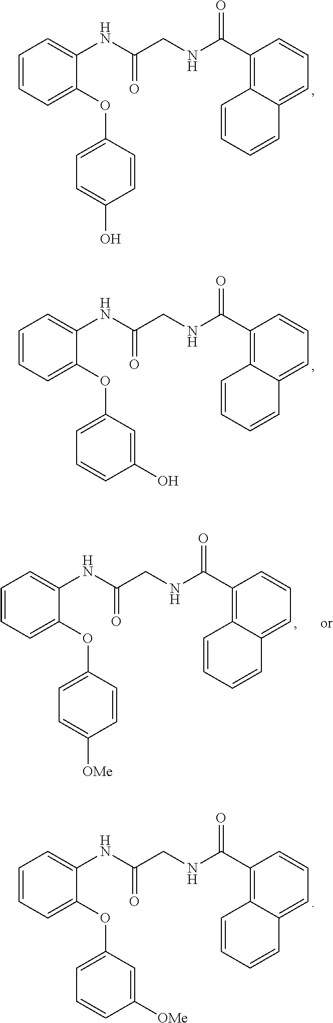 [SMILES: O=C(CNC(=O)c1cccc2ccccc12)Nc1ccccc1Oc1ccc(O)cc1 ; O=C(CNC(=O)c1cccc2ccccc12)Nc1ccccc1Oc1cccc(O)c1 ; COc1ccc(Oc2ccccc2NC(=O)CNC(=O)c2cccc3ccccc23)cc1 ; COc1cccc(Oc2ccccc2NC(=O)CNC(=O)c2cccc3ccccc23)c1]

In embodiments, the PCNS inhibitor is a compound having the formula:

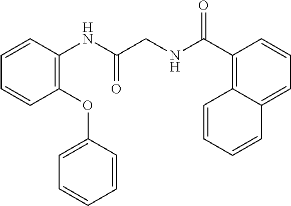 [SMILES: O=C(CNC(=O)c1cccc2ccccc12)Nc1ccccc1Oc1ccccc1]

In embodiments, the PCNS inhibitor is a compound having the formula:

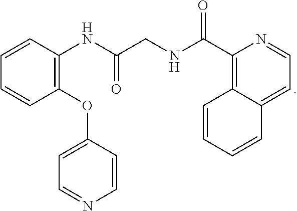 [SMILES: O=C(CNC(=O)c1nccc2ccccc12)Nc1ccccc1Oc1ccncc1]

In embodiments, the PCNS inhibitor is a compound having the formula:

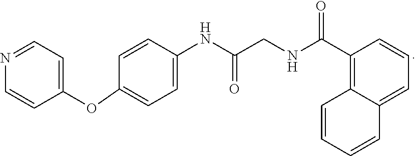 [SMILES: O=C(CNC(=O)c1cccc2ccccc12)Nc1ccc(Oc2ccncc2)cc1]

In embodiments, the PCNS inhibitor is a compound having the formula:

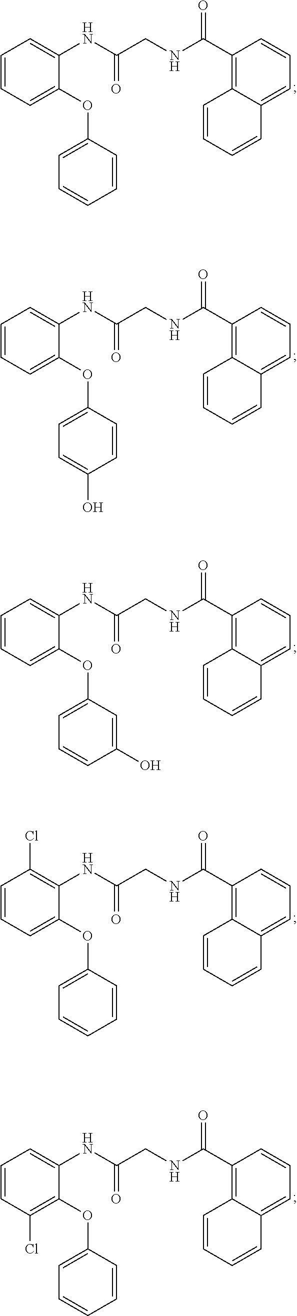 [SMILES: O=C(CNC(=O)c1cccc2ccccc12)Nc1ccccc1Oc1ccccc1 ; O=C(CNC(=O)c1cccc2ccccc12)Nc1ccccc1Oc1ccc(O)cc1 ; O=C(CNC(=O)c1cccc2ccccc12)Nc1ccccc1Oc1cccc(O)c1 ; O=C(CNC(=O)c1cccc2ccccc12)Nc1c(Cl)cccc1Oc1ccccc1 ; O=C(CNC(=O)c1cccc2ccccc12)Nc1cccc(Cl)c1Oc1ccccc1]

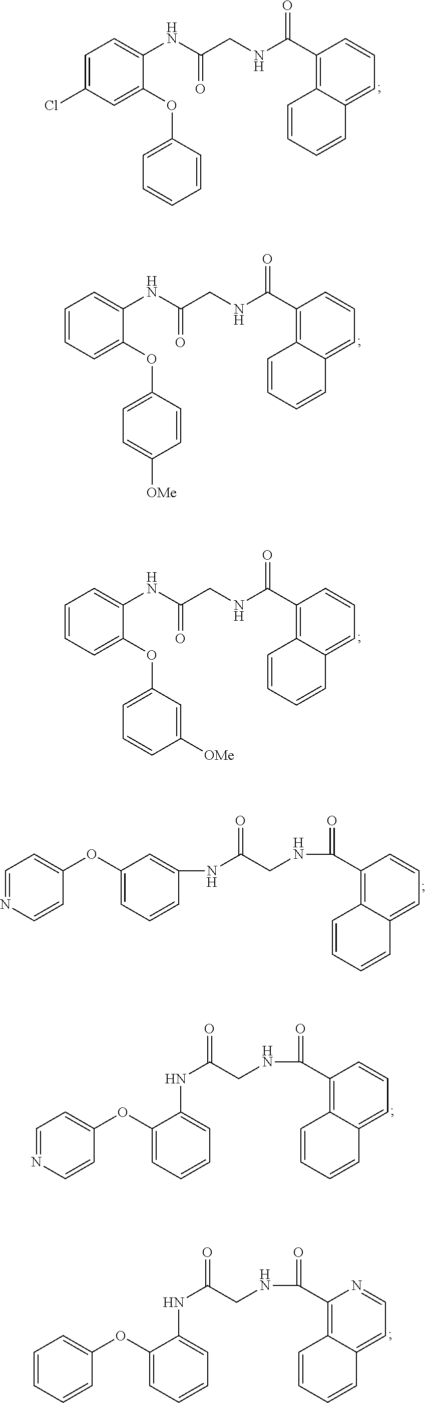 [SMILES: O=C(CNC(=O)c1cccc2ccccc12)Nc1ccc(Cl)cc1Oc1ccccc1 ; COc1ccc(Oc2ccccc2NC(=O)CNC(=O)c2cccc3ccccc23)cc1 ; COc1cccc(Oc2ccccc2NC(=O)CNC(=O)c2cccc3ccccc23)c1 ; O=C(CNC(=O)c1cccc2ccccc12)Nc1cccc(Oc2ccncc2)c1 ; O=C(CNC(=O)c1cccc2ccccc12)Nc1ccccc1Oc1ccncc1 ; O=C(CNC(=O)c1nccc2ccccc12)Nc1ccccc1Oc1ccccc1]

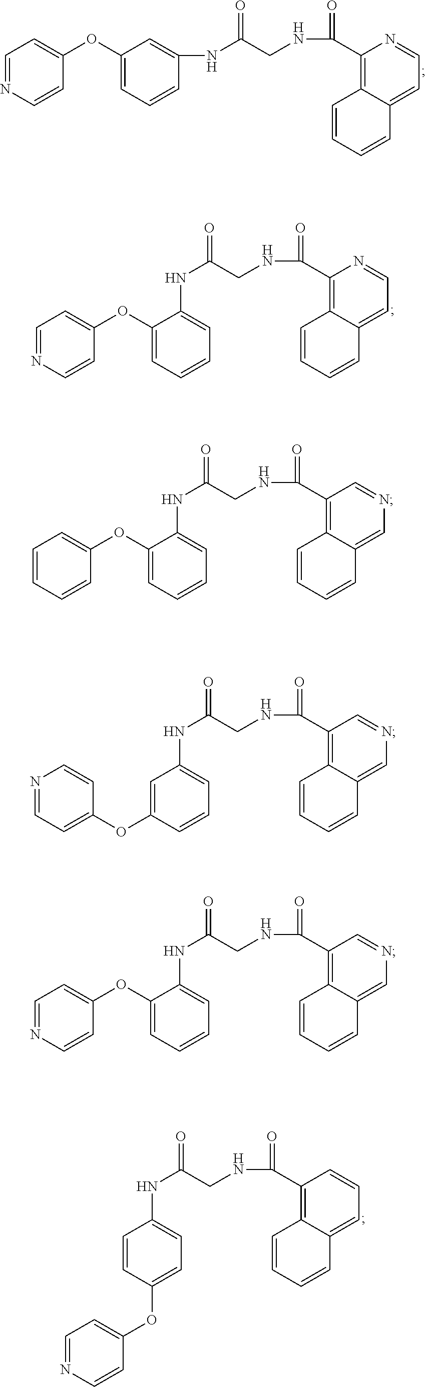 [SMILES: O=C(CNC(=O)c1nccc2ccccc12)Nc1cccc(Oc2ccncc2)c1 ; O=C(CNC(=O)c1nccc2ccccc12)Nc1ccccc1Oc1ccncc1 ; O=C(CNC(=O)c1cncc2ccccc12)Nc1ccccc1Oc1ccccc1 ; O=C(CNC(=O)c1cncc2ccccc12)Nc1cccc(Oc2ccncc2)c1 ; O=C(CNC(=O)c1cncc2ccccc12)Nc1ccccc1Oc1ccncc1 ; O=C(CNC(=O)c1cccc2ccccc12)Nc1ccc(Oc2ccncc2)cc1]

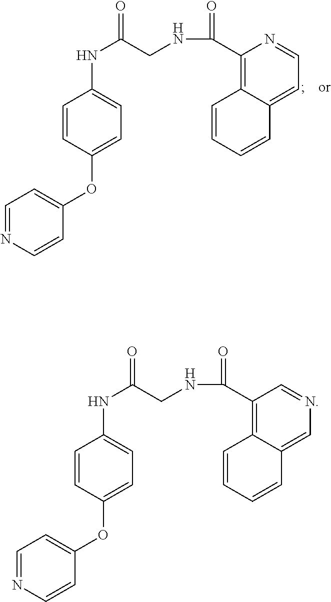 [SMILES: O=C(CNC(=O)c1nccc2ccccc12)Nc1ccc(Oc2ccncc2)cc1 ; O=C(CNC(=O)c1cncc2ccccc12)Nc1ccc(Oc2ccncc2)cc1]

### Pharmaceutical Compositions

Provided herein are pharmaceutical compositions comprising a PCNA inhibitor or pharmaceutically acceptable salt thereof described herein (including embodiments thereof) and a pharmaceutically acceptable excipient and a compound. Provided herein are pharmaceutical compositions comprising an EGFR-TK inhibitor or pharmaceutically acceptable salt thereof described herein (including embodiments thereof) and a pharmaceutically acceptable excipient and a compound. “Active ingredient” refers to a PCNA inhibitor and/or an EGFR-TK inhibitor. In the methods described herein, the active ingredients can be administered separately to the patient (e.g., a first pharmaceutical composition comprising a PCNA inhibitor and a second pharmaceutical composition comprising an EGFR-TK inhibitor, where the first and second pharmaceutical compositions are different) or the active ingredients can be administered to the patient as a single composition (e.g., a single pharmaceutical compositions comprising a PCNA inhibitor and an EGFR-TK inhibitor).

Pharmaceutical compositions include compositions wherein the active ingredient is contained in a therapeutically effective amount, i.e., in an amount effective to achieve its intended purpose. The actual amount effective for a particular application will depend, inter alia, on the condition being treated. When administered in methods to treat a disease (cancer), such compositions will contain an amount of active ingredient effective to achieve the desired result, e.g., treating cancer, inhibiting cell proliferation. Determination of a therapeutically effective amount of a compound is well within the capabilities of those skilled in the art, especially in light of the detailed disclosure herein.

For preparing pharmaceutical compositions from the compounds described herein, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material.

In powders, the carrier is a finely divided solid in a mixture with the finely divided active component (e.g., a compound provided herein). In tablets, the active component is mixed with the carrier having the necessary binding properties in suitable proportions and compacted in the shape and size desired. The powders and tablets preferably contain from 1% to 99% of the active compound.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen. If desired, disintegrating or solubilizing agents may be added, such as the cross-linked polyvinyl pyrrolidone, agar, alginic acid, or a salt thereof, such as sodium alginate.

Dragees cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, for example, push-fit capsules made of gelatin, as well as soft, sealed capsules made of gelatin and a coating such as glycerol or sorbitol.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

When parenteral application is needed or desired, particularly suitable admixtures for the compounds of the invention are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampules are convenient unit dosages. The compounds of the invention can also be incorporated into liposomes or administered via transdermal pumps or patches. Pharmaceutical admixtures suitable for use in the present invention are well-known to those of skill in the art and are described, for example, in Pharmaceutical Sciences (17th Ed., Mack Pub. Co., Easton, PA) and WO 96/05309.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. The pharmaceutical formulations can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compounds described herein can be administered alone or can be coadministered to the patient. Coadministration is meant to include simultaneous or sequential administration of the compounds individually or in combination (more than one compound). Thus, the preparations can also be combined, when desired, with other active substances.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg, more typically 1.0 mg to 1000 mg, most typically 10 mg to 500 mg, according to the particular application and the potency of the active component. The quantity of active compound may also be defined as mg/kg, ranging from about 0.1 mg/kg to 500 mg/kg. The composition can, if desired, also contain other compatible therapeutic agents.

The ratio between toxicity and therapeutic effect for a particular compound is its therapeutic index and can be expressed as the ratio between LD₅₀ (the amount of compound lethal in 50% of the population) and ED₅₀ (the amount of compound effective in 50% of the population). Compounds that exhibit high therapeutic indices are preferred. Therapeutic index data obtained from cell culture assays and/or animal studies can be used in formulating a range of dosages for use in humans. The dosage of such compounds preferably lies within a range of plasma concentrations that include the ED₅₀ with little or no toxicity. The dosage may vary within this range depending upon the dosage form employed and the route of administration utilized. The exact formulation, route of administration and dosage can be chosen by the individual physician in view of the patient's condition and the particular method in which the compound is used.

### Methods of Treatment

The disclosure provides methods of treating cancer in a patient in need thereof by administering an effective amount of AOH1996 or a pharmaceutically acceptable salt thereof and an effective amount of an EGFR-TK inhibitor or a pharmaceutically acceptable salt thereof. In embodiments, the disclosure provides methods of treating cancer in a patient in need thereof by administering an effective amount of a first pharmaceutical composition comprising AOH1996 or a pharmaceutically acceptable salt thereof and a pharmaceutically acceptable excipient and an effective amount of a second pharmaceutical composition comprising an EGFR-TK inhibitor and a pharmaceutically acceptable excipient. In embodiments, the disclosure provides methods of treating cancer in a patient in need thereof by administering an effective amount of a pharmaceutical composition comprising AOH1996 or a pharmaceutically acceptable salt thereof, an EGFR-TK inhibitor, and a pharmaceutically acceptable excipient. In embodiments, the cancer is lung cancer, colorectal cancer, colon cancer, pancreatic cancer, breast cancer, squamous cell carcinoma, thyroid cancer, or head and neck cancer. In embodiments, the cancer is non-small cell lung cancer, colorectal cancer, colon cancer, pancreatic cancer, breast cancer, squamous cell carcinoma, thyroid cancer, or head and neck cancer. n embodiments, the cancer is non-small cell lung cancer, colorectal cancer, pancreatic cancer, breast cancer, squamous cell carcinoma, thyroid cancer, or head and neck cancer. In embodiments, the cancer is lung cancer. In embodiments, the cancer is non-small cell lung cancer. In embodiments, the cancer is colorectal cancer. In embodiments, the cancer is colon cancer. In embodiments, the cancer is pancreatic cancer. In embodiments, the cancer is breast cancer. In embodiments, the cancer is squamous cell carcinoma. In embodiments, the cancer is squamous cell carcinoma. In embodiments, the cancer is thyroid cancer. In embodiments, the cancer is head and neck cancer. In embodiments, the cancer has an EGFR mutation; a KRAS mutation, a BRAF mutation, or a combination of two or more thereof. In embodiments, the cancer has an EGFR mutation. In embodiments, the EGFR mutation comprises L858R, ex19del, Ex10ins, T790M, or a combination of two or more thereof. In embodiments, the EGFR mutation comprises L858R and ex19del. In embodiments, the EGFR mutation comprises L858R. In embodiments, the EGFR mutation comprises ex19del. In embodiments, the EGFR mutation comprises Ex10ins. In embodiments, the EGFR mutation comprises L858R, ex19del, and T790M. In embodiments, the EGFR mutation comprises L858R and T790M. In embodiments, the EGFR mutation comprises T790M. In embodiments, the cancer has a KRAS mutation. In embodiments, the KRAS mutation is G12, G13, or Q61. In embodiments, the KRAS mutation is G12C, G12S, G12V, G12D, G12A, G13D, G13C, Q61H, or Q61R. In embodiments, the KRAS mutation is a G12 mutation. In embodiments, the KRAS mutation is a G12C mutation. In embodiments, the KRAS mutation is a G12S mutation. In embodiments, the KRAS mutation is a G12V mutation. In embodiments, the KRAS mutation is a G12D mutation. In embodiments, the KRAS mutation is a G12A mutation. In embodiments, the KRAS mutation is a G13 mutation. In embodiments, the KRAS mutation is a G13D mutation. In embodiments, the KRAS mutation is a G13C mutation. In embodiments, the KRAS mutation is a Q61 mutation. In embodiments, the KRAS mutation is a Q61H mutation. In embodiments, the KRAS mutation is a Q61R mutation. In embodiments, the cancer has a BRAF mutation. In embodiments, the BRAF mutation is a V600E mutation. In embodiments, the cancer is a EGFR-TK-resistant cancer. In embodiments, the methods of treating cancer further comprise administering to the patient an effective amount of one or more anti-cancer agents.

The disclosure provides methods of treating cancer in a patient in need thereof by administering an effective amount of AOH1996 or a pharmaceutically acceptable salt thereof and an effective amount of osimertinib, gefitinib, afatinib, neratinib, erlotinib, rociletinib, olmutinib, lazertinib, nazartinib, naquotinib, mavelertinib, abivertinib, olafertinib, alflutinib, amivantamb, tarloxitinib, mobocertinib, savolitinib, capmatinib, cetuximab, panitumumab, lapatinib, dacomitinib, necitumumab, vandetanib, icotininib, canertinib, allitinib, varlitinib, tesevatinib, pelitinib, sapitinib, EAI045, TAK-285, AG-1478, AEE788, CUDC-101, WZ8040, WZ4002, WZ3146, AG-490, PD153035, or a pharmaceutically acceptable salt of any one of the foregoing. In embodiments, the cancer is lung cancer, colorectal cancer, colon cancer, pancreatic cancer, breast cancer, squamous cell carcinoma, thyroid cancer, or head and neck cancer. In embodiments, the cancer is non-small cell lung cancer, colorectal cancer, colon cancer, pancreatic cancer, breast cancer, squamous cell carcinoma, thyroid cancer, or head and neck cancer. In embodiments, the cancer is lung cancer. In embodiments, the cancer is non-small cell lung cancer. In embodiments, the cancer is colorectal cancer. In embodiments, the cancer is colon cancer. In embodiments, the cancer is pancreatic cancer. In embodiments, the cancer is breast cancer. In embodiments, the cancer is squamous cell carcinoma. In embodiments, the cancer is squamous cell carcinoma. In embodiments, the cancer is thyroid cancer. In embodiments, the cancer is head and neck cancer. In embodiments, the cancer has an EGFR mutation, a KRAS mutation, a BRAF mutation, or a combination of two or more thereof. In embodiments, the cancer has an EGFR mutation. In embodiments, the EGFR mutation comprises L858R, ex19del, Ex10ins, T790M, or a combination of two or more thereof. In embodiments, the EGFR mutation comprises L858R and ex19del. In embodiments, the EGFR mutation comprises L858R. In embodiments, the EGFR mutation comprises ex19del. In embodiments, the EGFR mutation comprises Ex10ins. In embodiments, the EGFR mutation comprises L858R, ex19del, and T790M. In embodiments, the EGFR mutation comprises L858R and T790M. In embodiments, the EGFR mutation comprises T790M. In embodiments, the cancer has a KRAS mutation. In embodiments, the KRAS mutation is G12, G13, or Q61. In embodiments, the KRAS mutation is G12C, G12S, G12V, G12D, G12A, G13D, G13C, Q61H, or Q61R. In embodiments, the KRAS mutation is a G12 mutation. In embodiments, the KRAS mutation is a G12C mutation. In embodiments, the KRAS mutation is a G12S mutation. In embodiments, the KRAS mutation is a G12V mutation. In embodiments, the KRAS mutation is a G12D mutation. In embodiments, the KRAS mutation is a G12A mutation. In embodiments, the KRAS mutation is a G13 mutation. In embodiments, the KRAS mutation is a G13D mutation. In embodiments, the KRAS mutation is a G13C mutation. In embodiments, the KRAS mutation is a Q61 mutation. In embodiments, the KRAS mutation is a Q61H mutation. In embodiments, the KRAS mutation is a Q61R mutation. In embodiments, the cancer has a BRAF mutation. In embodiments, the BRAF mutation is a V600E mutation. In embodiments, the cancer is a EGFR-TK-resistant cancer. In embodiments, the EGFR-TK inhibitor and the compound of AOH1996 or the pharmaceutically acceptable salt thereof are separately administered to the patient. In embodiments, the methods of treating cancer comprising administering to the patient a first pharmaceutical composition comprising the EGFR-TK inhibitor and a pharmaceutically acceptable excipient and a second pharmaceutical composition comprising AOH1996 or the pharmaceutically acceptable salt thereof and a pharmaceutically acceptable excipient (i.e., the first pharmaceutical composition and the second pharmaceutical compositions are different). In embodiments, the methods comprise administering to the patient a pharmaceutical composition comprising the EGFR-TK inhibitor, AOH1996 or the pharmaceutically acceptable salt thereof, and a pharmaceutically acceptable excipient. In embodiments, the methods of treating cancer further comprise administering to the patient an effective amount of one or more anti-cancer agents.

In embodiments, the methods comprise treating cancer by administering an effective amount of AOH1996 or a pharmaceutically acceptable salt thereof and an effective amount of osimertinib. In embodiments, the methods comprise treating cancer by administering an effective amount of AOH1996 or a pharmaceutically acceptable salt thereof and an effective amount of osimertinib. In embodiments, the methods comprise treating cancer by administering an effective amount of AOH1996 or a pharmaceutically acceptable salt thereof and an effective amount of gefitinib. In embodiments, the methods comprise treating cancer by administering an effective amount of AOH1996 or a pharmaceutically acceptable salt thereof and an effective amount of afatinib. In embodiments, the methods comprise treating cancer by administering an effective amount of AOH1996 or a pharmaceutically acceptable salt thereof and an effective amount of erlotinib. In embodiments, the methods comprise treating cancer by administering an effective amount of AOH1996 or a pharmaceutically acceptable salt thereof and an effective amount of rociletinib. In embodiments, the methods comprise treating cancer by administering an effective amount of AOH1996 or a pharmaceutically acceptable salt thereof and an effective amount of olmutinib. In embodiments, the methods comprise treating cancer by administering an effective amount of AOH1996 or a pharmaceutically acceptable salt thereof and an effective amount of lazertinib. In embodiments, the methods comprise treating cancer by administering an effective amount of AOH1996 or a pharmaceutically acceptable salt thereof and an effective amount of nazartinib. In embodiments, the methods comprise treating cancer by administering an effective amount of AOH1996 or a pharmaceutically acceptable salt thereof and an effective amount of naquotinib. In embodiments, the methods comprise treating cancer by administering an effective amount of AOH1996 or a pharmaceutically acceptable salt thereof and an effective amount of mavelertinib. In embodiments, the methods comprise treating cancer by administering an effective amount of AOH1996 or a pharmaceutically acceptable salt thereof and an effective amount of abivertinib. In embodiments, the methods comprise treating cancer by administering an effective amount of AOH1996 or a pharmaceutically acceptable salt thereof and an effective amount of olafertinib. In embodiments, the methods comprise treating cancer by administering an effective amount of AOH1996 or a pharmaceutically acceptable salt thereof and an effective amount of alflutinib. In embodiments, the methods comprise treating cancer by administering an effective amount of AOH1996 or a pharmaceutically acceptable salt thereof and an effective amount of amivantamb. In embodiments, the methods comprise treating cancer by administering an effective amount of AOH1996 or a pharmaceutically acceptable salt thereof and an effective amount of tarloxitinib. In embodiments, the methods comprise treating cancer by administering an effective amount of AOH1996 or a pharmaceutically acceptable salt thereof and an effective amount of mobocertinib. In embodiments, the methods comprise treating cancer by administering an effective amount of AOH1996 or a pharmaceutically acceptable salt thereof and an effective amount of EAI045. In embodiments, the methods comprise treating cancer by administering an effective amount of AOH1996 or a pharmaceutically acceptable salt thereof and an effective amount of savolitinib. In embodiments, the methods comprise treating cancer by administering an effective amount of AOH1996 or a pharmaceutically acceptable salt thereof and an effective amount of capmatinib. In embodiments, the methods comprise treating cancer by administering an effective amount of AOH1996 or a pharmaceutically acceptable salt thereof and an effective amount of cetuximab. In embodiments, the methods comprise treating cancer by administering an effective amount of AOH1996 or a pharmaceutically acceptable salt thereof and an effective amount of panitumumab. In embodiments, the methods comprise treating cancer by administering an effective amount of AOH1996 or a pharmaceutically acceptable salt thereof and an effective amount of lapatinib. In embodiments, the methods comprise treating cancer by administering an effective amount of AOH1996 or a pharmaceutically acceptable salt thereof and an effective amount of dacomitinib. In embodiments, the methods comprise treating cancer by administering an effective amount of AOH1996 or a pharmaceutically acceptable salt thereof and an effective amount of necitumumab. In embodiments, the methods comprise treating cancer by administering an effective amount of AOH1996 or a pharmaceutically acceptable salt thereof and an effective amount of vandetanib. In embodiments, the methods comprise treating cancer by administering an effective amount of AOH1996 or a pharmaceutically acceptable salt thereof and an effective amount of icotininib. In embodiments, the methods comprise treating cancer by administering an effective amount of AOH1996 or a pharmaceutically acceptable salt thereof and an effective amount of canertinib. In embodiments, the methods comprise treating cancer by administering an effective amount of AOH1996 or a pharmaceutically acceptable salt thereof and an effective amount of allitinib. In embodiments, the methods comprise treating cancer by administering an effective amount of AOH1996 or a pharmaceutically acceptable salt thereof and an effective amount of varlitinib. In embodiments, the methods comprise treating cancer by administering an effective amount of AOH1996 or a pharmaceutically acceptable salt thereof and an effective amount of tesevatinib. In embodiments, the methods comprise treating cancer by administering an effective amount of AOH1996 or a pharmaceutically acceptable salt thereof and an effective amount of pelitinib. In embodiments, the methods comprise treating cancer by administering an effective amount of AOH1996 or a pharmaceutically acceptable salt thereof and an effective amount of sapitinib. In embodiments, the methods comprise treating cancer by administering an effective amount of AOH1996 or a pharmaceutically acceptable salt thereof and an effective amount of TAK-285. In embodiments, the methods comprise treating cancer by administering an effective amount of AOH1996 or a pharmaceutically acceptable salt thereof and an effective amount of AG-1478. In embodiments, the methods comprise treating cancer by administering an effective amount of AOH1996 or a pharmaceutically acceptable salt thereof and an effective amount of AEE788. In embodiments, the methods comprise treating cancer by administering an effective amount of AOH1996 or a pharmaceutically acceptable salt thereof and an effective amount of CUDC-101. In embodiments, the methods comprise treating cancer by administering an effective amount of AOH1996 or a pharmaceutically acceptable salt thereof and an effective amount of WZ8040. In embodiments, the methods comprise treating cancer by administering an effective amount of AOH1996 or a pharmaceutically acceptable salt thereof and an effective amount of WZ4002. In embodiments, the methods comprise treating cancer by administering an effective amount of AOH1996 or a pharmaceutically acceptable salt thereof and an effective amount of WZ3146. In embodiments, the methods comprise treating cancer by administering an effective amount of AOH1996 or a pharmaceutically acceptable salt thereof and an effective amount of AG-490. In embodiments, the methods comprise treating cancer by administering an effective amount of AOH1996 or a pharmaceutically acceptable salt thereof and an effective amount of PD153035. In embodiments, the EGFR-TK inhibitor described herein is in the form of a pharmaceutically acceptable salt. In embodiments, the cancer is lung cancer, colorectal cancer, colon cancer, pancreatic cancer, breast cancer, squamous cell carcinoma, thyroid cancer, or head and neck cancer. In embodiments, the cancer is non-small cell lung cancer, colorectal cancer, colon cancer, pancreatic cancer, breast cancer, squamous cell carcinoma, thyroid cancer, or head and neck cancer. In embodiments, the cancer is non-small cell lung cancer, colorectal cancer, pancreatic cancer, breast cancer, squamous cell carcinoma, thyroid cancer, or head and neck cancer. In embodiments, the cancer is lung cancer. In embodiments, the cancer is non-small cell lung cancer. In embodiments, the cancer is colorectal cancer. In embodiments, the cancer is colon cancer. In embodiments, the cancer is pancreatic cancer. In embodiments, the cancer is breast cancer. In embodiments, the cancer is squamous cell carcinoma. In embodiments, the cancer is squamous cell carcinoma. In embodiments, the cancer is thyroid cancer. In embodiments, the cancer is head and neck cancer. In embodiments, the cancer has an EGFR mutation, a KRAS mutation, a BRAF mutation, or a combination of two or more thereof. In embodiments, the cancer has an EGFR mutation. In embodiments; the EGFR mutation comprises L858R, ex19del, Ex10ins, T790M, or a combination of two or more thereof. In embodiments, the EGFR mutation comprises L858R and ex19del. In embodiments, the EGFR mutation comprises L858R. In embodiments, the EGFR mutation comprises ex19del. In embodiments, the EGFR mutation comprises Ex10ins. In embodiments, the EGFR mutation comprises L858R, ex19del, and T790M. In embodiments, the EGFR mutation comprises L858R and T790M. In embodiments, the EGFR mutation comprises T790M. In embodiments, the cancer has a KRAS mutation. In embodiments, the KRAS mutation is G12, G13, or Q61. In embodiments, the KRAS mutation is G12C, G12S, G12V, G12D, G12A, G13D, G13C, Q61H, or Q61R. In embodiments, the KRAS mutation is a G12 mutation. In embodiments, the KRAS mutation is a G12C mutation. In embodiments, the KRAS mutation is a G12S mutation. In embodiments, the KRAS mutation is a G12V mutation. In embodiments, the KRAS mutation is a G12D mutation. In embodiments, the KRAS mutation is a G12A mutation. In embodiments, the KRAS mutation is a G13 mutation. In embodiments, the KRAS mutation is a G13D mutation. In embodiments, the KRAS mutation is a G13C mutation. In embodiments, the KRAS mutation is a Q61 mutation. In embodiments, the KRAS mutation is a Q61H mutation. In embodiments, the KRAS mutation is a Q61R mutation. In embodiments, the cancer has a BRAF mutation. In embodiments, the BRAF mutation is a V600E mutation. In embodiments, the cancer is a EGFR-TK-resistant cancer. In embodiments, the EGFR-TK inhibitor and the compound of AOH1996 or the pharmaceutically acceptable salt thereof are separately administered to the patient. In embodiments, the methods of treating cancer comprising administering to the patient a first pharmaceutical composition comprising the EGFR-TK inhibitor and a pharmaceutically acceptable excipient and a second pharmaceutical composition comprising AOH1996 or the pharmaceutically acceptable salt thereof and a pharmaceutically acceptable excipient (i.e., the first pharmaceutical composition and the second pharmaceutical compositions are different). In embodiments, the methods comprise administering to the patient a pharmaceutical composition comprising the EGFR-TK inhibitor, AOH1996 or the pharmaceutically acceptable salt thereof, and a pharmaceutically acceptable excipient. In embodiments, the methods of treating cancer further comprise administering to the patient an effective amount of one or more anti-cancer agents.

The disclosure provides methods of treating non-small cell lung cancer in a patient in need thereof by administering an effective amount of AOH1996 or a pharmaceutically acceptable salt thereof and an effective amount of an EGFR-TK inhibitor. In embodiments, the methods comprise treating non-small cell lung cancer by administering an effective amount of AOH1996 or a pharmaceutically acceptable salt thereof and an effective amount of osimertinib, gefitinib, afatinib, neratinib, erlotinib, rociletinib, olmutinib, lazertinib, nazartinib, naquotinib, mavelertinib, abivertinib, olafertinib, alflutinib, amivantamb, tarloxitinib, mobocertinib, savolitinib, capmatinib, cetuximab, panitumumab, lapatinib, dacomitinib, necitumumab, vandetanib, icotininib, canertinib, allitinib, varlitinib, tesevatinib, pelitinib, sapitinib, EAI045, TAK-285, AG-1478, AEE788, CUDC-101, WZ8040, WZ4002, WZ3146, AG-490, or PD153035. In embodiments, the EGFR-TK inhibitor is in the form of a pharmaceutically acceptable salt. In embodiments, the methods comprise treating non-small cell lung cancer by administering an effective amount of AOH1996 or a pharmaceutically acceptable salt thereof and an effective amount of osimertinib. In embodiments, the methods comprise treating non-small cell lung cancer by administering an effective amount of AOH1996 or a pharmaceutically acceptable salt thereof and an effective amount of osimertinib. In embodiments, the methods comprise treating non-small cell lung cancer by administering an effective amount of AOH1996 or a pharmaceutically acceptable salt thereof and an effective amount of gefitinib. In embodiments, the methods comprise treating non-small cell lung cancer by administering an effective amount of AOH1996 or a pharmaceutically acceptable salt thereof and an effective amount of afatinib. In embodiments, the methods comprise treating non-small cell lung cancer by administering an effective amount of AOH1996 or a pharmaceutically acceptable salt thereof and an effective amount of erlotinib. In embodiments, the methods comprise treating non-small cell lung cancer by administering an effective amount of AOH1996 or a pharmaceutically acceptable salt thereof and an effective amount of rociletinib. In embodiments, the methods comprise treating non-small cell lung cancer by administering an effective amount of AOH1996 or a pharmaceutically acceptable salt thereof and an effective amount of olmutinib. In embodiments, the methods comprise treating non-small cell lung cancer by administering an effective amount of AOH1996 or a pharmaceutically acceptable salt thereof and an effective amount of lazertinib. In embodiments, the methods comprise treating non-small cell lung cancer by administering an effective amount of AOH1996 or a pharmaceutically acceptable salt thereof and an effective amount of nazartinib. In embodiments, the methods comprise treating non-small cell lung cancer by administering an effective amount of AOH1996 or a pharmaceutically acceptable salt thereof and an effective amount of naquotinib. In embodiments, the methods comprise treating non-small cell lung cancer by administering an effective amount of AOH1996 or a pharmaceutically acceptable salt thereof and an effective amount of mavelertinib. In embodiments, the methods comprise treating non-small cell lung cancer by administering an effective amount of AOH1996 or a pharmaceutically acceptable salt thereof and an effective amount of abivertinib. In embodiments, the methods comprise treating non-small cell lung cancer by administering an effective amount of AOH1996 or a pharmaceutically acceptable salt thereof and an effective amount of olafertinib. In embodiments, the methods comprise treating non-small cell lung cancer by administering an effective amount of AOH1996 or a pharmaceutically acceptable salt thereof and an effective amount of alflutinib. In embodiments, the methods comprise treating non-small cell lung cancer by administering an effective amount of AOH1996 or a pharmaceutically acceptable salt thereof and an effective amount of amivantamb. In embodiments, the methods comprise treating non-small cell lung cancer by administering an effective amount of AOH1996 or a pharmaceutically acceptable salt thereof and an effective amount of tarloxitinib. In embodiments, the methods comprise treating non-small cell lung cancer by administering an effective amount of AOH1996 or a pharmaceutically acceptable salt thereof and an effective amount of mobocertinib. In embodiments, the methods comprise treating non-small cell lung cancer by administering an effective amount of AOH1996 or a pharmaceutically acceptable salt thereof and an effective amount of EA1045. In embodiments, the methods comprise treating non-small cell lung cancer by administering an effective amount of AOH1996 or a pharmaceutically acceptable salt thereof and an effective amount of savolitinib. In embodiments, the methods comprise treating non-small cell lung cancer by administering an effective amount of AOH1996 or a pharmaceutically acceptable salt thereof and an effective amount of capmatinib. In embodiments, the methods comprise treating non-small cell lung cancer by administering an effective amount of AOH1996 or a pharmaceutically acceptable salt thereof and an effective amount of cetuximab. In embodiments, the methods comprise treating non-small cell lung cancer by administering an effective amount of AOH1996 or a pharmaceutically acceptable salt thereof and an effective amount of panitumumab. In embodiments, the methods comprise treating non-small cell lung cancer by administering an effective amount of AOH1996 or a pharmaceutically acceptable salt thereof and an effective amount of lapatinib. In embodiments, the methods comprise treating non-small cell lung cancer by administering an effective amount of AOH1996 or a pharmaceutically acceptable salt thereof and an effective amount of dacomitinib. In embodiments, the methods comprise treating non-small cell lung cancer by administering an effective amount of AOH1996 or a pharmaceutically acceptable salt thereof and an effective amount of necitumumab. In embodiments, the methods comprise treating non-small cell lung cancer by administering an effective amount of AOH1996 or a pharmaceutically acceptable salt thereof and an effective amount of vandetanib. In embodiments, the methods comprise treating non-small cell lung cancer by administering an effective amount of AOH1996 or a pharmaceutically acceptable salt thereof and an effective amount of icotininib. In embodiments, the methods comprise treating non-small cell lung cancer by administering an effective amount of AOH1996 or a pharmaceutically acceptable salt thereof and an effective amount of canertinib. In embodiments, the methods comprise treating non-small cell lung cancer by administering an effective amount of AOH1996 or a pharmaceutically acceptable salt thereof and an effective amount of allitinib. In embodiments, the methods comprise treating non-small cell lung cancer by administering an effective amount of AOH1996 or a pharmaceutically acceptable salt thereof and an effective amount of varlitinib. In embodiments, the methods comprise treating non-small cell lung cancer by administering an effective amount of AOH1996 or a pharmaceutically acceptable salt thereof and an effective amount of tesevatinib. In embodiments, the methods comprise treating non-small cell lung cancer by administering an effective amount of AOH1996 or a pharmaceutically acceptable salt thereof and an effective amount of pelitinib. In embodiments, the methods comprise treating non-small cell lung cancer by administering an effective amount of AOH1996 or a pharmaceutically acceptable salt thereof and an effective amount of sapitinib. In embodiments, the methods comprise treating non-small cell lung cancer by administering an effective amount of AOH1996 or a pharmaceutically acceptable salt thereof and an effective amount of TAK-285. In embodiments, the methods comprise treating non-small cell lung cancer by administering an effective amount of AOH1996 or a pharmaceutically acceptable salt thereof and an effective amount of AG-1478. In embodiments, the methods comprise treating non-small cell lung cancer by administering an effective amount of AOH1996 or a pharmaceutically acceptable salt thereof and an effective amount of AEE788. In embodiments, the methods comprise treating non-small cell lung cancer by administering an effective amount of AOH1996 or a pharmaceutically acceptable salt thereof and an effective amount of CUDC-101. In embodiments, the methods comprise treating non-small cell lung cancer by administering an effective amount of AOH1996 or a pharmaceutically acceptable salt thereof and an effective amount of WZ8040. In embodiments, the methods comprise treating non-small cell lung cancer by administering an effective amount of AOH1996 or a pharmaceutically acceptable salt thereof and an effective amount of WZ4002. In embodiments, the methods comprise treating non-small cell lung cancer by administering an effective amount of AOH1996 or a pharmaceutically acceptable salt thereof and an effective amount of WZ3146. In embodiments, the methods comprise treating non-small cell lung cancer by administering an effective amount of AOH1996 or a pharmaceutically acceptable salt thereof and an effective amount of AG-490. In embodiments, the methods comprise treating non-small cell lung cancer by administering an effective amount of AOH1996 or a pharmaceutically acceptable salt thereof and an effective amount of PD153035. In embodiments, the non-small cell lung cancer has an EGFR mutation, a KRAS mutation, a BRAF mutation, or a combination of two or more thereof. In embodiments, the non-small cell lung cancer has an EGFR mutation. In embodiments, the EGFR mutation comprises L858R, ex19del, Ex10ins, T790M, or a combination of two or more thereof. In embodiments, the EGFR mutation comprises L858R and ex19del. In embodiments, the EGFR mutation comprises L858R. In embodiments, the EGFR mutation comprises ex19del. In embodiments, the EGFR mutation comprises Ex10ins. In embodiments, the EGFR mutation comprises L858R, ex19del, and T790M. In embodiments, the EGFR mutation comprises L858R and T790M. In embodiments, the EGFR mutation comprises T790M. In embodiments, the non-small cell lung cancer has a KRAS mutation. In embodiments, the KRAS mutation is G12, G13, or Q61. In embodiments, the KRAS mutation is G12C, G12S, G12V, G12D, G12A, G13D, G13C, Q61H, or Q61R. In embodiments, the KRAS mutation is a G12 mutation. In embodiments, the KRAS mutation is a G12C mutation. In embodiments, the KRAS mutation is a G12S mutation. In embodiments, the KRAS mutation is a G12V mutation. In embodiments, the KRAS mutation is a G12D mutation. In embodiments, the KRAS mutation is a G12A mutation. In embodiments, the KRAS mutation is a G13 mutation. In embodiments, the KRAS mutation is a G13D mutation. In embodiments, the KRAS mutation is a G13C mutation. In embodiments, the KRAS mutation is a Q61 mutation. In embodiments, the KRAS mutation is a Q61H mutation. In embodiments, the KRAS mutation is a Q61R mutation. In embodiments, the non-small cell lung cancer has a BRAF mutation. In embodiments, the BRAF mutation is a V600E mutation. In embodiments, the cancer is a EGFR-TK-resistant cancer. In embodiments, the EGFR-TK inhibitor and the compound of Formula (A) are separately administered to the patient. In embodiments, the methods of treating non-small cell lung cancer comprise administering to the patient a first pharmaceutical composition comprising the EGFR-TK inhibitor and a pharmaceutically acceptable excipient and a second pharmaceutical composition comprising A0H1996 or the pharmaceutically acceptable salt thereof and a pharmaceutically acceptable excipient (i.e., the first pharmaceutical composition and the second pharmaceutical compositions are different). In embodiments, the methods of treating non-small cell lung cancer comprise administering to the patient a pharmaceutical composition comprising the EGFR-TK inhibitor, A0H1996 or the pharmaceutically acceptable salt thereof, and a pharmaceutically acceptable excipient. In embodiments, the methods of treating cancer further comprise administering to the patient an effective amount of one or more anti-cancer agents.

The disclosure provides methods of treating cancer in a patient in need thereof by administering an effective amount of a compound of Formula (I) or a pharmaceutically acceptable salt thereof as described herein (including all embodiments thereof) and an effective amount of an EGFR-TK inhibitor. In embodiments, the methods comprise treating cancer by administering an effective amount of a compound of Formula (I) or a pharmaceutically acceptable salt thereof as described herein (including all embodiments thereof) and an effective amount of osimertinib, gefitinib, afatinib, neratinib, erlotinib, rociletinib, olmutinib, lazertinib, nazartinib, naquotinib, mavelertinib, abivertinib, olafertinib, alflutinib, amivantamb, tarloxitinib, mobocertinib, savolitinib, capmatinib, cetuximab, panitumumab, lapatinib, dacomitinib, necitumumab, vandetanib, icotininib, canertinib, allitinib, varlitinib, tesevatinib, pelitinib, sapitinib, EAI045, TAK-285, AG-1478, AEE788, CUDC-101, WZ8040, WZ4002, WZ3146, AG-490, or PD153035. In embodiments, the EGFR-TK inhibitor is in the form of a pharmaceutically acceptable salt. In embodiments, the methods comprise treating cancer by administering an effective amount of a compound of Formula (I) or a pharmaceutically acceptable salt thereof as described herein (including all embodiments thereof) and an effective amount of osimertinib. In embodiments, the methods comprise treating cancer by administering an effective amount of a compound of Formula (I) or a pharmaceutically acceptable salt thereof as described herein (including all embodiments thereof) and an effective amount of osimertinib. In embodiments, the methods comprise treating cancer by administering an effective amount of a compound of Formula (I) or a pharmaceutically acceptable salt thereof as described herein (including all embodiments thereof) and an effective amount of gefitinib. In embodiments, the methods comprise treating cancer by administering an effective amount of a compound of Formula (I) or a pharmaceutically acceptable salt thereof as described herein (including all embodiments thereof) and an effective amount of afatinib. In embodiments, the methods comprise treating cancer by administering an effective amount of a compound of Formula (I) or a pharmaceutically acceptable salt thereof as described herein (including all embodiments thereof) and an effective amount of erlotinib. In embodiments, the methods comprise treating cancer by administering an effective amount of a compound of Formula (I) or a pharmaceutically acceptable salt thereof as described herein (including all embodiments thereof) and an effective amount of rociletinib. In embodiments, the methods comprise treating cancer by administering an effective amount of a compound of Formula (I) or a pharmaceutically acceptable salt thereof as described herein (including all embodiments thereof) and an effective amount of olmutinib. In embodiments, the methods comprise treating cancer by administering an effective amount of a compound of Formula (I) or a pharmaceutically acceptable salt thereof as described herein (including all embodiments thereof) and an effective amount of lazertinib. In embodiments, the methods comprise treating cancer by administering an effective amount of a compound of Formula (I) or a pharmaceutically acceptable salt thereof as described herein (including all embodiments thereof) and an effective amount of nazartinib. In embodiments, the methods comprise treating cancer by administering an effective amount of a compound of Formula (I) or a pharmaceutically acceptable salt thereof as described herein (including all embodiments thereof) and an effective amount of naquotinib. In embodiments, the methods comprise treating cancer by administering an effective amount of a compound of Formula (I) or a pharmaceutically acceptable salt thereof as described herein (including all embodiments thereof) and an effective amount of mavelertinib. In embodiments, the methods comprise treating cancer by administering an effective amount of a compound of Formula (I) or a pharmaceutically acceptable salt thereof as described herein (including all embodiments thereof) and an effective amount of abivertinib. In embodiments, the methods comprise treating cancer by administering an effective amount of a compound of Formula (I) or a pharmaceutically acceptable salt thereof as described herein (including all embodiments thereof) and an effective amount of olafertinib. In embodiments, the methods comprise treating cancer by administering an effective amount of a compound of Formula (I) or a pharmaceutically acceptable salt thereof as described herein (including all embodiments thereof) and an effective amount of alflutinib. In embodiments, the methods comprise treating cancer by administering an effective amount of a compound of Formula (I) or a pharmaceutically acceptable salt thereof as described herein (including all embodiments thereof) and an effective amount of amivantamb. In embodiments, the methods comprise treating cancer by administering an effective amount of a compound of Formula (I) or a pharmaceutically acceptable salt thereof as described herein (including all embodiments thereof) and an effective amount of tarloxitinib. In embodiments, the methods comprise treating cancer by administering an effective amount of a compound of Formula (I) or a pharmaceutically acceptable salt thereof as described herein (including all embodiments thereof) and an effective amount of mobocertinib. In embodiments, the methods comprise treating cancer by administering an effective amount of a compound of Formula (I) or a pharmaceutically acceptable salt thereof as described herein (including all embodiments thereof) and an effective amount of EAI045. In embodiments, the methods comprise treating cancer by administering an effective amount of a compound of Formula (I) or a pharmaceutically acceptable salt thereof as described herein (including all embodiments thereof) and an effective amount of savolitinib. In embodiments, the methods comprise treating cancer by administering an effective amount of a compound of Formula (I) or a pharmaceutically acceptable salt thereof as described herein (including all embodiments thereof) and an effective amount of capmatinib. In embodiments, the methods comprise treating cancer by administering an effective amount of a compound of Formula (I) or a pharmaceutically acceptable salt thereof as described herein (including all embodiments thereof) and an effective amount of cetuximab. In embodiments, the methods comprise treating cancer by administering an effective amount of a compound of Formula (I) or a pharmaceutically acceptable salt thereof as described herein (including all embodiments thereof) and an effective amount of panitumumab. In embodiments, the methods comprise treating cancer by administering an effective amount of a compound of Formula (I) or a pharmaceutically acceptable salt thereof as described herein (including all embodiments thereof) and an effective amount of lapatinib. In embodiments, the methods comprise treating cancer by administering an effective amount of a compound of Formula (I) or a pharmaceutically acceptable salt thereof as described herein (including all embodiments thereof) and an effective amount of dacomitinib. In embodiments, the methods comprise treating cancer by administering an effective amount of a compound of Formula (I) or a pharmaceutically acceptable salt thereof as described herein (including all embodiments thereof) and an effective amount of necitumumab. In embodiments, the methods comprise treating cancer by administering an effective amount of a compound of Formula (I) or a pharmaceutically acceptable salt thereof as described herein (including all embodiments thereof) and an effective amount of vandetanib. In embodiments, the methods comprise treating cancer by administering an effective amount of a compound of Formula (I) or a pharmaceutically acceptable salt thereof as described herein (including all embodiments thereof) and an effective amount of icotininib. In embodiments, the methods comprise treating cancer by administering an effective amount of a compound of Formula (I) or a pharmaceutically acceptable salt thereof as described herein (including all embodiments thereof) and an effective amount of canertinib. In embodiments, the methods comprise treating cancer by administering an effective amount of a compound of Formula (I) or a pharmaceutically acceptable salt thereof as described herein (including all embodiments thereof) and an effective amount of allitinib. In embodiments, the methods comprise treating cancer by administering an effective amount of a compound of Formula (I) or a pharmaceutically acceptable salt thereof as described herein (including all embodiments thereof) and an effective amount of varlitinib. In embodiments, the methods comprise treating cancer by administering an effective amount of a compound of Formula (I) or a pharmaceutically acceptable salt thereof as described herein (including all embodiments thereof) and an effective amount of tesevatinib. In embodiments, the methods comprise treating cancer by administering an effective amount of a compound of Formula (I) or a pharmaceutically acceptable salt thereof as described herein (including all embodiments thereof) and an effective amount of pelitinib. In embodiments, the methods comprise treating cancer by administering an effective amount of a compound of Formula (I) or a pharmaceutically acceptable salt thereof as described herein (including all embodiments thereof) and an effective amount of sapitinib. In embodiments, the methods comprise treating cancer by administering an effective amount of a compound of Formula (I) or a pharmaceutically acceptable salt thereof as described herein (including all embodiments thereof) and an effective amount of TAK-285. In embodiments, the methods comprise treating cancer by administering an effective amount of a compound of Formula (I) or a pharmaceutically acceptable salt thereof as described herein (including all embodiments thereof) and an effective amount of AG-1478. In embodiments, the methods comprise treating cancer by administering an effective amount of a compound of Formula (I) or a pharmaceutically acceptable salt thereof as described herein (including all embodiments thereof) and an effective amount of AEE788. In embodiments, the methods comprise treating cancer by administering an effective amount of a compound of Formula (I) or a pharmaceutically acceptable salt thereof as described herein (including all embodiments thereof) and an effective amount of CUDC-101. In embodiments, the methods comprise treating cancer by administering an effective amount of a compound of Formula (I) or a pharmaceutically acceptable salt thereof as described herein (including all embodiments thereof) and an effective amount of WZ8040. In embodiments, the methods comprise treating cancer by administering an effective amount of a compound of Formula (I) or a pharmaceutically acceptable salt thereof as described herein (including all embodiments thereof) and an effective amount of WZ4002. In embodiments, the methods comprise treating cancer by administering an effective amount of a compound of Formula (I) or a pharmaceutically acceptable salt thereof as described herein (including all embodiments thereof) and an effective amount of WZ3146. In embodiments, the methods comprise treating cancer by administering an effective amount of a compound of Formula (I) or a pharmaceutically acceptable salt thereof as described herein (including all embodiments thereof) and an effective amount of AG-490. In embodiments, the methods comprise treating cancer by administering an effective amount of a compound of Formula (I) or a pharmaceutically acceptable salt thereof as described herein (including all embodiments thereof) and an effective amount of PD153035. In embodiments, the cancer is lung cancer, colorectal cancer, colon cancer, pancreatic cancer, breast cancer, squamous cell carcinoma, thyroid cancer, or head and neck cancer. In embodiments, the cancer is non-small cell lung cancer, colorectal cancer, colon cancer, pancreatic cancer, breast cancer, squamous cell carcinoma, thyroid cancer, or head and neck cancer. In embodiments, the cancer is lung cancer. In embodiments, the cancer is non-small cell lung cancer. In embodiments, the cancer is colorectal cancer. In embodiments, the cancer is colon cancer. In embodiments, the cancer is pancreatic cancer. In embodiments, the cancer is breast cancer. In embodiments, the cancer is squamous cell carcinoma. In embodiments, the cancer is squamous cell carcinoma. In embodiments, the cancer is thyroid cancer. In embodiments, the cancer is head and neck cancer. In embodiments, the cancer has an EGFR mutation, a KRAS mutation, a BRAF mutation, or a combination of two or more thereof. In embodiments, the cancer has an EGFR mutation. In embodiments, the EGFR mutation comprises L858R, ex19del, Ex10ins, T790M, or a combination of two or more thereof. In embodiments, the EGFR mutation comprises L858R and ex19del. In embodiments, the EGFR mutation comprises L858R. In embodiments, the EGFR mutation comprises ex19del. In embodiments, the EGFR mutation comprises Ex10ins. In embodiments, the EGFR mutation comprises L858R, ex19del, and T790M. In embodiments, the EGFR mutation comprises L858R and T790M. In embodiments, the EGFR mutation comprises T790M. In embodiments, the cancer has a KRAS mutation. In embodiments, the KRAS mutation is G12, G13, or Q61. In embodiments, the KRAS mutation is G12C, G12S, G12V, G12D, G12A, G13D, G13C, Q61H, or Q61R. In embodiments, the KRAS mutation is a G12 mutation. In embodiments, the KRAS mutation is a G12C mutation. In embodiments, the KRAS mutation is a G12S mutation. In embodiments, the KRAS mutation is a G12V mutation. In embodiments, the KRAS mutation is a G12D mutation. In embodiments, the KRAS mutation is a G12A mutation. In embodiments, the KRAS mutation is a G13 mutation. In embodiments, the KRAS mutation is a G13D mutation. In embodiments, the KRAS mutation is a G13C mutation. In embodiments, the KRAS mutation is a Q61 mutation. In embodiments, the KRAS mutation is a Q61H mutation, In embodiments, the KRAS mutation is a Q61R mutation. In embodiments, the cancer has a BRAF mutation. In embodiments, the BRAF mutation is a V600E mutation. In embodiments, the cancer is a EGFR-TK-resistant cancer. In embodiments, the EGFR-TK inhibitor and the compound of Formula (A) are separately administered to the patient. In embodiments, the methods of treating cancer comprise administering to the patient a first pharmaceutical composition comprising the EGFR-TK inhibitor and a pharmaceutically acceptable excipient and a second pharmaceutical composition comprising the compound of Formula (I) or the pharmaceutically acceptable salt thereof as described herein (including all embodiments thereof) and a pharmaceutically acceptable excipient (i.e., the first pharmaceutical composition and the second pharmaceutical compositions are different). In embodiments, the methods of treating cancer comprise administering to the patient a pharmaceutical composition comprising the EGFR-TK inhibitor, the compound of Formula (I) or the pharmaceutically acceptable salt thereof as described herein (including all embodiments thereof), and a pharmaceutically acceptable excipient. In embodiments, the methods of treating cancer further comprise administering to the patient an effective amount of one or more anti-cancer agents.

“Patient” or “subject in need thereof” or “subject” refers to a living organism suffering from or prone to a disease or condition that can be treated by administration of a compound or pharmaceutical composition or by a method, as provided herein. Non-limiting examples include humans, other mammals, bovines, rats, mice, dogs, monkeys, goat, sheep, cows, deer, and other non-mammalian animals. In embodiments, a patient is human. In embodiments, a subject is human. In embodiments, a subject is a human child (e.g., less than 18).

“Disease” or “condition” refer to a state of being or health status of a patient or subject capable of being treated with a compound, pharmaceutical composition, or method provided herein. In embodiments, the disease is a disease having the symptom of cell hyperproliferation. In embodiments, the disease is a disease having the symptom of an aberrant level of PCNA activity. In embodiments, the disease is a cancer.

As used herein, the term “cancer” refers to all types of cancer, neoplasm or malignant tumors found in mammals (e.g. humans), including leukemia, carcinomas and sarcomas. Exemplary cancers that may be treated with a compound or method provided herein include cancer of the prostate, thyroid, endocrine system, brain, breast, cervix, colon, head and neck, liver, kidney, lung, non-small cell lung, melanoma, mesothelioma, ovary, sarcoma, stomach, uterus, medulloblastoma, colorectal cancer, pancreatic cancer. Additional examples may include, Hodgkin's disease, Non-Hodgkin's lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

In embodiments of the methods described herein (including embodiments thereof), the cancer is cervical cancer, colon cancer, thyroid cancer, gastric cancer, ovarian cancer, breast cancer, lung cancer, uterine cancer, or ductal carcinoma in situ (DCIS). In embodiments, the cancer is neuroblastoma. In embodiments, the cancer is metastatic cancer. In embodiments, the cancer is breast cancer. In embodiments, the cancer is triple negative breast cancer. In embodiments, the cancer is metastatic breast cancer. In embodiments, the cancer is brain cancer. In embodiments, the cancer is glioblastoma. In embodiments, the cancer is astrocytoma. In embodiments, the cancer is glioma. In embodiments, the cancer is pancreatic cancer. In embodiments, the cancer is lymphoma. In embodiments, the cancer is chronic lymphoid leukemia (CLL). In embodiments, the cancer is non-Hodgkin's lymphoma. In embodiments, the cancer is skin cancer. In embodiments, the cancer is squamous cell carcinoma. In embodiments, the cancer is T lymphotrophic leukemia. In embodiments, the cancer is melanoma. In embodiments, the cancer is malignant melanoma. In embodiments, the cancer is lung cancer. In embodiments, the cancer is non-small cell lung cancer. In embodiments, the cancer is colon cancer. In embodiments, the cancer is prostate cancer. In embodiments, the cancer is ovarian cancer. In embodiments, the cancer is leukemia. In embodiments, the cancer is kidney cancer. In embodiments, the cancer is prostate, thyroid, endocrine system, brain, breast, cervix, colon, head and neck, liver, kidney, lung, non-small cell lung, melanoma, mesothelioma, ovary, sarcoma, stomach, uterus, medulloblastoma, colorectal cancer, pancreatic cancer.

Additional examples of cancers that can be treated with the methods (including all embodiments thereof) and compounds described herein (including all embodiments thereof), include, but are not limited to Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer. In embodiments, the cancer is leukemia, myeloma, non-small cell lung cancer, colon cancer, central nervous system cancer, melanoma, ovarian cancer, renal cancer, prostate cancer, or breast cancer. In embodiments, the cancer is neuroblastoma. In embodiments, the cancer is triple negative breast cancer. In embodiments, the cancer is a central nervous system (CNS) cancer. In embodiments, the cancer is a sympathetic nervous system (SNS) cancer. In embodiments, the cancer is an adrenal gland cancer. In embodiments, the cancer is a cancer of a neuron in the neck, chest, abdomen, or pelvis. In embodiments, the cancer is an esthesioneuroblastoma. In embodiments, the cancer is a stage 1 neuroblastoma (e.g., localized tumor confined to an area near the origin). In embodiments, the cancer is a stage 2A neuroblastoma (e.g., Unilateral tumor with incomplete gross resection and/or identifiable ipsilateral and contralateral lymph node negative for tumor). In embodiments, the cancer is a stage 2B neuroblastoma (e.g., Unilateral tumor with complete or incomplete gross resection; with ipsilateral lymph node positive for tumor; identifiable contralateral lymph node negative for tumor). In embodiments, the cancer is a stage 3 neuroblastoma (e.g., Tumor infiltrating across midline with or without regional lymph node involvement; or unilateral tumor with contralateral lymph node involvement; or midline tumor with bilateral lymph node involvement). In embodiments, the cancer is a stage 4 neuroblastoma (e.g., Dissemination of tumor to distant lymph nodes, bone marrow, bone, liver, or other organs except as defined by Stage 4S). In embodiments, the cancer is a stage 4S neuroblastoma (e.g., Age <1 year old with localized primary tumor as described in Stage 1 or Stage 2 above, with dissemination limited to liver, skin, or bone marrow (less than 10 percent of nucleated bone marrow cells are tumors). In embodiments, the cancer is a stage L1 neuroblastoma (e.g., localized cancer without image-defined risk factors) according to the International Neuroblastoma Risk Group (INRG) staging system. In embodiments, the cancer is a stage L2 neuroblastoma (e.g., localized cancer with image-defined risk factors) according to the International Neuroblastoma Risk Group (INRG) staging system. In embodiments, the cancer is a stage M neuroblastoma (e.g., metastatic cancer) according to the International Neuroblastoma Risk Group (1NRG) staging system. In embodiments, the cancer is a stage MS neuroblastoma (e.g., metastatic cancer “special” where MS is equivalent to stage 4S as described above) according to the International Neuroblastoma Risk Group (INRG) staging system. In embodiments, the cancer is a neuroblastoma risk stratification pre-treatment group, according to the International Neuroblastoma Risk Group (INRG) staging system, of very low. In embodiments, the cancer is a neuroblastoma risk stratification pre-treatment group, according to the International Neuroblastoma Risk Group (INRG) staging system, of low. In embodiments, the cancer is a neuroblastoma risk stratification pre-treatment group, according to the International Neuroblastoma Risk Group (INRG) staging system, of intermediate. In embodiments, the cancer is a neuroblastoma risk stratification pre treatment group, according to the International Neuroblastoma Risk Group (INRG) staging system, of high risk.

The terms “treating” or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In aspects, treating is preventing. In aspects, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms (e.g., ocular pain, seeing halos around lights, red eye, very high intraocular pressure), fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is not prophylactic treatment.

A “effective amount,” as used herein, is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). In these methods, the effective amount of the compounds described herein is an amount effective to accomplish the stated purpose of the method. An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%.

Therapeutic efficacy can also be expressed as “-fold” increase or decrease. For example, a therapeutically effective amount can have at least a 1.2-fold, 1.5-fold, 2-fold, 5-fold, or more effect over a control. For any compound described herein, the therapeutically effective amount can be initially determined from cell culture assays. Target concentrations will be those concentrations of active compound(s) that are capable of achieving the methods described herein, as measured using the methods described herein or known in the art. As is well known in the art, therapeutically effective amounts for use in humans can also be determined from animal models. For example, a dose for humans can be formulated to achieve a concentration that has been found to be effective in animals.

As used herein, the term “administering” means oral administration, administration as a suppository, topical contact, intravenous, parenteral, intrapentoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Co-administer” it is meant that a composition described herein is administered at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols.

“Anti-cancer agent” or “anti-cancer drug” is used in accordance with its plain ordinary meaning and refers to a composition (e.g. compound, drug, antagonist, inhibitor, modulator) having antineoplastic properties or the ability to inhibit the growth or proliferation of cells. In embodiments, an anti-cancer agent is a chemotherapeutic. In embodiments, an anti-cancer agent is an agent approved by the FDA or similar regulatory agency of a country other than the USA, for treating cancer. Examples of anti-cancer agents include, but are not limited to, anti-androgens (e.g., Casodex, Flutamide, MDV3100, or ARN-509), MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43 9006, wortmannin, or LY294002), mTOR inhibitors, antibodies (e.g., rituxan), 5-aza-2′-deoxycytidine, doxorubicin, vincristine, etoposide, gemcitabine, imatinib (Gleevec®), geldanamycin, 17-N-Allylamino-17-Demethoxygeldanamycin (17-AAG), bortezomib, trastuzumab, anastrozole; angiogenesis inhibitors; antiandrogen, antiestrogen; antisense oligonucleotides; apoptosis gene modulators; apoptosis regulators; arginine deaminase; BCR/ABL antagonists; beta lactam derivatives; bFGF inhibitor; bicalutamide; camptothecin derivatives; casein kinase inhibitors (ICOS); clomifene analogues; cytarabine dacliximab; dexamethasone; estrogen agonists; estrogen antagonists; etanidazole; etoposide phosphate; exemestane; fadrozole; finasteride; fludarabine; fluorodaunorunicin hydrochloride; gadolinium texaphyrin; gallium nitrate; gelatinase inhibitors; gemcitabine; glutathione inhibitors; hepsulfam; immunostimulant peptides; insulin-like growth factor-1 receptor inhibitor; interferon agonists; interferons; interleukins; letrozole; leukemia inhibiting factor; leukocyte alpha interferon; leuprolide+estrogen+progesterone; leuprorelin; matrilysin inhibitors; matrix metalloproteinase inhibitors; MIF inhibitor; mifepristone; mismatched double stranded RNA; monoclonal antibody; mycobacterial cell wall extract; nitric oxide modulators; oxaliplatin; panomifene; pentrozole; phosphatase inhibitors; plasminogen activator inhibitor; platinum complex; platinum compounds; prednisone; proteasome inhibitors; protein A-based immune modulator; protein kinase C inhibitor; protein tyrosine phosphatase inhibitors; purine nucleoside phosphorylase inhibitors; ras farnesyl protein transferase inhibitors; ras inhibitors; ras-GAP inhibitor; ribozymes; signal transduction inhibitors; signal transduction modulators; single chain antigen-binding protein; stem cell inhibitor; stem-cell division inhibitors; stromelysin inhibitors; synthetic glycosaminoglycans; tamoxifen methiodide; telomerase inhibitors; thyroid stimulating hormone; translation inhibitors; tyrosine kinase inhibitors; urokinase receptor antagonists; steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹¹In, ⁹⁰Y, or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, sorafenib, imatinib, sunitinib, dasatinib, pyrrolo benzodiazepines (e.g. tomaymycin), carboplatin, CC-1065 and CC-1065 analogs including amino-CBIs, nitrogen mustards (such as chlorambucil and melphalan), dolastatin and dolastatin analogs (including auristatins: eg. monomethyl auristatin E), anthracycline antibiotics (such as doxorubicin, daunorubicin, etc.), duocarmycins and duocarmycin analogs, enediynes (such as neocarzinostatin and calicheamicins), leptomycin derivaties, maytansinoids and maytansinoid analogs (e.g. mertansine), methotrexate, mitomycin C, taxoids, vinca alkaloids (such as vinblastine and vincristine), epothilones (e.g. epothilone B), camptothecin and its clinical analogs topotecan and irinotecan, or the like. In embodiments, the anti-cancer agent is not an EGFR-TK inhibitor and is not a PCNA inhibitor.

“Chemotherapeutic” or “chemotherapeutic agent” is used in accordance with its plain ordinary meaning and refers to a chemical composition or compound having antineoplastic properties or the ability to inhibit the growth or proliferation of cells.

### EMBODIMENTS

Embodiment 1. A method of treating cancer in a subject in need thereof, the method comprising administering to the subject an effective amount of an EGFR-TK inhibitor and an effective amount of a compound of Formula (A) or a pharmaceutically acceptable salt thereof; wherein the compound of Formula (A) is:

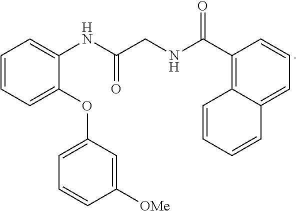 [SMILES: COc1cccc(Oc2ccccc2NC(=O)CNC(=O)c2cccc3ccccc23)c1]

Embodiment 2. The method of Embodiment 1, wherein the EGFR-TK inhibitor is osimertinib, gefitinib, afatinib, neratinib, erlotinib, rociletinib, olmutinib, lazertinib, nazartinib, naquotinib, mavelertinib, abivertinib, olafertinib, alflutinib, amivantamb, tarloxitinib, mobocertinib, savolitinib, capmatinib, cetuximab, panitumumab, lapatinib, dacomitinib, necitumumab, vandetanib, icotininib, canertinib, allitinib, varlitinib, tesevatinib, pelitinib, sapitinib, EAI045, TAK-285, AG-1478, AEE788, CUDC-101, WZ8040, WZ4002, WZ3146, AG-490, or PD153035.

Embodiment 3. The method of Embodiment 1, wherein the EGFR-TK inhibitor is osimertinib.

Embodiment 4. The method of Embodiment 1, wherein the EGFR-TK inhibitor is gefitinib.

Embodiment 5. The method of Embodiment 1, wherein the EGFR-TK inhibitor is afatinib.

Embodiment 6. The method of Embodiment 1, wherein the EGFR-TK inhibitor is neratinib.

Embodiment 7. The method of Embodiment 1, wherein the EGFR-TK inhibitor is erlotinib.

Embodiment 8. The method of any one of Embodiments 1 to 7, wherein the cancer is non-small cell lung cancer.

Embodiment 9. The method of any one of Embodiments 1 to 7, wherein the cancer is colorectal cancer.

Embodiment 10. The method of any one of Embodiments 1 to 7, wherein the cancer is colon cancer.

Embodiment 11. The method of any one of Embodiments 1 to 7, wherein the cancer is pancreatic cancer.

Embodiment 12. The method of any one of Embodiments 1 to 7, wherein the cancer is breast cancer.

Embodiment 13. The method of any one of Embodiments 1 to 7, wherein the cancer is thyroid cancer.

Embodiment 14. The method of any one of Embodiments 1 to 7, wherein the cancer is head and neck cancer.

Embodiment 15. The method of any one of Embodiments 1 to 14, wherein the cancer has an EGFR mutation, a KRAS mutation, a BRAF mutation, or a combination of two or more thereof.

Embodiment 16. The method of any one of Embodiments 1 to 14, wherein the cancer has an EGFR mutation comprising L858R, ex19del, Exl0ins, T790M, or a combination of two or more thereof.

Embodiment 17. The method of any one of Embodiments 1 to 14, wherein the cancer has a KRAS mutation comprising a G12 mutation, a G13 mutation, or a Q61 mutation, or a combination of two or more thereof.

Embodiment 18. The method of any one of Embodiments 1 to 17, comprising administering to the subject an effective amount of a first pharmaceutical composition comprising the EGFR-TK inhibitor and a pharmaceutically acceptable excipient and an effective amount of a second pharmaceutical composition comprising the compound of Formula (A) or the pharmaceutically acceptable salt thereof and a pharmaceutically acceptable excipient.

Embodiment 19. The method of any one of Embodiments 1 to 17, comprising administering to the subject an effective amount of a pharmaceutical composition comprising the EGFR-TK inhibitor, the compound of Formula (A) or the pharmaceutically acceptable salt thereof, and a pharmaceutically acceptable excipient.

Embodiment 20. A pharmaceutical composition comprising an EGFR-TK inhibitor, a compound of Formula (A) or a pharmaceutically acceptable salt thereof, and a pharmaceutically acceptable excipient; wherein the compound of Formula (A) is:

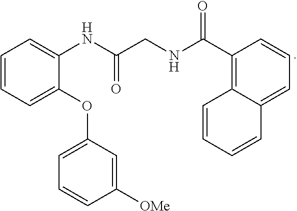 [SMILES: COc1cccc(Oc2ccccc2NC(=O)CNC(=O)c2cccc3ccccc23)c1]

Embodiment 21. The pharmaceutical composition of Embodiment 20, wherein the EGFR-TK inhibitor is osimertinib, gefitinib, afatinib, neratinib, erlotinib, rociletinib, olmutinib, lazertinib, nazartinib, naquotinib, mavelertinib, abivertinib, olafertinib, alflutinib, amivantamb, tarloxitinib, mobocertinib, savolitinib, capmatinib, cetuximab, panitumumab, lapatinib, dacomitinib, necitumumab, vandetanib, icotininib, canertinib, allitinib, varlitinib, tesevatinib, pelitinib, sapitinib, EAI045, TAK-285, AG-1478, AEE788, CUDC-101, WZ8040, WZ4002, WZ3146, AG-490, orPD153035.

Embodiment 22. The pharmaceutical composition of Embodiment 21, wherein the EGFR-TK inhibitor is osimertinib, gefitinib, afatinib, erlotinib, or neratinib.

Embodiment 23. A method of treating cancer in a subject in need thereof, the method comprising administering to the subject an effective amount of an EGFR-TK inhibitor and an effective amount of a compound of Formula (I) or a pharmaceutically acceptable salt thereof; wherein the compound of Formula (I) is:

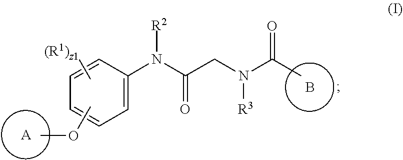

wherein: Ring A is substituted or unsubstituted phenyl or substituted or unsubstituted 5 to 6 membered heteroaryl; Ring B is substituted or unsubstituted naphthyl, substituted or unsubstituted quinolinyl, or substituted or unsubstituted isoquinolinyl; R¹ is independently a halogen, —CX¹₃, —CHX¹₂, —CH₂X¹, —CN, —SOₙ₁R¹⁰, —SOᵥ₁NR⁷R⁸, —NHNH₂, —ONR⁷R⁸, —NHC═(O)NHNH₂, —NHC═(O)NR⁷R⁸, —N(O)ₘ₁, —NR⁷R⁸, —C(O)R⁹, —C(O)—OR⁹, —C(O)NR⁷R⁸, —OR¹⁰, —NR⁷SO₂R¹⁰, —NR⁷C═(O)R⁹, —NR⁷C(O)—OR⁹, —NR⁷OR⁹, —OCX¹₃, —OCHX¹₂, —OCH₂X¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl; R² is independently hydrogen, halogen, —CX²₃, —CHX²₂, —CH₂X², —CN, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R³ is independently hydrogen, halogen, —CX³₃, —CHX³₂, —CH₂X³, —CN, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁷, R⁸, R⁹, and R¹⁰ are independently hydrogen, halogen, —CX^{A}3, —CHX^{A}₂, —CH₂X^{A}, —CN, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁷ and R⁸ substituents bonded to the same nitrogen atom may optionally be joined to form a substituted or unsubstituted heterocycloalkyl or substituted or unsubstituted heteroaryl; z1 is independently an integer from 0 to 4; m1 and v1 are independently 1 or 2; n1 is independently an integer from 0 to 4; and X¹, X², X³, and X^{A} are independently —Cl, —Br, —I, or —F.

Embodiment 24. The method of Embodiment 23, wherein the compound of Formula (I) is a compound of Formula (II) or a pharmaceutically acceptable salt thereof; wherein the compound of Formula (II) is:

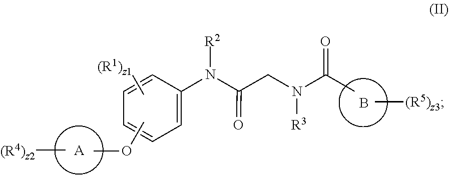

wherein: R⁴ is independently a halogen, —CX⁴₃, —CHX⁴₂, —CH₂X⁴, —CN, —SOₙ₄R¹⁴, —SOᵥ₄NR¹¹R¹², —NHNH₂, —ONR¹¹R¹², —NHC═(O)NHNH₂, —NHC═(O)NR¹¹R¹², —N(O)ₘ₄, —NR¹¹R¹², —C(O)R¹³, —C(O)—OR¹³, —C(O)NR¹¹R¹², —OR¹⁴, —NR¹¹SO₂R¹⁴, —NR¹¹C═(O)R¹³, —NR¹¹C(O)—OR¹³, —NR¹¹OR¹³, —OCX⁴₃, —OCHX⁴₂, —OCH₂X⁴, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R⁴ substituents may optionally be joined to form a substituted or unsubstituted cycloalkyl, substituted or unsubstituted heterocycloalkyl, substituted or unsubstituted aryl, or substituted or unsubstituted heteroaryl; R⁵ is independently a halogen, —CX⁵₃, —CHX⁵₂, —CH₂X⁵, —CN, —SOₙ₅R¹⁸, —SOᵥ₅NR¹⁵R¹⁶, —NHNH₂, —ONR¹⁵R¹⁶, —NHC═(O)NHNH₂, —NHC═(O)NR¹⁵R¹⁶, —N(O)ₘ₅, —NR¹⁵R¹⁶, —C(O)R¹⁷, —C(O)—OR¹⁷, —C(O)NR¹⁵R¹⁶, —OR¹⁸, —NR¹⁵SO₂R¹⁸, —NR¹⁵C═(O)R¹⁷, —NR¹⁵C(O)—OR¹⁷, —NR¹⁵OR¹⁷, —OCX⁵₃, —OCHX⁵₂, —OCH₂X⁵, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R⁵ substituents may optionally be joined to form a substituted or unsubstituted cycloalkyl, substituted or unsubstituted heterocycloalkyl, substituted or unsubstituted aryl, or substituted or unsubstituted heteroaryl; R¹¹, R¹², R¹³, and R¹⁴ are independently hydrogen, halogen. —CX^{B}₃, —CHX^{B}₂, —CH₂X^{B}, —CN, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹¹ and R¹² substituents bonded to the same nitrogen atom may optionally be joined to form a substituted or unsubstituted heterocycloalkyl or substituted or unsubstituted heteroaryl; R¹⁵, R¹⁶, R¹⁷, and R¹⁸ are independently hydrogen, halogen, —CX^{C}₃, —CHX^{C}₂, —CH₂X^{C}, —CN, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹⁵ and R¹⁶ substituents bonded to the same nitrogen atom may optionally be joined to form a substituted or unsubstituted heterocycloalkyl or substituted or unsubstituted heteroaryl; z2 is independently an integer from 0 to 5; z3 is independently an integer from 0 to 7; m4, m5, v4 and v5 are independently 1 or 2; n4 and n5 are independently an integer from 0 to 4; and X⁴, X⁵, X^{B}, and X^{C} are independently —Cl, —Br, —I, or —F.

Embodiment 25. The method of Embodiment 23, wherein the compound of Formula (I) is a compound of Formula (III) or a pharmaceutically acceptable salt thereof, wherein the compound of Formula (III) is:

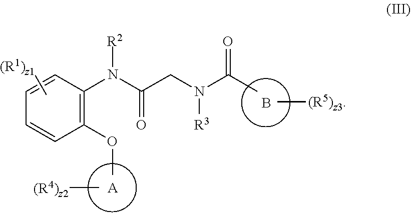

Embodiment 26. The method of Embodiment 23, wherein the compound of Formula (I) is a compound of Formula (IV) or a pharmaceutically acceptable salt thereof; wherein the compound of Formula (IV) is:

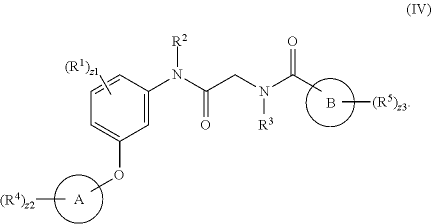

Embodiment 27. The method of Embodiment 23, wherein the compound of Formula (I) is a compound of Formula (V) or a pharmaceutically acceptable salt thereof; wherein the compound of Formula (V) is:

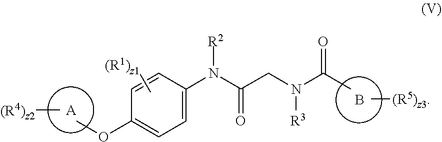

Embodiment 28. The method of any one of Embodiments 23 to 27, wherein R¹ is independently halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

Embodiment 29. The method of Embodiment 28, wherein R¹ is independently halogen, —CF₃, —OH, —NH₂, —SH, substituted or unsubstituted C₁-C₄ alkyl, substituted or unsubstituted 2 to 4 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl.

Embodiment 30. The method of Embodiment 29, wherein R¹ is independently halogen, —CF₃, —CHF₂, —CH₂F, —OCF₃, —OCHF₂, —OCH₂F, —OH, —NH₂, —SH, unsubstituted C₁-C₄ alkyl, or unsubstituted 2 to 4 membered heteroalkyl.

Embodiment 31. The method of Embodiment 30, wherein R¹ is independently halogen, —OH, —CF₃, —CHF₂, —CH₂F, —OCF₃, —OCHF₂, —OCH₂F, unsubstituted methyl, or unsubstituted methoxy.

Embodiment 32. The method of any one of Embodiments 23 to 31, wherein z1 is 1.

Embodiment 33. The method of any one of Embodiments 23 to 31, wherein z1 is 0.

Embodiment 34. The method of any one of Embodiments 23 to 33, wherein R⁴ is independently halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

Embodiment 35. The method of Embodiment 34, wherein R⁴ is independently halogen, —CF₃, —OH, —NH₂, —SH, substituted or unsubstituted C₁-C₄ alkyl, substituted or unsubstituted 2 to 4 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl.

Embodiment 36. The method of Embodiment 35, wherein R⁴ is independently halogen, —CF₃, —CHF₂, —CH₂F, —OCF₃, —OCHF₂, —OCH₂F, —OH, —NH₂, —SH, unsubstituted C₁-C₄ alkyl, or unsubstituted 2 to 4 membered heteroalkyl.

Embodiment 37. The method of Embodiment 36, wherein R⁴ is independently halogen, —CF₃, —CHF₂, —CH₂F, —OCF₃, —OCHF₂, —OCH₂F, —OH, unsubstituted methyl, or unsubstituted methoxy.

Embodiment 38. The method of Embodiment 37, wherein R⁴ is independently —OR¹⁴.

Embodiment 39. The method of any one of Embodiments 24 to 38, wherein R¹⁴ is hydrogen or substituted or unsubstituted alkyl.

Embodiment 40. The method of Embodiment 39, wherein R¹⁴ is hydrogen or unsubstituted alkyl.

Embodiment 41. The method of Embodiment 40, wherein R¹⁴ is hydrogen or unsubstituted C₁-C₅ alkyl.

Embodiment 42. The method of Embodiment 41, wherein R¹⁴ is hydrogen or unsubstituted C₁-C₃ alkyl.

Embodiment 43. The method of Embodiment 42, wherein R¹⁴ is hydrogen or unsubstituted methyl.

Embodiment 44. The method of Embodiment 43, wherein R¹⁴ is unsubstituted methyl.

Embodiment 45. The method of any one of Embodiments 24 to 44, wherein z2 is 1.

Embodiment 46. The method of any one of Embodiments 24 to 44, wherein z2 is 0.

Embodiment 47. The method of any one of Embodiments 24 to 46, wherein R⁵ is independently halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

Embodiment 48. The method of Embodiment 47, wherein R⁵ is independently halogen, —CF₃,

—OH, —NH₂, —SH, substituted or unsubstituted C₁-C₄ alkyl, substituted or unsubstituted 2 to 4 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl.

Embodiment 49. The method of Embodiment 48, wherein R⁵ is independently halogen, —CF₃,

—CHF₂, —CH₂F, —OCF₃, —OCHF₂, —OCH₂F, —OH, —NH₂, —SH, unsubstituted C₁-C₄ alkyl, or unsubstituted 2 to 4 membered heteroalkyl.

Embodiment 50. The method of Embodiment 49, wherein R⁵ is independently halogen, —CF₃, —CHF₂, —CH₂F, —OCF₃, —OCHF₂, —OCH₂F, —OH, unsubstituted methyl, or unsubstituted methoxy.

Embodiment 51. The method of any one of Embodiments 24 to 50, wherein z3 is 1.

Embodiment 52. The method of any one of Embodiments 24 to 50, wherein z3 is 0.

Embodiment 53. The method of any one of Embodiments 23 to 52, wherein R² is hydrogen,

—CX²₃, —CHX²₂, —CH₂X², —CN, —C(O)H, —C(O)OH, —C(O)NH₂, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl.

Embodiment 54. The method of Embodiment 53, wherein R² is hydrogen, unsubstituted methyl, unsubstituted ethyl, or unsubstituted isopropyl.

Embodiment 55. The method of Embodiment 54, wherein R² is hydrogen.

Embodiment 56. The method of any one of Embodiments 23 to 55, wherein R³ is hydrogen, —CX³₃, —CHX³₂, —CH₂X³, —CN, —C(O)H, —C(O)OH, —C(O)NH₂, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl.

Embodiment 57. The method of Embodiment 56, wherein R³ is hydrogen, unsubstituted methyl, unsubstituted ethyl, or unsubstituted isopropyl.

Embodiment 58. The method of Embodiment 57, wherein R³ is hydrogen.

Embodiment 59. The method of any one of Embodiments 23 to 58, wherein Ring A is a substituted or unsubstituted phenyl.

Embodiment 60. The method of Embodiment 59, wherein Ring A is phenyl.

Embodiment 61. The method of any one of Embodiments 23 to 58, wherein Ring A is a substituted or unsubstituted 5 to 6 membered heteroaryl.

Embodiment 62. The method of Embodiment 61, wherein Ring A is a 5 to 6 membered heteroaryl.

Embodiment 63. The method of any one of Embodiments 23 to 58, wherein Ring A is a substituted or unsubstituted thienyl.

Embodiment 64. The compound of Embodiment 24, wherein Ring A is a thienyl.

Embodiment 65. The method of Embodiment 64, wherein Ring A is a substituted or unsubstituted 2-thienyl.

Embodiment 66. The method of Embodiment 65, wherein Ring A is a 2-thienyl.

Embodiment 67. The method of any one of Embodiments 23 to 58, wherein Ring A is a substituted or unsubstituted 3-thienyl.

Embodiment 68. The method of Embodiment 67, wherein Ring A is a 3-thienyl.

Embodiment 69. The method of any one of Embodiments 23 to 58, wherein Ring A is a substituted or unsubstituted pyridyl.

Embodiment 70. The method of Embodiment 69, wherein Ring A is a pyridyl.

Embodiment 71. The method of any one of Embodiments 23 to 58, wherein Ring A is a substituted or unsubstituted 2-pyridyl.

Embodiment 72. The method of Embodiment 71, wherein Ring A is a 2-pyridyl.

Embodiment 73. The method of any one of Embodiments 23 to 58, wherein Ring A is a substituted or unsubstituted 3-pyridyl.

Embodiment 74. The method of Embodiment 73, wherein Ring A is a 3-pyridyl.

Embodiment 75. The method of any one of Embodiments 23 to 58, wherein Ring A is a substituted or unsubstituted 4-pyridyl.

Embodiment 76. The method of Embodiment 75, wherein Ring A is a 4-pyridyl.

Embodiment 77. The method of any one of Embodiments 23 to 76, wherein Ring B is a substituted or unsubstituted naphthyl.

Embodiment 78. The method of Embodiment 77, wherein Ring B is a naphthyl.

Embodiment 79. The method of Embodiment 78, wherein Ring B is a substituted or unsubstituted 1-naphthyl.

Embodiment 80. The method of Embodiment 79, wherein Ring B is a 1-naphthyl.

Embodiment 81. The method of any one of Embodiments 23 to 76, wherein Ring B is a substituted or unsubstituted 2-naphthyl.

Embodiment 82. The method of Embodiment 81, wherein Ring B is a 2-naphthyl.

Embodiment 83. The method of any one of Embodiments 23 to 76, wherein Ring B is a substituted or unsubstituted quinolinyl.

Embodiment 84. The method of Embodiment 83, wherein Ring B is a quinolinyl.

Embodiment 85. The method of any one of Embodiments 23 to 76, wherein Ring B is a substituted or unsubstituted isoquinolinyl.

Embodiment 86. The method of Embodiment 85, wherein Ring B is a isoquinolinyl.

Embodiment 87. The method of any one of Embodiments 23 to 76, wherein Ring B is a substituted or unsubstituted 1-isoquinolinyl.

Embodiment 88. The method of Embodiment 87, wherein Ring B is a 1-isoquinolinyl.

Embodiment 89. The method of any one of Embodiments 23 to 76, wherein Ring B is a substituted or unsubstituted 3-isoquinolinyl.

Embodiment 90. The method of Embodiment 89 wherein Ring B is a 3-isoquinolinyl.

Embodiment 91. The method of any one of Embodiments 23 to 76, wherein Ring B is a substituted or unsubstituted 4-isoquinolinyl.

Embodiment 92. The method of Embodiment 90, wherein Ring B is a 4-isoquinolinyl.

Embodiment 93. The method of any one of Embodiments 23 to 58, wherein the compound has the formula:

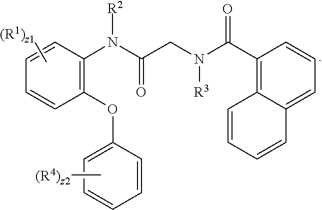

Embodiment 94. The method of any one of Embodiments 23 to 58, wherein the compound has the formula:

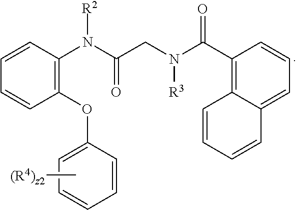

Embodiment 95. The method of any one of Embodiments 23 to 58, wherein the compound has the formula:

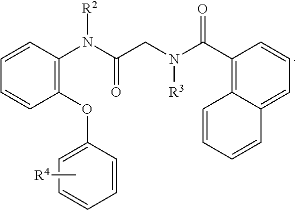

Embodiment 96. The method of any one of Embodiments 23 to 58, wherein the compound has the formula:

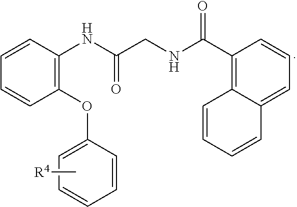

Embodiment 97. The method of any one of Embodiments 23 to 58, wherein the compound has the formula:

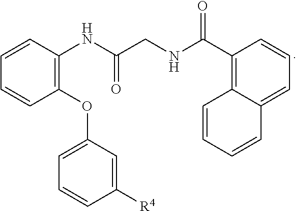

Embodiment 98. The method of any one of Embodiments 23 to 58, wherein the compound has the formula:

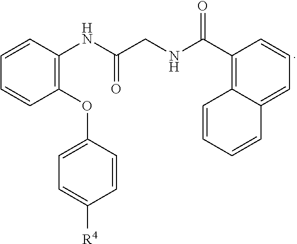

Embodiment 99. The method of Embodiment 23, wherein the compound has the formula:

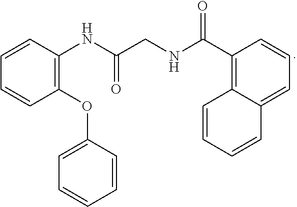 [SMILES: O=C(CNC(=O)c1cccc2ccccc12)Nc1ccccc1Oc1ccccc1]

Embodiment 100. The method of Embodiment 23, wherein the compound has the formula:

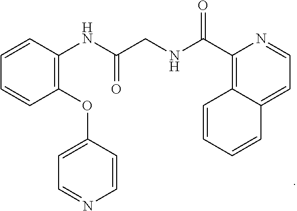 [SMILES: O=C(CNC(=O)c1nccc2ccccc12)Nc1ccccc1Oc1ccncc1]

Embodiment 101. The method of Embodiment 23, wherein the compound has the formula:

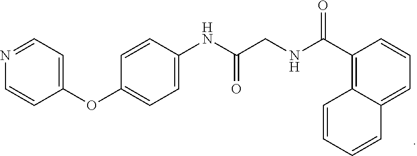 [SMILES: O=C(CNC(=O)c1cccc2ccccc12)Nc1ccc(Oc2ccncc2)cc1]

Embodiment 102. The method of Embodiment 23, wherein the compound has the formula:

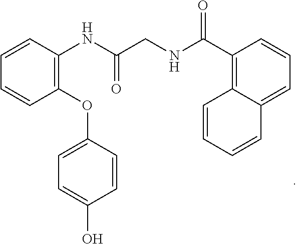 [SMILES: O=C(CNC(=O)c1cccc2ccccc12)Nc1ccccc1Oc1ccc(O)cc1]

Embodiment 103. The method of Embodiment 23, wherein the compound has the formula:

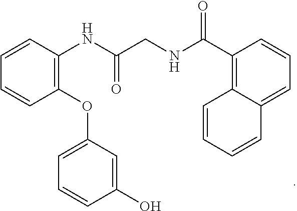 [SMILES: O=C(CNC(=O)c1cccc2ccccc12)Nc1ccccc1Oc1cccc(O)c1]

Embodiment 104. The method of Embodiment 23, wherein the compound has the formula:

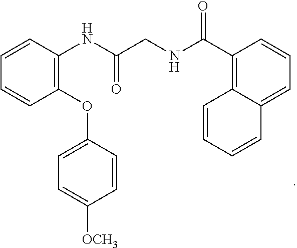 [SMILES: COc1ccc(Oc2ccccc2NC(=O)CNC(=O)c2cccc3ccccc23)cc1]

Embodiment 105. The method of Embodiment 23, wherein the compound has the formula:

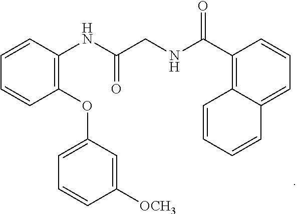 [SMILES: COc1cccc(Oc2ccccc2NC(=O)CNC(=O)c2cccc3ccccc23)c1]

Embodiment 106. The method of Embodiment 23, wherein the compound has the formula:

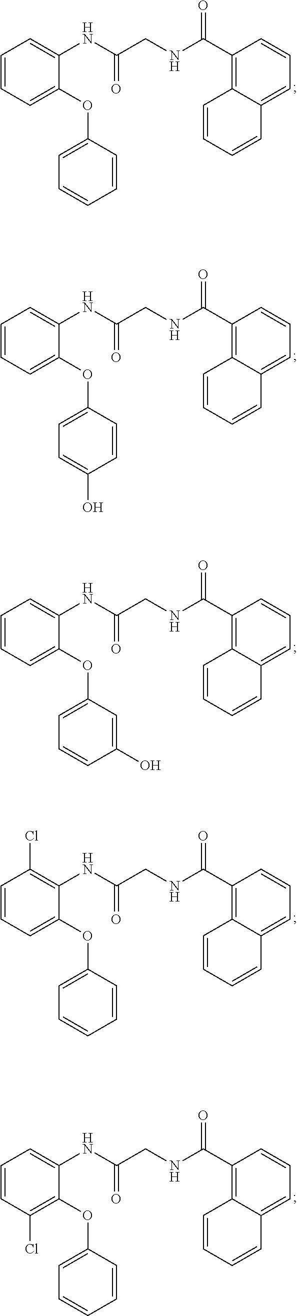 [SMILES: O=C(CNC(=O)c1cccc2ccccc12)Nc1ccccc1Oc1ccccc1 ; O=C(CNC(=O)c1cccc2ccccc12)Nc1ccccc1Oc1ccc(O)cc1 ; O=C(CNC(=O)c1cccc2ccccc12)Nc1ccccc1Oc1cccc(O)c1 ; O=C(CNC(=O)c1cccc2ccccc12)Nc1c(Cl)cccc1Oc1ccccc1 ; O=C(CNC(=O)c1cccc2ccccc12)Nc1cccc(Cl)c1Oc1ccccc1]

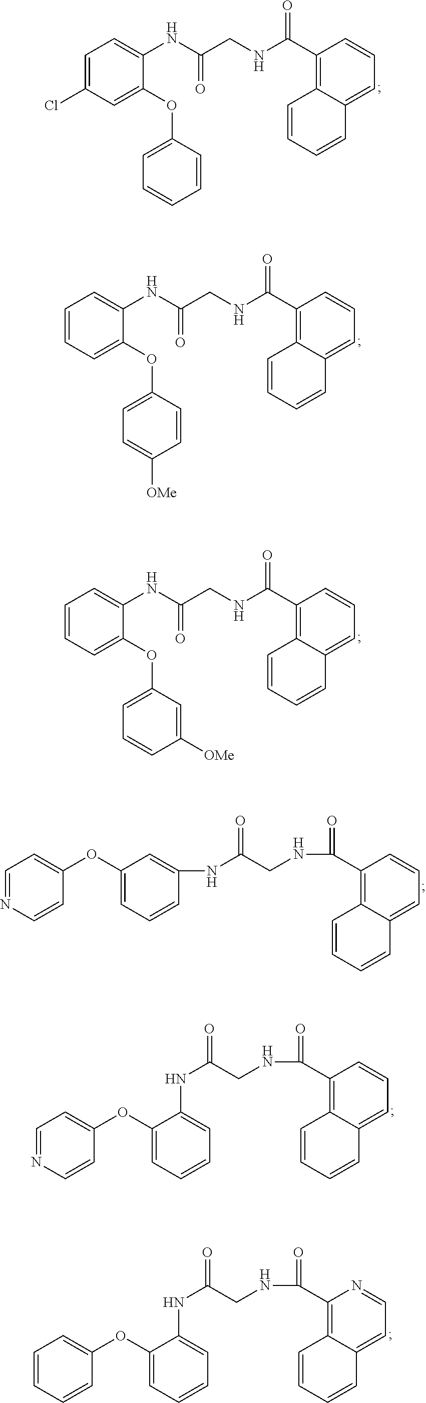 [SMILES: O=C(CNC(=O)c1cccc2ccccc12)Nc1ccc(Cl)cc1Oc1ccccc1 ; COc1ccc(Oc2ccccc2NC(=O)CNC(=O)c2cccc3ccccc23)cc1 ; COc1cccc(Oc2ccccc2NC(=O)CNC(=O)c2cccc3ccccc23)c1 ; O=C(CNC(=O)c1cccc2ccccc12)Nc1cccc(Oc2ccncc2)c1 ; O=C(CNC(=O)c1cccc2ccccc12)Nc1ccccc1Oc1ccncc1 ; O=C(CNC(=O)c1nccc2ccccc12)Nc1ccccc1Oc1ccccc1]

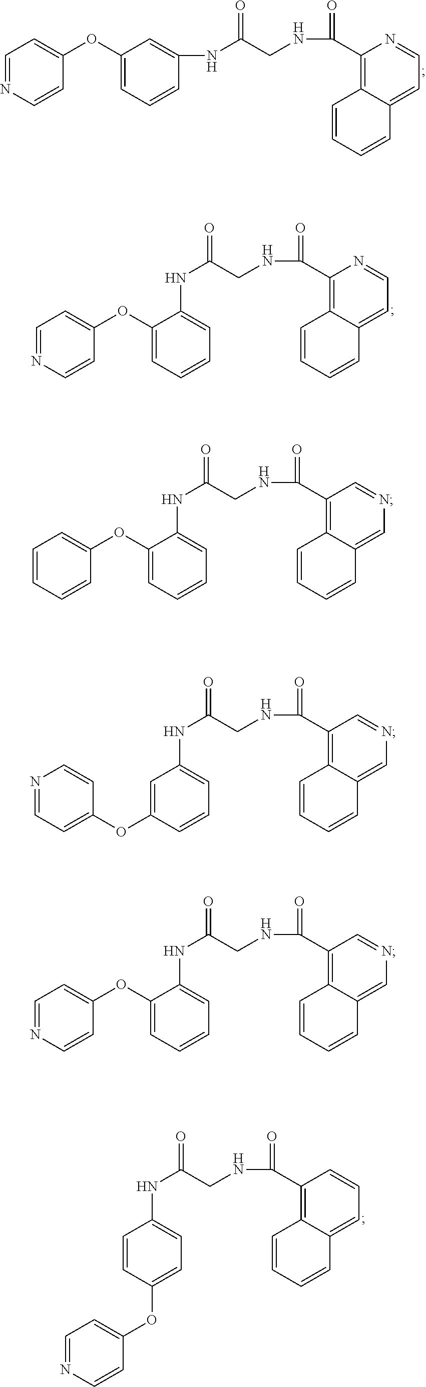 [SMILES: O=C(CNC(=O)c1nccc2ccccc12)Nc1cccc(Oc2ccncc2)c1 ; O=C(CNC(=O)c1nccc2ccccc12)Nc1ccccc1Oc1ccncc1 ; O=C(CNC(=O)c1cncc2ccccc12)Nc1ccccc1Oc1ccccc1 ; O=C(CNC(=O)c1cncc2ccccc12)Nc1cccc(Oc2ccncc2)c1 ; O=C(CNC(=O)c1cncc2ccccc12)Nc1ccccc1Oc1ccncc1 ; O=C(CNC(=O)c1cccc2ccccc12)Nc1ccc(Oc2ccncc2)cc1]

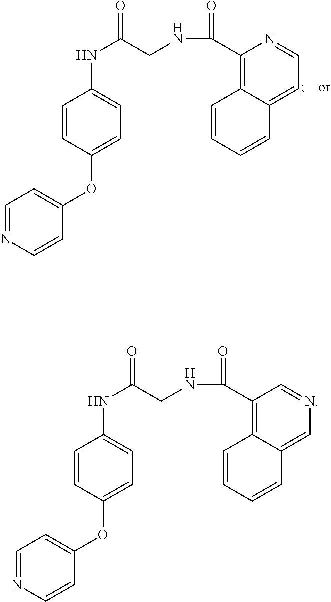 [SMILES: O=C(CNC(=O)c1nccc2ccccc12)Nc1ccc(Oc2ccncc2)cc1 ; O=C(CNC(=O)c1cncc2ccccc12)Nc1ccc(Oc2ccncc2)cc1]

Embodiment 107. The method of any one of Embodiments 23 to 106, wherein the EGFR-TK inhibitor is osimertinib, gefitinib, afatinib, neratinib, erlotinib, rociletinib, olmutinib, lazertinib, nazartinib, naquotinib, mavelertinib, abivertinib, olafertinib, alflutinib, amivantamb, tarloxitinib, mobocertinib, savolitinib, capmatinib, cetuximab, panitumumab, lapatinib, dacomitinib, necitumumab, vandetanib, icotininib, canertinib, allitinib, varlitinib, tesevatinib, pelitinib, sapitinib, EAI045, TAK-285, AG-1478, AEE788, CUDC-101, WZ8040, WZ4002, WZ3146, AG-490, orPD153035.

Embodiment 108. The method of any one of Embodiments 23 to 106, wherein the EGFR-TK inhibitor is osimertinib.

Embodiment 109. The method of any one of Embodiments 23 to 106, wherein the EGFR-TK inhibitor is gefitinib.

Embodiment 110. The method of any one of Embodiments 23 to 106, wherein the EGFR-TK inhibitor is afatinib.

Embodiment 111. The method of any one of Embodiments 23 to 106, wherein the EGFR-TK inhibitor is neratinib.

Embodiment 112. The method of any one of Embodiments 23 to 106, wherein the EGFR-TK inhibitor is erlotinib.

Embodiment 113. The method of any one of Embodiments 23 to 112, wherein the cancer is non-small cell lung cancer.

Embodiment 114. The method of any one of Embodiments 23 to 112, wherein the cancer is colorectal cancer.

Embodiment 115. The method of any one of Embodiments 23 to 112, wherein the cancer is colon cancer.

Embodiment 116. The method of any one of Embodiments 23 to 112, wherein the cancer is pancreatic cancer.

Embodiment 117. The method of any one of Embodiments 23 to 112, wherein the cancer is breast cancer

Embodiment 118. The method of any one of Embodiments 23 to 112, wherein the cancer is thyroid cancer.

Embodiment 119. The method of any one of Embodiments 23 to 112, wherein the cancer is head and neck cancer.

Embodiment 120. The method of any one of Embodiments 23 to 119, wherein the cancer has an EGFR mutation, a KRAS mutation, a BRAF mutation, or a combination thereof.

Embodiment 121. The method of any one of Embodiments 23 to 119, wherein the cancer has an EGFR mutation comprising L858R, ex19del, Ex10ins, T790M, or a combination of two or more thereof.

Embodiment 122. The method of any one of Embodiments 23 to 119, wherein the cancer has a KRAS mutation comprising a G12 mutation, a G13 mutation, a Q61 mutation, or a combination of two or more thereof.

Embodiment 123. The method of any one of Embodiments 23 to 122, wherein the EGFR-TK inhibitor and the compound or the pharmaceutically acceptable salt thereof are separately administered to the patient.

Embodiment 124. The method of any one of Embodiments 23 to 122, wherein the EGFR-TK inhibitor and the compound or the pharmaceutically acceptable salt thereof are administered to the patient in a single pharmaceutical composition.

Embodiment 125. A pharmaceutical composition comprising an EGFR-TK inhibitor, a compound of Formula (I) or a pharmaceutically acceptable salt thereof, and a pharmaceutically acceptable excipient; wherein the compound of Formula (I) is:

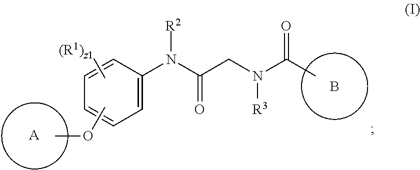

wherein: Ring A is substituted or unsubstituted phenyl or substituted or unsubstituted 5 to 6 membered heteroaryl; Ring B is substituted or unsubstituted naphthyl, substituted or unsubstituted quinolinyl, or substituted or unsubstituted isoquinolinyl; R¹ is independently a halogen, —CX¹₃, —CHX¹₂, —CH₂X¹, —CN, —SOₙ₁R¹⁰, —SOᵥ₁NR⁷R⁸, —NHNH₂, —ONR⁷R⁸, —NHC═(O)NHNH₂, —NHC═(O)NR⁷R⁸, —N(O)ₘ₁, —NR⁷R⁸, —C(O)R⁹, —C(O)—OR⁹, —C(O)NR⁷R⁸, —OR¹⁰, —NR¹SO₂R¹⁰, —NR⁷C═(O)R⁹, —NR⁷C(O)—OR⁹, —NR⁷OR⁹, —OCX¹₃, —OCHX¹₂, —OCH₂X¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R′ substituents may optionally be joined to form a substituted or unsubstituted cycloalkyl, substituted or unsubstituted heterocycloalkyl, substituted or unsubstituted aryl, or substituted or unsubstituted heteroaryl; R² is independently hydrogen, halogen, —CX²₃, —CHX²₂, —CH₂X², —CN, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R³ is independently hydrogen, halogen, —CX³₃, —CHX³₂, —CH₂X³, —CN, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁷, R⁸, R⁹, and R¹⁰ are independently hydrogen, halogen, —CX^{A}₃, —CHX^{A}₂, —CH₂X^{A}, —CN, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁷ and R⁸ substituents bonded to the same nitrogen atom may optionally be joined to form a substituted or unsubstituted heterocycloalkyl or substituted or unsubstituted heteroaryl; z1 is independently an integer from 0 to 4; m1 and v1 are independently 1 or 2; n1 is independently an integer from 0 to 4; and X¹, X², X³, and X^{A} are independently —Cl, —Br, —I, or —F

Embodiment 126. The pharmaceutical composition of Embodiment 125, wherein the EGFR-TK inhibitor is osimertinib, gefitinib, afatinib, neratinib, erlotinib, rociletinib, olmutinib, lazertinib, nazartinib, naquotinib, mavelertinib, abivertinib, olafertinib, alflutinib, amivantamb, tarloxitinib, mobocertinib, savolitinib, capmatinib, cetuximab, panitumumab, lapatinib, dacomitinib, necitumumab, vandetanib, icotininib, or EAI045.

Embodiment 127. The pharmaceutical composition of Embodiment 126, wherein the EGFR-TK inhibitor is osimertinib, gefitinib, afatinib, erlotinib, or neratinib.

### EXAMPLES

EGFR is localized to the cell membrane and nucleus. Cell membrane localized EGFR initiates signaling through various signaling pathways; the PI3K/AKT and the Ras-Raf-Mek-Erk signaling pathways are depicted here. Several functions have been attributed to nuclear localized EGFR. It can act as a transcription factor, it can phosphorylate PCNA which is important for stabilizing PCNA on chromatin, and it can interact with DNA-PK which is important for non-homologous end joining (NHEJ) and has been associated with radioresistance and chemoresistance. AOH1996 inhibits PCNA functions including replication and homologous recombination (HR) through direct interaction. EGFR TKIs bind to the ATP binding pocket of EGFR to block EGFR functions. An important consequence of this drug combination is that resistance to EGFR TKIs will be forestalled through the compounded effects on inhibition of PCNA through two different mechanisms and through the suppression of both major DNA double-strand break (DSB) repair pathways (NHEJ by EGFR TKIs and HR by AOH1996).

### Example 1

Methods of making the compounds described herein, including AOH1996, are set forth in U.S. Pat. Nos. 10,550,070 and 10,913,706.

### Example 2

Dose response assays were performed by plating 10,000 cells per well of a 96-well tissue culture plate. Triplicate wells were plated for each experimental condition. Plated cells were treated with 2-fold serial dilutions of either single drug or drugs in combination. Samples were incubated for 72 hours. At the end of the incubation the number of cells in each well was quantified by Sulforhodamine B (SRB) assay. In brief, the cells were fixed in 5% Trichloroacetic acid for 2 hrs at 4° C. Following fixing, plates were rinsed with water 4 times and dried under a heat lamp. The plated cells were then stained with 0.057% sulforhodamine B in 1% acetic acid for 30 minutes at room temperature. The cells were then washed 4 times with 1% acetic acid and dried under a heat lamp. When dry, the stained cells were resuspended in 10 mM unbuffered tris base. A multiplate reader tuned to read the signal emitted at 510 nM was used to quantify each experimental dosage point. The results were processed by subtracting background and normalizing samples to the signal from untreated cells. Single drug dose curves and the drug combination curve were aligned so that the single drug doses reflected the amount of each drug in the combination.

Growth curve. Two thousand HCC827 were plated in triplicate for each experimental condition and treated with DMSO, 500 nM AOH1996, 4 nM osimertinib or with 500 nM AOH1996 combined with 4 nM Osimertinib. Cells were fixed and quantified by SRB assay as described above. For each dosing condition samples were quantified at 24, 48, 72 and 96 hours.

Chromatin isolation. Two million HCC827 cells were plated in 6 cm tissue culture dishes. The plated cells were treated with DMSO, 500 nM AOH1996, 4 nM osimertinib and 500 nM AOH1996 combined with 4 nM osimertinib. The cells were trypsinized after 24 hours and processed using a subcellular protein fractionation kit (Thermo Scientific, cat no. 78840) and following the manufacturer's protocol. The chromatin-bound fraction was quantified for protein concentration and then separated by electrophoresis on a polyacrylamide gel. The protein was transferred to a nitrocellulose membrane and stained with Ponceau S to detect total protein on the blot. The blot was washed to destain and then blocked and hybridized with an antibody conjugated to IR-800 fluorescent dye and specific for detection of PCNA. Detection and imaging was done using an Azure c600 gel imaging system.

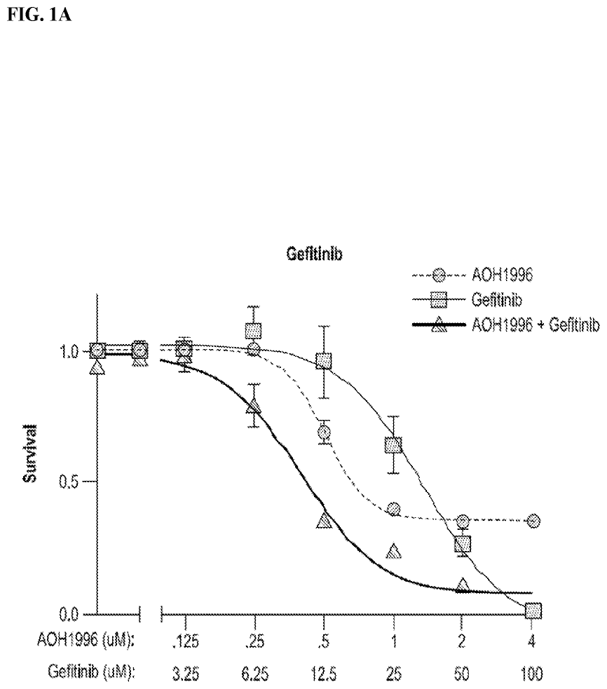

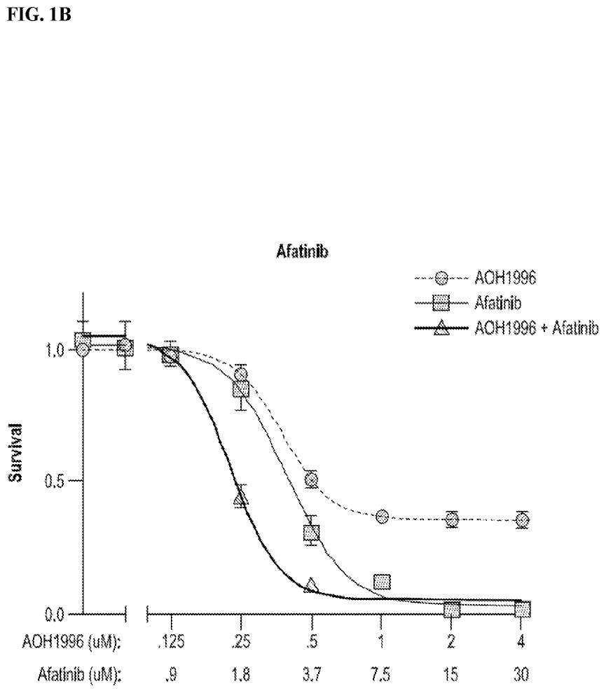

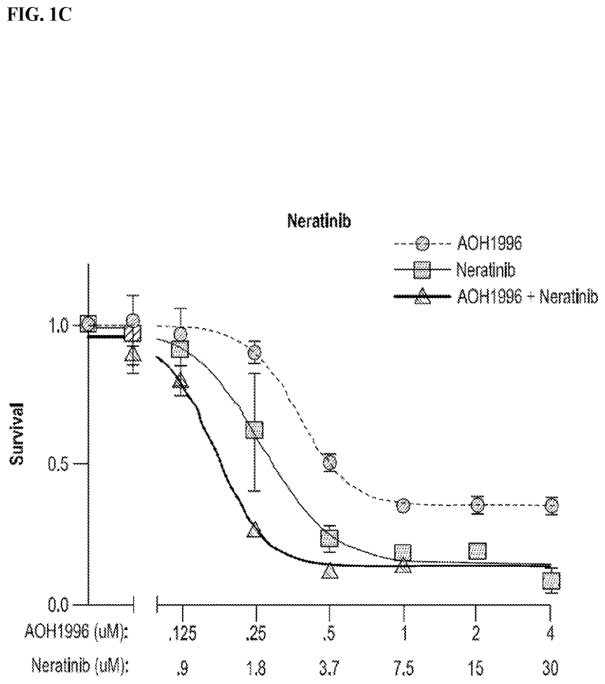

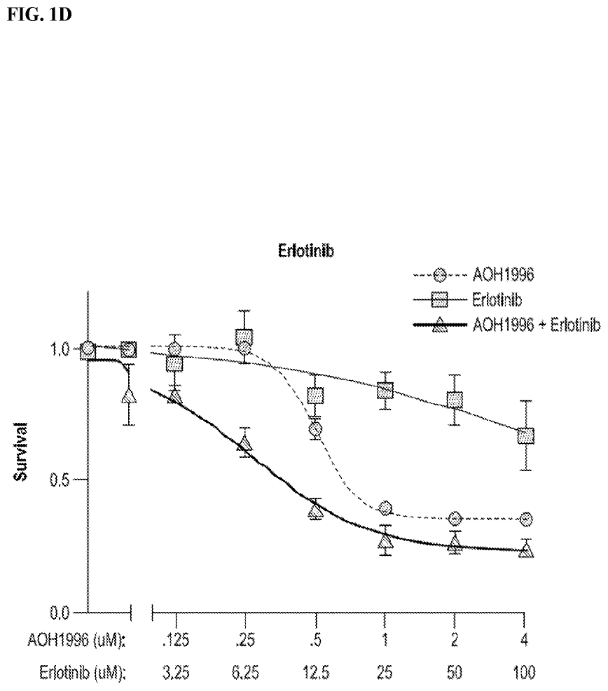

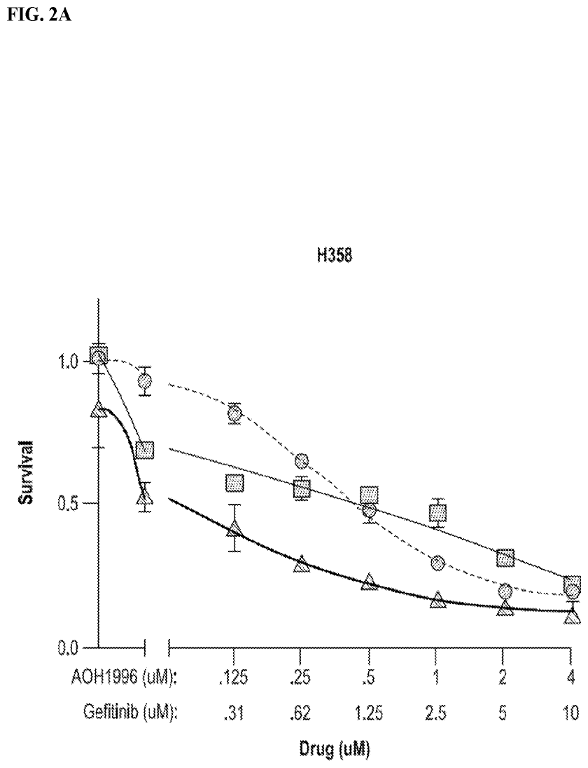

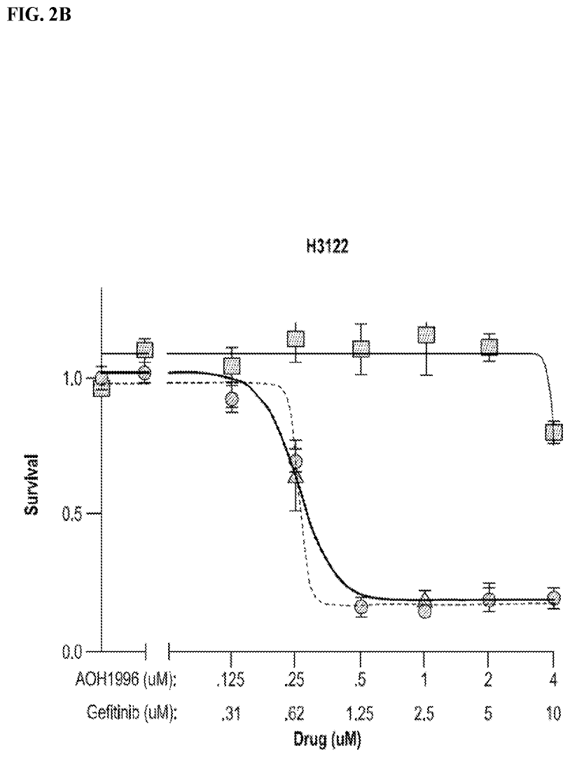

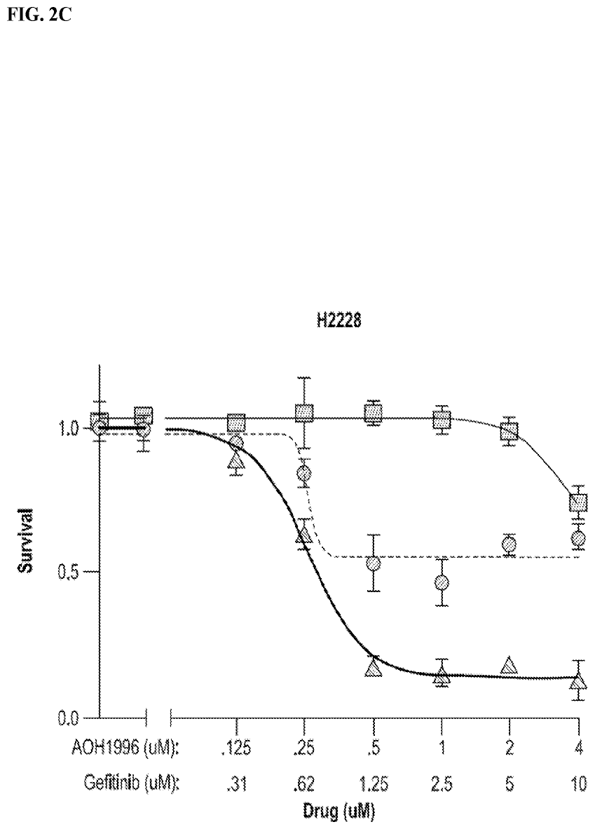

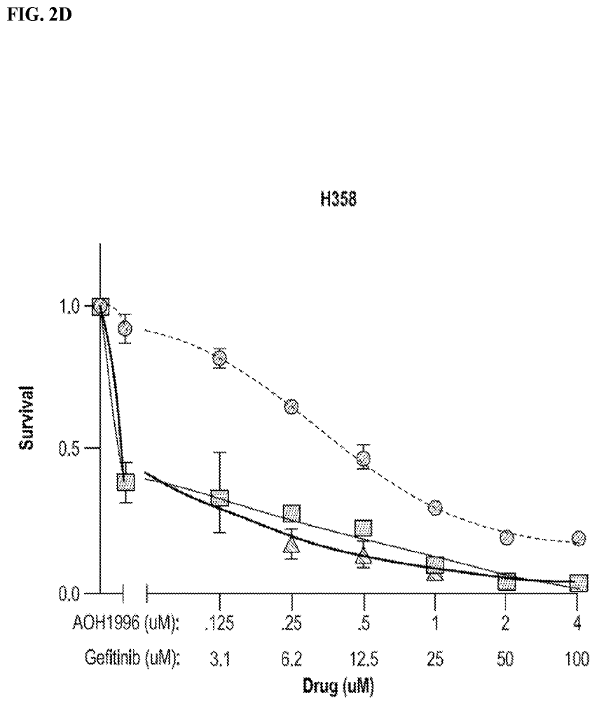

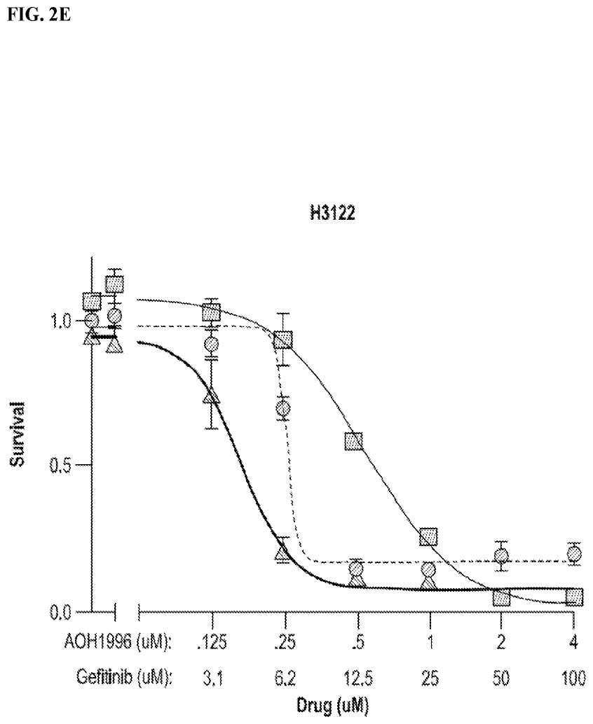

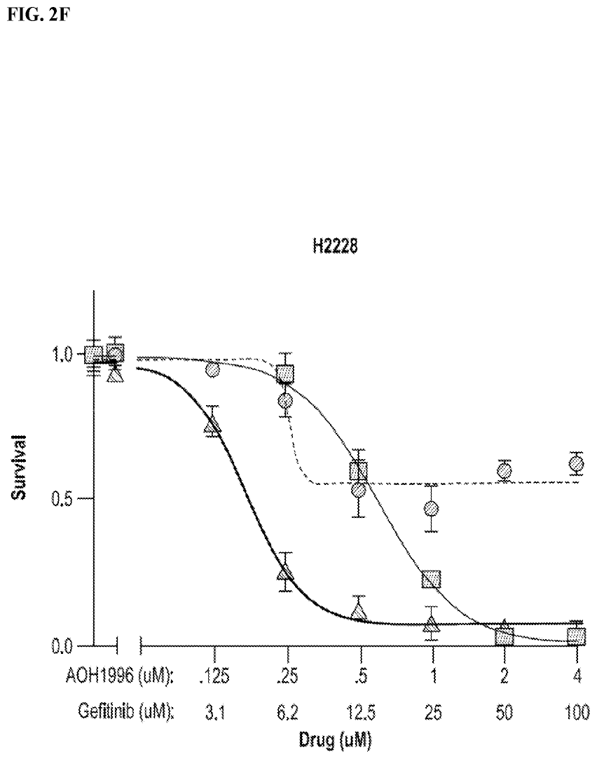

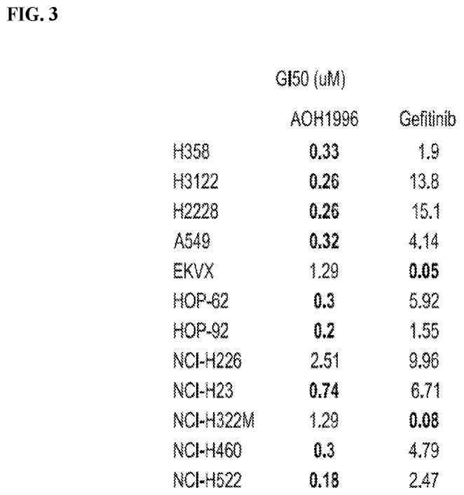

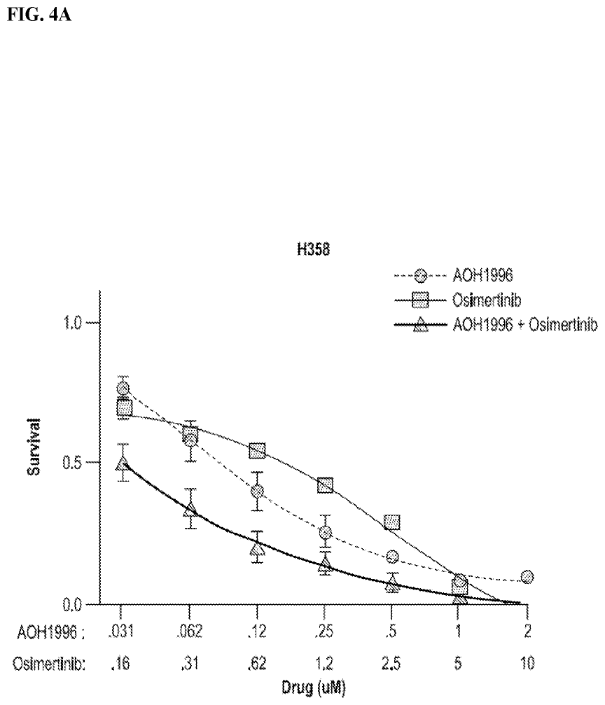

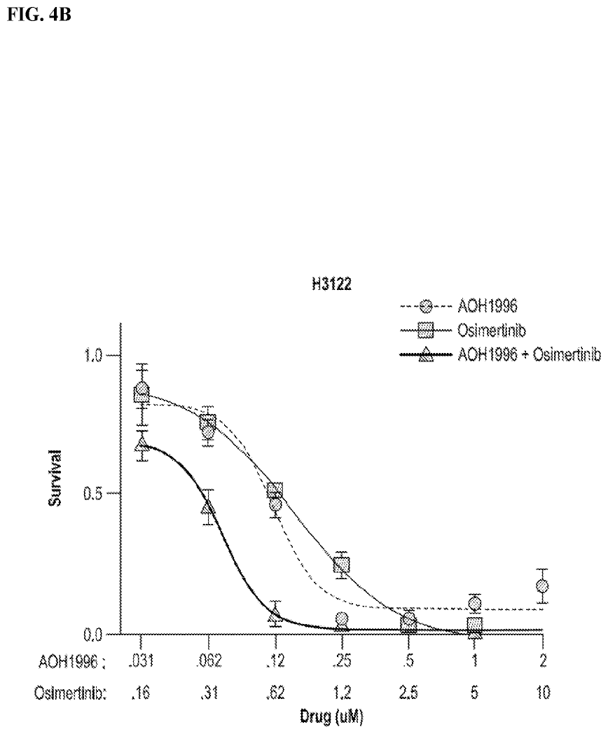

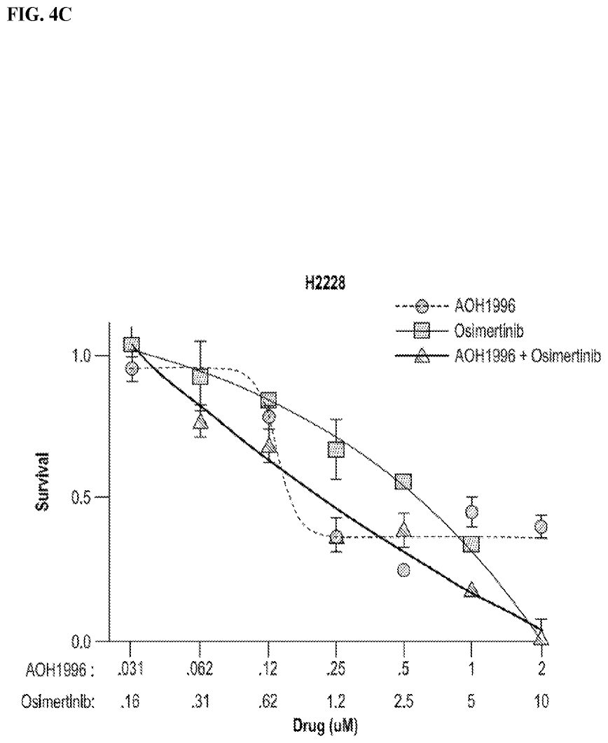

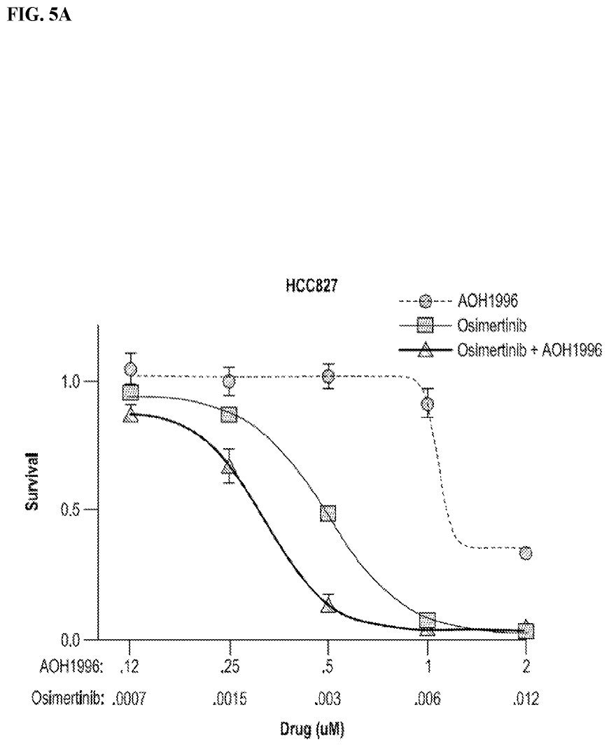

The experiments above were repeated using AOH1996 alone; each of gefitinib, afatinib, neratinib, erlotinib, and osimertinib alone; and a combination of AOH1996 with each of gefitinib, afatinib, neratinib, erlotinib, and osimertinib. The results are shown in FIGS. 1-6.

### Example 3

AOH1996 is a novel small molecule inhibitor of PCNA that preferentially targets cancer cells over normal cells by inserting into a pocket on PCNA that is conformationally distinct in cancer cells. The binding pocket is located proximal to the interdomain connecting loop (IDCL) of PCNA, which is the main docking site for many of PCNA binding partners. As a result of AOH1996 binding, DNA replication, HR, and translesion synthesis (TLS) are inhibited, which results in apoptosis and cell cycle arrest. In addition, AOH1996 increases TRCs, which results in the loss of PCNA from chromatin and an increase in DSBs. In vivo, AOH1996 is orally administrable and effectively kills and suppresses tumors, while having no discernable side effects at more than 6 times its effective dose.

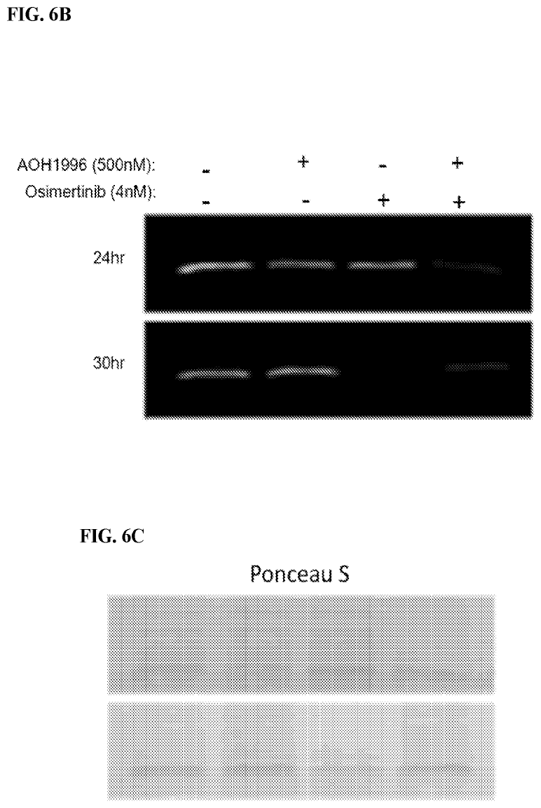

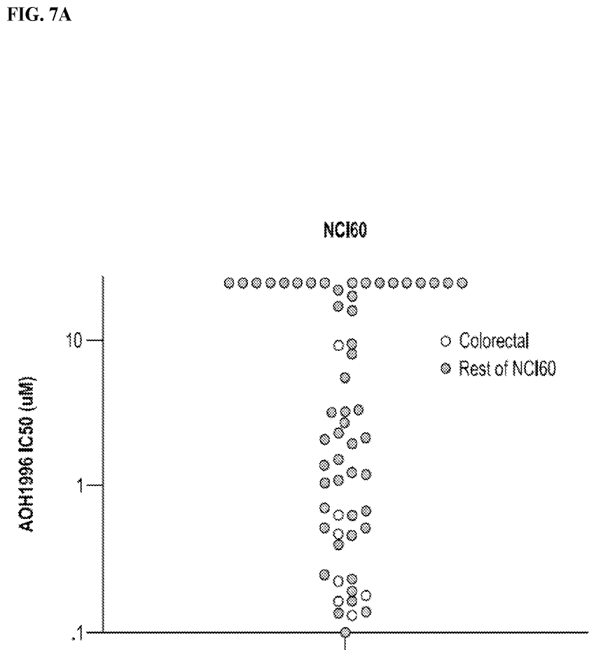

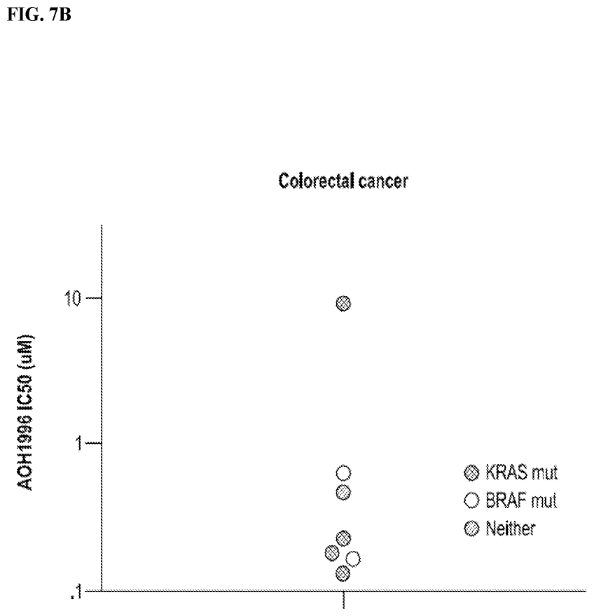

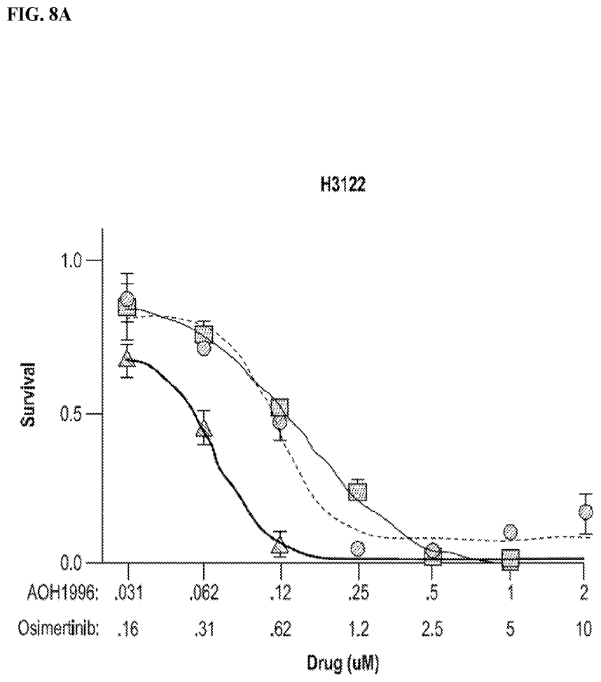

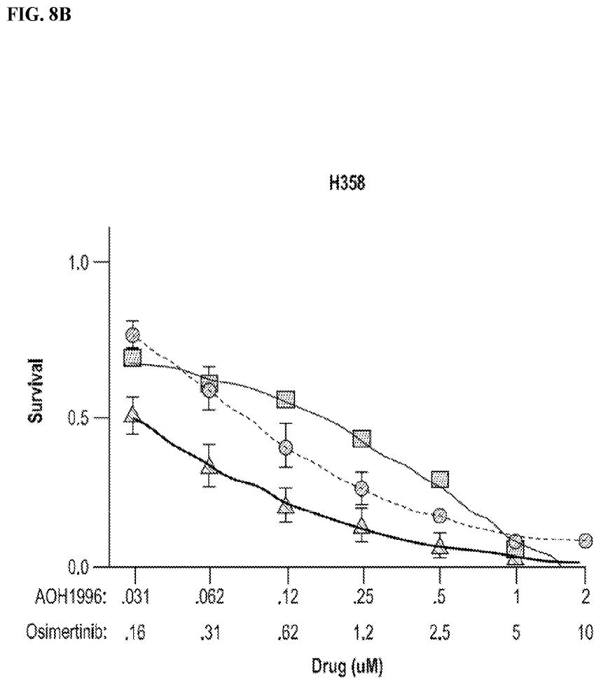

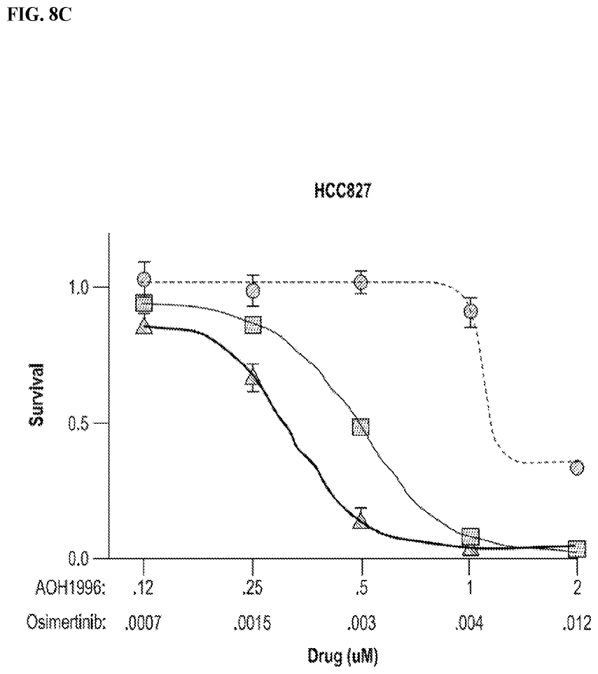

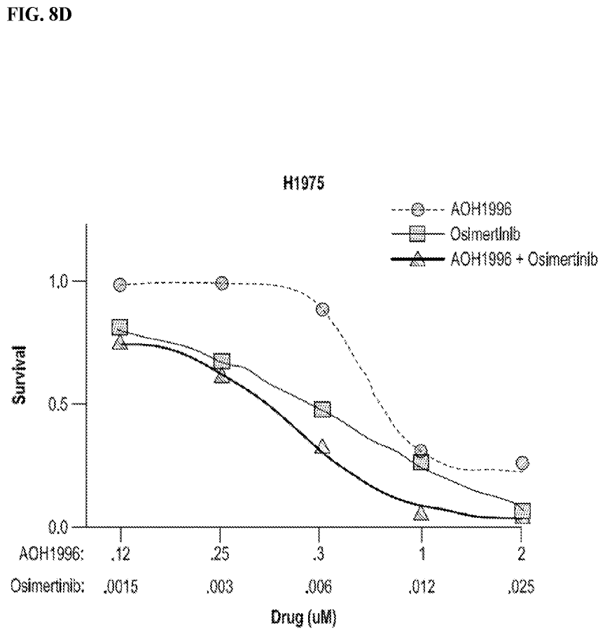

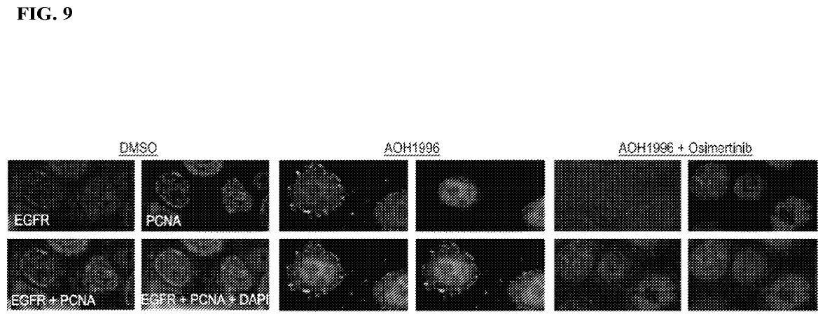

Osimertinib (Osi) is a third generation EGFR tyrosine kinase inhibitor (TKI) that is used to effectively treat non-small cell lung cancer (NSCLC) patients with tumors containing activating EGFR mutations. Combining AOH1996 with Osi results in improved killing of EGFR wild type and mutant NSCLC cell lines (FIGS. 8A-8D). AOH1996 combined with Osi enhances the destabilization of PCNA on chromatin perhaps a result of Osi inhibition of EGFR mediated Y211 phosphorylation and AOH1996 inhibitory functions on PCNA (FIGS. 6A-6B). Immunofluorescence imaging of cells treated with Osi and AOH1996 found distinct differences in localization of EGFR and PCNA in drug treated cells (FIG. 9). Serum starved cells were treated for 30 minutes with DMSO, AOH1996, Osi, or AOH1996 and Osi followed by 15 minutes of EGF stimulation. DMSO treatment resulted in normal localization of PCNA and EGFR in cells that were in early, mid and late S-phase. PCNA in early and mid S phase cells was largely localized to the edge of nuclei while in late S phase cells, PCNA localized to small distinct foci and larger more flocculent foci within the nucleus. EGFR in DMSO+EGF treated cells largely translocated to the nucleus. In AOH1996 treated cells, EGFR was localized to the cell membrane. Nuclear PCNA foci were absent and PCNA was apparent outside of the nucleus. Cells treated with Osi (not shown) or AOH1996 and Osi had less EGFR signal and the EGFR present had disorganized localization. PCNA was localized to foci in the nucleus that were often organized into patches in AOH1996 and Osi treated cells, an organization that was less apparent in cells treated with Osi alone.

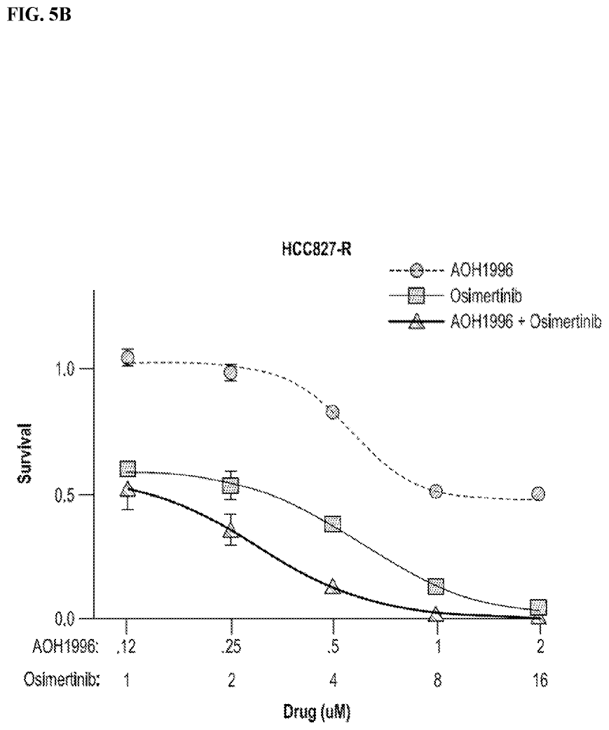

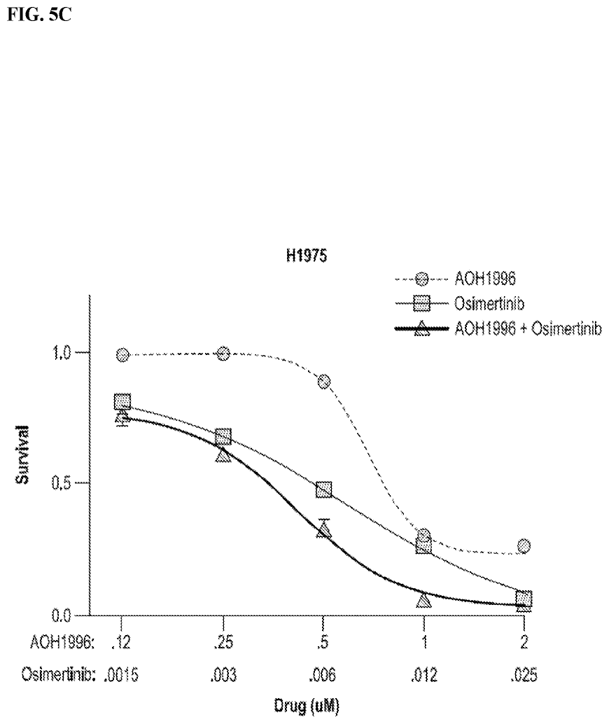

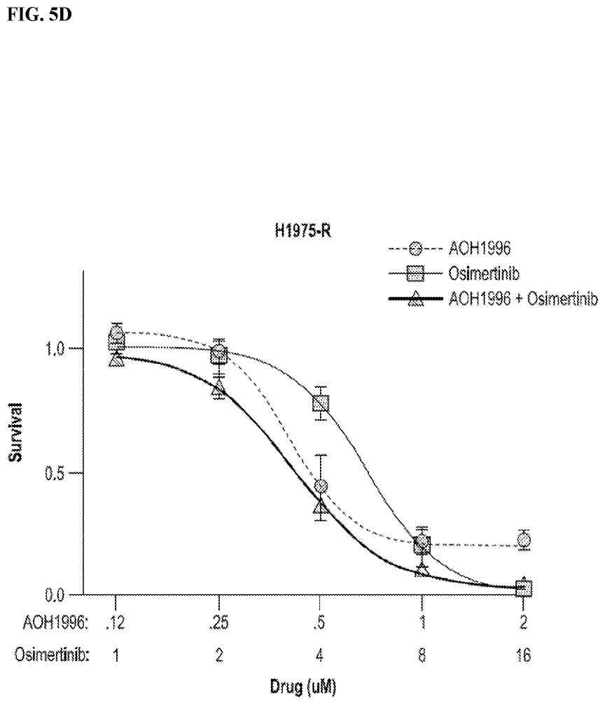

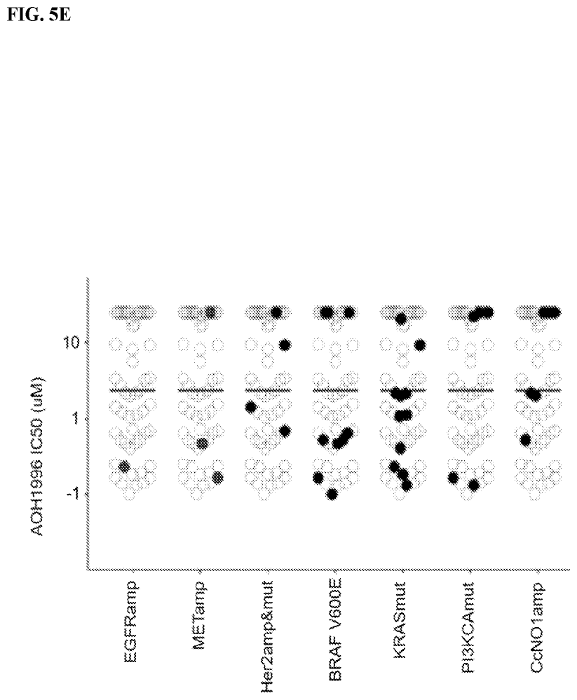

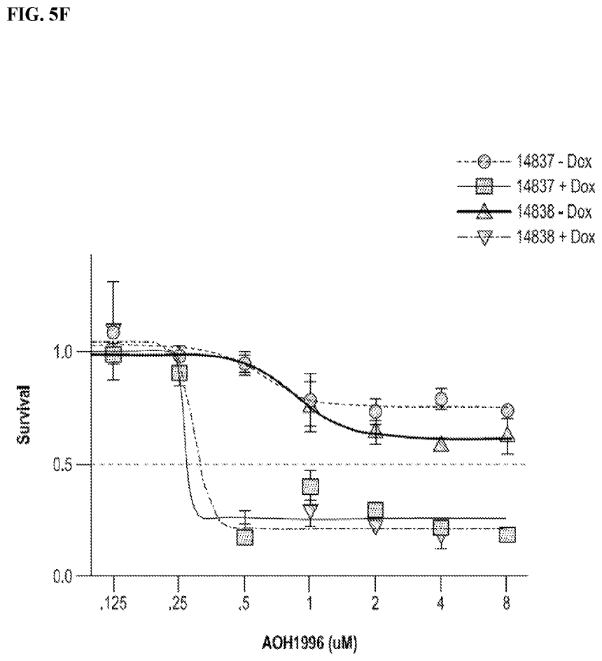

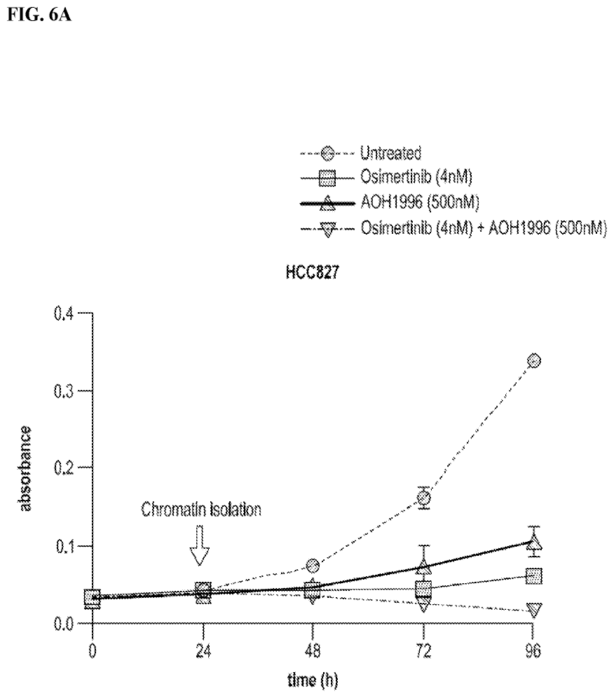

Combining AOH1996 with Osi was also more effective at killing NSCLC cell lines with activating EGFR mutations that had acquired resistance to Osi (FIGS. 5B, 5D). Studies that explored acquisition of resistance to Osi have identified many genomic alterations that contribute to resistance. These alterations are present in some of the cell lines in the NCI60 cell line panel. The growth inhibitory effects of AOH1996 on the NCI60 panel has been measured through services offered by the NCI's Development Therapeutics Program. The alterations conferring resistance to Osi did not confer resistance to AOH1996 (FIG. 5E). Notably, cell lines with BRAF V600E and activating KRAS mutations often appeared to confer greater sensitivity to AOH1996, In additional experiments, two cell lines with doxycycline-inducible mutant KRAS were found to be more sensitive to AOH1996 when the mutant KRAS was expressed versus when expression was turned off by removing doxycycline from the cells (FIG. 5F).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of treating cancer in a subject in need thereof, the method comprising administering to the subject an effective amount of an EGFR-TK inhibitor and an effective amount of a compound of Formula (A) or a pharmaceutically acceptable salt thereof; wherein the compound of Formula (A) is:

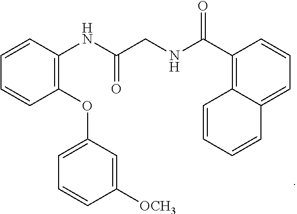 [SMILES: COc1cccc(Oc2ccccc2NC(=O)CNC(=O)c2cccc3ccccc23)c1]

2. The method of claim 1, wherein the EGFR-TK inhibitor is osimertinib, gefitinib, afatinib, neratinib, erlotinib, rociletinib, olmutinib, lazertinib, nazartinib, naquotinib, mavelertinib, abivertinib, olafertinib, alflutinib, amivantamb, tarloxitinib, mobocertinib, savolitinib, capmatinib, cetuximab, panitumumab, lapatinib, dacomitinib, necitumumab, vandetanib, icotininib, canertinib, allitinib, varlitinib, tesevatinib, pelitinib, sapitinib, EAI045, TAK-285, AG-1478, AEE788, CUDC-101, WZ8040, WZ4002, WZ3146, AG-490, or PD153035.

3. The method of claim 1, wherein the EGFR-TK inhibitor is osimertinib, gefitinib, afatinib, neratinib, or erlotinib.

4. The method of claim 1, wherein the EGFR-TK inhibitor is osimertinib and the cancer is non-small cell lung cancer.

5. The method of claim 1, wherein the cancer is non-small cell lung cancer, colorectal cancer, pancreatic cancer, breast cancer, thyroid cancer, or head and neck cancer.

6. The method of claim 1, wherein the cancer is leukemia, lung cancer, brain cancer, neuroblastoma, melanoma, ovarian cancer, renal cancer, or prostate cancer.

7. The method of claim 1, wherein the cancer has an EGFR mutation.

8. The method of claim 7, wherein the EGFR mutation comprises L858R, ex19del, Ex10ins, T790M, or a combination of two or more thereof.

9. The method of claim 1, wherein the cancer has a KRAS mutation.

10. The method of claim 9, wherein the KRAS mutation comprises a G12 mutation, a G13 mutation, or a Q61 mutation, or a combination of two or more thereof.

11. The method of claim 1, wherein the cancer has a BRAF mutation.

12. The method of claim 11, wherein the BRAF mutation is a V600E mutation.

13. The method of claim 1, wherein the cancer is a EGFR-TK-resistant cancer.

14. The method of claim 1, comprising administering to the subject: (i) an effective amount of a first pharmaceutical composition comprising the EGFR-TK inhibitor and a pharmaceutically acceptable excipient, and (ii) an effective amount of a second pharmaceutical composition comprising the compound of Formula (A) or the pharmaceutically acceptable salt thereof and a pharmaceutically acceptable excipient.

15. The method of claim 1, comprising administering to the subject an effective amount of a pharmaceutical composition comprising the EGFR-TK inhibitor, the compound of Formula (A) or the pharmaceutically acceptable salt thereof, and a pharmaceutically acceptable excipient.

16. A pharmaceutical composition comprising an EGFR-TK inhibitor, a compound of Formula (A) or a pharmaceutically acceptable salt thereof, and a pharmaceutically acceptable excipient; wherein the compound of Formula (A) is:

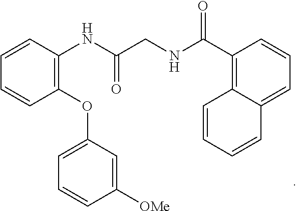 [SMILES: COc1cccc(Oc2ccccc2NC(=O)CNC(=O)c2cccc3ccccc23)c1]

17. The pharmaceutical composition of claim 16, wherein the EGFR-TK inhibitor is osimertinib, gefitinib, afatinib, neratinib, erlotinib, rociletinib, olmutinib, lazertinib, nazartinib, naquotinib, mavelertinib, abivertinib, olafertinib, alflutinib, amivantamb, tarloxitinib, mobocertinib, savolitinib, capmatinib, cetuximab, panitumumab, lapatinib, dacomitinib, necitumumab, vandetanib, icotininib, canertinib, allitinib, varlitinib, tesevatinib, pelitinib, sapitinib, EAI045, TAK-285, AG-1478, AEE788, CUDC-101, WZ8040, WZ4002, WZ3146, AG-490, or PD153035.

18. The pharmaceutical composition of claim 16, wherein the EGFR-TK inhibitor is osimertinib, gefitinib, afatinib, erlotinib, or neratinib.

19. A method of treating cancer in a subject in need thereof, the method comprising administering to the subject an effective amount of an EGFR-TK inhibitor and an effective amount of a proliferating cell nuclear antigen inhibitor.

20. The method of claim 19, wherein the proliferating cell nuclear antigen inhibitor is a compound of Formula (I) or a pharmaceutically acceptable salt thereof; wherein the compound of Formula (I) is: 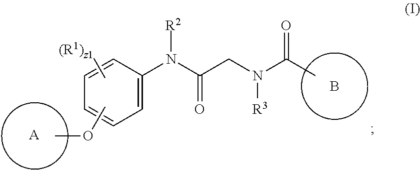
wherein (I);
Ring A is substituted or unsubstituted phenyl or substituted or unsubstituted 5 to 6 membered heteroaryl;
Ring B is substituted or unsubstituted naphthyl, substituted or unsubstituted quinolinyl, or substituted or unsubstituted isoquinolinyl;
R¹ is independently a halogen, —CX¹₃, —CHX¹₂, —CH₂X¹, —CN, —SOₙ₁R¹⁰, —SOᵥ₁NR⁷R⁸, —NHNH₂, —ONR⁷R⁸, —NHC═(O)NHNH₂, —NHC═(O)NR⁷R⁸, —N(O)ₘ₁, —NR⁷R⁸, —C(O)R⁹, —C(O)—OR⁹, —C(O)NR⁷R⁸, —OR¹⁰, —NR⁷SO₂R¹⁰, —NR⁷C═(O)R⁹, —NR⁷C(O)—OR⁹, —NR⁷OR⁹, —OCX¹₃, —OCHX¹₂, —OCH₂X¹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl;
R² is independently hydrogen, halogen, —CX²₃, —CHX²₂, —CH₂X², —CN, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is independently hydrogen, halogen, —CX³₃, —CHX³₂, —CH₂X³, —CN, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷, R⁸, R⁹, and R¹⁰ are independently hydrogen, halogen, —CX^{A}₃, —CHX^{A}₂, —CH₂X^{A}, —CN, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁷ and R⁸ substituents bonded to the same nitrogen atom may optionally be joined to form a substituted or unsubstituted heterocycloalkyl or substituted or unsubstituted heteroaryl;
z1 is independently an integer from 0 to 4;
m1 and v1 are independently 1 or 2;
n1 is independently an integer from 0 to 4; and
X¹, X², X³, and X^{A} are independently —Cl, —Br, —I, or —F.

21. The method of claim 20, wherein the compound of Formula (I) is a compound of Formula (II) or a pharmaceutically acceptable salt thereof; wherein the compound of Formula (II) is: 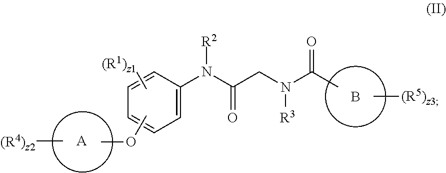
wherein
R⁴ is independently a halogen, —CX⁴₃, —CHX⁴₂, —CH₂X⁴, —CN, —SOₙ₄R¹⁴, —SOᵥ₄NR¹¹R¹², —NHNH₂, —ONR¹¹R¹², —NHC═(O)NHNH₂, —NHC═(O)NR¹¹R¹², —N(O)ₘ₄, —NR″R¹², —C(O)R¹³, —C(O)—OR¹³, —C(O)NR¹¹R¹², —OR¹⁴, —NR¹¹SO₂R¹⁴, —NR¹¹C═(O)R¹³, —NR¹¹C(O)—OR¹³, —NR₁₁OR¹³, —OCX⁴₃, —OCHX⁴₂, —OCH₂X⁴ substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R⁴ substituents may optionally be joined to form a substituted or unsubstituted cycloalkyl, substituted or unsubstituted heterocycloalkyl, substituted or unsubstituted aryl, or substituted or unsubstituted heteroaryl;
R⁵ is independently a halogen, —CX⁵₃, —CHX⁵₂, —CH₂X⁵, —CN, —SOₙ₅R¹⁸, —SOᵥ₅NR¹⁵R¹⁶, —NHNH₂, —ONR¹⁵R¹⁶, —NHC═(O)NHNH₂, —NHC═(O)NR¹⁵R¹⁶, —N(O)ₘ₅, —NR¹⁵R¹⁶, —C(O)R¹⁷, —C(O)—OR¹⁷, —C(O)NR¹⁵R¹⁶, —OR¹⁸, —NR¹⁵SO₂R¹⁸, —NR¹⁵C═(O)R¹⁷, NR¹⁵C(O)—OR¹⁷, —NR¹⁵OR¹⁷, —OCX⁵₃, —OCHX⁵₂, —OCH₂X⁵, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R⁵ substituents may optionally be joined to form a substituted or unsubstituted cycloalkyl, substituted or unsubstituted heterocycloalkyl, substituted or unsubstituted aryl, or substituted or unsubstituted heteroaryl;
R¹¹, R¹², R¹³ and R¹⁴ are independently hydrogen, halogen, —CX^{B}₃, —CHX^{B}₂, —CH₂X^{B}, —CN, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹¹ and R¹² substituents bonded to the same nitrogen atom may optionally be joined to form a substituted or unsubstituted heterocycloalkyl or substituted or unsubstituted heteroaryl;
R¹⁵, R¹⁶, R¹⁷, and R¹⁸ are independently hydrogen, halogen, —CX^{C}₃, —CHX^{C}₂, —CH₂X^{C}, —CN, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹⁵ and R¹⁶ substituents bonded to the same nitrogen atom may optionally be joined to form a substituted or unsubstituted heterocycloalkyl or substituted or unsubstituted heteroaryl;
z2 is independently an integer from 0 to 5;
z3 is independently an integer from 0 to 7;
m4, m5, v4 and v5 are independently 1 or 2;
n4 and n5 are independently an integer from 0 to 4; and
X⁴, X⁵, X^{B}, and X^{C} are independently —Cl, —Br, —I, or —F.

22. The method of claim 20, wherein the compound of Formula (I) is a compound of Formula (III) or a pharmaceutically acceptable salt thereof; a compound of Formula (IV) or a pharmaceutically acceptable salt thereof; or a compound of Formula (V) or a pharmaceutically acceptable salt thereof;
wherein the compound of Formula (III) is: 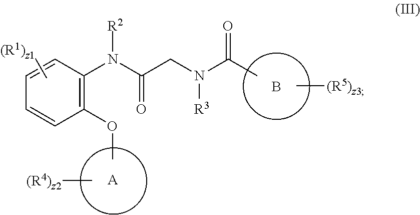
wherein the compound of Formula (IV) is: 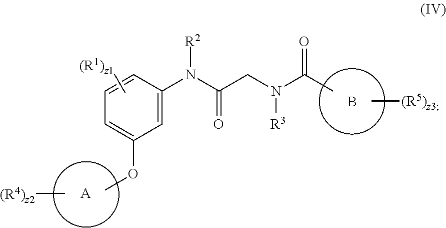
wherein the compound of Formula (V) is:

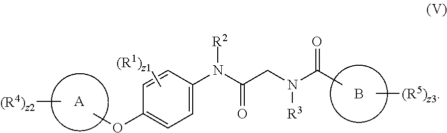

23. The method of any one of claims 20 to 22, wherein R¹ is independently halogen, —CF₃, —CHF₂, —CH₂F, —OCF₃, —OCHF₂, —OCH₂F, —OH, —NH₂, —SH, unsubstituted C₁-C₄ alkyl, or unsubstituted 2 to 4 membered heteroalkyl.

24. The method of any one of claims 20 to 23, wherein R⁴ is independently —OR¹⁴ halogen, —CF₃, —CHF₂, —CH₂F, —OCF₃, —OCHF₂, —OCH₂F, —NH₂, —SH, unsubstituted C₁-C₄ alkyl, or unsubstituted 2 to 4 membered heteroalkyl; wherein R¹⁴ is hydrogen or unsubstituted C₁-C₅ alkyl.

25. The method of any one of claims 20 to 24, wherein R⁵ is independently halogen, —CF₃, —CHF₂, —CH₂F, —OCF₃, —OCHF₂, —OCH₂F, —OH, —NH₂, —SH, unsubstituted C₁-C₄ alkyl, or unsubstituted 2 to 4 membered heteroalkyl.

26. The method of any one of claims 20 to 25, wherein R² is hydrogen, unsubstituted methyl, unsubstituted ethyl, or unsubstituted isopropyl.

27. The method of any one of claims 20 to 26, wherein R³ is hydrogen, unsubstituted methyl, unsubstituted ethyl, or unsubstituted isopropyl.

28. The method of any one of claims 20 to 27, wherein Ring A is phenyl, thienyl, or pyridyl, and Ring B is naphthyl, quinolinyl, or isoquinolinyl.

29. The method of any one of claims 20 to 27, wherein the compound has the formula:

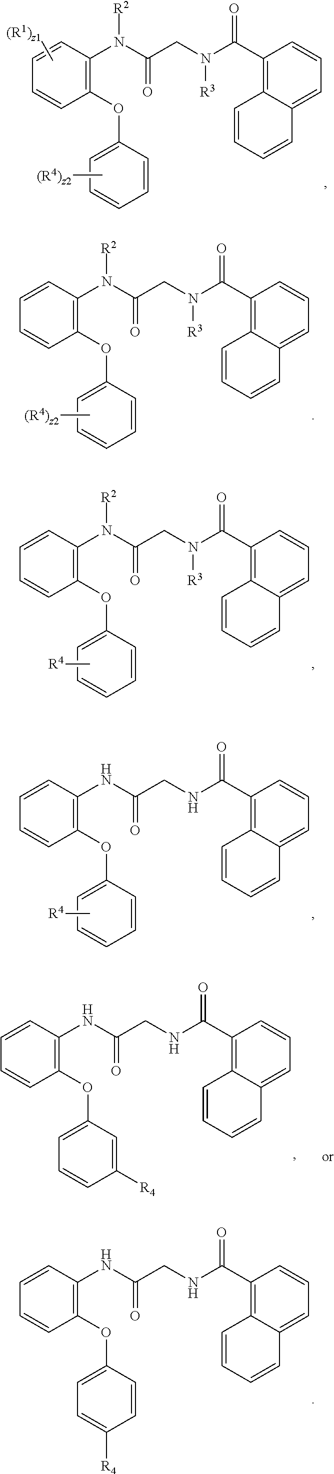

30. The method of claim 20, wherein the compound has the formula:

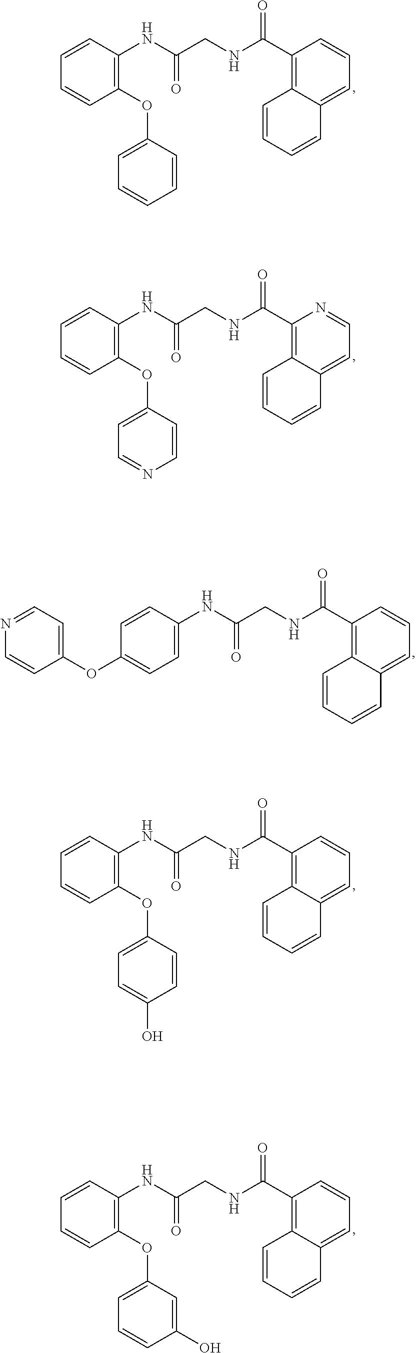 [SMILES: O=C(CNC(=O)c1cccc2ccccc12)Nc1ccccc1Oc1ccccc1 ; O=C(CNC(=O)c1nccc2ccccc12)Nc1ccccc1Oc1ccncc1 ; O=C(CNC(=O)c1cccc2ccccc12)Nc1ccc(Oc2ccncc2)cc1 ; O=C(CNC(=O)c1cccc2ccccc12)Nc1ccccc1Oc1ccc(O)cc1 ; O=C(CNC(=O)c1cccc2ccccc12)Nc1ccccc1Oc1cccc(O)c1]

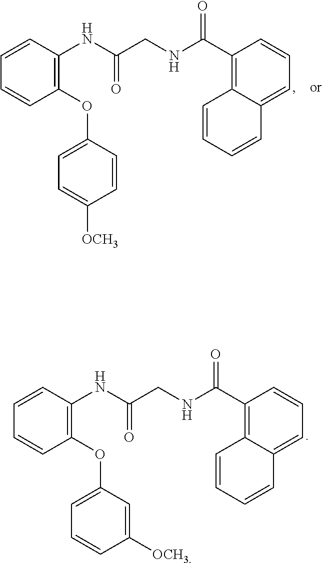 [SMILES: COc1ccc(Oc2ccccc2NC(=O)CNC(=O)c2cccc3ccccc23)cc1 ; COc1cccc(Oc2ccccc2NC(=O)CNC(=O)c2cccc3ccccc23)c1]

31. The method of any one of claims 19 to 30, wherein the EGFR-TK inhibitor is osimertinib, gefitinib, afatinib, neratinib, erlotinib, rociletinib, olmutinib, lazertinib, nazartinib, naquotinib, mavelertinib, abivertinib, olafertinib, alflutinib, amivantamb, tarloxitinib, mobocertinib, savolitinib, capmatinib, cetuximab, panitumumab, lapatinib, dacomitinib, necitumumab, vandetanib, icotininib, canertinib, allitinib, varlitinib, tesevatinib, pelitinib, sapitinib, EAI045, TAK-285, AG-1478, AEE788, CUDC-101, WZ8040, WZ4002, WZ3146, AG-490, or PD153035.

32. The method of claim 31, wherein the EGFR-TK inhibitor is osimertinib, gefitinib, afatinib, neratinib, or erlotinib.

33. The method of any one of claims 19 to 32, wherein the cancer is non-small cell lung cancer, colorectal cancer, pancreatic cancer, breast cancer, thyroid cancer, or head and neck cancer.

34. The method of any one of claims 19 to 32, wherein the cancer is leukemia, lung cancer, brain cancer, neuroblastoma, melanoma, ovarian cancer, renal cancer, or prostate cancer.

35. The method of any one of claims 19 to 34, wherein the cancer has an EGFR mutation comprising L858R, ex19del, Ex10ins, T790M, or a combination of two or more thereof.

36. The method of any one of claims 19 to 35, wherein the cancer has a KRAS mutation comprising a G12 mutation, a G13 mutation, a Q61 mutation, or a combination of two or more thereof.

37. The method of any one of claims 19 to 36, wherein the cancer has a BRAF mutation comprising a V600E mutation.

38. The method of any one of claims 19 to 34, wherein the cancer has an EGFR mutation, a KRAS mutation, a BRAF mutation, or a combination of two or more thereof.
